(19) United States
(12) Patent Application Publication
(10) Pub. No.: US 2024/0148828 A1
(43) Pub. Date: May 9, 2024

(54) GAMMA DELTA T-CELL COSTIMULATION

(71) Applicant: Shattuck Labs, Inc., Austin, TX (US)

(72) Inventors: Taylor SCHREIBER, Austin, TX (US); Suresh DE SILVA, Austin, TX (US); George FROMM, Austin, TX (US); Anne LAI, Austin, TX (US)

(21) Appl. No.: 18/282,329

(22) PCT Filed: Mar. 16, 2022
(86) PCT No.: PCT/US22/20580

§ 371 Date: Sep. 15, 2023

Related U.S. Application Data
(60) Provisional application No. 63/161,512, filed on Mar. 16, 2021.

(51) Int. Cl. A61K 38/17; A61P 35/00; C07K 16/28; C07K 16/30; C07K 16/40; A61K 39/00

(52) U.S. Cl. CPC A61K 38/1774; A61P 35/00; C07K 16/2818; C07K 16/2827; C07K 16/2851; C07K 16/2887; C07K 16/3069; C07K 16/40; A61K 2039/507; A61K 2039/545

(57) ABSTRACT

The present disclosure relates, inter alia, to compositions and methods, including heterodimeric proteins, including a heterodimeric protein comprising BTN2A1/3A1-Fc- and an scFv that specifically binds to a cancer targeting domain, that find use in the treatment of disease, such as immunotherapies for cancer and autoimmunity.

### PRIORITY

This application claims the benefit of, and priority to, U.S. Provisional Application No. 63/161,512, filed Mar. 16, 2021, the contents of which are hereby incorporated by reference in their entirety.

### TECHNICAL FIELD

The present disclosure relates to, inter alia, compositions and methods, including heterodimeric proteins that find use in the treatment of disease, such as immunotherapies for cancer and autoimmunity.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: SHK-042PC_116981-5042_ST25; date created: Mar. 16, 2022; file size: 457,485 bytes).

### BACKGROUND

Gamma delta (γδ) T cells amount to up to 5% of all T cells in a human, but they play an important role against cancer. Recent research has indicated that the amount of gamma delta T cells that infiltrate a tumor is an excellent predictor of a favorable outcome for the patient. Further, unlike the alpha beta T cells commonly used in CAR-T therapy, gamma delta T cells play a role in the innate immune response. The prognostic significance of gamma delta T cells in cancer has prompted an effort to manipulate gamma delta T cells as a therapeutic strategy for cancer. Current approaches are limited to ex vivo strategies, where a patients gamma delta T cells are either harvested and modified to express a chimeric antigen receptor, and/or expanded to greater numbers in cell culture, followed by infusion of the modified gamma delta T cells back into the cancer patient (Front Immunol. 2018; 9:1409). Strategies to manipulate gamma delta T cells directly in cancer patients have been hampered by an inability to conclusively identify the molecular entities directly recognized by the gamma delta T cell receptor (Nat Immunol. 20(2):121-128 (2019)). In fact, the most widely accepted activators of gamma delta T cells include largely intracellular molecules such as heat shock proteins, intermediates of the non-mevalonate pathway of isopentyl pyrophosphate (IPP) biosynthesis (including HMB-PP), intracellular bacteria (e.g., mycobacteria and listeria), viruses (e.g. cytomegalovirus), and other lipid antigens. Accordingly, there remains a need for novel compositions and methods gammadelta T cell engagement.

### SUMMARY

Accordingly, in various aspects, the present disclosure provides compositions and methods that are useful for cancer immunotherapy. For instance, the present disclosure, in part, relates to methods for treating cancer comprising administering (either simultaneously or sequentially) (1) a heterodimeric chimeric protein comprising portions of BTN2A1 and/or BTN3A1 that is capable of targeting to cancer cells and activating γδ T cells (e.g. Vγ9δ2-expressing T cell) and (2) at least one agent (e.g. an antibody) capable of costimulating γδ T cells.

Accordingly, in one aspect, the present disclosure relates to a method for treating a cancer in a subject in need thereof comprising: (i) administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains and (ii) administering to the subject a second pharmaceutical composition that costimulates γδ T cells. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains; wherein the subject has undergone or is undergoing treatment with a second pharmaceutical composition comprising a costimulatory molecule. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In one aspect, the present disclosure relates to a method for treating a cancer in a subject in need thereof comprising: administering to the subject a second pharmaceutical composition that costimulates γδ T cells, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In any of the embodiments disclosed herein, the second pharmaceutical composition costimulates a receptor selected from CD28, NKG2D, CD27, CD30, 4-1BB (CD137), IL-2R, IL-15R, IL-7R, IL-21R, NKp30, NKp44, DNAM-1 (CD226), IL-2R, IL-7R, IL-15R, dectins, NLRs, killer Ig-like receptors (e.g., KIR2D, KIR3D), C-type lectins (CD94/NKG2A-C, NKG2D), LFA1, CD2, CD46, Junctional Adhesion Molecule-Like (JAML). In embodiments, the second pharmaceutical composition comprises a ligand of the receptor, or a receptor-binding portion thereof. In embodiments, the second pharmaceutical composition comprises a fusion protein comprising the receptor, ligand binding portion thereof, or ligand of the receptor, or a receptor-binding portion thereof. In embodiments, the second pharmaceutical composition comprises an antibody, antibody-like molecule or a receptor-binding portion thereof. In embodiments, the second pharmaceutical composition comprises an agonistic antibody.

In embodiments, the second pharmaceutical composition costimulates CD28 and/or NKG2D. In embodiments, the second pharmaceutical composition comprises a CD28 ligand, a CD28-binding portion thereof, an NKG2D ligand, or an NKG2D-binding portion thereof. In embodiments, the NKG2D ligand is selected from MICA, MICB, ULBP1, ULBP2, ULBP3, ULBP4, ULBP5, ULBP6, H60, MULT1, and RAE1. In embodiments, the NKG2D ligand is an antibody, an antibody-like molecule, or a binding fragment thereof. In embodiments, the binding fragment is selected from Fab fragment, heavy variable chain, and single chain variable fragments (scFV). In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6.

In embodiments, the CD28 ligand is selected from CD80 and CD86. In embodiments, the CD28 ligand of is an antibody, an antibody-like molecule, or a binding fragment thereof. In embodiments, the binding fragment is selected from Fab fragment, heavy variable chain, and single chain variable fragments (scFV). In embodiments, the antibody is an agonistic antibody. In embodiments, the antibody is a monoclonal antibody.

In embodiments, the antibody is an anti-CD28 monoclonal antibody selected from JJ316, D665, 5.11A1, TGN1412, 37.51, E18, and PV-1.

Additionally or alternatively, in embodiments, the second pharmaceutical composition inhibits a receptor selected from a receptor selected from PD-1, PD-L1 and BTLA. In embodiments, the second pharmaceutical composition comprises a soluble receptor. In embodiments, the second pharmaceutical composition comprises an extracellular domain of PD-1, an extracellular domain of BTLA, or a receptor binding domain thereof. In embodiments, the second pharmaceutical composition comprises an antibody, an antibody-like molecule, or a binding fragment thereof. In embodiments, the binding fragment is selected from Fab fragment, heavy variable chain, and single chain variable fragments (scFV). In embodiments, the antibody is an antagonistic antibody. In embodiments, the antibody is a monoclonal antibody. In embodiments, the monoclonal antibody is an anti-PD-1 antibody selected from pembrolizumab, nivolumab, and cemiplimab.

In any of the embodiments disclosed herein, the BTN2A1 protein, or a fragment thereof comprises a variable Ig-like V-type domain. In embodiments, the variable Ig-like V-type domain of the BTN2A1 protein comprises an amino acid sequence that is at least 90%, or 93%, or 95%, or 97%, or 98%, or 99% identical to the amino acid sequence of SEQ ID NO: 28. In embodiments, the BTN2A1 protein, or a fragment thereof comprise a extracellular domain (ECD). In embodiments, the variable ECD of the BTN2A1 protein comprises an amino acid sequence that is at least 90%, or 93%, or 95%, or 97%, or 98%, or 99% identical to the amino acid sequence of SEQ ID NO: 27.

In any of the embodiments disclosed herein, the BTN3A1 protein, or a fragment thereof comprise a variable Ig-like V-type domain. In embodiments, the variable Ig-like V-type domain of the BTN3A1 protein comprises an amino acid sequence that is at least 90%, or 93%, or 95%, or 97%, or 98%, or 99% identical to the amino acid sequence of SEQ ID NO: 30. In embodiments, the BTN3A1 protein, or a fragment thereof comprise a extracellular domain (ECD). In embodiments, variable ECD of the BTN3A1 protein comprises an amino acid sequence that is at least 90%, or 93%, or 95%, or 97%, or 98%, or 99% identical to the amino acid sequence of SEQ ID NO: 29.

In embodiments, the first domain comprises an amino acid sequence having an amino acid sequence of selected from one or more of SEQ ID NOs: 27-30.

In any of the embodiments disclosed herein, the targeting domain is an antibody, or antigen binding fragment thereof. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)2. In embodiments, the antibody-like molecule is an scFv.

Additionally or alternatively, in embodiments, the targeting domain is an extracellular domain. In embodiments, the targeting domain is capable of binding an antigen on the surface of a cancer cell. In embodiments, the targeting domain specifically binds one or more of CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, and GD3. In embodiments, the targeting domain comprises a portion of the extracellular domain of LAG-3, PD-1, TIGIT, CD19, and PCMA. In embodiments, the targeting domain specifically binds CD19. In embodiments, the targeting domain specifically binds PSMA. In embodiments, the targeting domain specifically binds B7H3. In embodiments, the targeting domain specifically binds FAP. In embodiments, the targeting domain specifically binds CD20. In embodiments, the targeting domain specifically binds CD33.

In embodiments, the targeting domain is a polypeptide having an amino acid sequence with at least 90%, or 95%, or 97%, or 98%, or 99% identity with a polypeptide selected from SEQ ID NOs: 31-71, 111 and 112. In embodiments, the targeting domain is a polypeptide having an amino acid sequence of selected from one or more of SEQ ID NOs: 31-71, 111 and 112.

In embodiments, the targeting domain is a polypeptide having an amino acid sequence with at least 90%, or 95%, or 97%, or 98%, or 99% identity with a polypeptide selected from SEQ ID NOs: 31-35, 41, 48, 111 and 112. In embodiments, the targeting domain is a polypeptide having an amino acid sequence of selected from one or more of SEQ ID NOs: 31-35, 41, 48, 111 and 112.

In embodiments, the targeting domain is a polypeptide having an amino acid sequence with at least 90%, or 95%, or 97%, or 98%, or 99% identity with a polypeptide selected from SEQ ID NOs: 31-35, 41, 48, 111 and 112. In embodiments, the targeting domain is a polypeptide having an amino acid sequence of selected from one or more of SEQ ID NOs: 31-35, 41, 48, 111 and 112.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains.

In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally from human IgG1. In embodiments, the linker comprises the hinge-CH2-CH3 Fc domain derived from IgG4, optionally from human IgG4.

In embodiments, the heterodimeric protein comprises an alpha chain and a beta chain, wherein, the alpha chain has an amino acid sequence that is at least 90%, or 93%, or 95%, or 97%, or 98%, or 99% identical to an amino acid sequence selected from SEQ ID NOs: 75, 113, 115, 117 and 119; and the alpha chain has an amino acid sequence that is at least 90%, or 93%, or 95%, or 97%, or 98%, or 99% identical to an amino acid sequence selected from SEQ ID NOs: 81, 114, 116, 118, and 120.

In embodiments, the first domain and/or the heterodimeric protein modulates or is capable of modulating a γδ (gamma delta) T cell. In embodiments, the gamma delta T cell is selected from a cell expressing Vγ4, Vγ9δ2, or Vγ7δ4. In embodiments, the first domain modulates a Vγ9δ2-expressing T cell.

In one aspect, the present disclosure relates to a method for treating a cancer in a subject in need thereof comprising: (i) administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains and (ii) administering to the subject a second pharmaceutical composition comprising an anti-CD28 antibody and/or and anti-NKG2D antibody. In embodiments, the antibody is an agonistic antibody. In embodiments, the antibody is a monoclonal antibody.

In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD19, PSMA, B7H3, FAP, CD20 or CD33; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds CD19, PSMA, B7H3, FAP, CD20 or CD33; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD19, PSMA, B7H3, FAP, CD20 or CD33; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains.

In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds PSMA; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds PSMA; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof 5 comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds PSMA; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds B7H3; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds B7H3; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds B7H3; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds FAP; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds FAP; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds FAP; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD20; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds CD20; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof 25 comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD20; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD33; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds CD33; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD33; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: (i) administering to the subject a first pharmaceutical composition comprising a heterodimeric protein comprising an alpha chain and a beta chain, wherein the alpha chain comprises: (a) a first domain comprising an amino acid sequence that is 90%, or 93%, or 95%, or 97%, or 98%, or 99% identical to the amino acid sequence of SEQ ID NO: 27 or 28; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 16; and wherein the beta chain comprises: (a) a first domain comprising an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 17; and (ii) administering to the subject a second pharmaceutical composition comprising an anti-CD28 antibody and/or and anti-NKG2D antibody. In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120. In embodiments, the alpha chain comprises an amino acid sequence that is at least 90%, or 93%, or 95%, or 97%, or 98%, or 99% identical to the amino acid sequence of SEQ ID NO: 75, and the beta chain comprises an amino acid sequence that is at least 90%, or 93%, or 95%, or 97%, or 98%, or 99% identical to the amino acid sequence of SEQ ID NO: 81. In embodiments, the alpha chain comprises an amino acid sequence that is at least 90%, or 93%, or 95%, or 97%, or 98%, or 99% identical to the amino acid sequence of SEQ ID NO: 113, and the beta chain comprises an amino acid sequence that is at least 90%, or 93%, or 95%, or 97%, or 98%, or 99% identical to the amino acid sequence of SEQ ID NO: 114. In embodiments, the alpha chain comprises an amino acid sequence that is at least 90%, or 93%, or 95%, or 97%, or 98%, or 99% identical to the amino acid sequence of SEQ ID NO: 115, and the beta chain comprises an amino acid sequence that is at least 90%, or 93%, or 95%, or 97%, or 98%, or 99% identical to the amino acid sequence of SEQ ID NO: 115. In embodiments, the alpha chain comprises an amino acid sequence that is at least 90%, or 93%, or 95%, or 97%, or 98%, or 99% identical to the amino acid sequence of SEQ ID NO: 117, and the beta chain comprises an amino acid sequence that is at least 90%, or 93%, or 95%, or 97%, or 98%, or 99% identical to the amino acid sequence of SEQ ID NO: 118. In embodiments, the alpha chain comprises an amino acid sequence that is at least 90%, or 93%, or 95%, or 97%, or 98%, or 99% identical to the amino acid sequence of SEQ ID NO: 119, and the beta chain comprises an amino acid sequence that is at least 90%, or 93%, or 95%, or 97%, or 98%, or 99% identical to the amino acid sequence of SEQ ID NO: 120.

In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising an amino acid sequence that is 90%, or 93%, or 95%, or 97%, or 98%, or 99% identical to the amino acid sequence of SEQ ID NO: 27 or 28; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 16; and wherein the beta chain comprises: (a) a first domain comprising an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 17; wherein the subject has undergone or is undergoing treatment with a second pharmaceutical composition comprising an anti-CD28 antibody and/or and anti-NKG2D antibody. In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising an amino acid sequence that is 90%, or 93%, or 95%, or 97%, or 98%, or 99% identical to the amino acid sequence of SEQ ID NO: 27 or 28; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 16; and wherein the beta chain comprises: (a) a first domain comprising an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 17. In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120.

In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

Any aspect or embodiment disclosed herein can be combined with any other aspect or embodiment as disclosed herein.

### BRIEF DESCRIPTION OF THE FIGURES

FIG. 1A to FIG. 1H show the generation and characterization of a gamma/delta T cell engager comprised of BTN2A1/3A1 heterodimer, charged polarized linkers and targeting domains. FIG. 1A shows a non-limiting schematic representation of the BTN2A1/3A1-Fc-CD19scFv heterodimeric protein, which comprises a heterodimer of i) a human butyrophilin BTN2A1 adjoined to a human CD19-specific scFv via a linker that comprises an inert Fc domain, and ii) a human butyrophilin BTN3A1 adjoined to a human CD19-specific scFv. This GAmma DELta T cell ENgager construct also is referred to herein as the BTN2A1/3A1-Fc-CD19scFv heterodimeric protein. The linker is a charged polarized linker (indicated by “+” or “−” on the two chains of the heterodimer), which favors formation of heterodimer because of charge-charge interation, and disfavors the formation of a homodimer. FIG. 1B show a western blot analysis of a purified BTN2A1/3A1-Fc-CD19scFv heterodimeric protein. The purified protein was analyzed by Western blot using non-reduced (lane “NR”), reduced (lane “R”) and both reduced and deglycosylated (lane “DG”) conditions, following detection with an anti-human BTN2A1 antibody, an anti-human BTN3A1 antibody, or an anti-Fc antibody. The results indicate the BTN2A1/3A1-Fc-CD19scFv heterodimeric protein is a disulfide-linked protein that reduces to two individual proteins (following disruption of the interchain disulfide bonds with R-mercaptoethanol) with molecular weights consistent with the predicted molecular weights for the alpha and beta chains. Based on the similarity between the reduced and both reduced and deglycosylated lanes, the BTN2A1/3A1-Fc-CD19scFv heterodimeric protein construct appears to have few glycosylations. FIG. 1C shows further western blot analysis of the purified BTN2A1/3A1-Fc-CD19scFv molecule under non-reduced (NR), reduced (R), and deglycosylated conditions. Chain A and chain B of the construct were detected using specific antibodies against BTN2A1 and BTN3A1, respectively, together with anti-species secondary antibodies conjugated to different IR dyes. FIG. 1D shows the confirmation of the formation of the BTN2A1/3A1-Fc-CD19scFv heterodimeric protein. A dual, antibody-based MSD method was used to confirm the formation of a heterodimeric fusion protein construct. The heterodimer was captured using an BTN3A1 antibody and detected via a BTN2A1 antibody in combination with a sulfo-tagged anti-species secondary antibody (see inset). FIG. 1E shows that the BTN2A1/3A1-Fc-CD19scFv binds to CD19 expressed on Daudi cells as confirmed using flow cytometry. A control heterodimer lacking the CD19scFv sequence was used as a negative control. FIG. 1F shows the binding of the BTN2A1/3A1-Fc-CD19scFv heterodimeric protein to Vγ9Vδ2+ T cells as confirmed using flow cytometry. A control homodimer lacking the BTN2A1 ECD sequence was used as a negative control. FIG. 1G shows the binding of the BTN2A1/3A1-Fc-CD19scFv to Vγ9Vδ2+ T cells was blocked by anti-pan TCRγδ and anti-TCR Vγ9 antibodies, indicating specificity to Vγ9 subunit of TCR. Vγ9Vδ2+ T cells were co-incubated with 100 μg/mL BTN2A1/3A1-Fc-CD19scFv and a saturating concentration of either anti-pan TCRγ5 and anti-TCR Vγ9 (purified mouse monoclonal antibodies), followed by staining with APC-anti-human Fc for detection of BTN2A1/3A1-Fc-CD19scFv binding. FIG. 1H shows the specificity of BTN2A1/3A1-Fc-CD19scFv binding to Vδ2+ T cells in PBMCs. Binding of BTN2A1/3A1-Fc-CD19scFv to T cell subsets was assessed by flow cytometry. PBMCs were incubated with 100 μg/mL BTN2A1/3A1-Fc-CD19scFv, followed by staining with antibodies against CD3, CD8, TCRVδ1, TCRVγ2 (all mouse monoclonal antibodies), and APC-anti-human Fc for detection of BTN2A1/3A1-Fc-CD19scFv binding. Only CD3⁺Vδ2⁺, but not CD3⁺Vδ1⁺ (predominately Vγ9⁻) or CD3⁺CD8⁺ T cells bound to BTN2A1/3A1-Fc-CD19scFv.

FIG. 2A to FIG. 2D show the identification of co-stimulatory signals that are required for BTN2A1 and BTN3A1-mediated phosphoantigen sensing. FIG. 2A shows that anti-CD3 and anti-TCRγ5 but not recombinant BTNs induced degranulation and cytokine production in Vγ9Vδ2⁺ T cells. In vitro expanded Vγ9Vδ2⁺ T cells were stimulated with varying concentrations of plate-bound anti-CD3, anti-TCRγ5, and recombinant BTN2A1-Fc and/or BTN3A1-Fc for 4 hours. Proportion of cells expressing CD107a, IFNγ, and TNFα were detected by flow cytometry. Mean±SD is shown. FIG. 2B shows the phenotypic analysis of NK receptors and T-cell co-stimulatory receptors on Vγ9Vδ2⁺ T cells by flow cytometry. Ex vivo Vγ9Vδ2⁺ T cells in PBMC (CD3⁺Vγ9Vδ2⁺ top panels) and in vitro expanded Vγ9Vδ2⁺ T cells (bottom panels) were analyzed. Data is representative of three different donors. FIG. 2C shows the cytokine production by Vγ9Vδ2⁺ T cells in response to different stimuli. In vitro expanded Vγ9Vδ2⁺ T cells were stimulated with BTN2A1+BTN3A1 (“BTN” 1:1 ratio, 5 μg/mL) with and without anti-NKG2D (1 μg/mL) and anti-CD28 (2.5 μg/mL) for 4 hours. Proportion of cells expressing CD107a, IFNγ, and TNFα were detected by flow cytometry. Mean±SD is shown. FIG. 2D shows the activation of a T-cell line expressing γδ TCR (TEG) (J76-Vγ9Vδ2+) or parental J76 when stimulated with plate-bound BTN2A1-Fc (5 μg/mL), BTN3A1-Fc (5 μg/mL), or BTN2A1+BTN3A1 (“BTN,” 1:1 ratio, 5 μg/mL) with and without anti-NKG2D (1 μg/mL) and anti-CD28 (2.5 μg/mL) for 24 hours. Proportion of cells expressing CD69 was detected by flow cytometry. Mean±SD is shown.

FIG. 3A to FIG. 3D show the generation of Vγ9Vδ2+ T-cell line expressing γδ TCR (TEG). FIG. 3A shows the confirmation of TCRVγ9, TCRVδ2, and CD3 expression on single-cell clone of Jurkat76 (J76) transduced with Vγ9Vδ2 lentiviral construct but not parental J76. FIG. 3B shows the expression of CD28 but not NKG2D on parental J76 and J76-Vγ9Vδ2⁺. FIG. 3C shows that the T-cell receptor complex on J76-Vγ9Vδ2⁺ is functional. Parental J76 or J76-J76-Vγ9Vδ2⁺ were stimulated with various concentration of plate-bound anti-CD3 alone or in combination with 1 μg/mL anti-NKG2D or 2.5 μg/mL anti-CD28 for 24 hours. TEG activation was assessed by CD69 expression by flow cytometry. FIG. 3D shows the representative FACS plots of CD69 expression of TEG stimulated with 5 μg/mL anti-CD3 alone or in combination with 1 μg/mL anti-NKG2D or 2.5 μg/mL anti-CD28 for 24 hours.

FIG. 4A to FIG. 4D demonstrate that the activation of γδ T cells by the BTN2A1/3A1-Fc-CD19scFv heterodimeric protein is greatly enhanced by the triggering of a co-stimulatory receptor, exemplified by NKG2D. FIG. 4A shows a schematic representation of the in vitro assay used to assess the activation of γ6 T cells. Briefly, γδ T cells were stimulated in vitro in the presence of inhibitors of protein transport to the Golgi complex. The γδ T cells were assayed by flow cytometry for the expression of cytokines TNFα and IFNγ, and the degranulation marker CD107a. FIG. 4B shows the expression of TNFα by the activated γδ T cells as assayed by flow cytometry. The percentage of γδ T cells expressing TNFα upon stimulation by anti-CD3 antibody alone, or in combination with the anti-NKG2D antibody is shown using dotted lines. FIG. 4C shows the expression of IFNγ by the activated γδ T cells as assayed by flow cytometry. The percentage of γδ T cells expressing IFNγ upon stimulation by anti-CD3 antibody alone, or in combination with the anti-NKG2D antibody is shown using dotted lines. FIG. 4D shows the expression of the degranulation marker CD107a by the activated γδ T cells as assayed by flow cytometry. The percentage of γδ T cells expressing CD107a upon stimulation by anti-CD3 antibody alone, or in combination with the anti-NKG2D antibody is shown using dotted lines.

FIG. 5 shows the comparison of co-stimulation by an anti-NKG2D antibody (Clone #149810) and an anti-CD28 antibody for the activation of γδ T cells induced by the BTN2A1/3A1-Fc-CD19scFv heterodimeric protein. The expression of the degranulation marker CD107a by the activated γδ T cells as a function of the amount of the costimulatory agent and as assayed by flow cytometry is shown.

FIG. 6A to FIG. 6C show the co-stimulation by an anti-NKG2D antibody (Clone #149810) or an anti-CD28 antibody activation of γδ T cells induced by the BTN2A1/3A1-Fc-CD19scFv heterodimeric protein in a solution binding format. FIG. 6A shows the expression of TNFα by the activated γδ T cells as assayed by flow cytometry. FIG. 6B shows the expression of IFNγ by the activated γδ T cells as assayed by flow cytometry.

FIG. 6C shows the expression of the degranulation marker CD107a by the activated γδ T cells as assayed by flow cytometry. Dotted lines show the level of activation induced by 10 μg/mL plate-bound BTN2A1/3A1-Fc-CD19scFv and 1 μg/mL plate-bound anti-NKG2D antibody.

FIG. 7A to FIG. 7F show the co-stimulation by an anti-NKG2D antibody (Clone #149810) or an anti-CD28 antibody of the activation of γδ T cells induced by the BTN2A1/3A1-Fc-CD19scFv heterodimeric protein in a plate-bound format in comparison with a solution binding format. FIG. 7A shows the expression of TNFα as assayed by flow cytometry by the activated γδ T cells that are activated by the BTN2A1/3A1-Fc-CD19scFv heterodimeric protein and co-stimulated by the anti-NKG2D antibody. FIG. 7B shows the expression of IFNγ as assayed by flow cytometry by the activated γδ T cells that are activated by the BTN2A1/3A1-Fc-CD19scFv heterodimeric protein and co-stimulated by the anti-NKG2D antibody. FIG. 7C shows the expression of the degranulation marker CD107a as assayed by flow cytometry by the activated γδ T cells that are activated by the BTN2A1/3A1-Fc-CD19scFv heterodimeric protein and co-stimulated by the anti-NKG2D antibody. FIG. 7D shows the expression of TNFα as assayed by flow cytometry by the activated γδ T cells that are activated by the BTN2A1/3A1-Fc-CD19scFv heterodimeric protein and co-stimulated by the anti-CD28 antibody. FIG. 7E shows the expression of IFNγ as assayed by flow cytometry by the activated γδ T cells that are activated by the BTN2A1/3A1-Fc-CD19scFv heterodimeric protein and co-stimulated by the anti-CD28 antibody. FIG. 7F shows the expression of the degranulation marker CD107a as assayed by flow cytometry by the activated γδ T cells that are activated by the BTN2A1/3A1-Fc-CD19scFv heterodimeric protein and co-stimulated by the anti-CD28 antibody.

FIG. 8A to FIG. 8D show the comparison of the activation of γδ T cells induced by increasing concentrations of the BTN2A1/3A1-Fc-CD19scFv heterodimeric protein in the combination with increasing concentrations of MICA, MICB, a mixture of MICA and MICB, or the anti-NKG2D antibody. FIG. 8A shows the expression of the degranulation marker CD107a by the activated γδ T cells as assayed by flow cytometry in the presence of the indicated concentrations of the BTN2A1/3A1-Fc-CD19scFv heterodimeric protein and MICA. FIG. 8B shows the expression of the degranulation marker CD107a by the activated γδ T cells as assayed by flow cytometry in the presence of the indicated concentrations of the BTN2A1/3A1-Fc-CD19scFv heterodimeric protein and MICB. FIG. 8C shows the expression of the degranulation marker CD107a by the activated γδT cells as assayed by flow cytometry in the presence of the indicated concentrations of the BTN2A1/3A1-Fc-CD19scFv heterodimeric protein and a 1:1 mixture of MICA and MICB. FIG. 8D shows the expression of the degranulation marker CD107a by the activated γδ T cells as assayed by flow cytometry in the presence of the indicated concentrations of the BTN2A1/3A1-Fc-CD19scFv heterodimeric protein and the anti-NKG2D antibody.

FIG. 9A and FIG. 9B demonstrate that the combination of BTN2A1 and BTN3A1 induce greater activation of γδ T cells compared to single BTN2A1 or BTN3A1 in the presence of a co-stimulatory signal. FIG. 9A is a bar graph comparing the extent of activation of Vγ9Vδ2 T cells by either BTN2A1-Fc, BTN3A1-Fc, a combination of the BTN2A1-Fc+BTN3A1-Fc fusion protein, or the BTN2A1/3A1-Fc-CD19scFv heterodimeric protein in the combination with an anti-NKG2D antibody (Clone #149810). FIG. 9B is a bar graph comparing the extent of activation of Vγ9Vδ2 T cells by either BTN2A1-Fc, BTN3A1-Fc, a combination of the BTN2A1-Fc+BTN3A1-Fc fusion protein, or the BTN2A1/3A1-Fc-CD19scFv heterodimeric protein in the combination with an anti-CD28 antibody.

FIG. 10A to FIG. 10C demonstrate that the BTN2A1V/3A1V-Fc-CD19scFv heterodimeric proteins, which comprise only the variable domains of BTN2A1 and BTN3A1, are capable of activating the γδ T cells in the presence of an anti-NKG2D antibody (Clone #149810), albeit to a lesser extent compared to the BTN2A1/3A1-Fc-CD19scFv heterodimeric proteins that comprise the complete extracellular domains of BTN2A1 and BTN3A1. FIG. 10A shows the expression of IFNγ by the activated γδ T cells as assayed by flow cytometry. The percentage of γδ T cells expressing IFNγ upon stimulation by the combination of the BTN2A1/3A1-Fc-CD19scFv heterodimeric protein and the anti-NKG2D antibody is shown using a dotted line.

FIG. 10B shows the expression of TNFα by the activated γδ T cells as assayed by flow cytometry. The percentage of γδ T cells expressing TNFα upon stimulation by the combination of the BTN2A1/3A1-Fc-CD19scFv heterodimeric protein and the anti-NKG2D antibody is shown using a dotted line. FIG. 10C shows the expression of the degranulation marker CD107a by the activated γδ T cells as assayed by flow cytometry. The percentage of γδ T cells expressing CD107a upon stimulation by the combination of the BTN2A1/3A1-Fc-CD19scFv heterodimeric protein and the anti-NKG2D antibody is shown using a dotted line.

FIG. 11A to FIG. 11D demonstrate that heterodimeric BTN2A1/3A1 activates Vγ9Vδ2⁺ T cells in the presence of co-stimulation via NK receptor or T cell costimulatory receptor. FIG. 11A shows the activation of TEG (J76-Vγ9Vδ2+) or parental J76 cells. TEG or parental J76 cells were stimulated with plate-bound BTN fusion proteins (10 μg/mL) containing BTN2A1 homodimers, BTN3A1 homodimers, or BTN2A1/BTN3A1 heterodimers with and without anti-NKG2D (1 μg/mL) or anti-CD28 (2.5 μg/mL) for 24 hours. Proportion of cells expressing CD69 was detected by flow cytometry. Mean±SD is shown. FIG. 11B shows the stimulation of in vitro expanded Vγ9Vδ2⁺ T cells with plate-bound BTN fusion proteins (10 μg/mL) containing BTN2A1 homodimers, BTN3A1 homodimers, or BTN2A1/BTN3A1 heterodimers with and without anti-NKG2D (1 μg/mL) or anti-CD28 (2.5 μg/mL) for 4 hours. Proportion of cells expressing CD107a, IFNγ, and TNFα were detected by flow cytometry. Mean±SD is shown. FIG. 11C shows the stimulation of in vitro expanded Vγ9Vδ2⁺ T cells derived from three different donors with plate-bound BTN fusion proteins (10 μg/mL) containing BTN2A1/BTN3A1 heterodimers with and without anti-NKG2D (1 μg/mL) or anti-CD28 (2.5 μg/mL) for 4 hours. Proportion of cells expressing CD107a, IFNγ, and TNFα were detected by flow cytometry. Median level of two technical replicates from three different donors shown. *p<0.05 by Wilcoxon matched-pairs signed rank test. FIG. 11D shows the stimulation of total γδ cells. Total γδ cells purified from PBMCs and labeled with CellTrace™ Violet (CTV) before being stimulated with plate-bound BTN fusion proteins (10 μg/mL) containing BTN2A1/BTN3A1 heterodimers with and without anti-NKG2D (1 μg/mL) or anti-CD28 (2.5 μg/mL) for 96 hours. CTV Mean fluorescence intensity (MFI) was analyzed in CD3⁺V62⁺ and CD3⁺Vδ1⁺ T cell populations. Increased proliferation is indicated by decrease in MFI. *p<0.05, **p<0.01, and ***p<0.001 by Student's t-test. Mean±SD is shown. Data is representative from three different donors.

FIG. 12A to FIG. 12D demonstrate the BTN2A1/3A1-Fc-CD19scFv-enhanced tumor cell killing by Vγ9Vδ2⁺ T cells. FIG. 12A shows the induction of apoptosis. In vitro expanded Vγ9Vδ2⁺ T cells were co-cultured with Daudi (left) or Raji (right) for 4 hours at 1:1 ratio in the presence of BTN2A1/3A1-Fc-CD19scFv at the indicated concentrations. The proportion of apoptotic Daudi or Raji cells were detected by Apotracker⁺ cells in CD3-CD20⁺ cells by flow cytometry. **p<0.01, ***p<0.001, ****p<0.0001 by Student's t-test. FIG. 12B shows the active granzyme activity. In vitro expanded Vγ9Vδ2⁺ T cells were co-cultured with Daudi (left) or Raji (right) labeled with substrate for granzyme B for 1 hour at 1:1 ratio in the presence of 100 μg/mL BTN2A1/3A1-Fc-CD19scFv or 10 μg/mL anti-BTN3A1/CD277 (positive control). The proportion of Daudi or Raji cells with active granzyme activity was detected by flow cytometry. *p<0.05, **p<0.01, and ***p<0.001 by Student's t-test. FIG. 12C shows the cytokine induction. In vitro expanded Vγ9Vδ2⁺ T cells were co-cultured with Daudi or Raji cells for 4 hours at 1:1 ratio in the presence of BTN2A1/3A1-Fc-CD19scFv at the indicated concentrations or 10 μg/mL anti-BTN3A1. IFNγ and TNFα levels in culture supernatant were quantified using a multiplex MSD immunoassay. FIG. 12D shows the apoptosis of tumor cells. In vitro expanded Vγ9Vδ2⁺ T cells were co-cultured with parental K562 (CD19⁻) or K562-CD19 at 1:1 ratio for 4 hours in the presence of BTN2A1/3A1-Fc-CD19scFv to determine the proportion of apoptotic tumor cells (left) or proportion of tumor cells with granzyme B activity (right). The proportion of apoptotic tumor cells was detected by Apotracker⁺ cells in CD3⁻ cell population by flow cytometry. *p<0.05, ****p<0.00001 by Student's t-test.

FIG. 13A to FIG. 13C show the expression of CD19 and ligands for NKG2D or CD28 on tumor cells. FIG. 13A shows the confirmation of CD19 expression on Daudi and Raji lymphoma cell lines and on CD19 lentiviral transduced K562 (K562) by flow cytometry. FIG. 13B shows the analysis of CD80 or CD86 (ligands for CD28) by flow cytometry on CD19⁺ Daudi and Raji lymphoma cells and on K562 leukemic cell line. FIG. 13C shows the analysis of MICA/B, ULBP1, and ULBP2/5/6 (ligands for NKG2D) by flow cytometry on CD19⁺ lymphoma cells Daudi and Raji and on K562 leukemic cell line.

FIG. 14A to FIG. 14J show the construction and characterization of a gamma/delta T cell engagers comprised of BTN2A1/3A1 heterodimers and that are specific to B7H3 or FAP. FIG. 14A shows a non-limiting schematic representation of the BTN2A1/3A1-Fc-B7H3scFv and BTN2A1/3A1-Fc-FAPscFv heterodimeric proteins. The BTN2A1/3A1-Fc-B7H3scFv heterodimeric protein comprises a heterodimer of i) a human butyrophilin BTN2A1 adjoined to a human B7H3-specific scFv via a linker that comprises an inert Fc domain, and ii) a human butyrophilin BTN3A1 adjoined to a human B7H3-specific scFv. The BTN2A1/3A1-Fc-FAPscFv heterodimeric protein comprises a heterodimer of i) a human butyrophilin BTN2A1 adjoined to a human FAP-specific scFv via a linker that comprises an inert Fc domain, and ii) a human butyrophilin BTN3A1 adjoined to a human FAP-specific scFv. The linkers in both cases comprise charged polarized domains (indicated by “+” or “−” on the two chains of the heterodimer), which favors formation of heterodimer because of charge-charge interation, and disfavors the formation of a homodimer. FIG. 14B shows the western blot analysis of the purified the BTN2A1/3A1-Fc-FAPscFv (left) and BTN2A1/3A1-Fc-B7H3scFv (right) heterodimeric proteins under non-reduced (NR), reduced (R), and deglycosylated conditions. Chain A and chain B of the construct were detected using specific antibodies against BTN2A1 and BTN3A1, respectively, together with anti-species secondary antibodies conjugated to different IR dyes. FIG. 14C shows the results of an MSD-based ELISA assay that confirms binding of the BTN2A1/3A1-Fc-B7H3scFv to recombinant B7H3 heterodimeric protein. FIG. 14D shows the results of an MSD-based ELISA assay that confirms binding of the BTN2A1/3A1-Fc-FAPscFv to recombinant FAP heterodimeric protein. FIG. 14E shows a schematic representation of a plate-based assay used to assess the activation of γδ T cells. Briefly, γδ T cells were stimulated in vitro with heterodimeric protein, a co-stimulator or a combination of the two, in the presence of inhibitors of protein transport to the Golgi complex, and assayed by flow cytometry for the expression of cytokines TNFα and IFNγ, and the degranulation marker CD107a. FIG. 14F shows the % γδ T cells that are undergoing degranulation as indicated by the expression of CD107a in an assay that is represented in FIG. 14E. FIG. 14G shows the % γδ T cells expressing IFNγ in an assay that is represented in FIG. 14E. FIG. 14H shows the % γδ T cells expressing TNFα in an assay that is represented in FIG. 14E. FIG. 14G shows the % γδ T cells expressing IFNγ in an assay that is represented in FIG. 14E. FIG. 14H shows the % γδ T cells expressing TNFα in an assay that is represented in FIG. 14E. FIG. 14I shows that the BTN2A1/3A1-Fc-B7H3scFv heterodimeric protein binds B7H3+ OV90 tumor cells but not B7H3-RAJI tumor cells. FIG. 14J shows the results of a killing of the B7H3+ OVCAR3 cells by BTN2A1/3A1-Fc-B7H3scFv.

FIG. 15A and FIG. 15B show the construction and characterization of a gamma/delta T cell engager comprised of BTN2A1/3A1 heterodimer and that is specific to CD20 (the BTN2A1/3A1-Fc-CD20scFv heterodimeric protein). FIG. 15A shows the western blot analysis of the purified the BTN2A1/3A1-Fc-CD20scFv heterodimeric protein under non-reduced (NR), reduced (R), and deglycosylated conditions. Chain A and chain B of the construct were detected using specific antibodies against BTN2A1 and BTN3A1, respectively, together with anti-species secondary antibodies conjugated to different IR dyes. FIG. 15B shows the results of a plate-based assay used to assess the activation of γδ T cells. Briefly, γδ T cells were stimulated in vitro with the BTN2A1/3A1-Fc-CD20scFv heterodimeric protein, a co-stimulator or a combination of the two, in the presence of inhibitors of protein transport to the Golgi complex, and assayed by flow cytometry for the degranulation marker CD107a.

FIG. 16A and FIG. 16B show the construction and characterization of a gamma/delta T cell engager comprised of BTN2A1/3A1 heterodimer and that is specific to CD33 (the BTN2A1/3A1-Fc-CD33scFv heterodimeric protein). FIG. 16A shows the western blot analysis of the purified the BTN2A1/3A1-Fc-CD33scFv heterodimeric protein under non-reduced (NR), reduced (R), and deglycosylated conditions. Chain A and chain B of the construct were detected using specific antibodies against BTN2A1 and BTN3A1, respectively, together with anti-species secondary antibodies conjugated to different IR dyes. FIG. 16B shows the results of a plate-based assay used to assess the activation of γδ T cells. Briefly, γδ T cells were stimulated in vitro with the BTN2A1/3A1-Fc-CD33scFv heterodimeric protein, a co-stimulator or a combination of the two, in the presence of inhibitors of protein transport to the Golgi complex, and assayed by flow cytometry for the degranulation marker CD107a.

### DETAILED DESCRIPTION

The present disclosure is based, in part, on the discovery that the activation of γδ T cells by the BTN2A1/3A1-Fc-CD19scFv heterodimeric protein is considerably enhanced by triggering of a co-stimulatory receptor.

Accordingly, in embodiments, the methods of treatment disclosed herein activate γδ T cells to recognize and kill cancer cells, based on the targeting of the BTN2A1/3A1-Fc-targeting domain heterodimeric protein to cancer cells, and activation by BTN2A1/3A1 and costimulation by triggering of a co-stimulatory receptor. In embodiments, BTN2A1/3A1 domain of the BTN2A1/3A1-Fc-domain activates Vγ9Vδ2 γδ T cells.

### The Heterodimeric Proteins Suitable in the Methods Disclosed Herein

In one aspect, the present disclosure relates to a method for treating a cancer in a subject in need thereof comprising: (i) administering to the subject a first pharmaceutical composition comprising a heterodimeric protein; and (ii) administering to the subject a second pharmaceutical composition that costimulates γδT cells.

In one aspect, the heterodimeric proteins suitable to the methods disclosed herein comprise: (a) a first domain comprises a BTN2A1 protein, a BTN3A1 protein, and/or a fragment thereof; (b) a second domain comprising a targeting domain; and (c) a linker that adjoins the first and second domain.

In one aspect, the heterodimeric proteins suitable to the methods disclosed herein comprise an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domain; and wherein the beta chain comprises: (a) a first domain comprising a BTN3A1 protein, or a fragment thereof; (b) a second domain comprising a targeting domain; and (c) a linker that adjoins the first and second domains. In embodiments, the alpha chain and the beta chain self-associate to form the heterodimer.

In one aspect, the heterodimeric proteins suitable to the methods disclosed herein comprise an alpha chain and a beta chain, wherein the alpha chain comprises: (a) a first domain comprising a BTN2A1 protein, or a fragment thereof; (b) a second domain comprising a targeting domain that is capable of binding CD19; and (c) a linker that adjoins the first and second domain; and wherein the beta chain comprises: (a) a first domain comprising a BTN3A1 protein, or a fragment thereof; (b) a second domain comprising a targeting domain that is capable of binding CD19; and (c) a linker that adjoins the first and second domains. In embodiments, the alpha chain and the beta chain self-associate to form the heterodimer.

In one aspect, the heterodimeric proteins suitable to the methods disclosed herein comprise an alpha chain and a beta chain, wherein the alpha chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domain; and wherein the beta chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a linker that adjoins the first and second domains. In embodiments, a second linker adjoins (i) the BTN2A1 protein, or the fragment thereof, and (ii) the BTN3A1 protein, or the fragment thereof. In embodiments, the second linker is a flexible amino acid sequence. In embodiments, the alpha chain and the beta chain self-associate to form the heterodimer of alpha and beta chains, which comprise a BTN2A12-BTN3A12 tetramer.

In one aspect, the heterodimeric proteins suitable to the methods disclosed herein comprise an alpha chain and a beta chain, wherein the alpha chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) an alpha chain linker that adjoins the first and second domain; and wherein the beta chain comprises: (a) (i) a first domain comprising a BTN2A1 protein, or a fragment thereof, and (ii) a BTN3A1 protein, or a fragment thereof; (b) a second domain comprising a targeting domain that specifically binds to CD19; and (c) a beta chain linker that adjoins the first and second domains. In embodiments, a second linker adjoins (i) the BTN2A1 protein, or the fragment thereof, and (ii) the BTN3A1 protein, or the fragment thereof. In embodiments, the second linker is a flexible amino acid sequence. In embodiments, the alpha chain linker and the beta chain linker self-associate. In embodiments, the alpha chain and the beta chain self-associate to form the heterodimer of alpha and beta chains, which comprise a BTN2A12-BTN3A12 tetramer. In embodiments, the alpha chain linker and the beta chain linker are charged polarized linkers, wherein one of the alpha chain linker and the beta chain linker is positively charged and the other is negatively charged. In embodiments, the alpha chain linker and the beta chain linker comprise an Fc domain comprising knob-in-hole (KIH) mutations. In embodiments, the alpha chain linker and the beta chain linker comprise an Fc domain comprising KIH mutations and FcRn mutations. In embodiments, the alpha chain and the beta chain self-associate to form the heterodimer.

In embodiments, the first domain of the alpha chain comprises the extracellular domain of BTN2A1 protein. In embodiments, the first domain of the alpha chain comprises a polypeptide having an amino acid sequence that has at least about 95% identity with the amino acid sequence of SEQ ID NO: 27 or SEQ ID NO: 28. In embodiments, the first domain of the alpha chain comprises a polypeptide having an amino acid sequence that is identical to the amino acid sequence of SEQ ID NO: 27 or SEQ ID NO: 28. In embodiments, the first domain of the beta chain comprises the extracellular domain of BTN3A1 protein. In embodiments, the first domain of the beta chain comprises a polypeptide having an amino acid sequence that has at least about 95% identity with the amino acid sequence of SEQ ID NO: 29 or SEQ ID NO: 30. In embodiments, the first domain of the beta chain comprises a polypeptide having an amino acid sequence that is identical to the amino acid sequence of SEQ ID NO: 29 or SEQ ID NO: 30.

In embodiments, the targeting domain is an antibody, or antigen binding fragment thereof. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains. In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally human IgG1. In embodiments, the linker comprises the hinge-CH2-CH3 Fc domain derived from IgG4, optionally human IgG4. In embodiments, the first and/or second charge polarized core domain further comprise peptides having positively and/or negatively charged amino acid residues at the amino and/or carboxy terminus of the charge polarized core domain. In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains. In embodiments, the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5). In embodiments, the peptide comprising positively charged amino acid residues comprises the sequence RKGGKR (SEQ ID NO: 11) or GSGSRKGGKRGS (SEQ ID NO: 12). In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID 5 NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine, and where each n is independently an integer 0 to 4) (SEQ ID NO: 6).

In embodiments, the linker of alpha chain and/or beta chain comprises a polypeptide having an amino acid sequence that has at least about 95% identity with an amino acid sequence selected from SEQ ID NOs: 15-26. In embodiments, the linker of alpha chain and/or beta chain comprises a polypeptide having an amino acid sequence that is identical to an amino acid sequence the amino acid sequence selected from SEQ ID NOs: 15-26. In embodiments, the linker of alpha chain and/or beta chain comprises a polypeptide having an amino acid sequence that has at least about 95% identity with an amino acid sequence selected from SEQ ID NOs: 16-17. In embodiments, the linker of alpha chain and/or beta chain comprises a polypeptide having an amino acid sequence that is identical to an amino acid sequence the amino acid sequence selected from SEQ ID NOs: 16-17.

In embodiments, the second domain of the alpha chain and/or beta chain comprises a polypeptide having an amino acid sequence that has at least about 95% identity with an amino acid sequence selected from SEQ ID NOs: 31-35, 41, 48, 111 and 112. In embodiments, the second domain of the alpha chain and/or beta chain comprises a polypeptide having an amino acid sequence that is identical to an amino acid sequence the amino acid sequence selected from SEQ ID NOs: 31-35, 41, 48, 111 and 112.

In embodiments, the alpha chain comprises a polypeptide having an amino acid sequence that has at least about 95% identity with an amino acid sequence selected from SEQ ID NOs: 42, 44, 46 and 48. In embodiments, the alpha chain comprises a polypeptide having an amino acid sequence that is identical to an amino acid sequence the amino acid sequence selected from SEQ ID NOs: 42, 44, 46 and 48. In embodiments, the alpha chain comprises a polypeptide having an amino acid sequence that has at least about 95% identity with an amino acid sequence of SEQ ID NOs: 42 or 48. In embodiments, the alpha chain comprises a polypeptide having an amino acid sequence that is identical to an amino acid sequence the amino acid sequence of SEQ ID NOs: 42 or 48.

In embodiments, the beta chain comprises a polypeptide having an amino acid sequence that has at least about 95% identity with an amino acid sequence selected from SEQ ID NOs: 50, 52, 54 and 56. In embodiments, the beta chain comprises a polypeptide having an amino acid sequence that is identical to an amino acid sequence the amino acid sequence selected from SEQ ID NOs: 50, 52, 54 and 56. In embodiments, the beta chain comprises a polypeptide having an amino acid sequence that has at least about 95% identity with an amino acid sequence of SEQ ID NOs: 50 or 52. In embodiments, the beta chain comprises a polypeptide having an amino acid sequence that is identical to an amino acid sequence the amino acid sequence of SEQ ID NOs: 50 or 52.

In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120.

In alternative embodiments, the heterodimeric protein is substituted with a homodimeric protein comprising an amino acid sequence that is at least 90%, or 93%, or 95%, or 97%, or 98%, or 99% identical to the amino acid sequences selected from SEQ ID NOs. 87-103 and 113-120. In embodiments, the homodimeric protein comprises amino acid sequence selected from SEQ ID NOs. 87-103 and 113-120.

The sequences of exemplary embodiments of heterodimeric protein fusion proteins are provided in the Table below (Leader sequence is indicated by a double underlined font (MEFGLSWVFLVAIIKGVQC, SEQ ID NO: 72), extracellular domain of human BTN2A1 is shown in bold-underlined-italicized font, extracellular domain of human BTN3A1 is shown in bold-underlined font, a core domain of the linker is shown in a single underlined font, and anti-CD19, ani-B7H3, anti-FAP, anti-CD20 or anti-CD33 scFv sequences are shown in a boldface font). In embodiments, the heterodimeric protein fusion proteins lack the leader sequence.

| Description | Sequence |
|---|---|
| Human | MEFGLSWVFLVAIIKGVQCQFSVLGPSGPILAMVGEDADLPCHLFPTMSA |
| BTN3A1-Alpha-scFvCD19 | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | RKGGKRGSKYGPPCPPCPAPEFLGGPSVFLFPPKPKDQLMISRTPEVTCV |
| | WDVSQEDPEVQFNWYVDGVEVHNAKTKPREEQFNSTYRVVSVLTVLHQ |
| | DWLSGKEYKCKVSSKGLPSSIEKTISNATGQPREPQVYTLPPSQEEMTKN |
| | QVSLTCLVKGFYPSDIAVEWESNGQPENNYKTTPPVLDSDGSFFLYSRLTV |
| | DKSRWQEGNVFSCSVLHEALHNHYTQKSLSLSLGKDEGGEDGSQVQLQ |
| | QSGAELVRPGSSVKISCKASGYAFSSYWMNWVKQRPGQGLEWIGQIWP |
| | GDGDTNYNGKFKGKATLTADESSSTAYMQLSSLASEDSAVYFCARRETT |
| | TVGRYYYAMDYWGQGTTVTVSSGGGGSGGGGSGGGGSDIQLTQSPAS |
| | LAVSLGQRATISCKASQSVDYDGDSYLNWYQQIPGQPPKLLIYDASNLVS |
| | GIPPRFSGSGSGTDFTLNIHPVEKVDAATYHCQQSTEDPWTFGGGTKLEI |
| | K (SEQ ID NO: 73) |
| Human | MEFGLSWVFLVAIIKGVQCQFSVLGPSGPILAMVGEDADLPCHLFPTMSA |
| BTN3A1-Alpha-19scFv3 | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | RKGGKRGSKYGPPCPPCPAPEFLGGPSVFLFPPKPKDQLMISRTPEVTCV |
| | WDVSQEDPEVQFNWYVDGVEVHNAKTKPREEQFNSTYRVVSVLTVLHQ |
| | DWLSGKEYKCKVSSKGLPSSIEKTISNATGQPREPQVYTLPPSQEEMTKN |
| | QVSLTCLVKGFYPSDIAVEWESNGQPENNYKTTPPVLDSDGSFFLYSRLTV |
| | DKSRWQEGNVFSCSVLHEALHNHYTQKSLSLSLGKDEGGEDGSDIQMTQ |
| | TTSSLSASLGDRVTISCRASQDISKYLNWYQQKPDGTVKLLIYHTSRLHS |
| | GVPSRFSGSGSGTDYSLTISNLEQEDIATYFCQQGNTLPYTFGGGTKLEIT |
| | GGGSGGGSGGGSEVKLQESGPGLVAPSQSLSVTCTVSGVSLPDYGVS |
| | WIRQPPRKGLEWLGVIWGSETTYYNSALKSRLTIIKDNSKSQVFLKMNSL |
| | QTDDTAIYYCAKHYYYGGSYAMDYWGQGTSVTVSS (SEQ ID NO: 74) |
| Human | MEFGLSWVFLVAIIKGVQCQFIVVGPTDPILATVGENTTLRCHLSPEKNAE |
| BTN2A1-Alpha-scFvCD19 | DMEVRWFRSQFSPAVFVYKGGRERTEEQMEEYRGRTTFVSKDISRGSVA |
| with a polarized linker | LVIHNITAQENGTYRCYFQEGRSYDEAILHLVVAGLGSKPLISMRGHEDGG |
| | IRLECISRGWYPKPLTVWRDPYGGVAPALKEVSMPDADGLFMVTTAVIIR |
| | DKSVRNMSCSINNTLLGQKKESVIFIPESFMPSVSPCAGSGSRKGGKRG |
| | SKYGPPCPPCPAPEFLGGPSVFLFPPKPKDQLMISRTPEVTCVVVDVSQE |
| | DPEVQFNWYVDGVEVHNAKTKPREEQFNSTYRVVSVLTVLHQDWLSGKE |
| | YKCKVSSKGLPSSIEKTISNATGQPREPQVYTLPPSQEEMTKNQVSLTCLV |
| | KGFYPSDIAVEWESNGQPENNYKTTPPVLDSDGSFFLYSRLTVDKSRWQE |
| | GNVFSCSVLHEALHNHYTQKSLSLSLGKDEGGEDGSDIQMTQTTSSLSA |
| | SLGDRVTISCRASQDISKYLNWYQQKPDGTVKLLIYHTSRLHSGVPSRFS |
| | GSGSGTDYSLTISNLEQEDIATYFCQQGNTLPYTFGGGTKLEITGGGSGG |
| | GSGGGSEVKLQESGPGLVAPSQSLSVTCTVSGVSLPDYGVSWIRQPPR |
| | KGLEWLGVIWGSETTYYNSALKSRLTIIKDNSKSQVFLKMNSLQTDDTAI |
| | YYCAKHYYYGGSYAMDYWGQGTSVTVSS (SEQ ID NO: 75) |
| | Nucleotide Sequence of Human BTN2A1-Alpha-scFvCD19 with |
| | a polarized linker: |
| | ATGGAGTTCGGCCTGAGCTGGGTGTTTCTGGTGGCCATCATCAAGGGC |
| | GTGCAGTGCCAGTTCATCGTGGTGGGCCCTACCGACCCAATCCTGGC |
| | CACAGTGGGCGAGAACACCACACTGAGGTGTCACCTGTCCCCAGAGA |
| | AGAATGCCGAGGATATGGAGGTGCGGTGGTTCAGATCTCAGTTTAGCC |
| | CCGCCGTGTTCGTGTATAAGGGCGGCCGGGAGAGAACCGAGGAGCAG |
| | ATGGAGGAGTACAGGGGCCGCACCACATTTGTGAGCAAGGACATCTCC |
| | CGCGGCTCTGTGGCCCTGGTCATCCACAACATCACCGCCCAGGAGAA |
| | TGGCACATATCGGTGCTACTTTCAGGAGGGCAGATCCTACGATGAGGC |
| | CATCCTGCACCTGGTGGTGGCAGGCCTGGGATCTAAGCCCCTGATCAG |
| | CATGAGGGGACACGAGGACGGAGGAATCAGGCTGGAGTGTATCAGCA |
| | GAGGCTGGTATCCCAAGCCTCTGACCGTGTGGAGAGATCCCTACGGA |
| | GGAGTGGCACCTGCCCTGAAGGAGGTGTCCATGCCAGACGCCGATGG |
| | CCTGTTCATGGTGACCACAGCCGTGATCATCCGGGACAAGTCTGTGAG |
| | AAATATGTCTTGCAGCATCAACAATACACTGCTGGGCCAGAAGAAGGAG |
| | AGCGTGATCTTCATCCCCGAGTCCTTTATGCCATCCGTGTCTCCATGTG |
| | CAGGAAGCGGCTCCAGGAAGGGAGGCAAGAGGGGAAGCAAGTATGG |
| | ACCACCTTGCCCACCATGTCCAGCACCAGAGTTTCTGGGAGGACCATC |
| | CGTGTTCCTGTTTCCTCCAAAGCCCAAGGACCAGCTGATGATCTCCAG |
| | GACCCCAGAGGTGACATGCGTGGTGGTGGACGTGTCTCAGGAGGATC |
| | CTGAGGTGCAGTTCAACTGGTACGTGGACGGCGTGGAGGTGCACAAT |
| | GCCAAGACCAAGCCCAGGGAGGAGCAGTTTAACTCCACCTATCGCGT |
| | GGTGTCTGTGCTGACAGTGCTGCACCAGGACTGGCTGAGCGGCAAGG |
| | AGTACAAGTGCAAGGTGAGCTCCAAGGGCCTGCCTTCTAGCATCGAGA |
| | AGACCATCTCCAACGCCACAGGCCAGCCCAGAGAGCCTCAGGTGTATA |
| | CCCTGCCCCCTAGCCAGGAGGAGATGACCAAGAATCAGGTGTCCCTG |
| | ACATGTCTGGTGAAGGGCTTCTACCCTTCCGATATCGCCGTGGAGTGG |
| | GAGTCTAACGGCCAGCCAGAGAACAATTATAAGACCACACCACCCGTG |
| | CTGGACAGCGATGGCTCCTTCTTTCTGTACTCTAGGCTGACCGTGGAC |
| | AAGAGCCGCTGGCAGGAGGGCAACGTGTTTTCTTGCAGCGTGCTGCA |
| | CGAGGCCCTGCACAATCACTACACACAGAAGTCCCTGTCTCTGAGCCT |
| | GGGCAAGGATGAGGGAGGAGAGGACGGAAGCGATATCCAGATGACCC |
| | AGACCACATCCTCTCTGTCCGCCTCTCTGGGCGACAGGGTGACAATCT |
| | CCTGTCGCGCCTCTCAGGATATCAGCAAGTATCTGAATTGGTATCAGCA |
| | GAAGCCTGACGGCACCGTGAAGCTGCTGATCTATCACACATCCCGGCT |
| | GCACTCTGGCGTGCCAAGCAGATTCAGCGGATCCGGATCTGGCACCG |
| | ACTACTCCCTGACAATCTCTAACCTGGAGCAGGAGGATATCGCCACCTA |
| | TTTCTGCCAGCAGGGCAATACCCTGCCTTACACATTTGGCGGCGGCAC |
| | CAAGCTGGAGATCACAGGCGGAGGAAGCGGAGGAGGATCCGGAGGA |
| | GGATCTGAGGTGAAGCTGCAGGAGAGCGGACCTGGCCTGGTGGCAC |
| | CAAGCCAGTCCCTGTCTGTGACCTGTACAGTGTCTGGCGTGAGCCTGC |
| | CCGATTACGGCGTGTCTTGGATCAGGCAGCCTCCAAGGAAGGGCCTG |
| | GAGTGGCTGGGCGTGATCTGGGGCAGCGAGACAACATACTATAACAGC |
| | GCCCTGAAGTCCCGGCTGACCATCATCAAGGACAACAGCAAGTCCCA |
| | GGTGTTCCTGAAGATGAATTCCCTGCAGACCGACGATACAGCCATCTAC |
| | TATTGTGCCAAGCACTACTATTACGGCGGCTCTTATGCCATGGATTACTG |
| | GGGCCAGGGCACCAGCGTGACAGTGAGCTCCTGA |
| | (SEQ ID NO: 76) |
| Human | MEFGLSWVFLVAIIKGVQCQFIVVGPTDPILATVGENTTLRCHLSPEKNAE |
| BTN2A1-Alpha-scFvCD19 | DMEVRWFRSQFSPAVFVYKGGRERTEEQMEEYRGRTTFVSKDISRGSVA |
| with linker having knob- | LVIHNITAQENGTYRCYFQEGRSYDEAILHLVVAGLGSKPLISMRGHEDGG |
| in-hole mutations | IRLECISRGWYPKPLTVWRDPYGGVAPALKEVSMPDADGLFMVTTAVIIR |
| | DKSVRNMSCSINNTLLGQKKESVIFIPESFMPSVSPCAEPKSCDKTHTCP |
| | PCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNK |
| | ALPAPIEKTISKAKGQPREPQVYTLPPCRDELTKNQVSLWCLVKGFYPSDI |
| | AVEWESNGQPENNYKTTPPVLDSDGSFFLYSKLTVDKSRWQQGNVFSCS |
| | VMHEALHNHYTQKSLSLSPGKIEGRMDDIQMTQTTSSLSASLGDRVTISC |
| | RASQDISKYLNWYQQKPDGTVKLLIYHTSRLHSGVPSRFSGSGSGTDYS |
| | LTISNLEQEDIATYFCQQGNTLPYTFGGGTKLEITGGGSGGGSGGGSEVK |
| | LQESGPGLVAPSQSLSVTCTVSGVSLPDYGVSWIRQPPRKGLEWLGVIW |
| | GSETTYYNSALKSRLTIIKDNSKSQVFLKMNSLQTDDTAIYYCAKHYYYG |
| | GSYAMDYWGQGTSVTVSS (SEQ ID NO: 77) |
| | Nucleotide sequence of human BTN2A1-Alpha-scFvCD19 with |
| | linker having knob-in-hole mutations: |
| | ATGGAGTTTGGCCTGTCTTGGGTGTTTCTGGTGGCTATCATCAAGGGA |
| | GTGCAGTGTCAGTTCATCGTTGTGGGACCTACCGATCCTATCTTGGCTA |
| | CAGTGGGCGAGAATACAACCCTGAGATGTCACTTGTCTCCTGAGAAGA |
| | ACGCCGAGGATATGGAGGTTAGGTGGTTCAGATCCCAGTTCTCTCCTG |
| | CCGTGTTTGTGTATAAGGGAGGCAGAGAGAGAACAGAAGAGCAGATG |
| | GAGGAGTACAGAGGAAGAACCACCTTCGTGTCTAAGGACATCAGCAGA |
| | GGCTCTGTGGCTCTGGTGATCCACAATATCACAGCTCAGGAGAATGGC |
| | ACCTACAGATGCTACTTTCAGGAGGGCAGGTCCTACGATGAGGCTATTT |
| | TGCATCTGGTGGTTGCTGGACTGGGATCTAAACCTCTGATCAGCATGA |
| | GGGGACACGAGGATGGAGGAATTAGACTGGAGTGCATCTCTAGAGGCT |
| | GGTATCCTAAACCACTGACAGTGTGGAGAGACCCTTATGGAGGAGTTG |
| | CTCCTGCTCTGAAAGAGGTGTCTATGCCTGATGCTGATGGCCTGTTTAT |
| | GGTGACAACAGCCGTGATCATCCGGGACAAATCCGTGAGGAACATGTC |
| | TTGCTCCATCAACAACACACTGTTGGGACAGAAGAAGGAGAGCGTGAT |
| | CTTCATCCCCGAGAGCTTCATGCCTAGCGTTTCTCCTTGTGCTGAACCT |
| | AAGTCTTGCGACAAGACCCATACATGCCCTCCTTGTCCTGCTCCTGAA |
| | GCTGCTGGAGGACCTTCTGTGTTTTTGTTTCCTCCTAAGCCTAAGGATA |
| | CCCTGATGATCTCCAGAACCCCCGAGGTGACCTGTGTGGTGGTTGATG |
| | TTTCTCATGAGGATCCTGAAGTGAAGTTCAACTGGTACGTGGACGGCG |
| | TGGAGGTGCACAACGCTAAGACAAAACCTAGAGAAGAGCAGTACAACT |
| | CTACCTACAGAGTGGTGTCCGTGCTGACCGTGCTGCACCAAGATTGGT |
| | TGAATGGAAAGGAGTACAAGTGCAAGGTGTCCAACAAGGCTCTGCCTG |
| | CCCCTATCGAGAAGACCATCTCTAAGGCTAAAGGACAACCTAGAGAAC |
| | CTCAGGTGTATACCCTGCCTCCCTGCAGAGATGAGCTGACCAAGAATC |
| | AGGTTTCTCTGTGGTGTCTGGTGAAGGGCTTTTACCCTAGCGACATCG |
| | CTGTGGAGTGGGAGTCTAATGGACAACCTGAGAACAACTACAAGACCA |
| | CACCTCCTGTGCTGGACTCTGATGGCTCCTTCTTCCTGTACTCTAAGCT |
| | GACCGTGGATAAGTCTAGATGGCAACAGGGCAACGTGTTCTCCTGCTC |
| | CGTGATGCATGAAGCTCTGCACAACCACTATACACAGAAGTCTCTGAGC |
| | CTGTCTCCTGGCAAGATCGAGGGCAGAATGGACGATATCCAGATGACA |
| | CAGACAACCTCTTCTCTGTCTGCTTCTCTGGGCGATAGAGTGACCATCA |
| | GCTGCAGAGCTTCTCAGGACATCAGCAAGTATCTGAACTGGTATCAGC |
| | AGAAGCCTGATGGCACCGTGAAGCTGCTGATCTACCACACCTCCAGAT |
| | TGCATTCTGGAGTTCCTTCCAGATTTTCTGGCTCTGGCTCTGGCACCG |
| | ACTATTCTCTGACCATCAGCAATCTGGAACAGGAGGACATCGCTACCTA |
| | CTTTTGCCAGCAGGGCAACACACTGCCTTACACATTTGGAGGAGGAAC |
| | AAAGCTGGAGATCACAGGAGGAGGATCTGGAGGAGGATCTGGAGGAG |
| | GATCTGAAGTTAAACTGCAGGAATCTGGACCAGGATTAGTGGCCCCAT |
| | CTCAGTCTCTGTCTGTGACCTGTACCGTTTCTGGAGTTTCTTTGCCTGA |
| | TTACGGAGTGTCCTGGATCAGACAGCCCCCTAGAAAGGGACTGGAATG |
| | GTTAGGAGTGATTTGGGGATCTGAGACCACCTACTACAACTCTGCCCT |
| | GAAGAGCAGACTGACCATCATCAAGGACAACAGCAAGTCTCAGGTGTT |
| | CCTGAAGATGAACTCCCTGCAGACCGACGATACCGCCATCTACTACTGT |
| | GCTAAGCACTACTACTATGGCGGCTCTTATGCCATGGACTATTGGGGAC |
| | AGGGCACCTCTGTGACAGTGTCTTCTTAA |
| | (SEQ ID NO: 78) |
| Human | MEFGLSWVFLVAIIKGVQCQFIVVGPTDPILATVGENTTLRCHLSPEKNAE |
| BTN2A1-Alpha-scFvCD19 | DMEVRWFRSQFSPAVFVYKGGRERTEEQMEEYRGRTTFVSKDISRGSVA |
| with linker having knob- | LVIHNITAQENGTYRCYFQEGRSYDEAILHLVVAGLGSKPLISMRGHEDGG |
| in-hole mutations and FcRn | IRLECISRGWYPKPLTVWRDPYGGVAPALKEVSMPDADGLFMVTTAVIIR |
| mutations | DKSVRNMSCSINNTLLGQKKESVIFIPESFMPSVSPCAEPKSCDKTHTCP |
| | PCPAPEAAGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSNK |
| | ALPAPIEKTISKAKGQPREPQVYTLPPCRDELTKNQVSLWCLVKGFYPSDI |
| | AVEWESNGQPENNYKTTPPVLDSDGSFFLYSKLTVDKSRWQQGNVFSCS |
| | VLHEALHSHYTQKSLSLSPGKIEGRMDDIQMTQTTSSLSASLGDRVTISCR |
| | ASQDISKYLNWYQQKPDGTVKLLIYHTSRLHSGVPSRFSGSGSGTDYSL |
| | TISNLEQEDIATYFCQQGNTLPYTFGGGTKLEITGGGSGGGSGGGSEVKL |
| | QESGPGLVAPSQSLSVTCTVSGVSLPDYGVSWIRQPPRKGLEWLGVIWG |
| | SETTYYNSALKSRLTIIKDNSKSQVFLKMNSLQTDDTAIYYCAKHYYYGG |
| | SYAMDYWGQGTSVTVSS (SEQ ID NO: 79) |
| | Nucleotide sequence of human BTN2A1-Alpha-scFvCD19 with |
| | linker having knob-in-hole mutations and FcRn mutations: |
| | ATGGAGTTTGGCCTGTCTTGGGTGTTTCTGGTGGCTATCATCAAGGGA |
| | GTGCAGTGTCAGTTCATCGTTGTGGGACCTACCGATCCTATCTTGGCTA |
| | CAGTGGGCGAGAATACAACCCTGAGATGTCACTTGTCTCCTGAGAAGA |
| | ACGCCGAGGATATGGAGGTTAGGTGGTTCAGATCCCAGTTCTCTCCTG |
| | CCGTGTTTGTGTATAAGGGAGGCAGAGAGAGAACAGAAGAGCAGATG |
| | GAGGAGTACAGAGGAAGAACCACCTTCGTGTCTAAGGACATCAGCAGA |
| | GGCTCTGTGGCTCTGGTGATCCACAATATCACAGCTCAGGAGAATGGC |
| | ACCTACAGATGCTACTTTCAGGAGGGCAGGTCCTACGATGAGGCTATTT |
| | TGCATCTGGTGGTTGCTGGACTGGGATCTAAACCTCTGATCAGCATGA |
| | GGGGACACGAGGATGGAGGAATTAGACTGGAGTGCATCTCTAGAGGCT |
| | GGTATCCTAAACCACTGACAGTGTGGAGAGACCCTTATGGAGGAGTTG |
| | CTCCTGCTCTGAAAGAGGTGTCTATGCCTGATGCTGATGGCCTGTTTAT |
| | GGTGACAACAGCCGTGATCATCCGGGACAAATCCGTGAGGAACATGTC |
| | TTGCTCCATCAACAACACACTGTTGGGACAGAAGAAGGAGAGCGTGAT |
| | CTTCATCCCCGAGAGCTTCATGCCTAGCGTTTCTCCTTGTGCTGAACCT |
| | AAGTCTTGCGACAAGACCCATACATGCCCTCCTTGTCCTGCTCCTGAA |
| | GCTGCTGGAGGACCTTCTGTGTTTTTGTTTCCTCCTAAGCCTAAGGATA |
| | CCCTGATGATCTCCAGAACCCCCGAGGTGACCTGTGTGGTGGTTGATG |
| | TTTCTCATGAGGATCCTGAAGTGAAGTTCAACTGGTACGTGGACGGCG |
| | TGGAGGTGCACAACGCTAAGACAAAACCTAGAGAAGAGCAGTACAACT |
| | CTACCTACAGAGTGGTGTCCGTGCTGACCGTGCTGCACCAAGATTGGT |
| | TGAATGGAAAGGAGTACAAGTGCAAGGTGTCCAACAAGGCTCTGCCTG |
| | CCCCTATCGAGAAGACCATCTCTAAGGCTAAAGGACAACCTAGAGAAC |
| | CTCAGGTGTATACCCTGCCTCCCTGCAGAGATGAGCTGACCAAGAATC |
| | AGGTTTCTCTGTGGTGTCTGGTGAAGGGCTTTTACCCTAGCGACATCG |
| | CTGTGGAGTGGGAGTCTAATGGACAACCTGAGAACAACTACAAGACCA |
| | CACCTCCTGTGCTGGACTCTGATGGCTCCTTCTTCCTGTACTCTAAGCT |
| | GACCGTGGATAAGTCTAGATGGCAACAGGGCAACGTGTTCTCCTGCTC |
| | TGTGCTGCATGAAGCTCTGCACTCTCACTATACACAGAAGTCTCTGTCC |
| | CTGTCTCCTGGCAAGATCGAGGGCAGAATGGACGATATCCAGATGACA |
| | CAGACAACCTCTTCTCTGTCTGCTTCTCTGGGCGATAGAGTGACCATCA |
| | GCTGCAGAGCTTCTCAGGACATCAGCAAGTATCTGAACTGGTATCAGC |
| | AGAAGCCTGATGGCACCGTGAAGCTGCTGATCTACCACACCTCCAGAT |
| | TGCATTCTGGAGTTCCTTCCAGATTTTCTGGCTCTGGCTCTGGCACCG |
| | ACTATTCTCTGACCATCAGCAATCTGGAACAGGAGGACATCGCTACCTA |
| | CTTTTGCCAGCAGGGCAACACACTGCCTTACACATTTGGAGGAGGAAC |
| | AAAGCTGGAGATCACAGGAGGAGGATCTGGAGGAGGATCTGGAGGAG |
| | GATCTGAAGTTAAACTGCAGGAATCTGGACCAGGATTAGTGGCCCCAT |
| | CTCAGTCTCTGTCTGTGACCTGTACCGTTTCTGGAGTTTCTTTGCCTGA |
| | TTACGGAGTGTCCTGGATCAGACAGCCCCCTAGAAAGGGACTGGAATG |
| | GTTAGGAGTGATTTGGGGATCTGAGACCACCTACTACAACTCTGCCCT |
| | GAAGAGCAGACTGACCATCATCAAGGACAACAGCAAGTCTCAGGTGTT |
| | CCTGAAGATGAACTCCCTGCAGACCGACGATACCGCCATCTACTACTGT |
| | GCTAAGCACTACTACTATGGCGGCTCTTATGCCATGGACTATTGGGGAC |
| | AGGGCACCTCTGTGACAGTGTCTTCTTAA (SEQ ID NO: 80) |
| Human | MEFGLSWVFLVAIIKGVQCQFSVLGPSGPILAMVGEDADLPCHLFPTMSA |
| BTN3A1-beta-scFvCD19 | ETMELKWVSSSLRQVVNVYADGKEVEDRQSAPYRGRTSILRDGITAGKA |
| with polarized linkers | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | DEGGEDGSKYGPPCPPCPAPEFLGGPSVFLFPPKPKDQLMISRTPEVTCV |
| | WDVSQEDPEVQFNWYVDGVEVHNAKTKPREEQFNSTYRVVSVLTVLHQ |
| | DWLSGKEYKCKVSSKGLPSSIEKTISNATGQPREPQVYTLPPSQEEMTKN |
| | QVSLTCLVKGFYPSDIAVEWESNGQPENNYKTTPPVLDSDGSFFLYSRLTV |
| | DKSRWQEGNVFSCSVLHEALHNHYTQKSLSLSLGKRKGGKRGSGSDIQM |
| | TQTTSSLSASLGDRVTISCRASQDISKYLNWYQQKPDGTVKLLIYHTSRL |
| | HSGVPSRFSGSGSGTDYSLTISNLEQEDIATYFCQQGNTLPYTFGGGTKL |
| | EITGGGSGGGSGGGSEVKLQESGPGLVAPSQSLSVTCTVSGVSLPDYGV |
| | SWIRQPPRKGLEWLGVIWGSETTYYNSALKSRLTIIKDNSKSQVFLKMNS |
| | LQTDDTAIYYCAKHYYYGGSYAMDYWGQGTSVTVSS (SEQ ID NO: 81) |
| | Nucleotide Sequence of Human BTN3A1-beta-scFvCD19 with |
| | polarized linkers: |
| | ATGGAGTTCGGCCTGTCCTGGGTGTTTCTGGTGGCCATCATCAAGGGC |
| | GTGCAGTGCCAGTTCTCCGTGCTGGGCCCTTCTGGCCCAATCCTGGC |
| | AATGGTGGGAGAGGACGCAGATCTGCCATGTCACCTGTTTCCCACCAT |
| | GAGCGCCGAGACAATGGAGCTGAAGTGGGTGAGCTCCTCTCTGCGGC |
| | AGGTGGTGAACGTGTACGCCGACGGCAAGGAGGTGGAGGATAGACAG |
| | TCCGCCCCCTACCGGGGCAGAACCTCTATCCTGAGGGACGGAATCACA |
| | GCAGGCAAGGCCGCCCTGAGAATCCACAACGTGACCGCCAGCGATTC |
| | CGGCAAGTATCTGTGCTACTTCCAGGACGGCGACTTCTACGAGAAGGC |
| | CCTGGTGGAGCTGAAGGTGGCCGCCCTGGGAAGCGACCTGCATGTG |
| | GATGTGAAGGGCTATAAGGACGGCGGCATCCACCTGGAGTGTCGGAG |
| | CACCGGCTGGTATCCCCAGCCTCAGATCCAGTGGTCCAACAATAAGGG |
| | CGAGAATATCCCTACAGTGGAGGCCCCAGTGGTGGCAGATGGAGTGG |
| | GCCTGTACGCAGTGGCCGCCTCTGTGATCATGAGGGGAAGCTCCGGA |
| | GAGGGCGTGAGCTGCACCATCCGCTCTAGCCTGCTGGGCCTGGAGAA |
| | GACAGCCTCTATCAGCATCGCCGACCCCTTCTTTAGGAGCGCCCAGCG |
| | GTGGATCGCCGCCCTGGCAGGCGGCTCCGGCTCTGACGAGGGCGGC |
| | GAGGATGGCTCCAAGTATGGACCACCTTGCCCACCATGTCCAGCACCA |
| | GAGTTCCTGGGAGGACCAAGCGTGTTCCTGTTTCCTCCAAAGCCCAAG |
| | GACCAGCTGATGATCTCCAGGACCCCAGAGGTGACCTGCGTGGTGGT |
| | GGACGTGTCTCAGGAGGATCCTGAGGTGCAGTTCAACTGGTACGTGG |
| | ATGGCGTGGAGGTGCACAATGCCAAGACCAAGCCTAGGGAGGAGCAG |
| | TTTAACAGCACCTATCGCGTGGTGTCCGTGCTGACAGTGCTGCACCAG |
| | GATTGGCTGTCCGGCAAGGAGTACAAGTGCAAGGTGTCCTCTAAGGG |
| | CCTGCCAAGCTCCATCGAGAAGACCATCAGCAACGCAACAGGCCAGC |
| | CCCGCGAGCCTCAGGTGTATACCCTGCCCCCTTCTCAGGAGGAGATGA |
| | CCAAGAATCAGGTGAGCCTGACATGTCTGGTGAAGGGCTTCTACCCTA |
| | GCGACATCGCAGTGGAGTGGGAGTCCAACGGACAGCCAGAGAACAAT |
| | TATAAGACCACACCACCCGTGCTGGACTCTGATGGCAGCTTCTTTCTGT |
| | ACTCTAGGCTGACCGTGGATAAGAGCCGCTGGCAGGAGGGCAACGTG |
| | TTTAGCTGCTCCGTGCTGCACGAGGCCCTGCACAATCACTACACACAG |
| | AAGTCTCTGAGCCTGTCCCTGGGCAAGAGGAAGGGAGGCAAGAGGG |
| | GATCTGGAAGCGACATCCAGATGACCCAGACCACATCTAGCCTGTCCG |
| | CCTCTCTGGGCGACCGGGTGACAATCAGCTGTAGAGCCTCCCAGGATA |
| | TCTCTAAGTATCTGAATTGGTATCAGCAGAAGCCAGATGGCACCGTGAA |
| | GCTGCTGATCTATCACACAAGCAGGCTGCACTCCGGCGTGCCCTCTAG |
| | ATTCAGCGGATCCGGATCTGGCACCGACTACAGCCTGACAATCTCCAA |
| | CCTGGAGCAGGAGGATATCGCCACCTATTTCTGCCAGCAGGGCAATAC |
| | CCTGCCCTACACATTTGGCGGCGGCACCAAGCTGGAGATCACAGGCG |
| | GAGGATCTGGAGGAGGAAGCGGAGGAGGCTCCGAGGTGAAGCTGCA |
| | GGAGTCCGGACCAGGCCTGGTGGCACCTAGCCAGTCCCTGTCTGTGA |
| | CCTGTACAGTGTCCGGCGTGTCTCTGCCTGACTACGGCGTGTCCTGGA |
| | TCCGGCAGCCTCCAAGAAAGGGCCTGGAGTGGCTGGGCGTGATCTGG |
| | GGCAGCGAGACAACATACTATAACTCTGCCCTGAAGAGCAGACTGACC |
| | ATCATCAAGGACAACAGCAAGTCCCAGGTGTTTCTGAAGATGAATAGCC |
| | TGCAGACCGACGATACAGCCATCTACTATTGCGCCAAGCACTACTATTA |
| | CGGCGGCTCCTATGCCATGGATTACTGGGGCCAGGGCACCTCTGTGA |
| | CAGTGTCCTCTTGA (SEQ ID NO: 82) |
| Human | MEFGLSWVFLVAIIKGVQCQFSVLGPSGPILAMVGEDADLPCHLFPTMSA |
| BTN3A1-beta-scFvCD19 | ETMELKWVSSSLRQVVNVYADGKEVEDRQSAPYRGRTSILRDGITAGKA |
| with linker having knob- | ALRIHNVTASDSGKYLCYFQDGDFYEKALVELKVAALGSDLHVDVKGYK |
| in-hole (KIH) mutations | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAEPKSC |
| | DKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGKEY |
| | KCKVSNKALPAPIEKTISKAKGQPREPQVCTLPPSRDELTKNQVSLSCAVK |
| | GFYPSDIAVEWESNGQPENNYKTTPPVLDSDGSFFLVSKLTVDKSRWQQ |
| | GNVFSCSVMHEALHNHYTQKSLSLSPGKIEGRMDDIQMTQTTSSLSASL |
| | GDRVTISCRASQDISKYLNWYQQKPDGTVKLLIYHTSRLHSGVPSRFSGS |
| | GSGTDYSLTISNLEQEDIATYFCQQGNTLPYTFGGGTKLEITGGGSGGGS |
| | GGGSEVKLQESGPGLVAPSQSLSVTCTVSGVSLPDYGVSWIRQPPRKGL |
| | EWLGVIWGSETTYYNSALKSRLTIIKDNSKSQVFLKMNSLQTDDTAIYYC |
| | AKHYYYGGSYAMDYWGQGTSVTVSS (SEQ ID NO: 83) |
| | Nucleotide sequence of human BTN3A1-beta-scFvCD19 with |
| | linker having knob-in-hole (KIH) mutations: |
| | ATGGAGTTTGGCCTGTCTTGGGTGTTTCTGGTGGCTATCATCAAGGGA |
| | GTGCAGTGTCAGTTCTCTGTTCTGGGACCTTCTGGACCTATCTTGGCTA |
| | TGGTGGGAGAAGATGCTGATCTGCCTTGTCACCTGTTTCCTACCATGTC |
| | TGCTGAGACCATGGAGCTGAAATGGGTGTCCTCTTCTCTGAGACAGGT |
| | GGTGAATGTGTACGCTGATGGAAAGGAGGTGGAGGACAGACAATCTG |
| | CCCCTTATAGAGGAAGAACCAGCATCCTGAGAGATGGCATCACAGCTG |
| | GAAAGGCTGCTCTGAGAATCCACAATGTGACCGCTTCTGATTCTGGCA |
| | AGTACCTGTGCTACTTCCAGGATGGCGACTTCTACGAGAAGGCTCTGG |
| | TTGAGCTGAAAGTTGCTGCTTTGGGCTCTGATCTGCATGTTGACGTGA |
| | AGGGCTACAAGGATGGAGGCATCCATCTGGAATGTAGATCTACCGGCT |
| | GGTATCCTCAACCTCAGATTCAGTGGAGCAACAACAAGGGCGAGAACA |
| | TCCCTACAGTTGAAGCCCCTGTTGTGGCTGATGGAGTTGGACTGTATG |
| | CTGTTGCTGCTAGCGTGATCATGAGAGGATCTTCTGGAGAAGGCGTGT |
| | CTTGCACCATCAGATCTTCTCTGTTGGGACTGGAGAAGACCGCTAGCA |
| | TCTCTATCGCTGACCCCTTCTTCAGATCTGCTCAAAGATGGATTGCTGC |
| | TCTGGCTGAGCCTAAGTCTTGCGATAAGACCCACACCTGTCCTCCTTG |
| | TCCTGCTCCTGAAGCTGCTGGAGGACCTTCTGTGTTTTTGTTTCCTCCT |
| | AAGCCTAAGGATACCCTGATGATCTCCAGAACCCCCGAGGTGACCTGT |
| | GTGGTGGTTGATGTTTCTCATGAGGATCCTGAAGTGAAGTTCAACTGGT |
| | ACGTGGACGGCGTGGAGGTGCACAACGCTAAGACAAAACCTAGAGAA |
| | GAGCAGTACAACTCTACCTACAGAGTGGTGTCCGTGCTGACCGTGCTG |
| | CACCAAGATTGGTTGAATGGAAAGGAGTACAAGTGCAAGGTGTCCAAC |
| | AAGGCTCTGCCTGCCCCTATCGAGAAGACCATCTCTAAGGCTAAAGGA |
| | CAACCTAGAGAACCTCAGGTGTGTACACTGCCCCCCTCTAGAGATGAG |
| | CTGACCAAGAATCAGGTTTCTCTGTCTTGTGCTGTGAAGGGCTTTTACC |
| | CCTCCGACATCGCTGTGGAATGGGAGTCTAATGGACAACCTGAGAACA |
| | ACTACAAGACCACACCTCCTGTGCTGGACTCTGACGGCTCCTTCTTTC |
| | TGGTGTCTAAGCTGACAGTGGATAAGTCTAGATGGCAACAGGGCAACG |
| | TGTTCAGCTGCTCCGTGATGCATGAAGCTCTGCACAACCACTATACACA |
| | GAAGTCTCTGAGCCTGTCTCCTGGCAAGATCGAGGGCAGAATGGACG |
| | ATATCCAGATGACACAGACAACCTCTTCTCTGTCTGCTTCTCTGGGCGA |
| | TAGAGTGACCATCAGCTGCAGAGCTTCTCAGGACATCAGCAAGTATCT |
| | GAACTGGTATCAGCAGAAGCCTGATGGCACCGTGAAGCTGCTGATCTA |
| | CCACACCTCCAGATTGCATTCTGGAGTTCCTTCCAGATTTTCTGGCTCT |
| | GGCTCTGGCACCGACTATTCTCTGACCATCAGCAATCTGGAACAGGAG |
| | GACATCGCTACCTACTTTTGCCAGCAGGGCAACACACTGCCTTACACAT |
| | TTGGAGGAGGAACAAAGCTGGAGATCACAGGAGGAGGATCTGGAGGA |
| | GGATCTGGAGGAGGATCTGAAGTTAAACTGCAGGAATCTGGACCAGGA |
| | TTAGTGGCCCCATCTCAGTCTCTGTCTGTGACCTGTACCGTTTCTGGAG |
| | TTTCTTTGCCTGATTACGGAGTGTCCTGGATCAGACAGCCCCCTAGAAA |
| | GGGACTGGAATGGTTAGGAGTGATTTGGGGATCTGAGACCACCTACTA |
| | CAACTCTGCCCTGAAGAGCAGACTGACCATCATCAAGGACAACAGCAA |
| | GTCTCAGGTGTTCCTGAAGATGAACTCCCTGCAGACCGACGATACCGC |
| | CATCTACTACTGTGCTAAGCACTACTACTATGGCGGCTCTTATGCCATGG |
| | ACTATTGGGGACAGGGCACCTCTGTGACAGTGTCTTCTTAA (SEQ ID |
| | NO: 84) |
| Human | MEFGLSWVFLVAIIKGVQCQFSVLGPSGPILAMVGEDADLPCHLFPTMSA |
| BTN3A1-beta-scFvCD19 | ETMELKWVSSSLRQVVNVYADGKEVEDRQSAPYRGRTSILRDGITAGKA |
| with knob-in-hole (KIH) | ALRIHNVTASDSGKYLCYFQDGDFYEKALVELKVAALGSDLHVDVKGYK |
| mutation and FcRn | DGGIHLECRSTGWYPQPQIQWSNNKGENIPTVEAPVVADGVGLYAVAAS |
| mutations | VIMRGSSGEGVSCTIRSSLLGLEKTASISIADPFFRSAQRWIAALAEPKSC |
| | DKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGKEY |
| | KCKVSNKALPAPIEKTISKAKGQPREPQVCTLPPSRDELTKNQVSLSCAVK |
| | GFYPSDIAVEWESNGQPENNYKTTPPVLDSDGSFFLVSKLTVDKSRWQQ |
| | GNVFSCSVLHEALHSHYTQKSLSLSPGKIEGRMDDIQMTQTTSSLSASLG |
| | DRVTISCRASQDISKYLNWYQQKPDGTVKLLIYHTSRLHSGVPSRFSGSG |
| | SGTDYSLTISNLEQEDIATYFCQQGNTLPYTFGGGTKLEITGGGSGGGSG |
| | GGSEVKLQESGPGLVAPSQSLSVTCTVSGVSLPDYGVSWIRQPPRKGLE |
| | WLGVIWGSETTYYNSALKSRLTIIKDNSKSQVFLKMNSLQTDDTAIYYCA |
| | KHYYYGGSYAMDYWGQGTSVTVSS (SEQ ID NO: 85) |
| | Nucleotide sequence of human BTN3A1-beta-scFvCD19 with |
| | knob-in-hole (KIH) mutation and FcRn mutations: |
| | ATGGAGTTTGGCCTGTCTTGGGTGTTTCTGGTGGCTATCATCAAGGGA |
| | GTGCAGTGTCAGTTCTCTGTTCTGGGACCTTCTGGACCTATCTTGGCTA |
| | TGGTGGGAGAAGATGCTGATCTGCCTTGTCACCTGTTTCCTACCATGTC |
| | TGCTGAGACCATGGAGCTGAAATGGGTGTCCTCTTCTCTGAGACAGGT |
| | GGTGAATGTGTACGCTGATGGAAAGGAGGTGGAGGACAGACAATCTG |
| | CCCCTTATAGAGGAAGAACCAGCATCCTGAGAGATGGCATCACAGCTG |
| | GAAAGGCTGCTCTGAGAATCCACAATGTGACCGCTTCTGATTCTGGCA |
| | AGTACCTGTGCTACTTCCAGGATGGCGACTTCTACGAGAAGGCTCTGG |
| | TTGAGCTGAAAGTTGCTGCTTTGGGCTCTGATCTGCATGTTGACGTGA |
| | AGGGCTACAAGGATGGAGGCATCCATCTGGAATGTAGATCTACCGGCT |
| | GGTATCCTCAACCTCAGATTCAGTGGAGCAACAACAAGGGCGAGAACA |
| | TCCCTACAGTTGAAGCCCCTGTTGTGGCTGATGGAGTTGGACTGTATG |
| | CTGTTGCTGCTAGCGTGATCATGAGAGGATCTTCTGGAGAAGGCGTGT |
| | CTTGCACCATCAGATCTTCTCTGTTGGGACTGGAGAAGACCGCTAGCA |
| | TCTCTATCGCTGACCCCTTCTTCAGATCTGCTCAAAGATGGATTGCTGC |
| | TCTGGCTGAGCCTAAGTCTTGCGATAAGACCCACACCTGTCCTCCTTG |
| | TCCTGCTCCTGAAGCTGCTGGAGGACCTTCTGTGTTTTTGTTTCCTCCT |
| | AAGCCTAAGGATACCCTGATGATCTCCAGAACCCCCGAGGTGACCTGT |
| | GTGGTGGTTGATGTTTCTCATGAGGATCCTGAAGTGAAGTTCAACTGGT |
| | ACGTGGACGGCGTGGAGGTGCACAACGCTAAGACAAAACCTAGAGAA |
| | GAGCAGTACAACTCTACCTACAGAGTGGTGTCCGTGCTGACCGTGCTG |
| | CACCAAGATTGGTTGAATGGAAAGGAGTACAAGTGCAAGGTGTCCAAC |
| | AAGGCTCTGCCTGCCCCTATCGAGAAGACCATCTCTAAGGCTAAAGGA |
| | CAACCTAGAGAACCTCAGGTGTGTACACTGCCCCCCTCTAGAGATGAG |
| | CTGACCAAGAATCAGGTTTCTCTGTCTTGTGCTGTGAAGGGCTTTTACC |
| | CCTCCGACATCGCTGTGGAATGGGAGTCTAATGGACAACCTGAGAACA |
| | ACTACAAGACCACACCTCCTGTGCTGGACTCTGACGGCTCCTTCTTTC |
| | TGGTGTCTAAGCTGACAGTGGATAAGTCTAGATGGCAACAGGGCAACG |
| | TGTTCAGCTGCAGCGTTCTGCATGAAGCTCTGCATTCCCACTATACACA |
| | GAAGTCTCTGTCCCTGTCTCCTGGCAAGATCGAGGGCAGAATGGATGA |
| | CATCCAGATGACACAGACAACCTCTTCTCTGTCTGCTTCTCTGGGCGAT |
| | AGAGTGACCATCAGCTGCAGAGCTTCTCAGGACATCAGCAAGTATCTG |
| | AACTGGTATCAGCAGAAGCCTGATGGCACCGTGAAGCTGCTGATCTAC |
| | CACACCTCCAGATTGCATTCTGGAGTTCCTTCCAGATTTTCTGGCTCTG |
| | GCTCTGGCACCGACTATTCTCTGACCATCAGCAATCTGGAACAGGAGG |
| | ACATCGCTACCTACTTTTGCCAGCAGGGCAACACACTGCCTTACACATT |
| | TGGAGGAGGAACAAAGCTGGAGATCACAGGAGGAGGATCTGGAGGAG |
| | GATCTGGAGGAGGATCTGAAGTTAAACTGCAGGAATCTGGACCAGGAT |
| | TAGTGGCCCCATCTCAGTCTCTGTCTGTGACCTGTACCGTTTCTGGAGT |
| | TTCTTTGCCTGATTACGGAGTGTCCTGGATCAGACAGCCCCCTAGAAA |
| | GGGACTGGAATGGTTAGGAGTGATTTGGGGATCTGAGACCACCTACTA |
| | CAACTCTGCCCTGAAGAGCAGACTGACCATCATCAAGGACAACAGCAA |
| | GTCTCAGGTGTTCCTGAAGATGAACTCCCTGCAGACCGACGATACCGC |
| | CATCTACTACTGTGCTAAGCACTACTACTATGGCGGCTCTTATGCCATGG |
| | ACTATTGGGGACAGGGCACCTCTGTGACAGTGTCTTCTTAA (SEQ ID |
| | NO: 86) |
| Human | Domain Linker: GSGGSGSGGSGGSG |
| BTN2A1-BTN3A1-alpha- | Linker to Fc: SKYGPP |
| scFvCD19 | CCGCCACCMEFGLSWVFLVAIIKGVQCQFIVVGPTDPILATVGENTTLRC |
| BTN2A1V-BTN3A1V-G4- | HLSPEKNAEDMEVRWFRSQFSPAVFVYKGGRERTEEQMEEYRGRTTFV |
| 19scFv3_A (IgG4 S228P, | SKDISRGSVALVIHNITAQENGTYRCYFQEGRSYDEAILHLVGSGGSGSG |
| T250Q, M428L) | GSGGSGAQFSVLGPSGPILAMVGEDADLPCHLFPTMSAETMELKWVSS |
| BTN2A1V-BTN3A1V-G1- | SLRQVVNVYADGKEVEDRQSAPYRGRTSILRDGITAGKAALRIHNVTAS |
| 19scFv3_A (IgG1, N297A) | DSGKYLCYFQDGDFYEKALVELKVAEPKSCDKTHTCPPCPAPELLGGPS |
| | VFLFPPKPKDTLMISRTPEVTCVVVDVSHEDPEVKFNWYVDGVEVHNAKT |
| | KPREEQYASTYRVVSVLTVLHQDWLNGKEYKCKVSNKALPAPIEKTISKAK |
| | GQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQ |
| | KSLSLSPGKIEGRMDDIQMTQTTSSLSASLGDRVTISCRASQDISKYLNW |
| | YQQKPDGTVKLLIYHTSRLHSGVPSRFSGSGSGTDYSLTISNLEQEDIAT |
| | YFCQQGNTLPYTFGGGTKLEITGGGSGGGSGGGSEVKLQESGPGLVAP |
| | SQSLSVTCTVSGVSLPDYGVSWIRQPPRKGLEWLGVIWGSETTYYNSAL |
| | KSRLTIIKDNSKSQVFLKMNSLQTDDTAIYYCAKHYYYGGSYAMDYWGQ |
| | GTSVTVSS* |
| | (SEQ ID NO: 87) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGSGGSGSG |
| Linker to Fc: | GSGGSGAQFSVLGPSGPILAMVGEDADLPCHLFPTMSAETMELKWVSS |
| GGGGSGGGGSGGGGS | SLRQVVNVYADGKEVEDRQSAPYRGRTSILRDGITAGKAALRIHNVTAS |
| BTN2A1V-BTN3A1V-G4- | DSGKYLCYFQDGDFYEKALVELKVAGGGGSGGGGSGGGGSCPPCPAP |
| 19scFv3_A2 (IgG4 S228P, | EFLGGPSVFLFPPKPKDQLMISRTPEVTCVVVDVSQEDPEVQFNWYVDG |
| T250Q, M428L) | VEVHNAKTKPREEQFNSTYRVVSVLTVLHQDWLSGKEYKCKVSSKGLPS |
| | SIEKTISNATGQPREPQVYTLPPSQEEMTKNQVSLTCLVKGFYPSDIAVEW |
| | ESNGQPENNYKTTPPVLDSDGSFFLYSRLTVDKSRWQEGNVFSCSVLHE |
| | ALHNHYTQKSLSLSLGKIEGRMDDIQMTQTTSSLSASLGDRVTISCRASQ |
| | DISKYLNWYQQKPDGTVKLLIYHTSRLHSGVPSRFSGSGSGTDYSLTISN |
| | LEQEDIATYFCQQGNTLPYTFGGGTKLEITGGGSGGGSGGGSEVKLQES |
| | GPGLVAPSQSLSVTCTVSGVSLPDYGVSWIRQPPRKGLEWLGVIWGSET |
| | TYYNSALKSRLTIIKDNSKSQVFLKMNSLQTDDTAIYYCAKHYYYGGSYA |
| | MDYWGQGTSVTVSS* (SEQ ID NO: 88) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Domain Linker: G(G3S)2 | SAQFSVLGPSGPILAMVGEDADLPCHLFPTMSAETMELKWVSSSLRQVV |
| Linker to Fc: SKYGPP | NVYADGKEVEDRQSAPYRGRTSILRDGITAGKAALRIHNVTASDSGKYL |
| BTN2A1V-BTN3A1V-G4- | CYFQDGDFYEKALVELKVASKYGPPCPPCPAPEFLGGPSVFLFPPKPKD |
| 19scFv3_B (IgG4 S228P | QLMISRTPEVTCVVVDVSQEDPEVQFNWYVDGVEVHNAKTKPREEQFNS |
| T250Q, M428L) | TYRVVSVLTVLHQDWLSGKEYKCKVSSKGLPSSIEKTISNATGQPREPQV |
| | YTLPPSQEEMTKNQVSLTCLVKGFYPSDIAVEWESNGQPENNYKTTPPVL |
| | DSDGSFFLYSRLTVDKSRWQEGNVFSCSVLHEALHNHYTQKSLSLSLGKI |
| | EGRMDDIQMTQTTSSLSASLGDRVTISCRASQDISKYLNWYQQKPDGTV |
| | KLLIYHTSRLHSGVPSRFSGSGSGTDYSLTISNLEQEDIATYFCQQGNTLP |
| | YTFGGGTKLEITGGGSGGGSGGGSEVKLQESGPGLVAPSQSLSVTCTV |
| | SGVSLPDYGVSWIRQPPRKGLEWLGVIWGSETTYYNSALKSRLTIIKDNS |
| | KSQVFLKMNSLQTDDTAIYYCAKHYYYGGSYAMDYWGQGTSVTVSS* |
| | (SEQ ID NO: 89) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| BTN2A1V-BTN3A1V-G1- | SAQFSVLGPSGPILAMVGEDADLPCHLFPTMSAETMELKWVSSSLRQVV |
| 19scFv3_B (IgG1, N297A) | NVYADGKEVEDRQSAPYRGRTSILRDGITAGKAALRIHNVTASDSGKYL |
| | CYFQDGDFYEKALVELKVAEPKSCDKTHTCPPCPAPELLGGPSVFLFPP |
| | KPKDTLMISRTPEVTCVVVDVSHEDPEVKFNWYVDGVEVHNAKTKPREE |
| | QYASTYRVVSVLTVLHQDWLNGKEYKCKVSNKALPAPIEKTISKAKGQPR |
| | EPQVYTLPPSRDELTKNQVSLTCLVKGFYPSDIAVEWESNGQPENNYKTT |
| | PPVLDSDGSFFLYSKLTVDKSRWQQGNVFSCSVMHEALHNHYTQKSLSL |
| | SPGKIEGRMDDIQMTQTTSSLSASLGDRVTISCRASQDISKYLNWYQQKP |
| | DGTVKLLIYHTSRLHSGVPSRFSGSGSGTDYSLTISNLEQEDIATYFCQQ |
| | GNTLPYTFGGGTKLEITGGGSGGGSGGGSEVKLQESGPGLVAPSQSLS |
| | VTCTVSGVSLPDYGVSWIRQPPRKGLEWLGVIWGSETTYYNSALKSRLTI |
| | IKDNSKSQVFLKMNSLQTDDTAIYYCAKHYYYGGSYAMDYWGQGTSVT |
| | VSS* |
| | (SEQ ID NO: 90) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Linker to Fc: | SAQFSVLGPSGPILAMVGEDADLPCHLFPTMSAETMELKWVSSSLRQVV |
| GGGGSGGGGSGGGGS | NVYADGKEVEDRQSAPYRGRTSILRDGITAGKAALRIHNVTASDSGKYL |
| BTN2A1V-BTN3A1V-G4- | CYFQDGDFYEKALVELKVAGGGGSGGGGSGGGGSCPPCPAPEFLGGP |
| 19scFv3_B2 (IgG4 S228P, | SVFLFPPKPKDQLMISRTPEVTCVVVDVSQEDPEVQFNWYVDGVEVHNA |
| T250Q, M428L) | KTKPREEQFNSTYRVVSVLTVLHQDWLSGKEYKCKVSSKGLPSSIEKTISN |
| | ATGQPREPQVYTLPPSQEEMTKNQVSLTCLVKGFYPSDIAVEWESNGQP |
| | ENNYKTTPPVLDSDGSFFLYSRLTVDKSRWQEGNVFSCSVLHEALHNHY |
| | TQKSLSLSLGKIEGRMDDIQMTQTTSSLSASLGDRVTISCRASQDISKYLN |
| | WYQQKPDGTVKLLIYHTSRLHSGVPSRFSGSGSGTDYSLTISNLEQEDIA |
| | TYFCQQGNTLPYTFGGGTKLEITGGGSGGGSGGGSEVKLQESGPGLVA |
| | PSQSLSVTCTVSGVSLPDYGVSWIRQPPRKGLEWLGVIWGSETTYYNSA |
| | LKSRLTIIKDNSKSQVFLKMNSLQTDDTAIYYCAKHYYYGGSYAMDYWG |
| | QGTSVTVSS* |
| | (SEQ ID NO: 91) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Domain Linker: G(G₃S)₃ | SGGGSAQFSVLGPSGPILAMVGEDADLPCHLFPTMSAETMELKWVSSS |
| Linker to Fc: SKYGPP | LRQVVNVYADGKEVEDRQSAPYRGRTSILRDGITAGKAALRIHNVTASD |
| BTN2A1V-BTN3A1V-G4- | SGKYLCYFQDGDFYEKALVELKVASKYGPPCPPCPAPEFLGGPSVFLFP |
| 19scFv3_C (IgG4 S228P, | PKPKDQLMISRTPEVTCVVVDVSQEDPEVQFNWYVDGVEVHNAKTKPRE |
| T250Q, M428L) | EQFNSTYRVVSVLTVLHQDWLSGKEYKCKVSSKGLPSSIEKTISNATGQP |
| | REPQVYTLPPSQEEMTKNQVSLTCLVKGFYPSDIAVEWESNGQPENNYK |
| | TTPPVLDSDGSFFLYSRLTVDKSRWQEGNVFSCSVLHEALHNHYTQKSLS |
| | LSLGKIEGRMDDIQMTQTTSSLSASLGDRVTISCRASQDISKYLNWYQQK |
| | PDGTVKLLIYHTSRLHSGVPSRFSGSGSGTDYSLTISNLEQEDIATYFCQ |
| | QGNTLPYTFGGGTKLEITGGGSGGGSGGGSEVKLQESGPGLVAPSQSL |
| | SVTCTVSGVSLPDYGVSWIRQPPRKGLEWLGVIWGSETTYYNSALKSRL |
| | TIIKDNSKSQVFLKMNSLQTDDTAIYYCAKHYYYGGSYAMDYWGQGTSV |
| | TVSS* |
| | (SEQ ID NO: 92) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| BTN2A1V-BTN3A1V-G1- | SGGGSAQFSVLGPSGPILAMVGEDADLPCHLFPTMSAETMELKWVSSS |
| 19scFv3_C (IgG1, N297A) | LRQVVNVYADGKEVEDRQSAPYRGRTSILRDGITAGKAALRIHNVTASD |
| | SGKYLCYFQDGDFYEKALVELKVAEPKSCDKTHTCPPCPAPELLGGPSV |
| | FLFPPKPKDTLMISRTPEVTCVVVDVSHEDPEVKFNWYVDGVEVHNAKTK |
| | PREEQYASTYRVVSVLTVLHQDWLNGKEYKCKVSNKALPAPIEKTISKAK |
| | GQPREPQVYTLPPSRDELTKNQVSLTCLVKGFYPSDIAVEWESNGQPEN |
| | NYKTTPPVLDSDGSFFLYSKLTVDKSRWQQGNVFSCSVMHEALHNHYTQ |
| | KSLSLSPGKIEGRMDDIQMTQTTSSLSASLGDRVTISCRASQDISKYLNW |
| | YQQKPDGTVKLLIYHTSRLHSGVPSRFSGSGSGTDYSLTISNLEQEDIAT |
| | YFCQQGNTLPYTFGGGTKLEITGGGSGGGSGGGSEVKLQESGPGLVAP |
| | SQSLSVTCTVSGVSLPDYGVSWIRQPPRKGLEWLGVIWGSETTYYNSAL |
| | KSRLTIIKDNSKSQVFLKMNSLQTDDTAIYYCAKHYYYGGSYAMDYWGQ |
| | GTSVTVSS* |
| | (SEQ ID NO: 93) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Linker to Fc: | SGGGSAQFSVLGPSGPILAMVGEDADLPCHLFPTMSAETMELKWVSSS |
| GGGGSGGGGSGGGGS | LRQVVNVYADGKEVEDRQSAPYRGRTSILRDGITAGKAALRIHNVTASD |
| BTN2A1V-BTN3A1V-G4- | SGKYLCYFQDGDFYEKALVELKVAGGGGSGGGGSGGGGSCPPCPAPE |
| 19scFv3_C2 (IgG4 S228P, | FLGGPSVFLFPPKPKDQLMISRTPEVTCVVVDVSQEDPEVQFNWYVDGV |
| T250Q, M428L) | EVHNAKTKPREEQFNSTYRVVSVLTVLHQDWLSGKEYKCKVSSKGLPSSI |
| | EKTISNATGQPREPQVYTLPPSQEEMTKNQVSLTCLVKGFYPSDIAVEWE |
| | SNGQPENNYKTTPPVLDSDGSFFLYSRLTVDKSRWQEGNVFSCSVLHEA |
| | LHNHYTQKSLSLSLGKIEGRMDDIQMTQTTSSLSASLGDRVTISCRASQDI |
| | SKYLNWYQQKPDGTVKLLIYHTSRLHSGVPSRFSGSGSGTDYSLTISNLE |
| | QEDIATYFCQQGNTLPYTFGGGTKLEITGGGSGGGSGGGSEVKLQESG |
| | PGLVAPSQSLSVTCTVSGVSLPDYGVSWIRQPPRKGLEWLGVIWGSETT |
| | YYNSALKSRLTIIKDNSKSQVFLKMNSLQTDDTAIYYCAKHYYYGGSYAM |
| | DYWGQGTSVTVSS (SEQ ID NO: 94) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Domain Linker: G(G₃S)₄ | SGGGSGGGSAQFSVLGPSGPILAMVGEDADLPCHLFPTMSAETMELKW |
| Linker to Fc: SKYGPP | VSSSLRQVVNVYADGKEVEDRQSAPYRGRTSILRDGITAGKAALRIHNVT |
| BTN2A1V-BTN3A1V-G4- | ASDSGKYLCYFQDGDFYEKALVELKVASKYGPPCPPCPAPEFLGGPSVF |
| 19scFv3_D (IgG4 S228P, | LFPPKPKDQLMISRTPEVTCVVVDVSQEDPEVQFNWYVDGVEVHNAKTK |
| T250Q, M428L) | PREEQFNSTYRVVSVLTVLHQDWLSGKEYKCKVSSKGLPSSIEKTISNAT |
| | GQPREPQVYTLPPSQEEMTKNQVSLTCLVKGFYPSDIAVEWESNGQPEN |
| | NYKTTPPVLDSDGSFFLYSRLTVDKSRWQEGNVFSCSVLHEALHNHYTQ |
| | KSLSLSLGKIEGRMDDIQMTQTTSSLSASLGDRVTISCRASQDISKYLNW |
| | YQQKPDGTVKLLIYHTSRLHSGVPSRFSGSGSGTDYSLTISNLEQEDIAT |
| | YFCQQGNTLPYTFGGGTKLEITGGGSGGGSGGGSEVKLQESGPGLVAP |
| | SQSLSVTCTVSGVSLPDYGVSWIRQPPRKGLEWLGVIWGSETTYYNSAL |
| | KSRLTIIKDNSKSQVFLKMNSLQTDDTAIYYCAKHYYYGGSYAMDYWGQ |
| | GTSVTVSS* |
| | (SEQ ID NO: 95) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| BTN2A1V-BTN3A1V-G1- | SGGGSGGGSAQFSVLGPSGPILAMVGEDADLPCHLFPTMSAETMELKW |
| 19scFv3_D (IgG1, N297A) | VSSSLRQVVNVYADGKEVEDRQSAPYRGRTSILRDGITAGKAALRIHNVT |
| | ASDSGKYLCYFQDGDFYEKALVELKVAEPKSCDKTHTCPPCPAPELLGG |
| | PSVFLFPPKPKDTLMISRTPEVTCVVVDVSHEDPEVKFNWYVDGVEVHNA |
| | KTKPREEQYASTYRVVSVLTVLHQDWLNGKEYKCKVSNKALPAPIEKTISK |
| | AKGQPREPQVYTLPPSRDELTKNQVSLTCLVKGFYPSDIAVEWESNGQP |
| | ENNYKTTPPVLDSDGSFFLYSKLTVDKSRWQQGNVFSCSVMHEALHNHY |
| | TQKSLSLSPGKIEGRMDDIQMTQTTSSLSASLGDRVTISCRASQDISKYLN |
| | WYQQKPDGTVKLLIYHTSRLHSGVPSRFSGSGSGTDYSLTISNLEQEDIA |
| | TYFCQQGNTLPYTFGGGTKLEITGGGSGGGSGGGSEVKLQESGPGLVA |
| | PSQSLSVTCTVSGVSLPDYGVSWIRQPPRKGLEWLGVIWGSETTYYNSA |
| | LKSRLTIIKDNSKSQVFLKMNSLQTDDTAIYYCAKHYYYGGSYAMDYWG |
| | QGTSVTVSS* |
| | (SEQ ID NO: 96) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Linker to Fc: | SGGGSGGGSAQFSVLGPSGPILAMVGEDADLPCHLFPTMSAETMELKW |
| GGGGSGGGGSGGGGS | VSSSLRQVVNVYADGKEVEDRQSAPYRGRTSILRDGITAGKAALRIHNVT |
| BTN2A1V-BTN3A1V-G4- | ASDSGKYLCYFQDGDFYEKALVELKVAGGGGSGGGGSGGGGSCPPCP |
| 19scFv3_D2 (IgG4 S228P, | APEFLGGPSVFLFPPKPKDQLMISRTPEVTCVVVDVSQEDPEVQFNWYV |
| T250Q, M428L) | DGVEVHNAKTKPREEQFNSTYRVVSVLTVLHQDWLSGKEYKCKVSSKGL |
| | PSSIEKTISNATGQPREPQVYTLPPSQEEMTKNQVSLTCLVKGFYPSDIAV |
| | EWESNGQPENNYKTTPPVLDSDGSFFLYSRLTVDKSRWQEGNVFSCSVL |
| | HEALHNHYTQKSLSLSLGKIEGRMDDIQMTQTTSSLSASLGDRVTISCRA |
| | SQDISKYLNWYQQKPDGTVKLLIYHTSRLHSGVPSRFSGSGSGTDYSLTI |
| | SNLEQEDIATYFCQQGNTLPYTFGGGTKLEITGGGSGGGSGGGSEVKLQ |
| | ESGPGLVAPSQSLSVTCTVSGVSLPDYGVSWIRQPPRKGLEWLGVIWG |
| | SETTYYNSALKSRLTIIKDNSKSQVFLKMNSLQTDDTAIYYCAKHYYYGG |
| | SYAMDYWGQGTSVTVSS* |
| | (SEQ ID NO: 97) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Domain Linker: (G₄S)₂ | GSAQFSVLGPSGPILAMVGEDADLPCHLFPTMSAETMELKWVSSSLRQ |
| Linker to Fc: SKYGPP | WVNVYADGKEVEDRQSAPYRGRTSILRDGITAGKAALRIHNVTASDSGK |
| BTN2A1V-BTN3A1V-G4- | YLCYFQDGDFYEKALVELKVASKYGPPCPPCPAPEFLGGPSVFLFPPKP |
| 19scFv3_E (IgG4 S228P, | KDQLMISRTPEVTCVVVDVSQEDPEVQFNWYVDGVEVHNAKTKPREEQF |
| T250Q, M428L) | NSTYRVVSVLTVLHQDWLSGKEYKCKVSSKGLPSSIEKTISNATGQPREP |
| | QVYTLPPSQEEMTKNQVSLTCLVKGFYPSDIAVEWESNGQPENNYKTTP |
| | PVLDSDGSFFLYSRLTVDKSRWQEGNVFSCSVLHEALHNHYTQKSLSLSL |
| | GKIEGRMDDIQMTQTTSSLSASLGDRVTISCRASQDISKYLNWYQQKPD |
| | GTVKLLIYHTSRLHSGVPSRFSGSGSGTDYSLTISNLEQEDIATYFCQQG |
| | NTLPYTFGGGTKLEITGGGSGGGSGGGSEVKLQESGPGLVAPSQSLSV |
| | TCTVSGVSLPDYGVSWIRQPPRKGLEWLGVIWGSETTYYNSALKSRLTII |
| | KDNSKSQVFLKMNSLQTDDTAIYYCAKHYYYGGSYAMDYWGQGTSVTV |
| | SS* |
| | (SEQ ID NO: 98) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| BTN2A1V-BTN3A1V-G1- | GSAQFSVLGPSGPILAMVGEDADLPCHLFPTMSAETMELKWVSSSLRQ |
| 19scFv3_E (IgG1, N297A) | WVNVYADGKEVEDRQSAPYRGRTSILRDGITAGKAALRIHNVTASDSGK |
| | YLCYFQDGDFYEKALVELKVAEPKSCDKTHTCPPCPAPELLGGPSVFLFP |
| | PKPKDTLMISRTPEVTCVVVDVSHEDPEVKFNWYVDGVEVHNAKTKPRE |
| | EQYASTYRVVSVLTVLHQDWLNGKEYKCKVSNKALPAPIEKTISKAKGQP |
| | REPQVYTLPPSRDELTKNQVSLTCLVKGFYPSDIAVEWESNGQPENNYKT |
| | TPPVLDSDGSFFLYSKLTVDKSRWQQGNVFSCSVMHEALHNHYTQKSLS |
| | LSPGKIEGRMDDIQMTQTTSSLSASLGDRVTISCRASQDISKYLNWYQQK |
| | PDGTVKLLIYHTSRLHSGVPSRFSGSGSGTDYSLTISNLEQEDIATYFCQ |
| | QGNTLPYTFGGGTKLEITGGGSGGGSGGGSEVKLQESGPGLVAPSQSL |
| | SVTCTVSGVSLPDYGVSWIRQPPRKGLEWLGVIWGSETTYYNSALKSRL |
| | TIIKDNSKSQVFLKMNSLQTDDTAIYYCAKHYYYGGSYAMDYWGQGTSV |
| | TVSS (SEQ ID NO: 99) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Linker to Fc: | GSAQFSVLGPSGPILAMVGEDADLPCHLFPTMSAETMELKWVSSSLRQ |
| GGGGSGGGGSGGGGS | VVNVYADGKEVEDRQSAPYRGRTSILRDGITAGKAALRIHNVTASDSGK |
| BTN2A1V-BTN3A1V-G4- | YLCYFQDGDFYEKALVELKVAGGGGSGGGGSGGGGSCPPCPAPEFLG |
| 19scFv3_E2 (IgG4 S228P, | GPSVFLFPPKPKDQLMISRTPEVTCVVVDVSQEDPEVQFNWYVDGVEVH |
| T250Q, M428L) | NAKTKPREEQFNSTYRVVSVLTVLHQDWLSGKEYKCKVSSKGLPSSIEKTI |
| | SNATGQPREPQVYTLPPSQEEMTKNQVSLTCLVKGFYPSDIAVEWESNG |
| | QPENNYKTTPPVLDSDGSFFLYSRLTVDKSRWQEGNVFSCSVLHEALHN |
| | HYTQKSLSLSLGKIEGRMDDIQMTQTTSSLSASLGDRVTISCRASQDISKY |
| | LNWYQQKPDGTVKLLIYHTSRLHSGVPSRFSGSGSGTDYSLTISNLEQE |
| | DIATYFCQQGNTLPYTFGGGTKLEITGGGSGGGSGGGSEVKLQESGPGL |
| | VAPSQSLSVTCTVSGVSLPDYGVSWIRQPPRKGLEWLGVIWGSETTYYN |
| | SALKSRLTIIKDNSKSQVFLKMNSLQTDDTAIYYCAKHYYYGGSYAMDY |
| | WGQGTSVTVSS |
| | (SEQ ID NO: 100) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| Domain Linker: (G₄S)₄ | GSGGGGSGGGGSAQFSVLGPSGPILAMVGEDADLPCHLFPTMSAETME |
| Linker to Fc: SKYGPP | LKWVSSSLRQVVNVYADGKEVEDRQSAPYRGRTSILRDGITAGKAALRI |
| BTN2A1V-BTN3A1V-G4- | HNVTASDSGKYLCYFQDGDFYEKALVELKVASKYGPPCPPCPAPEFLGG |
| 19scFv3_F (IgG4 S228P, | PSVFLFPPKPKDQLMISRTPEVTCVVVDVSQEDPEVQFNWYVDGVEVHN |
| T250Q, M428L) | AKTKPREEQFNSTYRVVSVLTVLHQDWLSGKEYKCKVSSKGLPSSIEKTIS |
| | NATGQPREPQVYTLPPSQEEMTKNQVSLTCLVKGFYPSDIAVEWESNGQ |
| | PENNYKTTPPVLDSDGSFFLYSRLTVDKSRWQEGNVFSCSVLHEALHNH |
| | YTQKSLSLSLGKIEGRMDDIQMTQTTSSLSASLGDRVTISCRASQDISKYL |
| | NWYQQKPDGTVKLLIYHTSRLHSGVPSRFSGSGSGTDYSLTISNLEQEDI |
| | ATYFCQQGNTLPYTFGGGTKLEITGGGSGGGSGGGSEVKLQESGPGLV |
| | APSQSLSVTCTVSGVSLPDYGVSWIRQPPRKGLEWLGVIWGSETTYYNS |
| | ALKSRLTIIKDNSKSQVFLKMNSLQTDDTAIYYCAKHYYYGGSYAMDYW |
| | GQGTSVTVSS (SEQ ID NO: 101) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| BTN2A1V-BTN3A1V-G1- | GSGGGGSGGGGSAQFSVLGPSGPILAMVGEDADLPCHLFPTMSAETME |
| 19scFv3_F (IgG1, N297A) | LKWVSSSLRQVVNVYADGKEVEDRQSAPYRGRTSILRDGITAGKAALRI |
| | HNVTASDSGKYLCYFQDGDFYEKALVELKVAEPKSCDKTHTCPPCPAPE |
| | LLGGPSVFLFPPKPKDTLMISRTPEVTCVVVDVSHEDPEVKFNWYVDGVE |
| | VHNAKTKPREEQYASTYRVVSVLTVLHQDWLNGKEYKCKVSNKALPAPIE |
| | KTISKAKGQPREPQVYTLPPSRDELTKNQVSLTCLVKGFYPSDIAVEWES |
| | NGQPENNYKTTPPVLDSDGSFFLYSKLTVDKSRWQQGNVFSCSVMHEAL |
| | HNHYTQKSLSLSPGKIEGRMDDIQMTQTTSSLSASLGDRVTISCRASQDI |
| | SKYLNWYQQKPDGTVKLLIYHTSRLHSGVPSRFSGSGSGTDYSLTISNLE |
| | QEDIATYFCQQGNTLPYTFGGGTKLEITGGGSGGGSGGGSEVKLQESG |
| | PGLVAPSQSLSVTCTVSGVSLPDYGVSWIRQPPRKGLEWLGVIWGSETT |
| | YYNSALKSRLTIIKDNSKSQVFLKMNSLQTDDTAIYYCAKHYYYGGSYAM |
| | DYWGQGTSVTVSS* |
| | (SEQ ID NO: 102) |
| Human | CCGCCACCMEFGLSWVFLVAIIKGVQCQFIVVGPTDPILATVGENTTLRC |
| BTN2A1-BTN3A1-alpha- | HLSPEKNAEDMEVRWFRSQFSPAVFVYKGGRERTEEQMEEYRGRTTFV |
| scFvCD19 | SKDISRGSVALVIHNITAQENGTYRCYFQEGRSYDEAILHLVGGGGSGGG |
| | GSGGGGSGGGGSAQFSVLGPSGPILAMVGEDADLPCHLFPTMSAETME |
| Linker to Fc: | LKWVSSSLRQVVNVYADGKEVEDRQSAPYRGRTSILRDGITAGKAALRI |
| GGGGSGGGGSGGGGS | HNVTASDSGKYLCYFQDGDFYEKALVELKVAGGGGSGGGGSGGGGSC |
| BTN2A1V-BTN3A1V-G4- | PPCPAPEFLGGPSVFLFPPKPKDQLMISRTPEVTCVVVDVSQEDPEVQFN |
| 19scFv3_F2 (IgG4 S228P, | WYVDGVEVHNAKTKPREEQFNSTYRVVSVLTVLHQDWLSGKEYKCKVSS |
| T250Q, M428L) | KGLPSSIEKTISNATGQPREPQVYTLPPSQEEMTKNQVSLTCLVKGFYPS |
| | DIAVEWESNGQPENNYKTTPPVLDSDGSFFLYSRLTVDKSRWQEGNVFS |
| | CSVLHEALHNHYTQKSLSLSLGKIEGRMDDIQMTQTTSSLSASLGDRVTIS |
| | CRASQDISKYLNWYQQKPDGTVKLLIYHTSRLHSGVPSRFSGSGSGTDY |
| | SLTISNLEQEDIATYFCQQGNTLPYTFGGGTKLEITGGGSGGGSGGGSEV |
| | KLQESGPGLVAPSQSLSVTCTVSGVSLPDYGVSWIRQPPRKGLEWLGVI |
| | WGSETTYYNSALKSRLTIIKDNSKSQVFLKMNSLQTDDTAIYYCAKHYYY |
| | GGSYAMDYWGQGTSVTVSS |
| | (SEQ ID NO: 103) |
| Human | MEFGLSWVFLVAIIKGVQCQFIVVGPTDPILATVGENTTLRCHLSPEKNAE |
| BTN2A1-alpha-B7H3scFv | DMEVRWFRSQFSPAVFVYKGGRERTEEQMEEYRGRTTFVSKDISRGSV |
| | ALVIHNITAQENGTYRCYFQEGRSYDEAILHLVVAGLGSKPLISMRGHED |
| | GGIRLECISRGWYPKPLTVWRDPYGGVAPALKEVSMPDADGLFMVTTA |
| | VIIRDKSVRNMSCSINNTLLGQKKESVIFIPESFMPSVSPCAGSGSRKGGK |
| | RGSKYGPPCPPCPAPEFLGGPSVFLFPPKPKDQLMISRTPEVTCVVVDVS |
| | QEDPEVQFNWYVDGVEVHNAKTKPREEQFNSTYRVVSVLTVLHQDWLS |
| | GKEYKCKVSSKGLPSSIEKTISNATGQPREPQVYTLPPSQEEMTKNQVSL |
| | TCLVKGFYPSDIAVEWESNGQPENNYKTTPPVLDSDGSFFLYSRLTVDKS |
| | RWQEGNVFSCSVLHEALHNHYTQKSLSLSLGKDEGGEDGSDIQMTQSPS |
| | FLSASVGDRVTITCKASQNVDTNVAWYQQKPGKAPKALIYSASYRYSGV |
| | PSRFSGSGSGTDFTLTISSLQPEDFAEYFCQQYNNYPFTFGQGTKLEIKS |
| | SGGGGSGGGGSGGGGSEVQLVESGGGLVQPGGSLRLSCAASGFTFSS |
| | FGMHWVRQAPGKGLEWVAYISSGSGTIYYADTVKGRFTISRDNAKNSLY |
| | LQMNSLRAEDTAVYYCARHGYRYEGFDYWGQGTTVTVSS |
| | (SEQ ID NO: 113) |
| Human | MEFGLSWVFLVAIIKGVQCQFSVLGPSGPILAMVGEDADLPCHLFPTMSA |
| BTN3A1-beta-B7H3scFv | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | DEGGEDGSKYGPPCPPCPAPEFLGGPSVFLFPPKPKDQLMISRTPEVTC |
| | WWVDVSQEDPEVQFNWYVDGVEVHNAKTKPREEQFNSTYRVVSVLTVLH |
| | QDWLSGKEYKCKVSSKGLPSSIEKTISNATGQPREPQVYTLPPSQEEMTK |
| | NQVSLTCLVKGFYPSDIAVEWESNGQPENNYKTTPPVLDSDGSFFLYSRL |
| | TVDKSRWQEGNVFSCSVLHEALHNHYTQKSLSLSLGKRKGGKRGSGSDI |
| | QMTQSPSFLSASVGDRVTITCKASQNVDTNVAWYQQKPGKAPKALIYSA |
| | SYRYSGVPSRFSGSGSGTDFTLTISSLQPEDFAEYFCQQYNNYPFTFGQ |
| | GTKLEIKSSGGGGSGGGGSGGGGSEVQLVESGGGLVQPGGSLRLSCA |
| | ASGFTFSSFGMHWVRQAPGKGLEWVAYISSGSGTIYYADTVKGRFTISR |
| | DNAKNSLYLQMNSLRAEDTAVYYCARHGYRYEGFDYWGQGTTVTVSS |
| | (SEQ ID NO: 114) |
| Human | MEFGLSWVFLVAIIKGVQCQFIVVGPTDPILATVGENTTLRCHLSPEKNAE |
| BTN2A1-alpha-FAPscFv | DMEVRWFRSQFSPAVFVYKGGRERTEEQMEEYRGRTTFVSKDISRGSV |
| | ALVIHNITAQENGTYRCYFQEGRSYDEAILHLVVAGLGSKPLISMRGHED |
| | GGIRLECISRGWYPKPLTVWRDPYGGVAPALKEVSMPDADGLFMVTTA |
| | VIIRDKSVRNMSCSINNTLLGQKKESVIFIPESFMPSVSPCAGSGSRKGGK |
| | RGSKYGPPCPPCPAPEFLGGPSVFLFPPKPKDQLMISRTPEVTCVVVDVS |
| | QEDPEVQFNWYVDGVEVHNAKTKPREEQFNSTYRVVSVLTVLHQDWLS |
| | GKEYKCKVSSKGLPSSIEKTISNATGQPREPQVYTLPPSQEEMTKNQVSL |
| | TCLVKGFYPSDIAVEWESNGQPENNYKTTPPVLDSDGSFFLYSRLTVDKS |
| | RWQEGNVFSCSVLHEALHNHYTQKSLSLSLGKDEGGEDGSEVQLLESG |
| | GGLVQPGGSLRLSCAASGFTFSSHAMSWVRQAPGKGLEWVSAIWASG |
| | EQYYADSVKGRFTISRDNSKNTLYLQMNSLRAEDTAVYYCAKGWLGNF |
| | DYWGQGTLVTVSSGGGGSGGGGSGGGGSEIVLTQSPGTLSLSPGERAT |
| | LSCRASQSVSRSYLAWYQQKPGQAPRLLIIGASTRATGIPDRFSGSGSG |
| | TDFTLTISRLEPEDFAVYYCQQGQVIPPTFGQGTKVEIK (SEQ ID NO: 115) |
| Human | MEFGLSWVFLVAIIKGVQCQFSVLGPSGPILAMVGEDADLPCHLFPTMSA |
| BTN3A1-beta-FAPscFv | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | DEGGEDGSKYGPPCPPCPAPEFLGGPSVFLFPPKPKDQLMISRTPEVTC |
| | VVVDVSQEDPEVQFNWYVDGVEVHNAKTKPREEQFNSTYRVVSVLTVLH |
| | QDWLSGKEYKCKVSSKGLPSSIEKTISNATGQPREPQVYTLPPSQEEMTK |
| | NQVSLTCLVKGFYPSDIAVEWESNGQPENNYKTTPPVLDSDGSFFLYSRL |
| | TVDKSRWQEGNVFSCSVLHEALHNHYTQKSLSLSLGKRKGGKRGSGSE |
| | VQLLESGGGLVQPGGSLRLSCAASGFTFSSHAMSWVRQAPGKGLEWV |
| | SAIWASGEQYYADSVKGRFTISRDNSKNTLYLQMNSLRAEDTAVYYCAK |
| | GWLGNFDYWGQGTLVTVSSGGGGSGGGGSGGGGSEIVLTQSPGTLSL |
| | SPGERATLSCRASQSVSRSYLAWYQQKPGQAPRLLIIGASTRATGIPDR |
| | FSGSGSGTDFTLTISRLEPEDFAVYYCQQGQVIPPTFGQGTKVEIK (SEQ |
| | ID NO: 116) |
| Human | MEFGLSWVFLVAIIKGVQCQFIVVGPTDPILATVGENTTLRCHLSPEKNAE |
| BTN2A1-alpha- CD20scFv | DMEVRWFRSQFSPAVFVYKGGRERTEEQMEEYRGRTTFVSKDISRGSV |
| | ALVIHNITAQENGTYRCYFQEGRSYDEAILHLVVAGLGSKPLISMRGHED |
| | GGIRLECISRGWYPKPLTVWRDPYGGVAPALKEVSMPDADGLFMVTTA |
| | VIIRDKSVRNMSCSINNTLLGQKKESVIFIPESFMPSVSPCAGSGSRKGGK |
| | RGSKYGPPCPPCPAPEFLGGPSVFLFPPKPKDQLMISRTPEVTCVVVDVS |
| | QEDPEVQFNWYVDGVEVHNAKTKPREEQFNSTYRVVSVLTVLHQDWLS |
| | GKEYKCKVSSKGLPSSIEKTISNATGQPREPQVYTLPPSQEEMTKNQVSL |
| | TCLVKGFYPSDIAVEWESNGQPENNYKTTPPVLDSDGSFFLYSRLTVDKS |
| | RWQEGNVFSCSVLHEALHNHYTQKSLSLSLGKDEGGEDGSQVQLVQSG |
| | AEVKKPGASVKVSCKASGYTFTSYNMHWVRQAPGQGLEWMGAIYPGN |
| | GDTSYNQKFQGRVTITADKSISTAYMELSSLRSEDTAVYYCARSTYYGG |
| | DWYFNVWGAGTLVTVSSGGGGSGGGGSGGGGSGGGGSQIVLTQSPSS |
| | LSASVGDRVTITCRASSSVSYIHWFQQKPGKSPKPLIYATSNLASGVPVR |
| | FSGSGSGTDYTLTISSLQPEDFATYYCQQWTSNPPTFGGGTKVEIK (SEQ |
| | ID NO: 117) |
| Human | MEFGLSWVFLVAIIKGVQCQFSVLGPSGPILAMVGEDADLPCHLFPTMSA |
| BTN3A1-beta-CD20scFv | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | DEGGEDGSKYGPPCPPCPAPEFLGGPSVFLFPPKPKDQLMISRTPEVTC |
| | WWVDVSQEDPEVQFNWYVDGVEVHNAKTKPREEQFNSTYRVVSVLTVLH |
| | QDWLSGKEYKCKVSSKGLPSSIEKTISNATGQPREPQVYTLPPSQEEMTK |
| | NQVSLTCLVKGFYPSDIAVEWESNGQPENNYKTTPPVLDSDGSFFLYSRL |
| | TVDKSRWQEGNVFSCSVLHEALHNHYTQKSLSLSLGKRKGGKRGSGSQ |
| | VQLVQSGAEVKKPGASVKVSCKASGYTFTSYNMHWVRQAPGQGLEWM |
| | GAIYPGNGDTSYNQKFQGRVTITADKSISTAYMELSSLRSEDTAVYYCAR |
| | STYYGGDWYFNVWGAGTLVTVSSGGGGSGGGGGGGGSGGGGSQIV |
| | LTQSPSSLSASVGDRVTITCRASSSVSYIHWFQQKPGKSPKPLIYATSNL |
| | ASGVPVRFSGSGSGTDYTLTISSLQPEDFATYYCQQWTSNPPTFGGGTK |
| | VEIK (SEQ ID NO: 118) |
| Human | MEFGLSWVFLVAIIKGVQCQFIVVGPTDPILATVGENTTLRCHLSPEKNAE |
| BTN2A1-alpha-CD33scFv | DMEVRWFRSQFSPAVFVYKGGRERTEEQMEEYRGRTTFVSKDISRGSV |
| | ALVIHNITAQENGTYRCYFQEGRSYDEAILHLVVAGLGSKPLISMRGHED |
| | GGIRLECISRGWYPKPLTVWRDPYGGVAPALKEVSMPDADGLFMVTTA |
| | VIIRDKSVRNMSCSINNTLLGQKKESVIFIPESFMPSVSPCAGSGSRKGGK |
| | RGSKYGPPCPPCPAPEFLGGPSVFLFPPKPKDQLMISRTPEVTCVVVDVS |
| | QEDPEVQFNWYVDGVEVHNAKTKPREEQFNSTYRVVSVLTVLHQDWLS |
| | GKEYKCKVSSKGLPSSIEKTISNATGQPREPQVYTLPPSQEEMTKNQVSL |
| | TCLVKGFYPSDIAVEWESNGQPENNYKTTPPVLDSDGSFFLYSRLTVDKS |
| | RWQEGNVFSCSVLHEALHNHYTQKSLSLSLGKDEGGEDGSQVQLVQSG |
| | GDLAQPGRSLRLSCAASGFTFDDYAMHWVRQAPGKGLEWVAVIWPDG |
| | GQKYYGDSVKGRFTVSRDNPKNTLYLQMNSLRAEDTAIYYCVRHFNAW |
| | DYWGQGTLVTVSSASGGGGSGGGGSGGGGSDIQLTQSPSSLSAYVGG |
| | RVTITCQASQGISQFLNWFQQKPGKAPKLLISDASNLEPGVPSRFSGSG |
| | SGTDFTFTITNLQPEDIATYYCQQYDDLPLTFGGGTKVEIK (SEQ ID NO: |
| | 119) |
| Human | MEFGLSWVFLVAIIKGVQCQFSVLGPSGPILAMVGEDADLPCHLFPTMSA |
| BTN3A1-beta-CD33scFv | ETMELKWVSSSLRQVVNVYADGKEVEDRQSAPYRGRTSILRDGITAGKA |
| | ALRIHNVTASDSGKYLCYFQDGDFYEKALVELKVAALGSDLHVDVKGYK |
| | DGGIHLECRSTGWYPQPQIQWSNNKGENIPTVEAPVVADGVGLYAVAAS |
| | VIMRGSSGEGVSCTIRSSLLGLEKTASISIADPFFRSAQRWIAALAGGSGS |
| | DEGGEDGSKYGPPCPPCPAPEFLGGPSVFLFPPKPKDQLMISRTPEVTC |
| | WVDVSQEDPEVQFNWYVDGVEVHNAKTKPREEQFNSTYRVVSVLTVLH |
| | QDWLSGKEYKCKVSSKGLPSSIEKTISNATGQPREPQVYTLPPSQEEMTK |
| | NQVSLTCLVKGFYPSDIAVEWESNGQPENNYKTTPPVLDSDGSFFLYSRL |
| | TVDKSRWQEGNVFSCSVLHEALHNHYTQKSLSLSLGKRKGGKRGSGSQ |
| | VQLVQSGGDLAQPGRSLRLSCAASGFTFDDYAMHWVRQAPGKGLEWV |
| | AVIWPDGGQKYYGDSVKGRFTVSRDNPKNTLYLQMNSLRAEDTAIYYCV |
| | RHFNAWDYWGQGTLVTVSSASGGGGSGGGGSGGGGSDIQLTQSPSSL |
| | SAYVGGRVTITCQASQGISQFLNWFQQKPGKAPKLLISDASNLEPGVPS |
| | RFSGSGSGTDFTFTITNLQPEDIATYYCQQYDDLPLTFGGGTKVEIK (SEQ |
| | ID NO: 120) |

### The First Domain

In embodiments, the first domain comprises two of the same butyrophilin family proteins. In embodiments, wherein the first domain comprises two different butyrophilin family proteins. In embodiments, the butyrophilin family proteins comprise a variable domain, which is also known as a V-type domain or a V-set domain.

Suitable butyrophilin family proteins or fragments thereof are derived from the native butyrophilin family proteins that comprise a B30.2 domain in the cytosolic tail of the full length protein.

In embodiments, the first domain is a portion of Butyrophilin subfamily 2 member A1 (BTN2A1). In embodiments, the first domain comprises substantially all the extracellular domain of BTN2A1. In embodiments, the first domain is capable of binding a gamma delta T cell receptor (e.g. Vγ9δ2). BTN2A1 is also known as BT2.1, BTF1. In embodiments, the portion of BTN2A1 is a portion of the extracellular domain of BTN2A1. In embodiments, the present heterodimeric protein further comprises a domain, e.g., the extracellular domain BTN2A1.

The amino acid sequence of extracellular domain of human BTN2A1, which is an illustrative amino acid sequence of human BTN2A1 suitable in the current disclosure is the following:

(SEQ ID NO: 27)
QFIVVGPTDPILATVGENTTLRCHLSPEKNAEDMEVRWFRSQFSPAVFV
YKGGRERTEEQMEEYRGRTTFVSKDISRGSVALVIHNITAQENGTYRCY
FQEGRSYDEAILHLVVAGLGSKPLISMRGHEDGGIRLECISRGWYPKPL
TVWRDPYGGVAPALKEVSMPDADGLFMVTTAVIIRDKSVRNMSCSINNT
LLGQKKESVIFIPESFMPSVSPCA

In some embodiments, the fragment of extracellular domain of human BTN2A1, which is a variable domain, which is also known as a V-type domain or a V-set domain amino acid sequence of human BTN2A1 suitable in the current disclosure is the following:

(SEQ ID NO: 28)
QFIVVGPTDPILATVGENTTLRCHLSPEKNAEDMEVRWFRSQFSPAVFV
YKGGRERTEEQMEEYRGRTTFVSKDISRGSVALVIHNITAQENGTYRCY
FQEGRSYDEAILHLV

In embodiments, the present heterodimeric protein comprises the extracellular domain of human BTN2A1 which has the amino acid sequence of SEQ ID NO: 27 or SEQ ID NO: 28. In embodiments, the present heterodimeric proteins may comprise the extracellular domain of BTN2A1 as described herein, or a variant or functional fragment thereof. For instance, the chimeric protein may comprise a sequence of the extracellular domain of BTN2A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of BTN2A1 as described herein.

BTN2A1 derivatives can be constructed from available structural data, including a homology model described by Karunakaran et al., Butyrophilin-2A1 Directly Binds Germline-Encoded Regions of the Vγ9V62 TCR and Is Essential for Phosphoantigen Sensing, Immunity. 52(3): 487-498 (2020); Rigau et al., Butyrophilin 2A1 is essential for phosphoantigen reactivity by γδ T cells, Science 367, eaay5516 (2020). Moreover, without wishing to be bound by theory, the protein structure homology-model of BTN2A1 is available at SWISS-MODEL repository. Bienert et al., “The SWISS-MODEL Repository-new features and functionality.” Nucleic Acids Research, 45(D1): D313-D319 (2017). Additional structural insight obtained from mutagenesis. Rigau et al., Butyrophilin 2A1 is essential for phosphoantigen reactivity by γδ T cells. Science 367(6478): eaay5516 (2020).

In embodiments, the first domain is a portion of Butyrophilin subfamily 3 member A1 (BTN3A1). In embodiments, the first domain comprises substantially all the extracellular domain of BTN3A1. In embodiments, the first domain is capable of binding a gamma delta T cell receptor (e.g. Vγ9δ2). BTN3A1 is also known as BTF5. In embodiments, the portion of BTN3A1 is a portion of the extracellular domain of BTN3A1. In embodiments, the present heterodimeric protein further comprises a domain, e.g., the extracellular domain BTN3A1.

The amino acid sequence of extracellular domain of human BTN3A1, which is an illustrative amino acid sequence of human BTN3A1 suitable in the current disclosure is the following:

(SEQ ID NO: 29)
QFSVLGPSGPILAMVGEDADLPCHLFPTMSAETMELKWVSSSLRQVVNV
YADGKEVEDRQSAPYRGRTSILRDGITAGKAALRIHNVTASDSGKYLCY
FQDGDFYEKALVELKVAALGSDLHVDVKGYKDGGIHLECRSTGWYPQPQ
IQWSNNKGENIPTVEAPVVADGVGLYAVAASVIMRGSSGEGVSCTIRSS
LLGLEKTASISIADPFFRSAQRWIAALAG

In some embodiments, the fragment of extracellular domain of human BTN3A1, which is a variable domain, which is also known as a V-type domain or a V-set domain amino acid sequence of human BTN3A1 suitable in the current disclosure is the following:

(SEQ ID NO: 30)
AQFSVLGPSGPILAMVGEDADLPCHLFPTMSAETMELKWVSSSLRQVVN
VYADGKEVEDRQSAPYRGRTSILRDGITAGKAALRIHNVTASDSGKYLC
YFQDGDFYEKALVELKVA

In embodiments, the present heterodimeric protein comprises the extracellular domain of human BTN3A1 which has the amino acid sequence of SEQ ID NO: 29 or SEQ ID NO: 30. In embodiments, the present heterodimeric proteins may comprise the extracellular domain of BTN3A1 as described herein, or a variant or functional fragment thereof. For instance, the chimeric protein may comprise a sequence of the extracellular domain of BTN3A1 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of BTN3A1 as described herein.

BTN3A1 derivatives can be constructed from available structural data, including the following: Palakodeti et al., The molecular basis for modulation of human V(gamma)9V(delta)2 T cell responses by CD277/Butyrophilin-3 (BTN3A)-specific antibodies, J Biol Chem 287: 32780-32790 (2012); Vavassori et al., Butyrophilin 3A1 binds phosphorylated antigens and stimulates human gamma delta T cells. Nat Immunol 14: 908-916 (2013); Sandstrom et al., The Intracellular B30.2 Domain of Butyrophilin 3A1 Binds Phosphoantigens to Mediate Activation of Human V gamma 9V delta 2 T Cells. Immunity 40: 490-500 (2014); Rhodes et al., Activation of Human Gammadelta T Cells by Cytosolic Interactions of Btn3A1 with Soluble Phosphoantigens and the Cytoskeletal Adaptor Periplakin. J Immunol 194: 2390 (2015); Gu et al., Phosphoantigen-induced conformational change of butyrophilin 3A1 (BTN3A1) and its implication on V gamma 9V delta 2 T cell activation. Proc Natl Acad Sci USA 114: E7311-E7320 (2017); Salim et al., BTN3A1 Discriminates gamma delta T Cell Phosphoantigens from Nonantigenic Small Molecules via a Conformational Sensor in Its B30.2 Domain. ACS Chem Biol 12: 2631-2643 (2017); Yang et al., A Structural Change in Butyrophilin upon Phosphoantigen Binding Underlies Phosphoantigen-Mediated V gamma 9V delta 2 T Cell Activation. Immunity 50: 1043 (2019); Wang et al., Critical Roles for Coiled-Coil Dimers of Butyrophilin 3A1 in the Sensing of Prenyl Pyrophosphates by Human Vγ2V62 T Cells, J Immuno/203(3):607-626 (2019).

In embodiments, the first domain comprises a portion of BTN2A1. In embodiments, the portion of BTN2A1 is an extracellular domain of BTN2A1, or a γδ T-cell receptor (e.g., γ9δ2)-binding fragment thereof.

In embodiments, the first domain comprises a portion of BTN3A1. In embodiments, the portion of BTN3A1 is an extracellular domain of BTN3A1, or a γδ T-cell receptor (e.g., γ9δ2)-binding fragment thereof.

In embodiments, first domain comprises a variable domain of BTN2A1 (BTN2A1V) and variable domain of BTN3A1 (BTN3A1V), joined by a flexible amino acid sequence (without limitation, e.g., one of SEQ ID NOs: 104-110). The first domain is fused to a targeting domain (without limitation, e.g., CD19scFv, B7H3scFv, FAPscFv, CD20scFv, CD33scFv, etc.) via a hinge-CH2-CH3 Fc domain from IgG1 or IgG4. In embodiments, BTN2A1V-BTN3A1V-Fc domain of IgG1-19scFv protein is selected from SEQ ID NOs: 87, 90, 93, 96, 99, and 102. In embodiments, BTN2A1V-BTN3A1V-Fc domain of IgG4-19scFv protein is selected from SEQ ID NOs: 88, 89, 91, 92, 94, 95, 97, 98, 100, 101 and 103.

In embodiments, the first domain comprises a portion of BTN2A1 and a portion of BTN3A1. In embodiments, the portion of BTN2A1 is an extracellular domain of BTN2A1, or a γδ T-cell receptor (e.g. γ9δ2)-binding fragment thereof. In embodiments, the portion of BTN3A1 is an extracellular domain of BTN3A1, or a γδ T-cell receptor (e.g. γ9δ2)-binding fragment thereof. In embodiments, a second linker adjoins (i) the BTN2A1 protein, or the fragment thereof, and (ii) the BTN3A1 protein, or the fragment thereof. In embodiments, the second linker is a flexible amino acid sequence. Exemplary second linkers are G(G₃S)ₘ, or GGGSₙ where m or n is 2-6, for example, GGGGSGGGS (SEQ ID NO: 104), GGGGSGGGGSGGGGS (SEQ ID NO: 105), GGGGSGGGSGGGS (SEQ ID NO: 106), GGGSGGGSGGGSGGGS (SEQ ID NO: 107), GGGGSGGGSGGGSGGGS (SEQ ID NO: 108), GGGGSGGGGS (SEQ ID NO: 109), and GGGGSGGGGSGGGGSGGGGS (SEQ ID NO: 110). In embodiments, two of the heterodimeric proteins associate to form a heterodimer of two chains, which comprise a BTN2A12-BTN3A12 tetramer.

### The Second Domain Comprising a Targeting Domain

The heterodimeric proteins of any of the embodiments disclosed herein comprise a second domain comprising a targeting domain. In some embodiments, the targeting domain is an antibody-like molecule, or antigen binding fragment thereof. In some embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In some embodiments, the antibody-like molecule is an scFv. In some embodiments, the targeting domain is an extracellular domain. In some embodiments, the targeting domain is capable of binding an antigen on the surface of a cancer cell. In some embodiments, the targeting domain specifically binds one of CD19, PSMA, GD2, PSCA, BCMA, CD123, B7-H3, CD20, CD30, CD33, CD38, CEA, CLEC12A, DLL3, EGFRvIII, EpCAM, CD307, FLT3, GPC3, gpA33, HER2, MUC16, P-cadherin, SSTR2, and mesothelin. In some embodiments, the targeting domain comprises a portion of the extracellular domain of LAG-3, PD-1, TIGIT, CD19, or PSMA. In some embodiments, the targeting domain specifically binds CD19. In some embodiments, the targeting domain specifically binds PSMA. In embodiments, the targeting domain specifically binds B7H3. In embodiments, the targeting domain specifically binds FAP. In embodiments, the targeting domain specifically binds CD20. In embodiments, the targeting domain specifically binds CD33.

Illustrative sequences of second domain comprising a targeting domain are provided below:

An illustrative targeting domain is scFVh19, which is the heavy chain variable domain of an scFV specific to human CD19, and has the following sequence:

(SEQ ID NO: 31)
DIQLTQSPASLAVSLGQRATISCKASQSVDYDGDSYLNWYQQIPGQPPK
LLIYDASNLVSGIPPRFSGSGSGTDFTLNIHPVEKVDAATYHCQQSTED
PWTFGGGTKLEIK

An illustrative targeting domain is scFVlh19, which is light chain variable domain of an scFV specific to human CD19, and has the following sequence:

(SEQ ID NO: 32)
EVQLVESGGGLVQPGGSLTLSCAASRFMISEYHMHWVRQAPGKGLEWVS
TINPAGTTDYAESVKGRFTISRDNAKNTLYLQMNSLKPEDTAVYYCDSY
GYRGQGTQVTV

An illustrative targeting domain is scFvCD19, which an scFV specific to human CD19, and has the following sequence:

(SEQ ID NO: 33)
QVQLQQSGAELVRPGSSVKISCKASGYAFSSYWMNWVKQRPGQGLEWIG
QIWPGDGDTNYNGKFKGKATLTADESSSTAYMQLSSLASEDSAVYFCAR
RETTTVGRYYYAMDYWGQGTTVTVSSGGGGSGGGGSGGGGSDIQLTQSP
ASLAVSLGQRATISCKASQSVDYDGDSYLNWYQQIPGQPPKLLIYDASN
LVSGIPPRFSGSGSGTDFTLNIHPVEKVDAATYHCQQSTEDPWTFGGGT
KLEIK

An illustrative targeting domain is scFvCD19VHVL, which an scFV specific to mouse CD19, and has the following sequence:

(SEQ ID NO: 34)
EVQLQQSGAELVRPGTSVKLSCKVSGDTITFYYMHFVKQRPGQGLEWIG
RIDPEDESTKYSEKFKNKATLTADTSSNTAYLKLSSLTSEDTATYFCIY
GGYYFDYWGQGVMVTVSSGGGGSGGGGSGGGGSDIQMTQSPASLSTSLG
ETVTIQCQASEDIYSGLAWYQQKPGKSPQLLIYGASDLQDGVPSRFSGS
GSGTQYSLKITSMQTEDEGVYFCQQGLTYPRTFGGGTKLELK

An illustrative targeting domain is 19scFv3, which an scFV specific to human CD19, and has the following sequence:

(SEQ ID NO: 35)
DIQMTQTTSSLSASLGDRVTISCRASQDISKYLNWYQQKPDGTVKLLIY
HTSRLHSGVPSRFSGSGSGTDYSLTISNLEQEDIATYFCQQGNTLPYTF
GGGTKLEITGGGSGGGSGGGSEVKLQESGPGLVAPSQSLSVTCTVSGVS
LPDYGVSWIRQPPRKGLEWLGVIWGSETTYYNSALKSRLTIIKDNSKSQ
VFLKMNSLQTDDTAIYYCAKHYYYGGSYAMDYWGQGTSVTVSS

An illustrative targeting domain is scFvCD19VLVH, which an scFV specific to mouse CD19, and has the following sequence:

(SEQ ID NO: 36)
DIQMTQSPASLSTSLGETVTIQCQASEDIYSGLAWYQQKPGKSPQLLIY
GASDLQDGVPSRFSGSGSGTQYSLKITSMQTEDEGVYFCQQGLTYPRTF
GGGTKLELKGGGGSGGGGSGGGGSEVQLQQSGAELVRPGTSVKLSCKVS
GDTITFYYMHFVKQRPGQGLEWIGRIDPEDESTKYSEKFKNKATLTADT
SSNTAYLKLSSLTSEDTATYFCIYGGYYFDYWGQGVMVTVSS

An illustrative targeting domain is scFVIPSMA, which is light chain variable domain of an scFV specific to human PSMA, and has the following sequence:

(SEQ ID NO: 37)
RKGGKRGSGSGQTVVTQEPSLTVSPGGTVTLTCASSTGAVTSGNYPNWV
QQKPGQAPRGLIGGTKFLVPGTPARFSGSLLGGKAALTLSGVQPEDEAE
YYCTLWYSNRWVFGGGTKLTVL

An illustrative targeting domain is GD2scFv3, which an scFV specific to human GD2, and has the following sequence

(SEQ ID NO: 38)
GTDFTLKISRVEAEDLGVYFCSQSTHVPPLTFGAGTKLELKGGGSGGGS
GGGSEVQLLQSGPELEKPGASVMISCKASGSSFTGYNMNWVRQNIGKSL
EWIGAIDPYYGGTSYNQKFKGRATLTVDKSSSTAYMHLKSLTSEDSAVY
YCVSGMKYWGQGTSVTVSS

An illustrative targeting domain is CD33scFv-3, which an scFV specific to human CD33, and has the following sequence (the linker joining the variable regions of the heavy (V_{H}) and light chains (V_{L}) is shown by an underline):

(SEQ ID NO: 39)
QVQLVQSGGGLVQPGGSLRLSCAASGFTFSSYAMSWVRQAPGKGLEWVS
AISGSGGSTYYADSVKGRFTISRDNSKNTLYLQMNSLRAEDTAVYYCAK
EDTIRGPNYYYYGMDVWGQGTTVTVSSASGGGGSGGGGSGGGGSETTLT
QSPSSVSASVGDRVSITCRASQDIDTWLAWYQLKPGKAPKLLMYAASNL
QGGVPSRFSGSGSGTDFILTISSLQPEDFATYYCQQASIFPPTFGGGTK
VDIK

An illustrative targeting domain is CD33scFv-4, which an scFV specific to human CD33, and has the following sequence (the linker joining the variable regions of the heavy (V_{H}) and light chains (V_{L}) is shown by an underline):

(SEQ ID NO: 40)
QVQLVQSGAEVKKPGESLKISCKGSGYSFTSYWIGWVRQMPGKGLEWMG
IIYPGDSDTRYSPSFQGQVTISADKSITTAYLQWSSLRASDSAMYYCAR
GGYSDYDYYFDFWGQGTLVTVSSASGGGGSGGGGSGGGGSEIVMTQSPL
SLPVTPGEPASISCRSSQSLLHSNGYNYLDWYLQKPGQSPQLLIYLGSN
RASGVPDRFSGSGSGTDFTLKISRVEAEDVGVYYCMQALQTPFTFGGGT
KVEIK

An illustrative targeting domain is CD33scFv-5, which an scFV specific to human CD33, and has the following sequence (the linker joining the variable regions of the heavy (V_{H}) and light chains (V_{L}) is shown by an underline):

(SEQ ID NO: 41)
QVQLVQSGGDLAQPGRSLRLSCAASGFTFDDYAMHWVRQAPGKGLEWVA
VIWPDGGQKYYGDSVKGRFTVSRDNPKNTLYLQMNSLRAEDTAIYYCVR
HFNAWDYWGQGTLVTVSSASGGGGSGGGGSGGGGSDIQLTQSPSSLSAY
VGGRVTITCQASQGISQFLNWFQQKPGKAPKLLISDASNLEPGVPSRFS
GSGSGTDFTFTITNLQPEDIATYYCQQYDDLPLTFGGGTKVEIK

An illustrative targeting domain is CD33scFv-6, which an scFV specific to human CD33, and has the following sequence (the linker joining the variable regions of the heavy (V_{H}) and light chains (V_{L}) is shown by an underline):

(SEQ ID NO: 42)
QVQLVQSGGGVVQPGKSLRLSCAASGFTFSIFAMHWVRQAPGKGLEWVA
TISYDGSNAFYADSVEGRFTISRDNSKDSLYLQMDSLRPEDTAVYYCVK
AGDGGYDVFDSWGQGTLVTVSSASGGGGSGGGGSGGGGSEIVMTQSPLS
LPVTPGEPASISCRSSQSLLHSNGYNYLDWYLQKPGQSPQLLIYLGSNR
ASGVPDRFSGSGSGTDFTLKISRVEAEDVGVYYCMQALQTPTFGPGTKV
DIK

An illustrative targeting domain is CD33scFv-7, which an scFV specific to human CD33, and has the following sequence (the linker joining the variable regions of the heavy (V_{H}) and light chains (V_{L}) is shown by an underline):

(SEQ ID NO: 43)
EVQLVESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
ETDYYGSGTFDYWGQGTLVTVSSASGGGGGGGGSGGGGSDIQMTQSPSS
LSASVGDRVTISCRASQGIGIYLAWYQQRSGKPPQLLIHGASTLQSGVP
SRFSGSGSGTDFTLTISSLQPEDFASYWCQQSNNFPPTFGQGTKVEIK

An illustrative targeting domain is CD33scFv-9, which an scFV specific to human CD33, and has the following sequence (the linker joining the variable regions of the heavy (V_{H}) and light chains (V_{L}) is shown by an underline):

(SEQ ID NO: 44)
QVQLVQSGAEVKKPGESLKISCKGSGYSFTNYWIGWVRQMPGKGLEWMG
IIYPGDSDTRYSPSFQGQVTISADKSISTAYLQWSSLKASDTAMYYCAR
HGPSSWGEFDYWGQGTLVTVSSASGGGGSGGGGGGGGSDIRLTQSPSSL
SASVGDRVTITCRASQSISSYLNWYQQKPGKAPKLLIYAASSLQSGVPS
RFSGSGSGTDFTLTISSLQPEDFATYYCQQSYSTPLTFGGGTKVDIK

An illustrative targeting domain is CD33scFv-10, which an scFV specific to human CD33, and has the following sequence (the linker joining the variable regions of the heavy (V_{H}) and light chains (V_{L}) is shown by an underline):

(SEQ ID NO: 45)
EVQLVQSGAEVKKPGSSVKVSCKASGYTITDSNIHWVRQAPGQSLEWIG
YIYPYNGGTDYNQKFKNRATLTVDNPTNTAYMELSSLRSEDTAFYYCVN
GNPWLAYWGQGTLVTVSSASGGGGSGGGGSGGGGSDIQLTQSPSTLSAS
VGDRVTITCRASESLDNYGIRFLTWFQQKPGKAPKLLMYAASNQGSGVP
SRFSGSGSGTEFTLTISSLQPDDFATYYCQQTKEVPWSFGQGTKVEVK

An illustrative targeting domain is CD20scFv-1, which an scFV specific to human CD20, and has the following sequence (the variable regions of the heavy chain (V_{H}) is shown in a boldface font, the variable regions of the light chain (V_{L}) is indicated in an italics font, and the linker joining V_{H} and V_{L} is shown by an underline):

(SEQ ID NO: 46)
EVQLVESGGGLVQPGRSLRLSCVASGFTFNDYAMHWVRQAPGKGLEWVS
VISWNSDSIGYADSVKGRFTISRDNAKNSLYLQMHSLRAEDTALYYCAK
DNHYGSGSYYYYQYGMDVWGQGTTVTVSSGGGGSGGGGSGGGGSGGGGS
AEIVMTQSPATLSVSPGERATLSCRASQSVSSNLAWYQQKPGQAPRLLI
YGASTRATGIPARFSGSGSGTEFTLTISSLQSEDFAVYYCQHYINWPLT
FGGGTKVEIK

An illustrative targeting domain is CD20scFv-2, which an scFV specific to human CD20, and has the following sequence (the variable regions of the heavy clan (V_{H}) is shown in a boldface font, the variable regions of the light chain (V_{L}) is indicated in an italics font, and the linker joining V_{H} and V_{L} is shown by an underline):

(SEQ ID NO: 47)
EVQLVQSGAEVKKPGESLKISCKGSGRTFTSYNMHWVRQMPGKGLEWMG
AIYPLTGDTSYNQKSKLQVTISADKSISTAYLQWSSLKASDTAMYYCAR
STYVGGDWQFDVWGKGTTVTVSSGGGGSGGGGSGGGGSGGGGSEIVLTQ
SPGTLSLSPGERATLSCRASSSVPYIHWYQQKPGQAPRLLIYATSALAS
GIPDRFSGSGSGTDFTLTISRLEPEDFAVYYCQQWLSHPPTFGQGTKLE
IK

An illustrative targeting domain is CD20scFv-3, which an scFV specific to human CD20, and has the following sequence (the linker joining the variable regions of the heavy chain (V_{H}) and the variable regions of the light chain (V_{L}) is shown by an underline):

(SEQ ID NO: 48)
QVQLVQSGAEVKKPGASVKVSCKASGYTFTSYNMHWVRQAPGQGLEWMG
AIYPGNGDTSYNQKFQGRVTITADKSISTAYMELSSLRSEDTAVYYCAR
STYYGGDWYFNVWGAGTLVTVSSGGGGSGGGGSGGGGSGGGGSQIVLTQ
SPSSLSASVGDRVTITCRASSSVSYIHWFQQKPGKSPKPLIYATSNLAS
GVPVRFSGSGSGTDYTLTISSLQPEDFATYYCQQWTSNPPTFGGGTKVE
IK

An illustrative targeting domain is CD20scFv-4, which an scFV specific to human CD20, and has the following sequence (the linker joining the variable regions of the heavy clan (V_{H}) and the variable regions of the light chain (V_{L}) is shown by an underline):

(SEQ ID NO: 49)
EVQLVESGGGLVQPDRSLRLSCAASGFTFHDYAMHWVRQAPGKGLEWVS
TISWNSGTIGYADSVKGRFTISRDNAKNSLYLQMNSLRAEDTALYYCAK
DIQYGNYYYGMDVWGQGTTVTVSSGGGGSGGGGSGGGGSGGGGSEIVLT
QSPATLSLSPGERATLSCRASQSVSSYLAWYQQKPGQAPRLLIYDASNR
ATGIPARFSGSGSGTDFTLTISSLEPEDFAVYYCQQRSNWPITFGQGTR
LEIK

An illustrative targeting domain is GPRC5DscFv-1, which an scFV specific to human GPRC5D, and has the following sequence (the linker joining the variable regions of the heavy chain (V_{H}) and the variable regions of the light chain (V_{L}) is shown by an underline:

(SEQ ID NO: 50)
SSELTQDPAVSVALGQTVRITCQGDSLRSYYASWYQQKPGQAPVLVIYG
KNNRPSGIPDRFSGSSSGNTASLTITGAQAEDEADYYCNSRDSSGNPPV
VFGGGTKLTVLGSRGGGGSGGGGSGGGGSLEMAQVQLVESGGGLVHPGG
SLRLSCAASGFTFRSHSMNWVRQAPGKGLEWVSSISSDSTYTYYADSVK
GRFTISRDNAKNSLYLQMNSLRAEDTAVYYCARSGGQWKYYDYWGQGTL
VTVSS

An illustrative targeting domain is GPRC5DscFv-2, which an scFV specific to human GPRC5D, and has the following sequence (the linker joining the variable regions of the heavy clan (V_{H}) and the variable regions of the light chain (V_{L}) is shown by an underline):

(SEQ ID NO: 51)
QSVVTQPPSMSAAPGQQVTISCSGGNSNIERNYVSWYLQLPGTAPKLVI
FDNDRRPSGIPDRFSGSKSGTSATLGITGLQTGDEADYYCGTWDSSLRG
WVFGGGTKLTVLGSRGGGGSGGGGSGGGGSLEMAEVQLVESGGGLIQPG
GSLRLSCAASGFTFSNYAMNWVRQAPGKGLEWVSTINGRGSSTIYADSV
KGRFTISRDNSKNTLYLQMNSLRAEDTATYYCARYISRGLGDSWGQGTL
VTV

An illustrative targeting domain is Trop2-1_vHvL, which an scFV specific to human Trop2, and has the following sequence (the linker joining the variable regions of the heavy chain (V_{H}) and the variable regions of the light chain (V_{L}) is shown by an underline):

(SEQ ID NO: 52)
QVQLQQSGSELKKPGASVKVSCKASGYTFTNYGMNWVKQAPGQGLKWMG
WINTYTGEPTYTDDFKGRFAFSLDTSVSTAYLQISSLKADDTAVYFCAR
GGFGSSYWYFDVWGQGSLVTVSSGGGGSGGGGSGGGGSDIQLTQSPSSL
SASVGDRVSITCKASQDVSIAVAWYQQKPGKAPKLLIYSASYRYTGVPD
RFSGSGSGTDFTLTISSLQPEDFAVYYCQQHYITPLTFGAGTKVEIKR

An illustrative targeting domain is Trop2-1_vLvH, which an scFV specific to human Trop2, and has the following sequence (the linker joining the variable regions of the heavy chain (V_{H}) and the variable regions of the light chain (V_{L}) is shown by an underline):

(SEQ ID NO: 53)
DIQLTQSPSSLSASVGDRVSITCKASQDVSIAVAWYQQKPGKAPKLLIY
SASYRYTGVPDRFSGSGSGTDFTLTISSLQPEDFAVYYCQQHYITPLTF
GAGTKVEIKRGGGGSGGGGSGGGGSQVQLQQSGSELKKPGASVKVSCKA
SGYTFTNYGMNWVKQAPGQGLKWMGWINTYTGEPTYTDDFKGRFAFSLD
TSVSTAYLQISSLKADDTAVYFCARGGFGSSYWYFDVWGQGSLVTVSS

An illustrative targeting domain is Trop2-2_vHvL, which an scFV specific to human Trop2, and has the following sequence (the linker joining the variable regions of the heavy chain (V_{H}) and the variable regions of the light chain (V_{L}) is shown by an underline):

(SEQ ID NO: 54)
QIQLVQSGPELKKPGETVKISCKASGYTFTNYGMNWVKQAPGKGLKWMG
WINTKTGEPTYAEEFKGRFAFSLETSASTAYLQINNLKKEDTATYFCGR
GGYGSSYWYFDVWGAGTTVTVSSGGGGSGGGGSGGGGSDIVMTQSHKFM
STSVGDRVSITCKASQDVSIAVAWYQQKPGQSPKVLIYSASYRYTGVPD
RFTGSGSGTDFTFTISRVQAEDLAVYYCQQHYITPLTFGAGTKLELK

An illustrative targeting domain is Trop2-2_vLvH, which an scFV specific to human Trop2, and has the following sequence (the linker joining the variable regions of the heavy chain (V_{H}) and the variable regions of the light chain (V_{L}) is shown by an underline):

(SEQ ID NO: 55)
DIVMTQSHKFMSTSVGDRVSITCKASQDVSIAVAWYQQKPGQSPKVLIY
SASYRYTGVPDRFTGSGSGTDFTFTISRVQAEDLAVYYCQQHYITPLTF
GAGTKLELKGGGGSGGGGSGGGGSQIQLVQSGPELKKPGETVKISCKAS
GYTFTNYGMNWVKQAPGKGLKWMGWINTKTGEPTYAEEFKGRFAFSLET
SASTAYLQINNLKKEDTATYFCGRGGYGSSYWYFDVWGAGTTVTVSS

An illustrative targeting domain is CEACAM5-1_vHvL, which an scFV specific to human CEACAM5, and has the following sequence (the linker joining the variable regions of the heavy chain (V_{H}) and the variable regions of the light chain (V_{L}) is shown by an underline):

(SEQ ID NO: 56)
EVQLVESGGGVVQPGRSLRLSCSASGFDFTTYWMSWVRQAPGKGLEWIG
EIHPDSSTINYAPSLKDRFTISRDNAKNTLFLQMDSLRPEDTGVYFCAS
LYFGFPWFAYWGQGTPVTVSSGGGGSGGGGSGGGGSDIQLTQSPSSLSA
SVGDRVTITCKASQDVGTSVAWYQQKPGKAPKLLIYWTSTRHTGVPSRF
SGSGSGTDFTFTISSLQPEDIATYYCQQYSLYRSFGQGTKVEIKR

An illustrative targeting domain is CEACAM5-1_vLvH, which an scFV specific to human CEACAM5, and has the following sequence (the linker joining the variable regions of the heavy chain (V_{H}) and the variable regions of the light chain (V_{L}) is shown by an underline):

(SEQ ID NO: 57)
DIQLTQSPSSLSASVGDRVTITCKASQDVGTSVAWYQQKPGKAPKLLIY
WTSTRHTGVPSRFSGSGSGTDFTFTISSLQPEDIATYYCQQYSLYRSFG
QGTKVEIKRGGGGSGGGGSGGGGSEVQLVESGGGVVQPGRSLRLSCSAS
GFDFTTYWMSWVRQAPGKGLEWIGEIHPDSSTINYAPSLKDRFTISRDN
AKNTLFLQMDSLRPEDTGVYFCASLYFGFPWFAYWGQGTPVTVSS

An illustrative targeting domain is CEACAM5-2_vHvL, which an scFV specific to human CEACAM5, and has the following sequence (the linker joining the variable regions of the heavy chain (V_{H}) and the variable regions of the light chain (V_{L}) is shown by an underline):

(SEQ ID NO: 58)
EVQLQESGPGLVKPGGSLSLSCAASGFVFSSYDMSWVRQTPERRLEWVA
YISSGGGITYFPSTVKGRFTVSRDNAKNTLYLQMNSLTSEDTAIYYCAA
HYFGSSGPFAYWGQGTLVTVSAGGGGSGGGGSGGGGSDIQMTQSPASLS
ASVGDTVTITCRASENIFSYLAWYQQKPGKSPKLLVYNTKTLAEGVPSR
FSGSGSGTQFSLTISSLQPEDFGSYYCQHHYGTPFTFGSGTKLEIK

An illustrative targeting domain is CEACAM5-2_vLvH, which an scFV specific to human CEACAM5, and has the following sequence (the linker joining the variable regions of the heavy chain (V_{H}) and the variable regions of the light chain (V_{L}) is shown by an underline):

(SEQ ID NO: 59)
DIQMTQSPASLSASVGDTVTITCRASENIFSYLAWYQQKPGKSPKLLVY
NTKTLAEGVPSRFSGSGSGTQFSLTISSLQPEDFGSYYCQHHYGTPFTF
GSGTKLEIKGGGGSGGGGSGGGGSVQLQESGPGLVKPGGSLSLSCAAS
GFVFSSYDMSWVRQTPERRLEWVAYISSGGGITYFPSTVKGRFTVSRDN
AKNTLYLQMNSLTSEDTAIYYCAAHYFGSSGPFAYWGQGTLVTVSA

An illustrative targeting domain is CEACAM5-3_vHvL, which an scFV specific to human CEACAM5, and has the following sequence (the linker joining the variable regions of the heavy chain (V_{H}) and the variable regions of the light chain (V_{L}) is shown by an underline):

(SEQ ID NO: 60)
EVQLVESGGGLVQPGRSLRLSCAASGFTVSSYWMHWVRQAPGKGLEWVG
FIRNKANGGTTEYAASVKGRFTISRDDSKNTLYLQMNSLRAEDTAVYYC
ARDRGLRFYFDYWGQGTTVTVSSGGGGSGGGGSGGGGSQAVLTQPASLS
ASPGASASLTCTLRRGINVGAYSIYWYQQKPGSPPQYLLRYKSDSDKQQ
GSGVSSRFSASKDASANAGILLISGLQSEDEADYYCMIWHSGASAVFGG
GTKLTVL

An illustrative targeting domain is CEACAM5-3_vLvH, which an scFV specific to human CEACAM5, and has the following sequence (the linker joining the variable regions of the heavy chain (V_{H}) and the variable regions of the light chain (V_{L}) is shown by an underline):

(SEQ ID NO: 61)
QAVLTQPASLSASPGASASLTCTLRRGINVGAYSIYWYQQKPGSPPQYL
LRYKSDSDKQQGSGVSSRFSASKDASANAGILLISGLQSEDEADYYCMI
WHSGASAVFGGGTKLTVLGGGGSGGGGSGGGGSEVQLVESGGGLVQPGR
SLRLSCAASGFTVSSYWMHWVRQAPGKGLEWVGFIRNKANGGTTEYAAS
VKGRFTISRDDSKNTLYLQMNSLRAEDTAVYYCARDRGLRFYFDYWGQG
TTVTVSS

An illustrative targeting domain is CLL1-1_vHvL, which an scFV specific to human CLL1, and has the following sequence (the linker joining the variable regions of the heavy chain (V_{H}) and the variable regions of the light chain (V_{L}) is shown by an underline):

(SEQ ID NO: 62)
QVQLVQSGGGVVQPGRSLRLSCVASGFTFSSYGMHWVRQAPGKGLEWVA
AIWYNGRKQDYADSVKGRFTISRDNSKNTLYLQMNSLRAEDTAVYYCTR
GTGYNWFDPWGQGTLVTVSSGGGGSGGGGSGGGGSDIQMTQSPSSLSAS
VGDRVTITCRASQSISSYLNWYQQKPGKAPKLLIYAASSLQSGVPSRFS
GSGSGTDFTLTISSLQPEDFATYYCQQSYSTPPTFGQGTKVEIK

An illustrative targeting domain is CLL1-1_vLvH, which an scFV specific to human CLL1, and has the following sequence (the linker joining the variable regions of the heavy chain (V_{H}) and the variable regions of the light chain (V_{L}) is shown by an underline):

(SEQ ID NO: 63)
DIQMTQSPSSLSASVGDRVTITCRASQSISSYLNWYQQKPGKAPKLLIY
AASSLQSGVPSRFSGSGSGTDFTLTISSLQPEDFATYYCQQSYSTPPTF
GQGTKVEIKGGGGSGGGGSGGGGSQVQLVQSGGGVVQPGRSLRLSCVAS
GFTFSSYGMHWVRQAPGKGLEWVAAIWYNGRKQDYADSVKGRFTISRDN
SKNTLYLQMNSLRAEDTAVYYCTRGTGYNWFDPWGQGTLVTVSS

An illustrative targeting domain is CLL1-2_vHvL, which an scFV specific to human CLL1, and has the following sequence (the linker joining the variable regions of the heavy chain (V_{H}) and the variable regions of the light chain (V_{L}) is shown by an underline):

(SEQ ID NO: 64)
QVQLQESGPGLVKPSETLSLTCTVSGGSISSYYWSWIRQPPGKGLEWIG
YIYYSGSTNYNPSLKSRVTISVDTSKNQFSLKLSSVTAADTAVYYCVSL
VYCGGDCYSGFDYWGQGTLVTVSSGGGGSGGGGSGGGGSDIQLTQSPSS
LSASVGDRVSFTCQASQDINNFLNWYQQKPGKAPKLLIYDASNLETGVP
SRFSGSGSGTDFTFTISSLQPEDIATYYCQQYGNLPFTFGGGTKVEIKR

An illustrative targeting domain is CLL1-2_vLvH, which an scFV specific to human CLL1, and has the following sequence (the linker joining the variable regions of the heavy chain (V_{H}) and the variable regions of the light chain (V_{L}) is shown by an underline):

(SEQ ID NO: 65)
DIQLTQSPSSLSASVGDRVSFTCQASQDINNFLNWYQQKPGKAPKLLIY
DASNLETGVPSRFSGSGSGTDFTFTISSLQPEDIATYYCQQYGNLPFTF
GGGTKVEIKRGGGGSGGGGSGGGGSQVQLQESGPGLVKPSETLSLTCTV
SGGSISSYYWSWIRQPPGKGLEWIGYIYYSGSTNYNPSLKSRVTISVDT
SKNQFSLKLSSVTAADTAVYYCVSLVYCGGDCYSGFDYWGQGTLVTVSS

An illustrative targeting domain is ROR1-vHvL-1, which an scFV specific to human ROR1, and has the following sequence (the linker joining the variable regions of the heavy chain (V_{H}) and the variable regions of the light chain (V_{L}) is shown by an underline):

(SEQ ID NO: 66)
QVQLVQSGAEVKKPGASVKVSCKASGYTFTSYYMHWVRQAPGQGLEWMG
IINPNGGSTSYAQKFQGRVTMTRDTSTSTVYMELSSLRSEDTAVYYCAR
DSSYDAFDIWGQGTMVTVSSGGGGSGGGGSGGGGSAIQLTQSPSTLSAS
VGDRVTICQASQDISNYLNWYQQKPGKAPKLLINDASYLETGVPSRFSG
SGSGTDFTLTISSLQPEDIATYYCQQYESLPYTFGQGTKLEIK

An illustrative targeting domain is ROR1-vLvH-1, which an scFV specific to human ROR1, and has the following sequence (the linker joining the variable regions of the heavy chain (V_{H}) and the variable regions of the light chain (V_{L}) is shown by an underline):

(SEQ ID NO: 67)
AIQLTQSPSTLSASVGDRVTICQASQDISNYLNWYQQKPGKAPKLLIND
ASYLETGVPSRFSGSGSGTDFTLTISSLQPEDIATYYCQQYESLPYTFG
QGTKLEIKGGGGSGGGGSGGGGSQVQLVQSGAEVKKPGASVKVSCKASG
YTFTSYYMHWVRQAPGQGLEWMGIINPNGGSTSYAQKFQGRVTMTRDTS
TSTVYMELSSLRSEDTAVYYCARDSSYDAFDIWGQGTMVTVSS

An illustrative targeting domain is ROR1-vLvH-2, which an scFV specific to human ROR1, and has the following sequence (the linker joining the variable regions of the heavy chain (V_{H}) and the variable regions of the light chain (V_{L}) is shown by an underline):

(SEQ ID NO: 68)
QVTLKESGGGLVKPGGSLRLSCAASGFTFSNAWMSWVRQAPGKGLEWVG
RIKSKTDGGTTDYAAPVKGRFTISRDDSKNTLYLQMNSLKTEDTAVYYC
ARDFGRWSYYFDYWSQGTLVTVSSGGGGSGGGGGGGGSQSVLTQPSSVS
GTPGQRVTISCSGSSSNIGSNYVYWYQQLPGTAPKWYRNNQRPSGVPDR
FSGSKSGTSASLAISGLRSEDEADYYCAAWDDSLSGVVFGGGTKLTVL

An illustrative targeting domain is ROR1-vHvL-2, which an scFV specific to human ROR1, and has the following sequence (the linker joining the variable regions of the heavy chain (V_{H}) and the variable regions of the light chain (V_{L}) is shown by an underline):

(SEQ ID NO: 69)
QSVLTQPSSVSGTPGQRVTISCSGSSSNIGSNYVYWYQQLPGTAPKWYR
NNQRPSGVPDRFSGSKSGTSASLAISGLRSEDEADYYCAAWDDSLSGVV
FGGGTKLTVLGGGGSGGGGSGGGGSQVTLKESGGGLVKPGGSLRLSCAA
SGFTFSNAWMSWVRQAPGKGLEWVGRIKSKTDGGTTDYAAPVKGRFTIS
RDDSKNTLYLQMNSLKTEDTAVYYCARDFGRWSYYFDYWSQGTLVTVSS

An illustrative targeting domain is ROR1-vHvL-3, which an scFV specific to human ROR1, and has the following sequence (the linker joining the variable regions of the heavy chain (V_{H}) and the variable regions of the light chain (V_{L}) is shown by an underline):

(SEQ ID NO: 70)
EVQLVESGGGLVQPGRSLRLSCTASGFTFGDYAMSWVRQAPGKGLEWVS
SISGSGRSTDHADYVKGRFTISRDNSKNTVYLQMNRLRAEDTAVYYCAK
VSNYEYYFDYWAQGTLTVSSGGGGSGGGGSGGGGSEIVLTQSPSVSVAP
GQTARITCGGSNIGSESVNWYQWKSGQVPVLVVSDTTDPRSGIPGRFTG
TRSGTTATLTISGVEAGDEADYHCQVWDDTGDHPVFGGGTKLTVL

An illustrative targeting domain is ROR1-vLvH-3, which an scFV specific to human ROR1, and has the following sequence (the linker joining the variable regions of the heavy chain (V_{H}) and the variable regions of the light chain (V_{L}) is shown by an underline):

(SEQ ID NO: 71)
EIVLTQSPSVSVAPGQTARITCGGSNIGSESVNWYQWKSGQVPVLVVSD
TTDPRSGIPGRFTGTRSGTTATLTISGVEAGDEADYHCQVWDDTGDHPV
FGGGTKLTVLGGGGSGGGGSGGGGSEVQLVESGGGLVQPGRSLRLSCTA
SGFTFGDYAMSWVRQAPGKGLEWVSSISGSGRSTDHADYVKGRFTISRD
NSKNTVYLQMNRLRAEDTAVYYCAKVSNYEYYFDYWAQGTLTVSS

An illustrative targeting domain is B7H3scFv, which an scFV specific to human B7H3, and has the following sequence (the linker joining the variable regions of the heavy chain (V_{H}) and the variable regions of the light chain (V_{L}) is shown by an underline):

(SEQ ID NO: 111)
DIQMTQSPSFLSASVGDRVTITCKASQNVDTNVAWYQQKPGKAPKALIY
SASYRYSGVPSRFSGSGSGTDFTLTISSLQPEDFAEYFCQQYNNYPFTF
GQGTKLEIKSSGGGGSGGGGSGGGGSEVQLVESGGGLVQPGGSLRLSCA
ASGFTFSSFGMHWVRQAPGKGLEWVAYISSGSGTIYYADTVKGRFTISR
DNAKNSLYLQMNSLRAEDTAVYYCARHGYRYEGFDYWGQGTTVTVSS

An illustrative targeting domain is FAPscFv, which an scFV specific to human FAP, and has the following sequence (the linker joining the variable regions of the heavy chain (V_{H}) and the variable regions of the light chain (V_{L}) is shown by an underline):

(SEQ ID NO: 112)
EVQLLESGGGLVQPGGSLRLSCAASGFTFSSHAMSWVRQAPGKGLEWVS
AIWASGEQYYADSVKGRFTISRDNSKNTLYLQMNSLRAEDTAVYYCAKG
WLGNFDYWGQGTLVTVSSGGGGSGGGGSGGGGSEIVLTQSPGTLSLSPG
ERATLSCRASQSVSRSYLAWYQQKPGQAPRLLIIGASTRATGIPDRFSG
SGSGTDFTLTISRLEPEDFAVYYCQQGQVIPPTFGQGTKVEIK

The Linker Domain that Adjoins the First and the Second Domain

In embodiments, the linker that adjoins the first and second domains comprises a charge polarized core domain. In various embodiments, each of the first and second charge polarized core domains comprises proteins having positively or negatively charged amino acid residues at the amino and carboxy terminus of the core domain. In an illustrative embodiment, the first charge polarized core domain may comprise a protein having positively charged amino acids at the amino terminus which are adjoined by a linker (e.g., a stabilizing domain) to a protein having negatively charged amino acid residues at the carboxy terminus. The second charge polarized core domain may comprise a protein having negatively charged amino acids at the amino terminus which are adjoined by a linker (e.g., a stabilizing domain) to a protein having positively charged amino acid residues at the carboxy terminus.

In another illustrative embodiment, the first charge polarized core domain may comprise a protein having negatively charged amino acids at the amino terminus which are adjoined by a linker (e.g., a stabilizing domain) to a protein having positively charged amino acid residues at the carboxy terminus. The second charge polarized core domain may comprise proteins having positively charged amino acids at the amino terminus which are adjoined by a linker (e.g., a stabilizing domain) to a protein having negatively charged amino acid residues at the carboxy terminus.

In various embodiments, formation of heterodimeric proteins is driven by electrostatic interactions between the positively charged and negatively charged amino acid residues located at the amino and carboxy termini of the first and second charge polarized core domains. Further, formation of homodimeric proteins is prevented by the repulsion between the positively charged amino acid residues or negatively charged amino acid residues located at the amino and carboxy termini of the first and second charge polarized core domains.

In various embodiments, the protein comprising positively and/or negatively charged amino acid residues at the amino or carboxy terminus of the charge polarized core domains is about 2 to about 50 amino acids long.

For example, the protein comprising positively and/or negatively charged amino acid residues at either terminus of the charge polarized core domain may be about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In various embodiments, the protein comprising positively charged amino acid residues may include one or more of amino acids selected from His, Lys, and Arg. In various embodiments, the protein comprising negatively charged amino acid residues may include one or more amino acids selected from Asp and Glu.

In various embodiments, each of the first and/or second charge polarized core domains may comprise a protein comprising an amino acid sequence as provided in the Table below or an amino acid sequence having at least 90%, or 93%, or 95%, or 97%, or 98%, or 99% identity thereto.

| SEQ ID | |
|---|---|
| NO. | Sequence |
| 1 | YₙXₙYₙXₙYₙ (where X is a positively charged amino acid such as arginine, histidine |
| | or lysine and Y is a spacer amino acid such as serine or glycine, and where each n |
| | is independently an integer 0 to 4) |
| 2 | YₙZₙYₙZₙYₙ (where Z is a negatively charged amino acid such as aspartic acid or |
| | glutamic acid and Y is a spacer amino acid such as serine or glycine, and where |
| | each n is independently an integer 0 to 4) |
| 3 | YYₙXXₙYYₙXXₙYYₙ (where X is a positively charged amino acid such as arginine, |
| | histidine or lysine and Y is a spacer amino acid such as serine or glycine, and |
| | where each n is independently an integer 0 to 4) |
| 4 | YYₙZZₙYYₙZZₙYYₙ (where Z is a negatively charged amino acid such as aspartic |
| | acid or glutamic acid and Y is a spacer amino acid such as serine or glycine, and |
| | where each n is independently an integer 0 to 4) |
| 5 | YₙXₙCYₙXₙYₙ (where X is a positively charged amino acid such as arginine, |
| | histidine or lysine and Y is a spacer amino acid such as serine or glycine, and |
| | where each n is independently an integer 0 to 4) |
| 6 | YₙZₙCYₙZₙYₙ (where Z is a negatively charged amino acid such as aspartic acid or |
| | glutamic acid and Y is a spacer amino acid such as serine or glycine, and where |
| | each n is independently an integer 0 to 4) |
| 7 | GSGSRKGGKRGS |
| 8 | GSGSRKCGKRGS |
| 9 | GSGSDEGGEDGS |
| 10 | GSGSDECGEDGS |

For example, in an embodiment, each of the first and second charge polarized core domains may comprise a peptide comprising the sequence YYₙXXₙYYₙXXₙYYₙ (where X is a positively charged amino acid such as arginine, histidine or lysine and Y is a spacer amino acid such as serine or glycine, and where each n is independently an integer 0 to 4; SEQ ID NO: 3). Illustrative peptide sequences include, but are not limited to, RKGGKR (SEQ ID NO: 11) or GSGSRKGGKRGS (SEQ ID NO: 12).

In another illustrative embodiment, each of the first and second charge polarized core domains may comprise a peptide comprising the sequence YYₙZZₙYYₙZZₙYYₙ (where Z is a negatively charged amino acid such as aspartic acid or glutamic acid and Y is a spacer amino acid such as serine or glycine, and where each n is independently an integer 0 to 4). Illustrative peptide sequences include, but are not limited to, DEGGED (SEQ ID NO: 13) or GSGSDEGGEDGS (SEQ ID NO: 14).

In one aspect, the current disclosure provides a heterodimeric protein comprising (a) a first domain comprising one or more butyrophilin family proteins, or a fragment thereof; (b) a second domain comprising a targeting domain, the targeting domain being selected from an (i) antibody, antibody-like molecule, or antigen binding fragment thereof, and (ii) a extracellular domain; and (c) a linker that adjoins the first and second domains. In embodiments, the heterodimeric protein comprises two individual polypeptide chains which self-associate. In embodiments, the linker facilitates heterodimerization. In embodiments, the heterodimeric protein comprises two of the same butyrophilin family proteins or two different butyrophilin family proteins. In embodiments, the butyrophilin family proteins comprise a V-type domain and/or a B30.2 domain. In embodiments, the first domain is a butyrophilin-like (BTNL) family protein, such as BTN2A1, BTN3A1, and a fragment thereof.

In embodiments, the first polypeptide chain and the second polypeptide chain heterodimers through electrostatic interactions between positively charged amino acid residues and negatively charged amino acid residues on the first and second charge polarized core domains. In embodiments, the positively charged amino acid residues may include one or more of amino acids selected from His, Lys, and Arg. In embodiments, the negatively charged amino acid residues may include one or more amino acids selected from Asp and Glu.

Accordingly, In embodiments, each of the first and/or second charge polarized core domains comprises proteins having positively or negatively charged amino acid residues at the amino and carboxy terminus of the core domain. In an illustrative embodiment, the first charge polarized core domain may comprise a protein having positively charged amino acids at the amino terminus which are adjoined by a linker (e.g., a stabilizing domain) to a protein having negatively charged amino acid residues at the carboxy terminus. In such an embodiment, the second charge polarized core domain may comprise a protein having negatively charged amino acids at the amino terminus which are adjoined by a linker (e.g., a stabilizing domain) to a protein having positively charged amino acid residues at the carboxy terminus. In another illustrative embodiment, the first charge polarized core domain may comprise a protein having negatively charged amino acids at the amino terminus which are adjoined by a linker (e.g., a stabilizing domain) to a protein having positively charged amino acid residues at the carboxy terminus. In such an embodiment, the second charge polarized core domain may comprise proteins having positively charged amino acids at the amino terminus which are adjoined by a linker (e.g., a stabilizing domain) to a protein having negatively charged amino acid residues at the carboxy terminus.

In various embodiments, each of the first and/or second charge polarized core domains further comprise a linker (e.g., a stabilizing domain) which adjoins the proteins having positively or negatively charged amino acids. In embodiments, the linker (e.g., a stabilizing domain) is optionally selected from a flexible amino acid sequence, IgG hinge region, or antibody sequence. In an embodiment, the linker (e.g., a stabilizing domain) comprises the hinge-CH2-CH3 Fc domain derived from IgG1, optionally human IgG1. In another embodiment, the linker (e.g., a stabilizing domain) comprises the hinge-CH2-CH3 Fc domain derived from IgG4, optionally human IgG4.

Illustrative sequences of linkers that adjoins the first and second domains, also referred to herein as a core domain are provided below:

In embodiments, the core domain comprises the following sequence:

(SEQ ID NO: 15)
SKYGPPCPPCPAPEFLGGPSVFLFPPKPKDQLMISRTPEVTCVVVDVSQ
EDPEVQFNWYVDGVEVHNAKTKPREEQFNSTYRVVSVLTVLHQDWLSGK
EYKCKVSSKGLPSSIEKTISNATGQPREPQVYTLPPSQEEMTKNQVSLT
CLVKGFYPSDIAVEWESNGQPENNYKTTPPVLDSDGSFFLYSRLTVDKS
RWQEGNVFSCSVLHEALHNHYTQKSLSLSLGKIEGRMD.

The sequence of an illustrative charge polarized core domain (positive-negative) is provided below (peptide comprising positively charged amino acids is shown with an underline, peptide comprising negatively charged amino acids is shown with italic font and IgG4 hinge-CH2-CH3 is shown in boldface font. Rest of the sequences are joining linkers disclosed herein):

(SEQ ID NO: 16)
GSGSRKGGKRGSKYGPPCPPCPAPEFLGGPSVFLFPPKPKDQLMISRTP
EVTCVVVDVSQEDPEVQFNWYVDGVEVHNAKTKPREEQFNSTYRVVSVL
TVLHQDWLSGKEYKCKVSSKGLPSSIEKTISNATGQPREPQVYTLPPSQ
EEMTKNQVSLTCLVKGFYPSDIAVEWESNGQPENNYKTTPPVLDSDGSF
FLYSRLTVDKSRWQEGNVFSCSVLHEALHNHYTQKSLSLSLGKDEGGED
GSGS.

The sequence of an illustrative charge polarized core domain (negative-positive) is provided below (peptide comprising positively charged amino acids is shown with an underline, peptide comprising negatively charged amino acids is shown with italic font and IgG4 hinge-CH2-CH3 is shown in boldface font. Rest of the sequences are joining linkers disclosed herein):

(SEQ ID NO: 17)
GSGSDEGGEDGSKYGPPCPPCPAPEFLGGPSVFLFPPKPKDQLMISRTP
EVTCVVVDVSQEDPEVQFNWYVDGVEVHNAKTKPREEQFNSTYRVVSVL
TVLHQDWLSGKEYKCKVSSKGLPSSIEKTISNATGQPREPQVYTLPPSQ
EEMTKNQVSLTCLVKGFYPSDIAVEWESNGQPENNYKTTPPVLDSDGSF
FLYSRLTVDKSRWQEGNVFSCSVLHEALHNHYTQKSLSLSLGKRKGGKR
GSGS.

In embodiments, the core domain comprises the following sequence (IgG4 hinge-CH2-CH3 is shown in boldface font, rest of the sequence is a joining linker disclosed herein):

(SEQ ID NO: 18)
CPPCPAPEFLGGPSVFLFPPKPKDQLMISRTPEVTCVVVDVSQEDPEVQ
FNWYVDGVEVHNAKTKPREEQFNSTYRVVSVLTVLHQDWLSGKEYKCKV
SSKGLPSSIEKTISNATGQPREPQVYTLPPSQEEMTKNQVSLTCLVKGF
YPSDIAVEWESNGQPENNYKTTPPVLDSDGSFFLYSRLTVDKSRWQEGN
VFSCSVLHEALHNHYTQKSLSLSLGK.

In embodiments, the core domain comprises a KIHT22Y protein comprising the following sequence (the knob in hole motif mutations are indicated by boldface, underlined font):

(SEQ ID NO: 19)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLYCLVKGFYPSDIAVEWESNGQPENNYKTTPPVLDSDGSFFLYSKLT
VDKSRWQQGNVFSCSVMHEALHNHYTQKSLSLSPGK.

In embodiments, the core domain is a KIHY86T protein comprising the following sequence (the knob in hole motif mutations are indicated by boldface, underlined font):

(SEQ ID NO: 20)
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLTSKLT
VDKSRWQQGNVFSCSVMHEALHNHYTQKSLSLSPGK .

In embodiments, the core domain comprises a IgG1 hinge-CH2-CH3 protein comprising the following sequence:

(SEQ ID NO: 21)
VPRDCGCKPCICTVPEVSSVFIFPPKPKDVLTITLTPKVTCVVVDISKD
DPEVQFSWFVDDVEVHTAQTQPREEQFNSTFRSVSELPIMHQDWLNGKE
FKCRVNSAAFPAPIEKTISKTKGRPKAPQVYTIPPPKEQMAKDKVSLTC
MITDFFPEDITVEWQWNGQPAENYKNTQPIMDTDGSYFVYSKLNVQKSN
WEAGNTFTCSVLHEGLHNHHTEKSLSHSPGI.

In embodiments, the core domain comprises the following sequence (IgG4 hinge-CH2-CH3 is shown in boldface font, rest of the sequence is a joining linker disclosed herein):

(SEQ ID NO: 22)
CPPCPAPEFLGGPSVFLFPPKPKDQLMISRTPEVTCVVVDVSQEDPEVQ
FNWYVDGVEVHNAKTKPREEQFNSTYRVVSVLTVLHQDWLSGKEYKCKV
SSKGLPSSIEKTISNATGQPREPQVYTLPPSQEEMTKNQVSLTCLVKGF
YPSDIAVEWESNGQPENNYKTTPPVLDSDGSFFLYSRLTVDKSRWQEGN
VFSCSVLHEALHNHYTQKSLSLSLGK.

The sequence of an illustrative Fc domains containing knob-in-hole (KIH) mutations are provided below:

(SEQ ID NO: 23)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPCRDELTKNQ
VSLWCLVKGFYPSDIAVEWESNGQPENNYKTTPPVLDSDGSFFLYSKLT
VDKSRWQQGNVFSCSVMHEALHNHYTQKSLSLSPGKIEGRMD.

The sequence of an illustrative Fc domains containing knob-in-hole (KIH) mutations are provided below:

(SEQ ID NO: 24)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVCTLPPSRDELTKNQ
VSLSCAVKGFYPSDIAVEWESNGQPENNYKTTPPVLDSDGSFFLVSKLT
VDKSRWQQGNVFSCSVMHEALHNHYTQKSLSLSPGKIEGRMD.

The sequence of an illustrative Fc domains containing knob-in-hole (KIH) mutations and FcRn mutations are provided below:

(SEQ ID NO: 25)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPCRDELTKNQ
VSLWCLVKGFYPSDIAVEWESNGQPENNYKTTPPVLDSDGSFFLYSKLT
VDKSRWQQGNVFSCSVLHEALHSHYTQKSLSLSPGKIEGRMD.

The sequence of an illustrative Fc domains containing knob-in-hole (KIH) mutations and FcRn mutations are provided below:

(SEQ ID NO: 26)
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVCTLPPSRDELTKNQ
VSLSCAVKGFYPSDIAVEWESNGQPENNYKTTPPVLDSDGSFFLVSKLT
VDKSRWQQGNVFSCSVLHEALHSHYTQKSLSLSPGKIEGRMD.

In various embodiments, the protein comprising the charged amino acid residues may further comprise one or more cysteine residues to facilitate disulfide bonding between the electrostatically charged core domains as an additional method to stabilize the heterodimer.

In various embodiments, each of the first and second charge polarized core domains comprises a linker sequence which may optionally function as a stabilizing domain. In various embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker (e.g., a stabilizing domain) is a synthetic linker such as PEG.

In other embodiments, the linker (e.g., a stabilizing domain) is a polypeptide. In embodiments, the linker (e.g., a stabilizing domain) is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker (e.g., a stabilizing domain) may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long.

In various embodiments, the linker (e.g., a stabilizing domain) is substantially comprised of glycine and serine residues (e.g., about 30%, or about 40%, or about 50%, or about 60%, or about 70%, or about 80%, or about 90%, or about 95%, or about 97% glycines and serines).

In various embodiments, the linker (e.g., a stabilizing domain) is a hinge region of an antibody (e.g., of IgG, IgA, IgD, and IgE, inclusive of subclasses (e.g., IgG1, IgG2, IgG3, and IgG4, and IgA1 and IgA2). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In other embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence Cys-Pro-Pro-Cys which, when dimerized by disulfide bond formation, results in a cyclic octapeptide believed to act as a pivot, thus conferring flexibility. In various embodiments, the present linker (e.g., a stabilizing domain) comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In various embodiments, the linker (e.g., a stabilizing domain) of the current disclosure comprises one or more glycosylation sites.

In various embodiments, the linker (e.g., a stabilizing domain) comprises an Fc domain of an antibody (e.g., of IgG, IgA, IgD, and IgE, inclusive of subclasses (e.g., IgG1, IgG2, IgG3, and IgG4, and IgA1 and IgA2)). In various embodiments, the linker (e.g., a stabilizing domain) comprises a hinge-CH2-CH3 Fc domain derived from a human IgG4 antibody. In various embodiments, the linker (e.g., a stabilizing domain) comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present heterodimeric proteins.

In embodiments, the Fc domain contains one or more amino acid substitutions at amino acid residue 250, 252, 254, 256, 308, 309, 311, 428, 433 or 434 (in accordance with Kabat numbering), or equivalents thereof.

In an embodiment, the amino acid substitution at amino acid residue 250 is a substitution with glutamine. In an embodiment, the amino acid substitution at amino acid residue 252 is a substitution with tyrosine, phenylalanine, tryptophan or threonine. In an embodiment, the amino acid substitution at amino acid residue 254 is a substitution with threonine. In an embodiment, the amino acid substitution at amino acid residue 256 is a substitution with serine, arginine, glutamine, glutamic acid, aspartic acid, orthreonine. In an embodiment, the amino acid substitution at amino acid residue 308 is a substitution with threonine. In an embodiment, the amino acid substitution at amino acid residue 309 is a substitution with proline. In an embodiment, the amino acid substitution at amino acid residue 311 is a substitution with serine. In an embodiment, the amino acid substitution at amino acid residue 385 is a substitution with arginine, aspartic acid, serine, threonine, histidine, lysine, alanine or glycine. In an embodiment, the amino acid substitution at amino acid residue 386 is a substitution with threonine, proline, aspartic acid, serine, lysine, arginine, isoleucine, or methionine. In an embodiment, the amino acid substitution at amino acid residue 387 is a substitution with arginine, proline, histidine, serine, threonine, or alanine. In an embodiment, the amino acid substitution at amino acid residue 389 is a substitution with proline, serine or asparagine. In an embodiment, the amino acid substitution at amino acid residue 428 is a substitution with leucine. In an embodiment, the amino acid substitution at amino acid residue 433 is a substitution with arginine, serine, isoleucine, proline, or glutamine. In an embodiment, the amino acid substitution at amino acid residue 434 is a substitution with histidine, phenylalanine, or tyrosine.

In embodiments, the Fc domain (e.g., comprising an IgG constant region) comprises one or more mutations such as substitutions at amino acid residue 252, 254, 256, 433, 434, or 436 (in accordance with Kabat numbering). In an embodiment, the IgG constant region includes a triple M252Y/S254T/T256E mutation or YTE mutation. In another embodiment, the IgG constant region includes a triple H433K/N434F/Y436H mutation or KFH mutation. In a further embodiment, the IgG constant region includes an YTE and KFH mutation in combination.

In embodiments, the modified humanized antibodies of the invention comprise an IgG constant region that contains one or more mutations at amino acid residues 250, 253, 307, 310, 380, 428, 433, 434, and 435. Illustrative mutations include T250Q, M428L, T307A, E380A, 1253A, H310A, M428L, H433K, N434A, N434F, N434S, and H435A. In an embodiment, the IgG constant region comprises a M428L/N434S mutation or LS mutation. In another embodiment, the IgG constant region comprises a T250Q/M428L mutation or QL mutation. In another embodiment, the IgG constant region comprises an N434A mutation. In another embodiment, the IgG constant region comprises a T307A/E380A/N434A mutation or AAA mutation. In another embodiment, the IgG constant region comprises an 1253A/H310A/H435A mutation or IHH mutation. In another embodiment, the IgG constant region comprises a H433K/N434F mutation. In another embodiment, the IgG constant region comprises a M252Y/S254T/T256E and a H433K/N434F mutation in combination.

In various embodiments, mutations are introduced to increase stability and/or half-life of the Fc domain. An illustrative Fc stabilizing mutant is S228P. Additional illustrative Fc half-life extending mutants are T250Q, M428L, V308T, L309P, and Q311S and the present linkers (e.g., stabilizing domains) may comprise 1, or 2, or 3, or 4, or 5 of these mutants.

Additional illustrative mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al., Nature (2014) 514:642-645, Grevys et aL, Journal of Immunology. (2015), 194(11):5497-508, and U.S. Pat. No. 7,083,784, the entire contents of which are hereby incorporated by reference.

In various embodiments, the linker may be flexible, including without limitation highly flexible. In various embodiments, the linker may be rigid, including without limitation a rigid alpha helix.

In various embodiments, the linker may be functional. For example, without limitation, the linker may function to improve the folding and/or stability, improve the expression, improve the pharmacokinetics, and/or improve the bioactivity of the present heterodimeric protein. In another example, the linker may function to target the heterodimeric protein to a particular cell type or location.

### The Heterodimeric Protein

In one aspect, the heterodimeric proteins suitable to the methods disclosed herein comprise an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the 15 first and second domains.

In any of the embodiments disclosed herein, the BTN2A1 protein, or a fragment thereof comprises a variable Ig-like V-type domain. In embodiments, the variable Ig-like V-type domain of the BTN2A1 protein comprises an amino acid sequence that is at least 90%, or 93%, or 95%, or 97%, or 98%, or 99% identical to the amino acid sequence of SEQ ID NO: 28. In embodiments, the BTN2A1 protein, or a fragment thereof comprise a extracellular domain (ECD). In embodiments, the variable ECD of the BTN2A1 protein comprises an amino acid sequence that is at least 90%, or 93%, or 95%, or 97%, or 98%, or 99% identical to the amino acid sequence of SEQ ID NO: 27.

In any of the embodiments disclosed herein, the BTN3A1 protein, or a fragment thereof comprise a variable Ig-like V-type domain. In embodiments, the variable Ig-like V-type domain of the BTN3A1 protein comprises an amino acid sequence that is at least 90%, or 93%, or 95%, or 97%, or 98%, or 99% identical to the amino acid sequence of SEQ ID NO: 30. In embodiments, the BTN3A1 protein, or a fragment thereof comprise a extracellular domain (ECD). In embodiments, variable ECD of the BTN3A1 protein comprises an amino acid sequence that is at least 90%, or 93%, or 95%, or 97%, or 98%, or 99% identical to the amino acid sequence of SEQ ID NO: 29.

In embodiments, the first domain comprises an amino acid sequence having an amino acid sequence of selected from one or more of SEQ ID NOs: 27-30.

In any of the embodiments disclosed herein, the targeting domain is an antibody, or antigen binding fragment thereof. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In embodiments, the antibody-like molecule is an scFv.

Additionally or alternatively, in embodiments, the targeting domain is an extracellular domain. In embodiments, the targeting domain is capable of binding an antigen on the surface of a cancer cell. In embodiments, the targeting domain specifically binds one or more of CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, and GD3. In embodiments, the targeting domain comprises a portion of the extracellular domain of LAG-3, PD-1, TIGIT, CD19, and PCMA. In embodiments, the targeting domain comprises an scFv that specifically binds CD19, PSMA, B7H3, FAP, CD20 or CD33. In embodiments, the targeting domain specifically binds CD19. In embodiments, the targeting domain specifically binds PSMA. In embodiments, the targeting domain specifically binds CD33. In embodiments, the targeting domain specifically binds CD20. In embodiments, the targeting domain specifically binds GPRC5D. In embodiments, the targeting domain specifically binds TROP-2. In embodiments, the targeting domain specifically binds CEACAM5. In embodiments, the targeting domain specifically binds CLL1. In embodiments, the targeting domain specifically binds ROR1. In embodiments, the targeting domain specifically binds B7H3. In embodiments, the targeting domain specifically binds FAP.

In embodiments, the targeting domain is a polypeptide having an amino acid sequence with at least 90%, or 95%, or 97%, or 98%, or 99% identity with a polypeptide selected from SEQ ID NOs: 31-71, 111 and 112. In embodiments, the targeting domain is a polypeptide having an amino acid sequence of selected from one or more of SEQ ID NOs: 31-71, 111 and 112. In embodiments, the targeting domain is a polypeptide having an amino acid sequence with at least 90%, or 95%, or 97%, or 98%, or 99% identity with a polypeptide selected from SEQ ID NOs: 31-38, 41, 48, 111 and 112. In embodiments, the targeting domain is a polypeptide having an amino acid sequence of selected from one or more of SEQ ID NOs: 31-38, 41, 48, 111 and 112. In embodiments, the targeting domain is a polypeptide having an amino acid sequence with at least 90%, or 95%, or 97%, or 98%, or 99% identity with a polypeptide selected from SEQ ID NOs: 39-45. In embodiments, the targeting domain is a polypeptide having an amino acid sequence of selected from one or more of SEQ ID NOs: 39-45. In embodiments, the targeting domain is a polypeptide having an amino acid sequence with at least 90%, or 95%, or 97%, or 98%, or 99% identity with a polypeptide selected from SEQ ID NOs: 46-49. In embodiments, the targeting domain is a polypeptide having an amino acid sequence of selected from one or more of SEQ ID NOs: 46-49. In embodiments, the targeting domain is a polypeptide having an amino acid sequence with at least 90%, or 95%, or 97%, or 98%, or 99% identity with a polypeptide selected from SEQ ID NOs: 50-51. In embodiments, the targeting domain is a polypeptide having an amino acid sequence of selected from one or more of SEQ ID NOs: 50-51. In embodiments, the targeting domain is a polypeptide having an amino acid sequence with at least 90%, or 95%, or 97%, or 98%, or 99% identity with a polypeptide selected from SEQ ID NOs: 52-55. In embodiments, the targeting domain is a polypeptide having an amino acid sequence of selected from one or more of SEQ ID NOs: 52-55. In embodiments, the targeting domain is a polypeptide having an amino acid sequence with at least 90%, or 95%, or 97%, or 98%, or 99% identity with a polypeptide selected from SEQ ID NOs: 56-61. In embodiments, the targeting domain is a polypeptide having an amino acid sequence of selected from one or more of SEQ ID NOs: 56-61. In embodiments, the targeting domain is a polypeptide having an amino acid sequence with at least 90%, or 95%, or 97%, or 98%, or 99% identity with a polypeptide selected from SEQ ID NOs: 62-65. In embodiments, the targeting domain is a polypeptide having an amino acid sequence of selected from one or more of SEQ ID NOs: 62-65. In embodiments, the targeting domain is a polypeptide having an amino acid sequence with at least 90%, or 95%, or 97%, or 98%, or 99% identity with a polypeptide selected from SEQ ID NOs: 66-71. In embodiments, the targeting domain is a polypeptide having an amino acid sequence of selected from one or more of SEQ ID NOs: 66-71. In embodiments, the targeting domain is a polypeptide having an amino acid sequence with at least 90%, or 95%, or 97%, or 98%, or 99% identity with a polypeptide having the amino acid sequence of SEQ ID NO: 41. In embodiments, the targeting domain is a polypeptide having the amino acid sequence of SEQ ID NO: 41. In embodiments, the targeting domain is a polypeptide having an amino acid sequence with at least 90%, or 95%, or 97%, or 98%, or 99% identity with a polypeptide having the amino acid sequence of SEQ ID NO: 46. In embodiments, the targeting domain is a polypeptide having the amino acid sequence of SEQ ID NO: 46. In embodiments, the targeting domain is a polypeptide having an amino acid sequence with at least 90%, or 95%, or 97%, or 98%, or 99% identity with a polypeptide having the amino acid sequence of SEQ ID NO: 111. In embodiments, the targeting domain is a polypeptide having the amino acid sequence of SEQ ID NO: 111. In embodiments, the targeting domain is a polypeptide having an amino acid sequence with at least 90%, or 95%, or 97%, or 98%, or 99% identity with a polypeptide having the amino acid sequence of SEQ ID NO: 112. In embodiments, the targeting domain is a polypeptide having the amino acid sequence of SEQ ID NO: 112.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains.

In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally from human IgG1. In embodiments, the linker comprises the hinge-CH2-CH3 Fc domain derived from IgG4, optionally from human IgG4.

In embodiments, the first and/or second charge polarized core domain further comprise peptides having positively and/or negatively charged amino acid residues at the amino and/or carboxy terminus of the charge polarized core domain. In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains. In embodiments, the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5). In embodiments, the peptide comprising positively charged amino acid residues comprises the sequence RKGGKR (SEQ ID NO: 11) or GSGSRKGGKRGS (SEQ ID NO: 12).

In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine, and where each n is independently an integer 0 to 4) (SEQ ID NO: 6). In embodiments, the peptide comprising negatively charged amino acid residues comprises the sequence DEGGED (SEQ ID NO: 13) or GSGSDEGGEDGS (SEQ ID NO: 14).

In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120.

In embodiments, the heterodimeric protein is substituted with a homodimeric protein comprising (a) (i) an extracellular domain of a BTN2A1 protein, or a fragment thereof, and (ii) an extracellular domain of a BTN3A1 protein or a fragment thereof; (b) a second domain comprising a targeting domain, and (c) a linker. in embodiments, the fragment is the variable domain of BTN2A1 and/or BTN3A1. In embodiments, linker comprises a CH2-CH3-Fc domain. In embodiments, the linker comprises a CH2-CH3-Fc domain of an IgG1 antibody or an IgG4 antibody. In embodiments, the IgG1 antibody is a human IgG1 antibody. In embodiments, the IgG4 antibody is a human IgG4 antibody. In embodiments, the heterodimeric protein is substituted with a homodimeric protein comprising (a) a first domain comprising (i) an amino acid sequence that is 90%, or 93%, or 95%, or 97%, or 98%, or 99% identical to the amino acid sequence of SEQ ID NO: 27 or 28; and (ii) an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to the amino acid sequence selected from SEQ ID NOs: SEQ ID NOs: 31-71, 111 and 112; and (c) a linker. In embodiments, the heterodimeric protein is substituted with a homodimeric protein comprising an amino acid sequence that is at least 90%, or 93%, or 95%, or 97%, or 98%, or 99% identical to the amino acid sequences selected from SEQ ID NOs. 87-103 and 113-120. In embodiments, the homodimeric protein comprises amino acid sequence selected from SEQ ID NOs. 87-103 and 113-120.

In embodiments, the first domain and/or the heterodimeric protein modulates or is capable of modulating a γδ (gamma delta) T cell. In embodiments, the gamma delta T cell is selected from a cell expressing Vγ4, Vγ9δ2, or Vγ7δ4. In embodiments, the first domain modulates a Vγ9δ2-expressing T cell.

The Second Pharmaceutical Composition that Costimulates γδ T Cells

In one aspect, the present disclosure relates to a method for treating a cancer in a subject in need thereof comprising: (i) administering to the subject a first pharmaceutical composition comprising the heterodimeric protein; and (ii) administering to the subject a second pharmaceutical composition that costimulates γδ T cells.

In any of the embodiments disclosed herein, the second pharmaceutical composition costimulates a receptor selected from CD28, NKG2D, CD27, CD30, 4-1BB (CD137), IL-2R, IL-15R, IL-7R, IL-21R, NKp30, NKp44, DNAM-1 (CD226), IL-2R, IL-7R, IL-15R, dectins, NLRs, killer Ig-like receptors (e.g., KIR2D, KIR3D), C-type lectins (CD94/NKG2A-C, NKG2D), LFA1, CD2, CD46, Junctional Adhesion Molecule-Like (JAML). In embodiments, the second pharmaceutical composition comprises a ligand of the receptor, or a receptor-binding portion thereof. In embodiments, the second pharmaceutical composition comprises a fusion protein (without limitation, e.g., an Fc fusion protein or an albumin fusion protein) comprising a co-stimulatory molecule or a binding portion thereof. In embodiments, the second pharmaceutical composition comprises a fusion protein (without limitation, e.g., an Fc fusion protein or an albumin fusion protein) comprising the ligand of the receptor, or receptor-binding portion thereof. In embodiments, the second pharmaceutical composition comprises a fusion protein (without limitation, e.g., an Fc fusion protein or an albumin fusion protein) comprising the receptor, or a ligand-binding portion thereof. In embodiments, the second pharmaceutical composition comprises an antibody, antibody-like molecule or a receptor-binding portion thereof. In embodiments, the second pharmaceutical composition comprises an agonistic antibody.

In embodiments, the second pharmaceutical composition costimulates CD28 and/or NKG2D. In embodiments, the second pharmaceutical composition comprises a CD28 ligand, a CD28-binding portion thereof, an NKG2D ligand, or an NKG2D-binding portion thereof. In embodiments, the NKG2D ligand is selected from MICA, MICB, ULBP1, ULBP2, ULBP3, ULBP4, ULBP5, ULBP6, H60, MULT1, and RAE1. In embodiments, the NKG2D ligand is a fusion protein (without limitation, e.g., Fc fusion protein) comprising MICA, MICB, ULBP1, ULBP2, ULBP3, ULBP4, ULBP5, ULBP6, H60, MULT1, or RAE1, or an NKG2D-binding portion thereof. In embodiments, the NKG2D ligand is an Fc fusion protein comprising MICA, MICB, ULBP1, ULBP2, ULBP3, ULBP4, ULBP5, ULBP6, H60, MULT1, or RAE1, or an NKG2D-binding portion thereof.

In embodiments, the NKG2D ligand is an antibody, an antibody-like molecule, or a binding fragment thereof. In embodiments, the binding fragment is selected from Fab fragment, heavy variable chain, and single chain variable fragments (scFV). In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. These antibodies are disclosed in Ehrlich et al., Engagement of NKG2D by Cognate Ligand or Antibody Alone Is Insufficient to Mediate Costimulation of Human and Mouse CD8+ T Cells, J Immunol 174 (4) 1922-1931 (2005); and Bauer, et al., Activation of NK cells and T cells by NKG2D, a receptor for stress-inducible MICA, Science 285(5428): pp. 727-729 (1999), which are hereby incorporated by reference in their entirety. In embodiments, the NKG2D ligand is an anti-NKG2D/anti-CD20 bispecific NK-cell engager (e.g. ULBP2:7D8). See Kellner et al., Enhancing natural killer cell-mediated lysis of lymphoma cells by combining therapeutic antibodies with CD20-specific immunoligands engaging NKG2D or NKp30, Oncoimmunology. 5(1): e1058459 (2016), which is hereby incorporated by reference in their entirety.

In embodiments, the second pharmaceutical composition is an NKG2D ligand modulator. In embodiments, the NKG2D ligand modulator is selected from CYAD-01 (Celdara Medical LLC), NKG2D-DARIC T-cells (bluebird bio Inc), NKG2D checkpoint inhibitor (Novelogics Biotechnology), KD-025 (NKG2D-derived CAR T-cell therapy) (Nanjing Kaedi Biotech/Chinese Academy of Sciences), NKX-101 (Nkarta Therapeutics Inc), NKG2D CAR-T (UWELL Biopharma), NKG2D-derived CAR T-cell therapy (AML) (Cellular Biomedicine), anti-NKG2D-ligand NK-cell therapy (The Third Affiliated Hospital of Guangzhou Medical University), CYAD-101 (Celyad Oncology), CYAD-231 (Celyad Oncology), LEU-006 (Leucid Bio Ltd), LEU-002 (Leucid Bio Ltd), LEU-005 (Leucid Bio Ltd), CT-101, Courier therapeutics (Courier Therapeutics Inc), CTM-N2D (CytoMed Therapeutics), sMIC-specific mAb (Medical University of South Carolina), CYAD-05 (Celyad Oncology), CYAD-200 series (Celyad Oncology; Celdara Medical LLC), KYK-2.0 IgG1 (National Cancer Institute), repurposed pamidronic acid (EBV-induced B cell lymphoproliferative disorders) (The University of Hong Kong), CYAD-02 (Celyad Oncology), CYAD-03 (Celyad Oncology), HG-1428 (Human Genome Sciences Inc), B7-H6:CD20 (Christian-Albrechts-University Kiel), IPH-43 (Innate Pharma SA), XYP-317 (Xyphos Inc), anti-CD4 MicAbody antibody+anti-MicAbody CAR T-cell therapy (iNKG2D CAR, HIV infection) (Xyphos Inc), FT-596 (Fate Therapeutics Inc), CLN-619 (Cullinan Oncology LLC), XYP-217 (AvidBiotics Corp), FT-536 (Fate Therapeutics Inc), MicAbody proteins (AvidBiotics Corp), SNK-01 (NKMax America Inc), FT-576 (Fate Therapeutics Inc), PDI-01 (PDI Therapeutics), Motolimod (Array BioPharma Inc), nogapendekin alfa (Altor BioScience Corp), Pidilizumab (CureTech Ltd), and PROSTVAC-VF-TRICOM (Therion Biologics Corp).

In embodiments, the CD28 ligand is selected from CD80 and CD86. In embodiments, the CD28 ligand of is an antibody, an antibody-like molecule, or a binding fragment thereof. In embodiments, the CD28 ligand is a fusion protein (without limitation, e.g., Fc fusion protein) comprising CD80, CD86, or a CD28-binding portion thereof. In embodiments, the CD28 ligand is an Fc fusion protein comprising CD80, CD86, or a CD28-binding portion thereof. In embodiments, the binding fragment is selected from Fab fragment, heavy variable chain, and single chain variable fragments (scFV). In embodiments, the antibody is an agonistic antibody. In embodiments, the antibody is a monoclonal antibody. In embodiments, the antibody is an anti-CD28 monoclonal antibody selected from JJ316, D665, 5.11A1, TGN1412, 37.51, E18, and PV-1. Poirier et al., CD28-Specific Immunomodulating Antibodies: What Can Be Learned From Experimental Models?, Am J Transplant 12(7):1682-90 (2012), which are hereby incorporated by reference in their entirety.

In embodiments, the CD27 ligand is CD70. In embodiments, the CD27 ligand is a fusion protein (without limitation, e.g., Fc fusion protein) comprising CD70, or a CD27-binding portion thereof. In embodiments, the CD27 ligand of is an antibody, an antibody-like molecule, or a binding fragment thereof.

In embodiments, the CD30 ligand is CD30L. In embodiments, the CD30 ligand is a fusion protein (without limitation, e.g., Fc fusion protein) comprising CD30L, or a CD30-binding portion thereof. In embodiments, the CD30 ligand of is an antibody, an antibody-like molecule, or a binding fragment thereof. In embodiments, the 4-1BB ligand is 4-1BBL. In embodiments, the 4-1BB ligand is a fusion protein (without limitation, e.g., Fc fusion protein) comprising 4-1BBL, or a 4-1BB-binding portion thereof. In embodiments, the 4-1BB ligand of is an antibody, an antibody-like molecule, or a binding fragment thereof. In embodiments, the IL-2R ligand is IL-2. In embodiments, the IL-2R ligand is a fusion protein (without limitation, e.g., Fc fusion protein) comprising IL-2, or an IL-2R-binding portion thereof. In embodiments, the IL-2R ligand of is an antibody, an antibody-like molecule, or a binding fragment thereof. In embodiments, the IL-15R ligand is IL-15. In embodiments, the IL-15R ligand is a fusion protein (without limitation, e.g., Fc fusion protein) comprising IL-15, or an IL-15R-binding portion thereof. In embodiments, the IL-15R ligand of is an antibody, an antibody-like molecule, or a binding fragment thereof. In embodiments, the IL-7R ligand is IL-7. In embodiments, the IL-7R ligand is a fusion protein (without limitation, e.g., Fc fusion protein) comprising IL-7, or an IL-7R-binding portion thereof. In embodiments, the IL-7R ligand of is an antibody, an antibody-like molecule, or a binding fragment thereof. In embodiments, the IL-21R ligand is IL-21. In embodiments, the IL-21R ligand is a fusion protein (without limitation, e.g., Fc fusion protein) comprising IL-21, or an IL-21R-binding portion thereof. In embodiments, the IL-21R ligand of is an antibody, an antibody-like molecule, or a binding fragment thereof. In embodiments, the NKp30 ligand is B7-H6 or BAT3. In embodiments, the NKp30 ligand is a fusion protein (without limitation, e.g., Fc fusion protein) comprising B7-H6, BAT3, or an NKp30-binding portion thereof. In embodiments, the NKp30 ligand of is an antibody, an antibody-like molecule, or a binding fragment thereof. In embodiments, the NKp44 ligand is NKp44L. In embodiments, the NKp44 ligand is a fusion protein (without limitation, e.g., Fc fusion protein) comprising NKp44L, or an NKp44-binding portion thereof. In embodiments, the NKp44 ligand of is an antibody, an antibody-like molecule, or a binding fragment thereof. In embodiments, the DNAM-1 (CD226) ligand is Nectin-like-5 or Nectin-2. In embodiments, the DNAM-1 (CD226) ligand is a fusion protein (without limitation, e.g., Fc fusion protein) comprising Nectin-like-5, Nectin-2, or a DNAM-1 (CD226)-binding portion thereof. In embodiments, the DNAM-1 (CD226) ligand of is an antibody, an antibody-like molecule, or a binding fragment thereof. In embodiments, the binding fragment is selected from Fab fragment, heavy variable chain, and single chain variable fragments (scFV). In embodiments, the antibody is an agonistic antibody. In embodiments, the antibody is a monoclonal antibody.

Additionally or alternatively, in embodiments, the second pharmaceutical composition inhibits a receptor selected from a receptor selected from PD-1, PD-L1 and BTLA. In embodiments, the second pharmaceutical composition comprises a soluble receptor. In embodiments, the second pharmaceutical composition comprises an extracellular domain of PD-1, an extracellular domain of BTLA, or a receptor binding domain thereof. In embodiments, the PD-1 ligand is PD-L1 (B7-H1) or PD-L2 (B7-DC). In embodiments, the PD-1 ligand of is an antibody, an antibody-like molecule, or a binding fragment thereof. In embodiments, the second pharmaceutical composition comprises an antibody, an antibody-like molecule, or a binding fragment thereof.

In embodiments, the binding fragment is selected from Fab fragment, heavy variable chain, and single chain variable fragments (scFV). In embodiments, the antibody is an antagonistic antibody. In embodiments, the antibody is a monoclonal antibody. In embodiments, the monoclonal antibody is an anti-PD-1 antibody selected from pembrolizumab, nivolumab, and cemiplimab. In embodiments, the antibody that is capable of binding PD-1 or binding a PD-1 ligand is selected from the group consisting of nivolumab (ONO-4538/BMS-936558, MDX1106, OPDIVO, BRISTOL MYERS SQUIBB), pembrolizumab (KEYTRUDA, MERCK), RMP1-14, AGEN2034 (AGENUS), cemiplimab (REGN-2810), MK-3475 (MERCK), BMS 936559 (BRISTOL MYERS SQUIBB), Ibrutinib (PHARMACYCLICS/ABBVIE), atezolizumab (TECENTRIQ, GENENTECH), and MPDL3280A (ROCHE). In embodiments, the BTLA ligand is HVEM. In embodiments, the BTLA ligand of is an antibody, an antibody-like molecule, or a binding fragment thereof.

In any of the embodiments disclosed herein, the second pharmaceutical composition comprises a heterologous chimeric protein capable of costimulating a receptor selected from CD28, NKG2D, CD27, CD30, 4-1BB (CD137), IL-2R, IL-15R, IL-7R, IL-21R, NKp30, NKp44, DNAM-1 (CD226), IL-2R, IL-7R, IL-15R, dectins, NLRs, killer Ig-like receptors (e.g., KIR2D, KIR3D), C-type lectins (CD94/NKG2A-C, NKG2D), LFA1, CD2, CD46, and Junctional Adhesion Molecule-Like (JAML) and/or inhibiting a receptor selected from a receptor selected from PD-1, PD-L1 and BTLA. In embodiments, the heterologous chimeric protein comprises (a) a first domain comprising an extracellular domain of a type I membrane protein; (b) a second domain comprising an extracellular domain of Type II transmembrane protein; and (c) a linker linking the first domain and the second domain. In embodiments, the linker comprises a hinge-CH2-CH3 Fc domain. In embodiments, the heterologous chimeric protein comprises an extracellular domain and/or a ligand binding domain of a receptor selected from CD28, NKG2D, CD27, CD30, 4-1BB (CD137), IL-2R, IL-15R, IL-7R, IL-21R, NKp30, NKp44, DNAM-1 (CD226), IL-2R, IL-7R, IL-15R, dectins, NLRs, killer Ig-like receptors (e.g., KIR2D, KIR3D), C-type lectins (CD94/NKG2A-C, NKG2D), LFA1, CD2, CD46, and Junctional Adhesion Molecule-Like (JAML). In embodiments, the heterologous chimeric protein comprises an extracellular domain and/or a ligand binding domain of a receptor selected from PD-1, PD-L1 and BTLA. In embodiments, the heterologous chimeric protein comprises an extracellular domain and/or aligand binding domain of a receptor selected from CD30L, 4-1BBL, B7-H6, BAT3, NKp44L, Nectin-like-5, and Nectin-2. In embodiments, the heterologous chimeric protein comprises an extracellular domain and/or a ligand binding domain of MICA, MICB, ULBP1, ULBP2, ULBP3, ULBP4, ULBP5, ULBP6, H60, MULT1, and RAE1. In embodiments, the heterologous chimeric protein comprises an extracellular domain and/or a ligand binding domain of CD80 and/or CD86. In embodiments, the heterologous chimeric protein comprises a receptor binding domain of a cytokine selected from IL-2, IL-15, IL-7, and IL-21. Suitable heterologous chimeric proteins are disclosed in PCT publications WO 2017/059168, WO 2017/059168, WO 2017/059168, WO 2017/059168, WO 2018/157165, WO 2018/157165, WO 2020/047319, WO 2020/047322, WO 2020/047325, WO 2020/047327, WO 2020/047328, WO 2020/047329, WO 2020/176718, WO 2020/232365, and WO 2021/041958, which are hereby incorporated by reference in their entirety.

In any of the embodiments disclosed herein, the second pharmaceutical composition includes a modulator of NKG2D ligand expression and/or function. In embodiments, the modulator of NKG2D ligand expression and/or function is an antibody. In embodiments, the modulator of NKG2D ligand expression and/or function is an anti-MICA α3 domain antibody. In embodiments, the anti-MICA α3 domain antibody is selected from 7C6, 6F11, and 1C2. These antibodies are disclosed in Ferrari de Andrade et al., Antibody-mediated inhibition of MICA and MICB shedding promotes NK cell-driven tumor immunity, Science 359(6383): 1537-1542 (2018), which are hereby incorporated by reference in their entirety. In embodiments, the modulator of NKG2D ligand expression and/or function is an agonistic an anti-MICA antibody. In embodiments, the agonistic anti-MICA antibody is CLN-619 (Cullinan Oncology LLC).

### Diseases; Methods of Treatment, and Patient Selections

In one aspect, the current disclosure provides a method of treating cancer, comprising administering to a subject in need thereof an effective amount of a pharmaceutical composition of any of the embodiments disclosed herein to a subject in need thereof. In embodiments, the cancer is a lymphoma. In embodiments, the cancer is a leukemia. In embodiments, the cancer is a Hodgkin's and non-Hodgkin's lymphoma, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; or chronic myeloblastic leukemia. In embodiments, the cancer is basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (e.g. that associated with brain tumors), and Meigs' syndrome. In embodiments, the cancer is prostate cancer. In embodiments, the cancer is an epithelial-derived carcinoma. In embodiments, the cancer is known to express the antigenic target of the second domain of the heterodimeric protein. In embodiments, the cancer is known to contain mutations which limit recognition by alpha beta T cells, including but not limited to mutations in MHC I, beta 2 microglobulin, TAP, etc.

In embodiments, the subject is further administered autologous or allogeneic gamma delta T cells that were expanded ex vivo. In embodiments, the autologous or allogeneic gamma delta T cells express a Chimeric Antigen Receptor. In embodiments, the subject is further administered autologous or allogeneic T cells that express a Chimeric Antigen Receptor.

In one aspect, the current disclosure provides a method of treating an autoimmune disease or disorder, comprising administering an effective amount of a pharmaceutical composition of any of the embodiments disclosed herein to a subject in need thereof, wherein the autoimmune disease or disorder is optionally selected from rheumatoid arthritis, systemic lupus erythematosus, diabetes mellitus, ankylosing spondylitis, Sjögren's syndrome, inflammatory bowel diseases (e.g., colitis ulcerosa, Crohn's disease), multiple sclerosis, sarcoidosis, psoriasis, Grave's disease, Hashimoto's thyroiditis, psoriasis, hypersensitivity reactions (e.g., allergies, hay fever, asthma, and acute edema cause type I hypersensitivity reactions), and vasculitis.

In various embodiments, the current disclosure pertains to the use of the heterodimeric proteins for the treatment of one or more autoimmune diseases or disorders. In various embodiments, the treatment of an autoimmune disease or disorder may involve modulating the immune system with the present heterodimeric proteins to favor immune inhibition over immune stimulation. Illustrative autoimmune diseases or disorders treatable with the present heterodimeric proteins include those in which the body's own antigens become targets for an immune response, such as, for example, rheumatoid arthritis, systemic lupus erythematosus, diabetes mellitus, ankylosing spondylitis, Sjögren's syndrome, inflammatory bowel diseases (e.g., colitis ulcerosa, Crohn's disease), multiple sclerosis, sarcoidosis, psoriasis, Grave's disease, Hashimoto's thyroiditis, psoriasis, hypersensitivity reactions (e.g., allergies, hay fever, asthma, and acute edema cause type I hypersensitivity reactions), and vasculitis.

Illustrative autoimmune diseases or conditions that may be treated or prevented using the heterodimeric protein of the invention include, but are not limited to, multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection), pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases.

In various embodiments, the current disclosure pertains to cancers and/or tumors; for example, the treatment or prevention of cancers and/or tumors. As described elsewhere herein, the treatment of cancer may involve in various embodiments, modulating the immune system with the present heterodimeric proteins to favor immune stimulation over immune inhibition.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis.

The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the current disclosure include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the cancer is an epithelial-derived carcinoma.

In embodiments, the heterodimeric protein is used to treat a subject that has a treatment-refractory cancer. In embodiments, the heterodimeric protein is used to treat a subject that is refractory to one or more immune-modulating agents. For example, In embodiments, the heterodimeric protein is used to treat a subject that presents no response to treatment, or even progress, after 12 weeks or so of treatment. For instance, In embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), Ibrutinib (PHARMACYCLICS/ABBVIE), atezolizumab (TECENTRIQ, GENENTECH), and/or MPDL3280A (ROCHE)-refractory patients. For instance, In embodiments, the subject is refractory to an anti-CTLA-4 agent, e.g., ipilimumab (YERVOY)-refractory patients (e.g., melanoma patients). Accordingly, in various embodiments the current disclosure provides methods of cancer treatment that rescue patients that are non-responsive to various therapies, including monotherapy of one or more immune-modulating agents.

In various embodiments, the current disclosure provides heterodimeric proteins which target a cell or tissue within the tumor microenvironment. In embodiments, the cell or tissue within the tumor microenvironment expresses one or more targets or binding partners of the heterodimeric protein. The tumor microenvironment refers to the cellular milieu, including cells, secreted proteins, physiological small molecules, and blood vessels in which the tumor exists. In embodiments, the cells or tissue within the tumor microenvironment are one or more of: tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor. In various embodiments, the present heterodimeric protein targets a cancer cell. In embodiments, the cancer cell expresses one or more of targets or binding partners of the heterodimeric protein.

In various embodiments, the heterodimeric protein of the invention may target a cell (e.g., cancer cell or immune cell) that expresses any of the receptors as described herein. For example, the heterodimeric protein of the invention may target a cell that expresses any of the receptors for a cytokine, growth factor, and/or hormone as described herein.

In embodiments, the present methods provide treatment with the heterodimeric protein in a patient who is refractory to an additional agent, such “additional agents” being described elsewhere herein, inclusive, without limitation, of the various chemotherapeutic agents described herein.

In some aspects, the present chimeric agents are used to eliminate intracellular pathogens. In some aspects, the present chimeric agents are used to treat one or more infections. In embodiments, the present heterodimeric proteins are used in methods of treating viral infections (including, for example, HIV and HCV), parasitic infections (including, for example, malaria), and bacterial infections. In various embodiments, the infections induce immunosuppression. For example, HIV infections often result in immunosuppression in the infected subjects. Accordingly, as described elsewhere herein, the treatment of such infections may involve, in various embodiments, modulating the immune system with the present heterodimeric proteins to favor immune stimulation over immune inhibition. Alternatively, the current disclosure provides methods for treating infections that induce immunoactivation. For example, intestinal helminth infections have been associated with chronic immune activation. In these embodiments, the treatment of such infections may involve modulating the immune system with the present heterodimeric proteins to favor immune inhibition over immune stimulation.

In various embodiments, the current disclosure provides methods of treating viral infections including, without limitation, acute or chronic viral infections, for example, of the respiratory tract, of papilloma virus infections, of herpes simplex virus (HSV) infection, of human immunodeficiency virus (HIV) infection, and of viral infection of internal organs such as infection with hepatitis viruses. In embodiments, the viral infection is caused by a virus of family Flaviviridae. In embodiments, the virus of family Flaviviridae is selected from Yellow Fever Virus, West Nile virus, Dengue virus, Japanese Encephalitis Virus, St. Louis Encephalitis Virus, and Hepatitis C Virus. In other embodiments, the viral infection is caused by a virus of family Picornaviridae, e.g., poliovirus, rhinovirus, coxsackievirus. In other embodiments, the viral infection is caused by a member of Orthomyxoviridae, e.g., an influenza virus. In other embodiments, the viral infection is caused by a member of Retroviridae, e.g., a lentivirus. In other embodiments, the viral infection is caused by a member of Paramyxoviridae, e.g., respiratory syncytial virus, a human parainfluenza virus, rubulavirus (e.g., mumps virus), measles virus, and human metapneumovirus. In other embodiments, the viral infection is caused by a member of Bunyaviridae, e.g., hantavirus. In other embodiments, the viral infection is caused by a member of Reoviridae, e.g., a rotavirus.

In various embodiments, the current disclosure provides methods of treating parasitic infections such as protozoan or helminths infections. In embodiments, the parasitic infection is by a protozoan parasite. In embodiments, the oritiziab parasite is selected from intestinal protozoa, tissue protozoa, or blood protozoa. Illustrative protozoan parasites include, but are not limited to, Entamoeba hystolytica, Giardia lamblia, Cryptosporidium muris, Trypanosomatida gambiense, Trypanosomatida rhodesiense, Trypanosomatida crusi, Leishmania mexicana, Leishmania braziliensis, Leishmania tropica, Leishmania donovani, Toxoplasma gondii, Plasmodium vivax, Plasmodium ovale, Plasmodium malariae, Plasmodium falcipanum, Trichomonas vaginalis, and Histomonas meleagridis. In embodiments, the parasitic infection is by a helminthic parasite such as nematodes (e.g., Adenophorea). In embodiments, the parasite is selected from Secementea (e.g., Trichuris trichiura, Ascaris lumbricoides, Enterobius vermicularis, Ancylostoma duodenale, Necator americanus, Strongyloides stercoralis, Wuchereria bancrofti, Dracunculus medinensis). In embodiments, the parasite is selected from trematodes (e.g., blood flukes, liver flukes, intestinal flukes, and lung flukes). In embodiments, the parasite is selected from: Schistosoma mansoni, Schistosoma haematobium, Schistosoma japonicum, Fasciola hepatica, Fasciola gigantica, Heterophyes, Paragonimus westermani. In embodiments, the parasite is selected from cestodes (e.g., Taenia solium, Taenia saginata, Hymenolepis nana, Echinococcus granulosus).

In various embodiments, the current disclosure provides methods of treating bacterial infections. In various embodiments, the bacterial infection is by gram-positive bacteria, gram-negative bacteria, aerobic and/or anaerobic bacteria. In various embodiments, the bacteria are selected from, but not limited to, Staphylococcus, Lactobacillus, Streptococcus, Sarcina, Escherichia, Enterobacter, Klebsiella, Pseudomonas, Acinetobacter, Mycobacterium, Proteus, Campylobacter, Citrobacter, Nisseria, Bacillus, Bacteroides, Peptococcus, Clostridium, Salmonella, Shigella, Serratia, Haemophilus, Brucella and other organisms. In embodiments, the bacteria is selected from, but not limited to, Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter baumannii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Kingella, Moraxella, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, or Staphylococcus saccharolyticus.

In still another other aspect, the current disclosure is directed toward methods of treating and preventing T cell-mediated diseases and disorders, such as, but not limited to diseases or disorders described elsewhere herein and inflammatory disease or disorder, graft-versus-host disease (GVHD), transplant rejection, and T cell proliferative disorder.

In some aspects, the present chimeric agents are used in methods of activating a T cell, e.g., via the extracellular domain having an immune stimulatory signal or antibody binding domain (e.g. CDR3, Fab, scFv domain, etc.) having an immune stimulatory signal.

In some aspects, the present chimeric agents are used in methods of preventing the cellular transmission of an immunosuppressive signal.

### Combination Therapies and Conjugation

In one aspect, the present disclosure relates to a method for treating a cancer in a subject in need thereof comprising: (i) administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains and (ii) administering to the subject a second pharmaceutical composition that costimulates γδ T cells. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains; wherein the subject has undergone or is undergoing treatment with a second pharmaceutical composition comprising a costimulatory molecule. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In one aspect, the present disclosure relates to a method for treating a cancer in a subject in need thereof comprising: administering to the subject a second pharmaceutical composition that costimulates γδ T cells, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In one aspect, the present disclosure relates to a method for treating a cancer in a subject in need thereof comprising: (A) administering to the subject a first pharmaceutical composition comprising a homodimeric protein comprising (a) a first domain comprising (i) an amino acid sequence that is 90%, or 93%, or 95%, or 97%, or 98%, or 99% identical to the amino acid sequence of SEQ ID NO: 27 or 28; and (ii) an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to the amino acid sequence selected from SEQ ID NOs: SEQ ID NOs: 31-71, 111 and 112; and (c) a linker; and (B) administering to the subject a second pharmaceutical composition comprising an anti-CD28 antibody and/or and anti-NKG2D antibody. In embodiments, the heterodimeric protein comprises amino acid sequences that is at least 90%, or 93%, or 95%, or 97%, or 98%, or 99% identical to an amino acid sequence selected from SEQ ID NOs. 87-103 and 113-120. In embodiments, the heterodimeric protein comprises amino acid sequences of an amino acid sequence selected from SEQ ID NOs. 87-103 and 113-120. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In one aspect, the present disclosure relates to a method for treating a cancer in a subject in need thereof comprising: administering to the subject a first pharmaceutical composition comprising a homodimeric protein comprising (a) a first domain comprising (i) an amino acid sequence that is 90%, or 93%, or 95%, or 97%, or 98%, or 99% identical to the amino acid sequence of SEQ ID NO: 27 or 28; and (ii) an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to the amino acid sequence selected from SEQ ID NOs: SEQ ID NOs: 31-71, 111 and 112; and (c) a linker, wherein the subject has undergone or is undergoing treatment with a second pharmaceutical composition comprising an anti-CD28 antibody and/or and anti-NKG2D antibody. In embodiments, the heterodimeric protein comprises amino acid sequences that is at least 90%, or 93%, or 95%, or 97%, or 98%, or 99% identical to an amino acid sequence selected from SEQ ID NOs. 87-103 and 113-120. In embodiments, the heterodimeric protein comprises amino acid sequences of an amino acid sequence selected from SEQ ID NOs. 87-103 and 113-120. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a homodimeric protein comprising (a) a first domain comprising (i) an amino acid sequence that is 90%, or 93%, or 95%, or 97%, or 98%, or 99% identical to the amino acid sequence of SEQ ID NO: 27 or 28; and (ii) an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to the amino acid sequence selected from SEQ ID NOs: SEQ ID NOs: 31-71, 111 and 112; and (c) a linker. In embodiments, the heterodimeric protein comprises amino acid sequences that is at least 90%, or 93%, or 95%, or 97%, or 98%, or 99% identical to an amino acid sequence selected from SEQ ID NOs. 87-103 and 113-120. In embodiments, the heterodimeric protein comprises amino acid sequences of an amino acid sequence selected from SEQ ID NOs. 87-103 and 113-120. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1. In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In any of the embodiments disclosed herein, the second pharmaceutical composition costimulates a receptor selected from CD28, NKG2D, CD27, CD30, 4-1BB (CD137), IL-2R, IL-15R, IL-7R, IL-21R, NKp30, NKp44, DNAM-1 (CD226), IL-2R, IL-7R, IL-15R, dectins, NLRs, killer Ig-like receptors (e.g., KIR2D, KIR3D), C-type lectins (CD94/NKG2A-C, NKG2D), LFA1, CD2, CD46, Junctional Adhesion Molecule-Like (JAML). In embodiments, the second pharmaceutical composition comprises a ligand of the receptor, or a receptor-binding portion thereof. In embodiments, the second pharmaceutical composition comprises a fusion protein (without limitation, e.g., an Fc fusion protein or an albumin fusion protein) comprising a co-stimulatory molecule or a binding portion thereof. In embodiments, the second pharmaceutical composition comprises a fusion protein (without limitation, e.g., an Fc fusion protein or an albumin fusion protein) comprising the ligand of the receptor, or receptor-binding portion thereof. In embodiments, the second pharmaceutical composition comprises a fusion protein (without limitation, e.g., an Fc fusion protein or an albumin fusion protein) comprising the receptor, or a ligand-binding portion thereof. In embodiments, the second pharmaceutical composition comprises an antibody, antibody-like molecule or a receptor-binding portion thereof. In embodiments, the second pharmaceutical composition comprises an agonistic antibody.

In embodiments, the second pharmaceutical composition costimulates CD28 and/or NKG2D. In embodiments, the second pharmaceutical composition comprises a CD28 ligand, a CD28-binding portion thereof, an NKG2D ligand, or an NKG2D-binding portion thereof. In embodiments, the NKG2D ligand is selected from MICA, MICB, ULBP1, ULBP2, ULBP3, ULBP4, ULBP5, ULBP6, H60, MULT1, and RAE1. In embodiments, the NKG2D ligand is an antibody, an antibody-like molecule, or a binding fragment thereof. In embodiments, the binding fragment is selected from Fab fragment, heavy variable chain, and single chain variable fragments (scFV). In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6.

In embodiments, the CD28 ligand is selected from CD80 and CD86. In embodiments, the CD28 ligand of is an antibody, an antibody-like molecule, or a binding fragment thereof. In embodiments, the binding fragment is selected from Fab fragment, heavy variable chain, and single chain variable fragments (scFV). In embodiments, the antibody is an agonistic antibody. In embodiments, the antibody is a monoclonal antibody. In embodiments, the antibody is an anti-CD28 monoclonal antibody selected from JJ316, D665, 5.11A1, TGN1412, 37.51, E18, and PV-1.

Additionally or alternatively, in embodiments, the second pharmaceutical composition inhibits a receptor selected from a receptor selected from PD-1, PD-L1 and BTLA. In embodiments, the second pharmaceutical composition comprises a soluble receptor. In embodiments, the second pharmaceutical composition comprises an extracellular domain of PD-1, an extracellular domain of BTLA, or a receptor binding domain thereof. In embodiments, the second pharmaceutical composition comprises an antibody, an antibody-like molecule, or a binding fragment thereof. In embodiments, the binding fragment is selected from Fab fragment, heavy variable chain, and single chain variable fragments (scFV). In embodiments, the antibody is an antagonistic antibody. In embodiments, the antibody is a monoclonal antibody. In embodiments, the monoclonal antibody is an anti-PD-1 antibody selected from pembrolizumab, nivolumab, and cemiplimab.

In any of the embodiments disclosed herein, the BTN2A1 protein, or a fragment thereof comprises a variable Ig-like V-type domain. In embodiments, the variable Ig-like V-type domain of the BTN2A1 protein comprises an amino acid sequence that is at least 90%, or 93%, or 95%, or 97%, or 98%, or 99% identical to the amino acid sequence of SEQ ID NO: 28. In embodiments, the BTN2A1 protein, or a fragment thereof comprise a extracellular domain (ECD). In embodiments, the variable ECD of the BTN2A1 protein comprises an amino acid sequence that is at least 90%, or 93%, or 95%, or 97%, or 98%, or 99% identical to the amino acid sequence of SEQ ID NO: 27.

In any of the embodiments disclosed herein, the BTN3A1 protein, or a fragment thereof comprise a variable Ig-like V-type domain. In embodiments, the variable Ig-like V-type domain of the BTN3A1 protein comprises an amino acid sequence that is at least 90%, or 93%, or 95%, or 97%, or 98%, or 99% identical to the amino acid sequence of SEQ ID NO: 30. In embodiments, the BTN3A1 protein, or a fragment thereof comprise a extracellular domain (ECD). In embodiments, variable ECD of the BTN3A1 protein comprises an amino acid sequence that is at least 90%, or 93%, or 95%, or 97%, or 98%, or 99% identical to the amino acid sequence of SEQ ID NO: 29.

In embodiments, the first domain comprises an amino acid sequence having an amino acid sequence of selected from one or more of SEQ ID NOs: 27-30.

In any of the embodiments disclosed herein, the targeting domain is an antibody, or antigen binding fragment thereof. In embodiments, the targeting domain is an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In embodiments, the antibody-like molecule is an scFv.

Additionally or alternatively, in embodiments, the targeting domain is an extracellular domain. In embodiments, the targeting domain is capable of binding an antigen on the surface of a cancer cell. In embodiments, the targeting domain specifically binds one or more of CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, and GD3. In embodiments, the targeting domain comprises a portion of the extracellular domain of LAG-3, PD-1, TIGIT, CD19, and PCMA. In embodiments, the targeting domain specifically binds CD19. In embodiments, the targeting domain specifically binds PSMA. In embodiments, the targeting domain specifically binds B7H3. In embodiments, the targeting domain specifically binds FAP. In embodiments, the targeting domain specifically binds CD20. In embodiments, the targeting domain specifically binds CD33.

In embodiments, the targeting domain is a polypeptide having an amino acid sequence with at least 90%, or 95%, or 97%, or 98%, or 99% identity with a polypeptide selected from SEQ ID NOs: 31-71, 111 and 112. In embodiments, the targeting domain is a polypeptide having an amino acid sequence of selected from one or more of SEQ ID NOs: 31-71, 111 and 112. In embodiments, the targeting domain is a polypeptide having an amino acid sequence with at least 90%, or 95%, or 97%, or 98%, or 99% identity with a polypeptide selected from SEQ ID NOs: 31-35, 41, 48, 111 and 112. In embodiments, the targeting domain is a polypeptide having an amino acid sequence of selected from one or more of SEQ ID NOs: 31-35, 41, 48, 111 and 112.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains.

In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally from human IgG1. In embodiments, the linker comprises the hinge-CH2-CH3 Fc domain derived from IgG4, optionally from human IgG4.

In embodiments, the first and/or second charge polarized core domain further comprise peptides having positively and/or negatively charged amino acid residues at the amino and/or carboxy terminus of the charge polarized core domain. In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains. In embodiments, the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5). In embodiments, the peptide comprising positively charged amino acid residues comprises the sequence RKGGKR (SEQ ID NO: 11) or GSGSRKGGKRGS (SEQ ID NO: 12).

In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino 5 acid such as aspartic acid or glutamic acid and Y is a spacer amino acid such as serine or glycine, and where each n is independently an integer 0 to 4) (where Z is a negatively charged amino acid such as aspartic acid or glutamic acid and Y is a spacer amino acid such as serine or glycine) (SEQ ID NO: 6). In embodiments, the peptide comprising negatively charged amino acid residues comprises the sequence DEGGED (SEQ ID NO: 13) or GSGSDEGGEDGS (SEQ ID NO: 14).

In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120.

In embodiments, the first domain and/or the heterodimeric protein modulates or is capable of modulating a γδ (gamma delta) T cell. In embodiments, the gamma delta T cell is selected from a cell expressing Vγ4, Vγ9δ2, or Vγ7δ4. In embodiments, the first domain modulates a Vγ9δ2-expressing T cell.

In one aspect, the present disclosure relates to a method for treating a cancer in a subject in need thereof comprising: (i) administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains and (ii) administering to the subject a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD19, PSMA, B7H3, FAP, CD20 or CD33; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds CD19, PSMA, B7H3, FAP, CD20 or CD33; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD19, PSMA, B7H3, FAP, CD20 or CD33; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof 30 comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD19, PSMA, B7H3, FAP, CD20 or CD33; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds PSMA; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds PSMA; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds PSMA; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds PSMA; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds B7H3; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds B7H3; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds B7H3; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds B7H3; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds FAP; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds FAP; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds FAP; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds FAP; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD20; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds CD20; and (c) a linker that adjoins the first and second domains; wherein the subject has undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD20; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof 5 comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD20; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD33; and (c) a linker that adjoins the first and second domains; and wherein the beta chain comprises: (a) a first domain comprising a BTN3A1 protein, or a fragment thereof; (b) a second domain comprising a targeting domain, wherein the targeting domain specifically binds CD33; and (c) a linker that adjoins the first and second domains; wherein the subject has 25 undergone or is undergoing treatment with a second pharmaceutical composition comprising an anti-CD28 antibody and/or and anti-NKG2D antibody. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD33; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof 15 comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising a BTN2A1 protein, or a fragment thereof; (b) a second domain comprising a targeting domain, wherein the targeting domain specifically binds CD33; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: (i) administering to the subject a first pharmaceutical composition comprising a heterodimeric protein comprising an alpha chain and a beta chain, wherein the alpha chain comprises: (a) a first domain comprising an amino acid sequence that is 90%, or 93%, or 95%, or 97%, or 98%, or 99% identical to the amino acid sequence of SEQ ID NO: 27 or 28; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 16; and wherein the beta chain comprises: (a) a first domain comprising an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 17; and (ii) administering to the subject a second pharmaceutical composition comprising an anti-CD28 antibody and/or and anti-NKG2D antibody. In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120. In embodiments, the alpha chain comprises an amino acid sequence that is at least 90%, or 93%, or 95%, or 97%, or 98%, or 99% identical to the amino acid sequence of SEQ ID NO: 75, and the beta chain comprises an amino acid sequence that is at least 90%, or 93%, or 95%, or 97%, or 98%, or 99% identical to the amino acid sequence of SEQ ID NO: 81. In embodiments, the alpha chain comprises an amino acid sequence that is at least 90%, or 93%, or 95%, or 97%, or 98%, or 99% identical to the amino acid sequence of SEQ ID NO: 113, and the beta chain comprises an amino acid sequence that is at least 90%, or 93%, or 95%, or 97%, or 98%, or 99% identical to the amino acid sequence of SEQ ID NO: 114. In embodiments, the alpha chain comprises an amino acid sequence that is at least 90%, or 93%, or 95%, or 97%, or 98%, or 99% identical to the amino acid sequence of SEQ ID NO: 115, and the beta chain comprises an amino acid sequence that is at least 90%, or 93%, or 95%, or 97%, or 98%, or 99% identical to the amino acid sequence of SEQ ID NO: 115. In embodiments, the alpha chain comprises an amino acid sequence that is at least 90%, or 93%, or 95%, or 97%, or 98%, or 99% identical to the amino acid sequence of SEQ ID NO: 117, and the beta chain comprises an amino acid sequence that is at least 90%, or 93%, or 95%, or 97%, or 98%, or 99% identical to the amino acid sequence of SEQ ID NO: 118. In embodiments, the alpha chain comprises an amino acid sequence that is at least 90%, or 93%, or 95%, or 97%, or 98%, or 99% identical to the amino acid sequence of SEQ ID NO: 119, and the beta chain comprises an amino acid sequence that is at least 90%, or 93%, or 95%, or 97%, or 98%, or 99% identical to the amino acid sequence of SEQ ID NO: 120. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In one aspect, the present disclosure relates to a method for treating a cancer in a subject in need thereof comprising: administering to the subject a first pharmaceutical composition comprising a heterodimeric protein comprising an alpha chain and a beta chain, wherein the alpha chain comprises: (a) a first domain comprising an amino acid sequence that is 90%, or 93%, or 95%, or 97%, or 98%, or 99% identical to the amino acid sequence of SEQ ID NO: 27 or 28; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 16; and wherein the beta chain comprises: (a) a first domain comprising an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 17; wherein the subject has undergone or is undergoing treatment with a second pharmaceutical composition comprising an anti-CD28 antibody and/or and anti-NKG2D antibody. In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120. In embodiments, the alpha chain comprises an amino acid sequence that is at least 90%, or 93%, or 95%, or 97%, or 98%, or 99% identical to the amino acid sequence of SEQ ID NO: 75, and the beta chain comprises an amino acid sequence that is at least 90%, or 93%, or 95%, or 97%, or 98%, or 99% identical to the amino acid sequence of SEQ ID NO: 81. In embodiments, the alpha chain comprises an amino acid sequence that is at least 90%, or 93%, or 95%, or 97%, or 98%, or 99% identical to the amino acid sequence of SEQ ID NO: 113, and the beta chain comprises an amino acid sequence that is at least 90%, or 93%, or 95%, or 97%, or 98%, or 99% identical to the amino acid sequence of SEQ ID NO: 114. In embodiments, the alpha chain comprises an amino acid sequence that is at least 90%, or 93%, or 95%, or 97%, or 98%, or 99% identical to the amino acid sequence of SEQ ID NO: 115, and the beta chain comprises an amino acid sequence that is at least 90%, or 93%, or 95%, or 97%, or 98%, or 99% identical to the amino acid sequence of SEQ ID NO: 115. In embodiments, the alpha chain comprises an amino acid sequence that is at least 90%, or 93%, or 95%, or 97%, or 98%, or 99% identical to the amino acid sequence of SEQ ID NO: 117, and the beta chain comprises an amino acid sequence that is at least 90%, or 93%, or 95%, or 97%, or 98%, or 99% identical to the amino acid sequence of SEQ ID NO: 118. In embodiments, the alpha chain comprises an amino acid sequence that is at least 90%, or 93%, or 95%, or 97%, or 98%, or 99% identical to the amino acid sequence of SEQ ID NO: 119, and the beta chain comprises an amino acid sequence that is at least 90%, or 93%, or 95%, or 97%, or 98%, or 99% identical to the amino acid sequence of SEQ ID NO: 120. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A, TGN1412, 37.51, E18, and PV-1.

In one aspect, the present disclosure relates to a method for treating a cancer in a subject in need thereof comprising: administering to the subject a second pharmaceutical composition comprising an anti-CD28 antibody and/or and anti-NKG2D antibody, wherein the subject has undergone or is undergoing treatment with a first pharmaceutical composition comprising a heterodimeric protein comprising an alpha chain and a beta chain, wherein the alpha chain comprises: (a) a first domain comprising an amino acid sequence that is 90%, or 93%, or 95%, or 97%, or 98%, or 99% identical to the amino acid sequence of SEQ ID NO: 27 or 28; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 16; and wherein the beta chain comprises: (a) a first domain comprising an amino acid sequence that is 90%, or 93%, or 95%, or 97%, or 98%, or 99% identical to the amino acid sequence of SEQ ID NO: 29 or 30; (b) a second domain comprising an amino acid sequence that is 90%, or 93%, or 95%, or 97%, or 98%, or 99% identical to an amino acid sequence selected from SEQ ID NOs: 35, 41, 48, 111 and 112; and (c) a linker comprising an amino acid sequence that is 90%, or 93%, or 95%, or 97%, or 98%, or 99% identical to the amino acid sequence of SEQ ID NO: 17. In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120. In embodiments, the alpha chain comprises an amino acid sequence that is at least 90%, or 93%, or 95%, or 97%, or 98%, or 99% identical to the amino acid sequence of SEQ ID NO: 75, and the beta chain comprises an amino acid sequence that is at least 90%, or 93%, or 95%, or 97%, or 98%, or 99% identical to the amino acid sequence of SEQ ID NO: 81. In embodiments, the alpha chain comprises an amino acid sequence that is at least 90%, or 93%, or 95%, or 97%, or 98%, or 99% identical to the amino acid sequence of SEQ ID NO: 113, and the beta chain comprises an amino acid sequence that is at least 90%, or 93%, or 95%, or 97%, or 98%, or 99% identical to the amino acid sequence of SEQ ID NO: 114. In embodiments, the alpha chain comprises an amino acid sequence that is at least 90%, or 93%, or 95%, or 97%, or 98%, or 99% identical to the amino acid sequence of SEQ ID NO: 115, and the beta chain comprises an amino acid sequence that is at least 90%, or 93%, or 95%, or 97%, or 98%, or 99% identical to the amino acid sequence of SEQ ID NO: 115. In embodiments, the alpha chain comprises an amino acid sequence that is at least 90%, or 93%, or 95%, or 97%, or 98%, or 99% identical to the amino acid sequence of SEQ ID NO: 117, and the beta chain comprises an amino acid sequence that is at least 90%, or 93%, or 95%, or 97%, or 98%, or 99% identical to the amino acid sequence of SEQ ID NO: 118. In embodiments, the alpha chain comprises an amino acid sequence that is at least 90%, or 93%, or 95%, or 97%, or 98%, or 99% identical to the amino acid sequence of SEQ ID NO: 119, and the beta chain comprises an amino acid sequence that is at least 90%, or 93%, or 95%, or 97%, or 98%, or 99% identical to the amino acid sequence of SEQ ID NO: 120. In embodiments, the antibody is an agonistic antibody. In embodiments, the antibody is a monoclonal antibody. In embodiments, the antibody is an anti-NKG2D monoclonal antibody selected from tesnatilimab (also known as JNJ 4500 or JNJ-64304500) 149810, 1D11 and 5C6. In embodiments, the antibody is an anti-CD28 monoclonal antibody selected from JJ316, D665, 5.11A1, TGN1412, 37.51, E18, and PV-1.

In embodiments, the first pharmaceutical composition and the second pharmaceutical composition are administered simultaneously or contemporaneously. In embodiments, the first pharmaceutical composition is administered after the second pharmaceutical composition is administered. In embodiments, the first pharmaceutical composition is administered before the second pharmaceutical composition is administered.

In embodiments, the dose of the first pharmaceutical composition is less than the dose of the first pharmaceutical composition administered to a subject who has not undergone or is not undergoing treatment with the second pharmaceutical composition. In embodiments, the dose of the second pharmaceutical composition administered is less than the dose of the second pharmaceutical composition administered to a subject who has not undergone or is not undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the first pharmaceutical composition. In embodiments, the subject has an increased chance of survival, without gastrointestinal inflammation and weight loss, and/or a reduction in tumor size or cancer prevalence when compared to a subject who has only undergone or is only undergoing treatment with the second pharmaceutical composition.

In embodiments, the BTN2A1 protein, or a fragment thereof comprises a variable Ig-like V-type domain. In embodiments, the variable Ig-like V-type domain of the BTN2A1 protein comprises an amino acid sequence that is at least 90%, or 93%, or 95%, or 97%, or 98%, or 99% identical to the amino acid sequence of SEQ ID NO: 28. In embodiments, the BTN2A1 protein, or a fragment thereof comprise a extracellular domain (ECD). In embodiments, the variable ECD of the BTN2A1 protein comprises an amino acid sequence that is at least 90%, or 93%, or 95%, or 97%, or 98%, or 99% identical to the amino acid sequence of SEQ ID NO: 27.

In embodiments, the BTN3A1 protein, or a fragment thereof comprise a variable Ig-like V-type domain. In embodiments, the variable Ig-like V-type domain of the BTN3A1 protein comprises an amino acid sequence that is at least 90%, or 93%, or 95%, or 97%, or 98%, or 99% identical to the amino acid sequence of SEQ ID NO: 30. In embodiments, the BTN3A1 protein, or a fragment thereof comprise a extracellular domain (ECD). In embodiments, the variable ECD of the BTN3A1 protein comprises an amino acid sequence that is at least 90%, or 93%, or 95%, or 97%, or 98%, or 99% identical to the amino acid sequence of SEQ ID NO: 29.

In embodiments, the first domain comprises an amino acid sequence having an amino acid sequence of selected from one or more of SEQ ID NOs: 27-30.

In embodiments, the targeting domain is an antibody, an antibody-like molecule, or antigen binding fragment thereof. In embodiments, the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)₂. In embodiments, the antibody-like molecule is an scFv. In embodiments, the targeting domain is a polypeptide having an amino acid sequence with at least 90%, or 95%, or 97%, or 98%, or 99% identity with a polypeptide selected from SEQ ID NOs: 31-38, 41, 48, 111 and 112. In embodiments, the targeting domain is a polypeptide having an amino acid sequence with at least 90%, or 95%, or 97%, or 98%, or 99% identity with a polypeptide selected from SEQ ID NOs: 31-35, 41, 48, 111 and 112. In embodiments, the targeting domain is a polypeptide having an amino acid sequence of selected from one or more of SEQ ID NOs: 31-38, 41, 48, 111 and 112. In embodiments, the targeting domain is a polypeptide having an amino acid sequence of selected from one or more of SEQ ID NOs: 31-35, 41, 48, 111 and 112.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus.

In embodiments, the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus. In embodiments, the linker forms a heterodimer through electrostatic interactions between positively charged amino acid residues and negatively charged amino acid residues on the first and second charge polarized core domains.

In embodiments, the first and/or second charge polarized core domain comprises a polypeptide linker, optionally selected from a flexible amino acid sequence, IgG hinge region, or antibody sequence. In embodiments, the linker is a synthetic linker, optionally PEG. In embodiments, the linker comprises the hinge-CH2-CH3 Fc domain derived from IgG1, optionally from human IgG1. In embodiments, the linker comprises the hinge-CH2-CH3 Fc domain derived from IgG4, optionally from human IgG4.

In embodiments, the first and/or second charge polarized core domain further comprise peptides having positively and/or negatively charged amino acid residues at the amino and/or carboxy terminus of the charge polarized core domain. In embodiments, the positively charged amino acid residues include one or more of amino acids selected from His, Lys, and Arg. In embodiments, the positively charged amino acid residues are present in a peptide comprising positively charged amino acid residues in the first and/or the second charge polarized core domains. In embodiments, the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5). In embodiments, the peptide comprising positively charged amino acid residues comprises the sequence RKGGKR (SEQ ID NO: 11) or GSGSRKGGKRGS (SEQ ID NO: 12).

In embodiments, the negatively charged amino acid residues may include one or more amino acids selected from Asp and Glu. In embodiments, the negatively charged amino acid residues are present in a peptide comprising negatively charged amino acid residues in the first and/or the second charge polarized core domains. In embodiments, the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine, and where 20 each n is independently an integer 0 to 4) (SEQ ID NO: 6). In embodiments, the peptide comprising negatively charged amino acid residues comprises the sequence DEGGED (SEQ ID NO: 13) or GSGSDEGGEDGS (SEQ ID NO: 14).

In embodiments, the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81,114,116, 118, and 120. In embodiments, the alpha chain comprises an amino acid sequence that is at least 90%, or 93%, or 95%, or 97%, or 98%, or 99% identical to the amino acid sequence of SEQ ID NO: 75, and the beta chain comprises an amino acid sequence that is at least 90%, or 93%, or 95%, or 97%, or 98%, or 99% identical to the amino acid sequence of SEQ ID NO: 81. In embodiments, the alpha chain comprises an amino acid sequence that is at least 90%, or 93%, or 95%, or 97%, or 98%, or 99% identical to the amino acid sequence of SEQ ID NO: 113, and the beta chain comprises an amino acid sequence that is at least 90%, or 93%, or 95%, or 97%, or 98%, or 99% identical to the amino acid sequence of SEQ ID NO: 114. In embodiments, the alpha chain comprises an amino acid sequence that is at least 90%, or 93%, or 95%, or 97%, or 98%, or 99% identical to the amino acid sequence of SEQ ID NO: 115, and the beta chain comprises an amino acid sequence that is at least 90%, or 93%, or 95%, or 97%, or 98%, or 99% identical to the amino acid sequence of SEQ ID NO: 115. In embodiments, the alpha chain comprises an amino acid sequence that is at least 90%, or 93%, or 95%, or 97%, or 98%, or 99% identical to the amino acid sequence of SEQ ID NO: 117, and the beta chain comprises an amino acid sequence that is at least 90%, or 93%, or 95%, or 97%, or 98%, or 99% identical to the amino acid sequence of SEQ ID NO: 118. In embodiments, the alpha chain comprises an amino acid sequence that is at least 90%, or 93%, or 95%, or 97%, or 98%, or 99% identical to the amino acid sequence of SEQ ID NO: 119, and the beta chain comprises an amino acid sequence that is at least 90%, or 93%, or 95%, or 97%, or 98%, or 99% identical to the amino acid sequence of SEQ ID NO: 120. In embodiments, the invention provides for heterodimeric proteins and methods that further comprise administering an additional agent to a subject. In embodiments, the invention pertains to co-administration and/or co-formulation. Any of the compositions described herein may be co-formulated and/or co-administered.

In embodiments, a heterodimeric protein described herein acts synergistically when co-administered with another agent and is administered at doses that are lower than the doses commonly employed when such agents are used as monotherapy, wherein the heterodimeric protein comprises an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. In various embodiments, any agent referenced herein may be used in combination with any of the heterodimeric proteins described herein.

In various embodiments, a of the heterodimeric proteins disclosed herein may be co-administered with another heterodimeric protein disclosed herein, wherein the heterodimeric protein comprises an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains. Without wishing to be bound by theory, it is believed that a combined regimen involving the administration of one or more heterodimeric proteins which induce an innate immune response and one or more heterodimeric proteins which induce an adaptive immune response may provide synergistic effects (e.g., synergistic anti-tumor effects).

In various embodiments, a heterodimeric protein which induces an innate immune response may be utilized in the current disclosure, wherein the heterodimeric protein comprises an alpha chain and a beta chain, wherein the alpha chain comprises: (a) a first domain comprising a BTN2A1 protein, or a fragment thereof; (b) a second domain comprising a targeting domain; and (c) a linker that adjoins the first and second domains; and wherein the beta chain comprises: (a) a first domain comprising a BTN3A1 protein, or a fragment thereof; (b) a second domain comprising a targeting domain; and (c) a linker that adjoins the first and second domains.

In embodiments, inclusive of, without limitation, cancer applications, the current disclosure pertains to chemotherapeutic agents as additional agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In various embodiments, inclusive of, without limitation, cancer applications, the present additional agent is one or more immune-modulating agents selected from an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), MK-3475 (MERCK), BMS 936559 (BRISTOL MYERS SQUIBB), atezolizumab (TECENTRIQ, GENENTECH), MPDL3280A (ROCHE), an agent that increases and/or stimulates CD137 (4-1BB) and/or the binding of CD137 (4-1BB) with one or more of 4-1BB ligand (byway of non-limiting example, urelumab (BMS-663513 and anti-4-1BB antibody), and an agent that blocks, reduces and/or inhibits the activity of CTLA-4 and/or the binding of CTLA-4 with one or more of AP2M1, CD80, CD86, SHP-2, and PPP2R5A and/or the binding of OX40 with OX40L (by way of non-limiting example GBR 830 (GLENMARK), MEDI6469 (MEDIMMUNE).

In embodiments, inclusive of, without limitation, infectious disease applications, the current disclosure pertains to anti-infectives as additional agents. In embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

In embodiments, inclusive, without limitation, of autoimmune applications, the additional agent is an immunosuppressive agent. In embodiments, the immunosuppressive agent is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the current disclosure include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the current disclosure, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin).

In embodiments, the heterodimeric proteins (and/or additional agents) described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids. In still other embodiments, the heterodimeric proteins (and/or additional agents) described herein further comprise a cytotoxic agent, comprising, in illustrative embodiments, a toxin, a chemotherapeutic agent, a radioisotope, and an agent that causes apoptosis or cell death. Such agents may be conjugated to a composition described herein.

The heterodimeric proteins (and/or additional agents) described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

### Formulations

In one aspect, the current disclosure provides a pharmaceutical composition, comprising the heterodimeric protein of any of the embodiments disclosed herein.

The heterodimeric proteins (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions described herein are in the form of a pharmaceutically acceptable salt.

Further, the heterodimeric protein (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the compositions described herein are resuspended in a saline buffer (including, without limitation TBS, PBS, and the like).

In various embodiments, the heterodimeric proteins may by conjugated and/or fused with another agent to extend half-life or otherwise improve pharmacodynamic and pharmacokinetic properties. In embodiments, the heterodimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In various embodiments, each of the individual heterodimeric proteins is fused to one or more of the agents described in BioDrugs (2015) 29:215-239, the entire contents of which are hereby incorporated by reference.

### Administration, Dosing, and Treatment Regimens

The current disclosure includes the described heterodimeric protein (and/or additional agents) in various formulations. The heterodimeric protein (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the formulations comprising the heterodimeric protein (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the heterodimeric protein (and/or additional agents) of the current disclosure may conveniently be presented in unit dosage forms and may be prepared by any of the methods well known in the art of pharmacy. Such methods generally include the step of bringing the therapeutic agents into association with a carrier, which constitutes one or more accessory ingredients. Typically, the formulations are prepared by uniformly and intimately bringing the therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, the heterodimeric protein (and/or additional agents) described herein is formulated in accordance with routine procedures as a composition adapted for a mode of administration described herein.

Routes of administration include, for example: intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In most instances, administration results in the release of any agent described herein into the bloodstream.

The heterodimeric protein (and/or additional agents) described herein can be administered orally. Such heterodimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In one embodiment, for instance in the treatment of cancer, the heterodimeric protein (and/or additional agents) are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In various embodiments, for instance in the treatment of cancer, the heterodimeric protein (and/or additional agents) are administered intratumorally.

In the various embodiments, the present heterodimeric protein allows for a dual effect that provides less side effects than are seen in conventional immunotherapy (e.g., treatments with one or more of OPDIVO, KEYTRUDA, YERVOY, and TECENTRIQ). For example, the present heterodimeric proteins reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease. Further, the present local administration, e.g., intratumorally, obviate adverse event seen with standard systemic administration, e.g., IV infusions, as are used with conventional immunotherapy (e.g., treatments with one or more of OPDIVO, KEYTRUDA, YERVOY, and TECENTRIQ).

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of the heterodimeric protein (and/or additional agents) described herein as well as the dosing schedule can depend on various parameters, including, but not limited to, the disease being treated, the subject's general health, and the administering physician's discretion. The heterodimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In various embodiments the heterodimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

In various embodiments, the current disclosure relates to the co-administration of a heterodimeric protein which induces an innate immune response and another heterodimeric protein which induces an adaptive immune response. In such embodiments, the heterodimeric protein which induces an innate immune response may be administered before, concurrently with, or subsequent to administration of the heterodimeric protein which induces an adaptive immune response. For example, the heterodimeric proteins may be administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart. In an illustrative embodiment, the heterodimeric protein which induces an innate immune response and the heterodimeric protein which induces an adaptive response are administered 1 week apart, or administered on alternate weeks (i.e., administration of the heterodimeric protein inducing an innate immune response is followed 1 week later with administration of the heterodimeric protein which induces an adaptive immune response and so forth).

The dosage of the heterodimeric protein (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected.

For administration of the heterodimeric protein (and/or additional agents) described herein by parenteral injection, the dosage may be about 0.1 mg to about 250 mg per day, about 1 mg to about 20 mg per day, or about 3 mg to about 5 mg per day. Generally, when orally or parenterally administered, the dosage of any agent described herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the heterodimeric protein (and/or additional agents) described herein is by parenteral injection at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the heterodimeric protein (and/or additional agents) is in a range of about 0.01 mg/kg to about 100 mg/kg of body weight, or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween.

In another embodiment, delivery can be in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989).

The heterodimeric protein (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In another embodiment, polymeric materials can be used (see Medical Applications of Controlled Release, Langer and Wise (eds.), CRC Pres., Boca Raton, Florida (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In another embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of the heterodimeric protein (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing the heterodimeric protein (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. The heterodimeric protein (and/or additional agents) described herein can be administered in a single daily dose, or the total daily dosage can be administered in divided doses of two, three or four times daily. Furthermore, the heterodimeric protein (and/or additional agents) described herein can be administered continuously rather than intermittently throughout the dosage regimen.

### Cells and Nucleic Acids

In one aspect, the current disclosure provides an expression vector, comprising a nucleic acid encoding the first and/or second polypeptide chains of the heterodimeric protein of any of any of the embodiments disclosed herein. In embodiments, the expression vector is a mammalian expression vector. In embodiments, the expression vector comprises DNA or RNA. In embodiments, In one aspect, the current disclosure provides a host cell comprising the expression vector of any one of the embodiments disclosed herein.

In various embodiments, the current disclosure provides an expression vector, comprising a nucleic acid encoding the heterodimeric protein (e.g., a heterodimeric protein comprising a first and second polypeptide chains) described herein. In various embodiments, the expression vector comprises DNA or RNA. In various embodiments, the expression vector is a mammalian expression vector.

Both prokaryotic and eukaryotic vectors can be used for expression of the heterodimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and λP_{L}. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the heterodimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the R-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the fusion proteins in recombinant host cells.

In embodiments, expression vectors of the invention comprise a nucleic acid encoding at least the first and/or second polypeptide chains of the heterodimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In an embodiment, the cell is a tumor cell. In another embodiment, the cell is a non-tumor cell. In an embodiment, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In an embodiment, the current disclosure contemplates the use of inducible promoters capable of effecting high level of expression transiently in response to a cue. For example, when in the proximity of a tumor cell, a cell transformed with an expression vector for the heterodimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et a., TIG 15:326-332, 1999; Kootstra et a., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), FIp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric fusion proteins including CRISPR/CAS9, zinc finger, TALEN, and meganuclease gene-editing technologies.

In one aspect, the invention provides expression vectors for the expression of the heterodimeric proteins (and/or additional agents) that are viral vectors. Many viral vectors useful for gene therapy are known (see, e.g., Lundstrom, Trends Biotechnol., 21: 117, 122, 2003. Illustrative viral vectors include those selected from Antiviruses (LV), retroviruses (RV), adenoviruses (AV), adeno-associated viruses (AAV), and α viruses, though other viral vectors may also be used. For in vivo uses, viral vectors that do not integrate into the host genome are suitable for use, such as α viruses and adenoviruses. Illustrative types of α viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In one embodiment, the invention provides methods of transducing a human cell in vivo, comprising contacting a solid tumor in vivo with a viral vector of the invention.

In various embodiments, the current disclosure provides a host cell, comprising the expression vector comprising the heterodimeric protein described herein.

Expression vectors can be introduced into host cells for producing the present heterodimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the fusion proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E. G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection, or from commercial suppliers.

Cells that can be used for production of the present heterodimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of tumor or infectious disease being treated or prevented, and can be determined by one of skill in the art.

Production and purification of Fc-containing macromolecules (such as Fc fusion proteins) has become a standardized process, with minor modifications between products. For example, many Fc containing macromolecules are produced by human embryonic kidney (HEK) cells (or variants thereof) or Chinese Hamster Ovary (CHO) cells (or variants thereof) or in some cases by bacterial or synthetic methods. Following production, the Fc containing macromolecules that are secreted by HEK or CHO cells are purified through binding to Protein A columns and subsequently ‘polished’ using various methods. Generally speaking, purified Fc containing macromolecules are stored in liquid form for some period of time, frozen for extended periods of time or in some cases lyophilized. In various embodiments, production of the heterodimeric proteins contemplated herein may have unique characteristics as compared to traditional Fc containing macromolecules. In certain examples, the heterodimeric proteins may be purified using specific chromatography resins, or using chromatography methods that do not depend upon Protein A capture. In other embodiments, the heterodimeric proteins may be purified in an oligomeric state, or in multiple oligomeric states, and enriched for a specific oligomeric state using specific methods. Without being bound by theory, these methods could include treatment with specific buffers including specified salt concentrations, pH and additive compositions. In other examples, such methods could include treatments that favor one oligomeric state over another. The heterodimeric proteins obtained herein may be additionally ‘polished’ using methods that are specified in the art. In embodiments, the heterodimeric proteins are highly stable and able to tolerate a wide range of pH exposure (between pH 3-12), are able to tolerate a large number of freeze/thaw stresses (greater than 3 freeze/thaw cycles) and are able to tolerate extended incubation at high temperatures (longer than 2 weeks at 40 degrees C.). In other embodiments, the heterodimeric proteins are shown to remain intact, without evidence of degradation, deamidation, etc. under such stress conditions.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In other embodiments, the subject and/or animal is a non-mammal, such, for example, a zebrafish. In embodiments, the subject and/or animal may comprise fluorescently-tagged cells (with e.g., GFP). In embodiments, the subject and/or animal is a transgenic animal comprising a fluorescent cell.

In embodiments, the subject and/or animal is a human. In embodiments, the human is a pediatric human. In other embodiments, the human is an adult human. In other embodiments, the human is a geriatric human. In other embodiments, the human may be referred to as a patient.

In certain embodiments, the human has an age in a range of from about 0 months to about 6 months old, 15 from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In other embodiments, the subject is a non-human animal, and therefore the invention pertains to veterinary use. In a specific embodiment, the non-human animal is a household pet. In another specific embodiment, the non-human animal is a livestock animal.

### Kits

The invention provides kits that can simplify the administration of any agent described herein. An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

### EXAMPLES

The examples herein are provided to illustrate advantages and benefits of the present disclosure and to further assist a person of ordinary skill in the art with preparing or using the chimeric proteins of the present disclosure. The examples herein are also presented in order to more fully illustrate the preferred aspects of the present disclosure. The examples should in no way be construed as limiting the scope of the present disclosure, as defined by the appended claims. The examples can include or incorporate any of the variations, aspects or embodiments of the present disclosure described above. The variations, aspects or embodiments described above may also further each include or incorporate the variations of any or all other variations, aspects or embodiments of the present disclosure.

### Example 1: Construction and Characterization of an Illustrative BTN2A1/3A1-Fc-CD19scFv Heterodimeric Protein

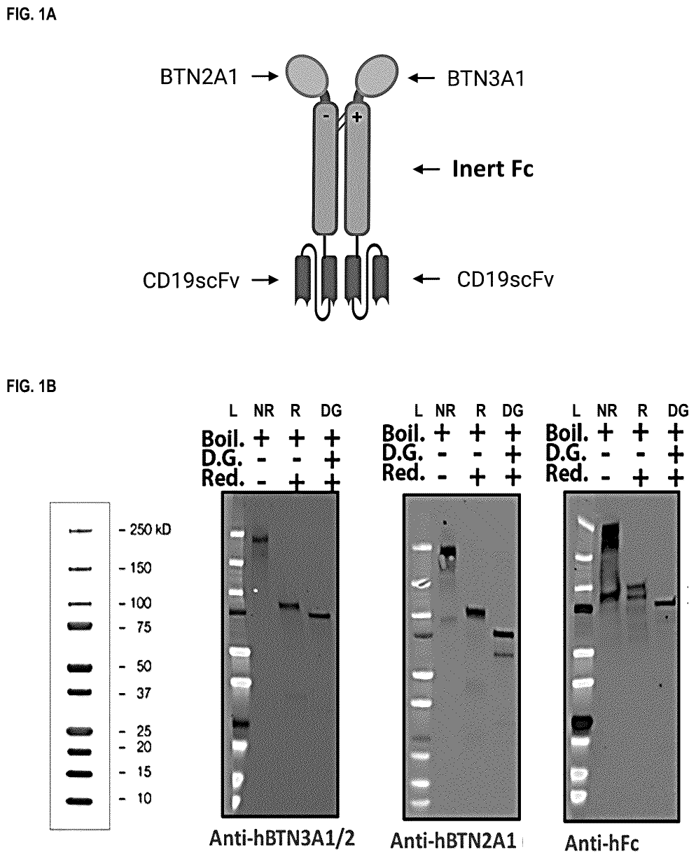

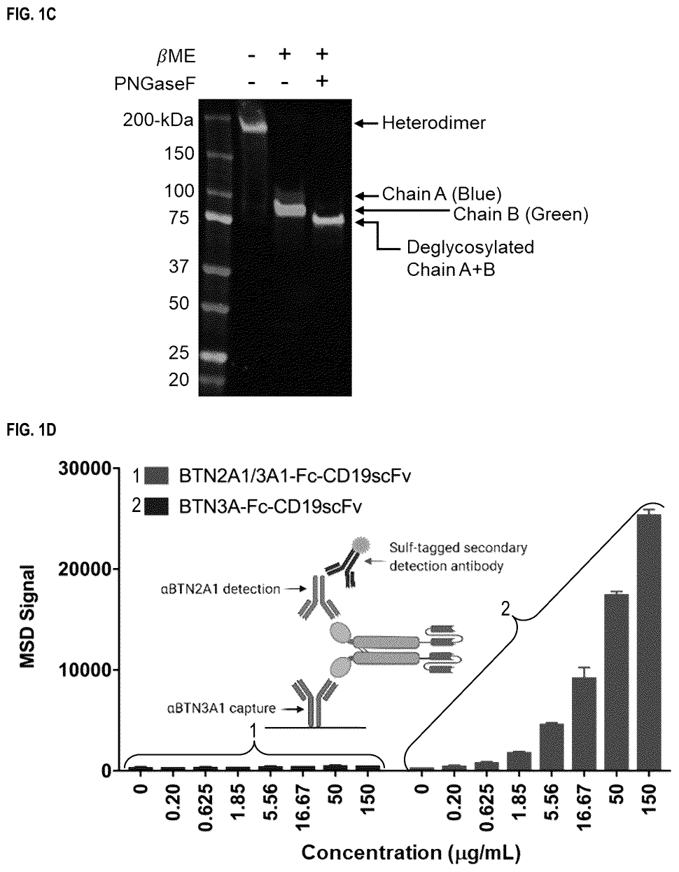

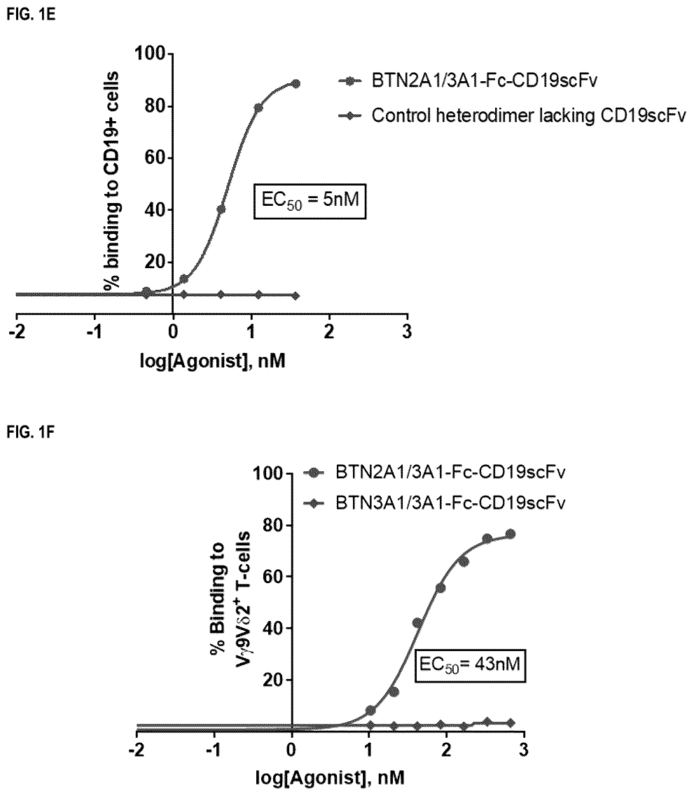

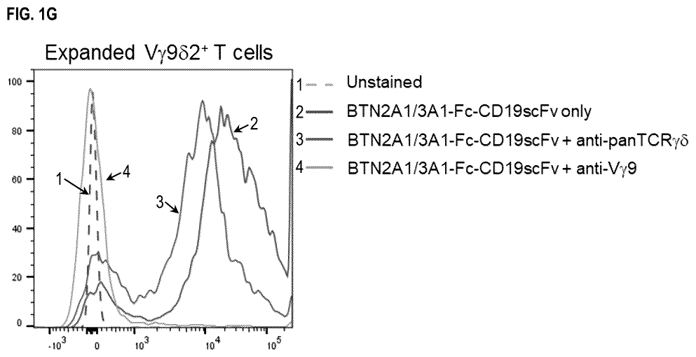

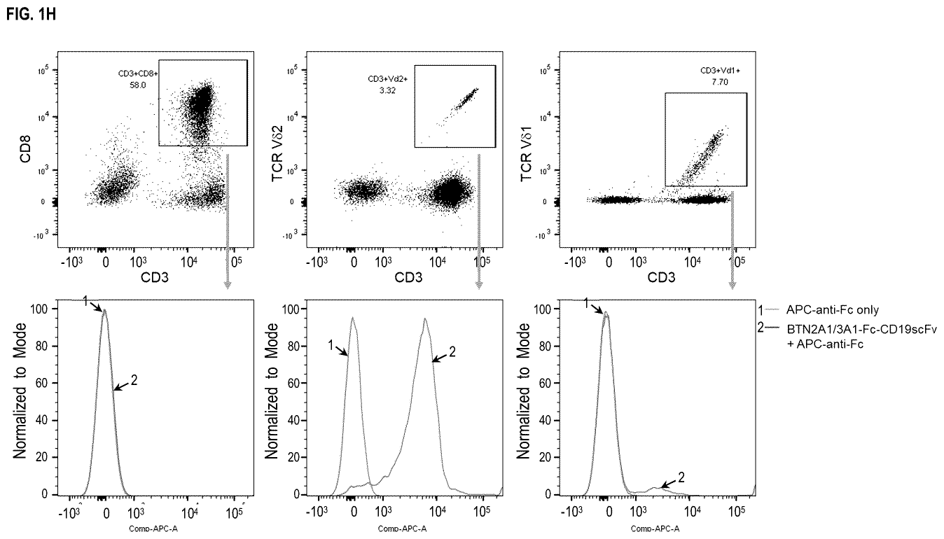

The heterodimeric proteins of the present disclosure comprise a dimer of two chimeric proteins, each comprising a butyrophilin family member, a core domain, and an antigen-targeting domain. The “BTN2A1/3A1-Fc-CD19scFv” construct included an alpha chain comprising an extracellular domain (ECD) of human BTN2A1 fused to a CD19scFv via a hinge-CH2-CH3 Fc domain, and a beta chain comprising an extracellular domain (ECD) of human BTN3A1 fused to a CD19scFv via a hinge-CH2-CH3 Fc domain. See, FIG. 1A. Constructs encoding BTN2A1-Fc-CD19scFv protein (alpha chain) and BTN3A1-Fc-CD19scFv protein (beta chain) were generated. This GAmma DELta T cell ENgager construct also is referred to herein as the BTN2A1/3A1-Fc-CD19scFv heterodimeric protein.

The BTN2A1/3A1-Fc-CD19scFv heterodimer protein that was produced via a transient co-transfection in Expi293 cells of two plasmids encoding 1) the BTN2A1-alpha-CD19scFv protein and 2) the BTN3A1-beta-CD19scFv protein. The alpha and beta constructs encoded a BTN2A1-Fc-CD19scFv (‘alpha’ chain) and a BTN3A1-Fc-CD19scFv (‘beta’ chain). The alpha and beta chains contained charged polarized linker domains which facilitated heterodimerization of the desired the BTN2A1/3A1-Fc-CD19scFv heterodimeric protein. The cell culture supernatant from the transient transfection was harvested 6 days following transfection and purified over an FcXL chromatography resin.

Purity of the protein was assessed using non-reducing sodium dodecyl sulfate polyacrylamide gel electrophoresis (SDS-PAGE). The purified protein was also analyzed by western blots using non-reducing, reducing, and reducing and deglycosylating conditions, following detection with an anti-human BTN2A1 antibody, an anti-human BTN3A1 antibody, or an anti-mouse Fc antibody. Non-reduced BTN2A1/3A1-Fc-CD19scFv heterodimeric protein ran as a single band (See lanes “NR” in FIG. 1B) indicative of covalent complex formation between the BTN2A1-alpha-CD19scFv and BTN3A1-beta-CD19scFv chains. As shown in FIG. 1B, the blots probed with the anti-Fc antibody revealed two bands with the protein prepared under reducing but non-deglycosylated condition (See lane “R” in FIG. 1B). Gels probed with the anti-human BTN2A1 and the anti-human BTN3A1 antibodies indicated bands with mobility corresponding to the two bands revealed in the anti Fc-probed blot. Interestingly, protein prepared under both reduced and deglycosylated (lane “DG”) conditions resulted in a single band, which could be detected with any of the anti-human BTN2A1, anti-human BTN3A1, or anti-mouse Fc antibodies.

### Example 2: Enhanced Activation of γδ T Cells by the Combination of the BTN2A1/3A1-Fc-CD19scFv Heterodimeric Protein and an Anti-NKG2D Antibody

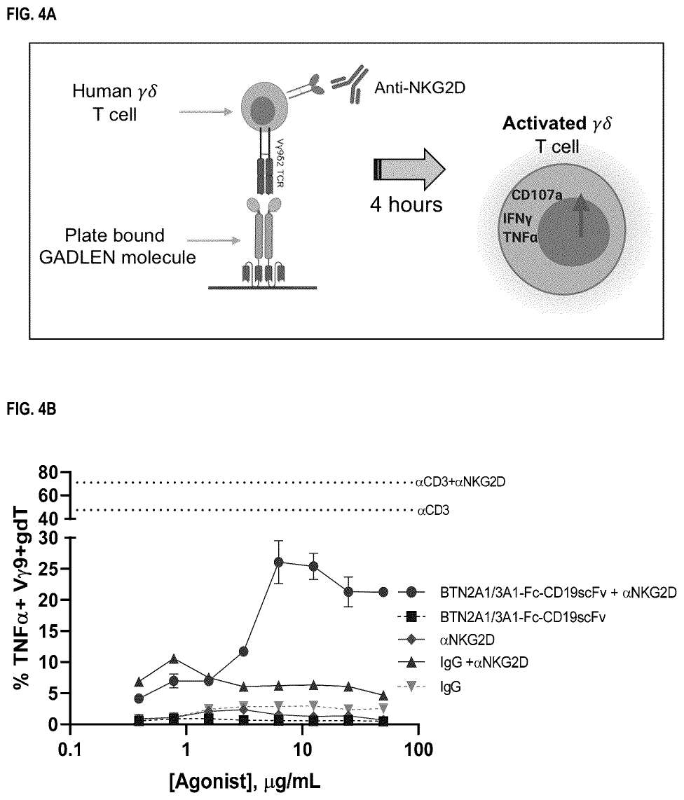

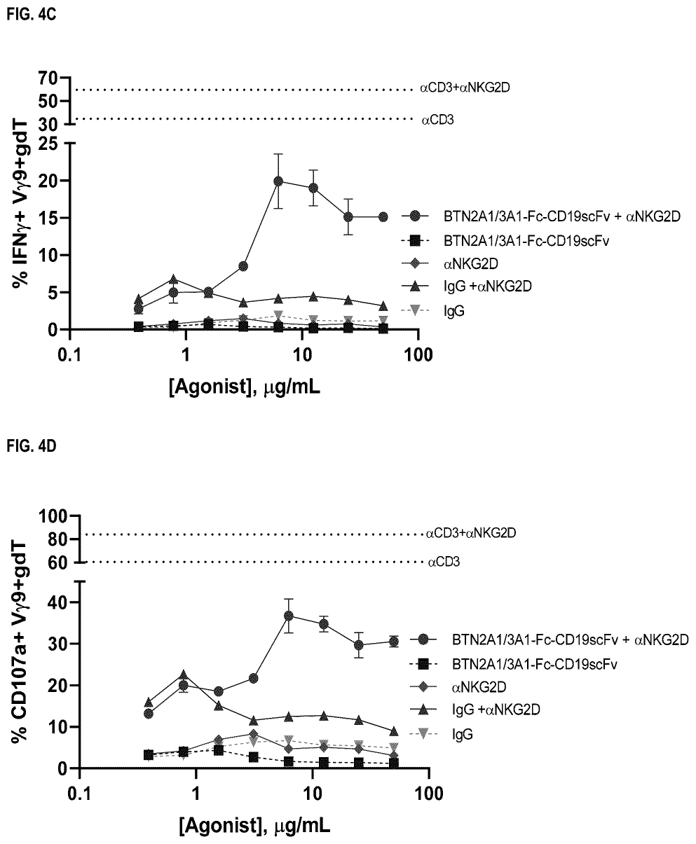

An in vitro assay was developed to study the activation of the γδ T cells by the BTN2A1/3A1-Fc-CD19scFv heterodimeric protein. Briefly, as shown in FIG. 4A, plates were coated with increasing amounts of (1) an anti-NKG2D antibody (Clone #149810) alone, (2) the BTN2A1/3A1-Fc-CD19scFv heterodimeric protein alone, (3) and the combination of the BTN2A1/3A1-Fc-CD19scFv heterodimeric protein in combination with the anti-NKG2D antibody (1 μg/ml). Increasing amounts of IgG alone or in combination of the anti-NKG2D antibody were used as controls. 1×10⁵ human γδ T cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with anti-TNFα, anti-IFNγ or anti-CD107a, the degranulation marker of the activated γδ T cells, and analyzed by flow cytometry. The frequency of Vγ9+ T cells expressing cytotoxic cytokines TNFα, IFNγ or the degranulation marker CD107a was determined by flow cytometry.

Surprisingly, as shown in in FIG. 4B, very few γδ T cells, if any, exhibited expression of TNFα when stimulated by the BTN2A1/3A1-Fc-CD19scFv heterodimeric protein alone or the anti-NKG2D antibody alone. In contrast, unexpectedly, an increased fraction of γδ T cells stimulated with the combination of the BTN2A1/3A1-Fc-CD19scFv heterodimeric protein and the anti-NKG2D antibody exhibited the expression of TNFα (FIG. 4B). As expected, very few γδ T cells, if any, exhibited expression of TNFα when stimulated by IgG, the negative control (FIG. 4B). While a slightly higher fraction of γδ T cells stimulated with the combination of IgG and the anti-NKG2D antibody exhibited the expression of TNFα, the effect was not dose-dependent (FIG. 4B).

The frequency of Vγ9+ T cells expressing IFNγ was then determined by flow cytometry. As shown in in FIG. 4C, very few γδ T cells, if any, exhibited expression of IFNγ when stimulated by the BTN2A1/3A1-Fc-CD19scFv heterodimeric protein alone or the anti-NKG2D antibody alone. In contrast, as observed for TNFα, an increased fraction of γδ T cells stimulated with the combination of the BTN2A1/3A1-Fc-CD19scFv heterodimeric protein and the anti-NKG2D antibody exhibited the expression of IFNγ (FIG. 4C). As expected, very few γδ T cells, if any, exhibited expression of IFNγ when stimulated by IgG, the negative control (FIG. 4C). While a slightly higher fraction of γδ T cells stimulated with the combination of IgG and the anti-NKG2D antibody exhibited the expression of IFNγ, the effect was not dose-dependent (FIG. 4C).

Then, the frequency of Vγ9+ T cells expressing degranulation marker CD107a was determined by flow cytometry to assess whether the. As shown in in FIG. 4D, very few γδ T cells, if any, exhibited expression of CD107a when stimulated by the BTN2A1/3A1-Fc-CD19scFv heterodimeric protein alone or the anti-NKG2D antibody alone. In contrast, as observed for TNFα and IFNγ, an increased fraction of γδ T cells stimulated with the combination of the BTN2A1/3A1-Fc-CD19scFv heterodimeric protein and the anti-NKG2D antibody exhibited the expression of CD107a (FIG. 4D). As expected, very few γδ T cells, if any, exhibited expression of CD107a when stimulated by IgG, the negative control (FIG. 4D). While a slightly higher fraction of γδT cells stimulated with the combination of IgG and the anti-NKG2D antibody exhibited the expression of CD107a, the effect was not dose-dependent (FIG. 4D).

These results surprisingly demonstrate that a combination of the BTN2A1/3A1-Fc-CD19scFv heterodimeric protein and a co-stimulation of the NKG2D pathway causes greater activation of γδ T cells compared to the BTN2A1/3A1-Fc-CD19scFv heterodimeric protein, by itself. These results also demonstrate that the activation of the NKG2D pathway, by itself, is less efficient in the in vitro assay for full activation of γδ T cells.

### Example 3: Enhanced Activation of γδ T Cells by the Combination of the BTN2A1/3A1-Fc-CD19scFv Heterodimeric Protein and an Anti-CD28 Antibody

Whether the combination of the BTN2A1/3A1-Fc-CD19scFv heterodimeric protein with the activation of CD28 pathway would cause enhanced activation of γδ T cells was next explored. Briefly, plates were coated with the 10 μg/mL BTN2A1/3A1-Fc-CD19scFv heterodimeric protein in combination with increasing amounts of the anti-NKG2D antibody, or an anti-CD28 antibody. 1×10⁵ human γδ T cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with anti-CD107a, the degranulation marker of the activated γ6 T cells, and analyzed by flow cytometry. The frequency of Vγ9+ T cells expressing cytotoxic cytokines TNFα, IFNγ or the degranulation marker CD107a was determined by flow cytometry.

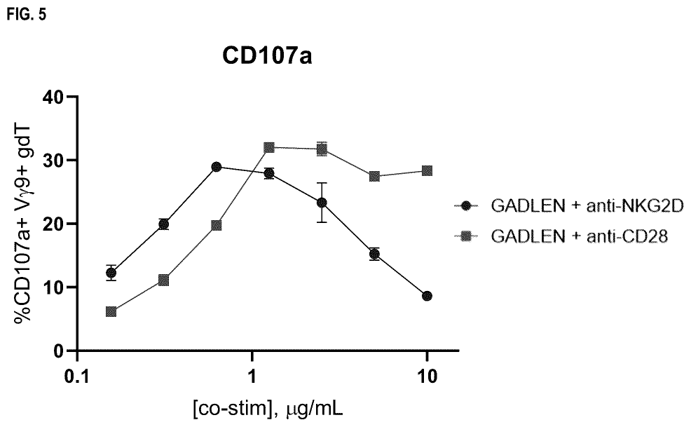

The frequency of Vγ9+ T cells expressing CD107a as determined by flow cytometry was plotted as a function of the amount of plate bound-anti-NKG2D antibody, or an anti-CD28 antibody. As shown in FIG. 5, co-stimulation by the anti-CD28 antibody resulted in CD107a expression that was comparable to that with the co-stimulation with the anti-NKG2D antibody.

These results demonstrate that the co-stimulation of γδ T cells by anti-CD28, in combination with the BTN2A1/3A1-Fc-CD19scFv heterodimeric protein, resulted in more robust cytokine production, but similar level of degranulation, compared to the anti-NKG2D antibody. These results further demonstrate that a combination of the BTN2A1/3A1-Fc-CD19scFv heterodimeric protein and a co-stimulation cause enhanced activation of γδ T cells.

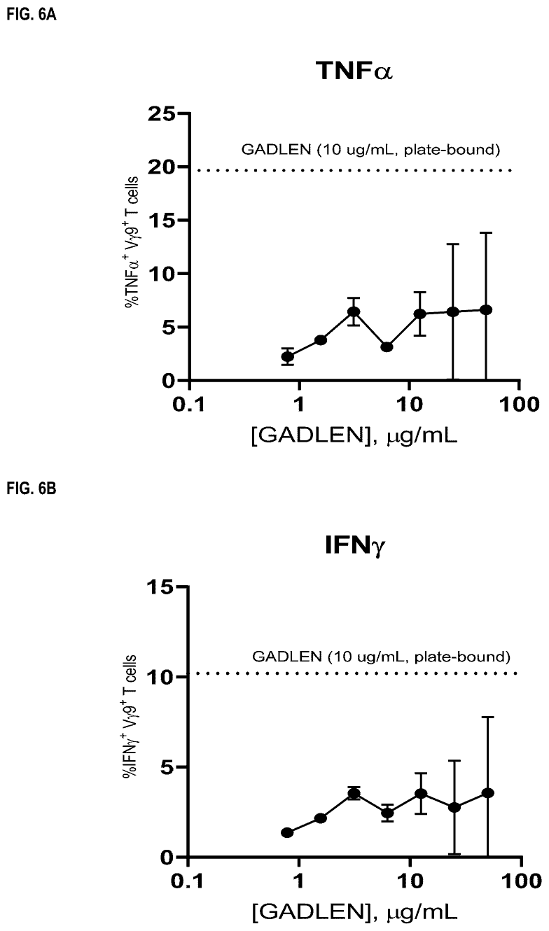

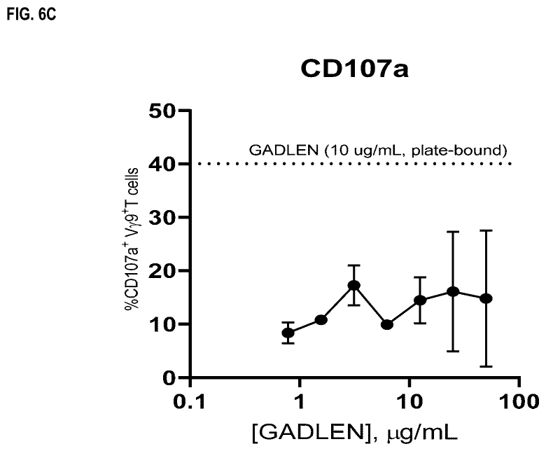

In a set of experiments, γδ T cells were stimulated with solutions of the BTN2A1/3A1-Fc-CD19scFv heterodimeric protein alone or in combination with soluble the anti-NKG2D antibody, or the anti-CD28 antibody in a soluble format. In one experiment, human γδ T cells (from donor 3) were stimulated with various concentration soluble BTN2A1/3A1-Fc-CD19scFv3 (2-fold serial dilution starting at 50 ug/mL) or 10 μg/mL plate-bound BTN2A1/3A1-Fc-CD19scFv+anti-NKG2D (1 μg/mL, plate-bound). The frequency of Vγ9+ T cells expressing IFNγ, TNFα, and CD107a was assessed by flow cytometry after 4 hours of stimulation. As shown in FIG. 6A, the γδ T cell activation, as judged by the fraction of cells expressing TNFα, was higher with plate-bound heterodimeric protein compared to the activation in soluble format. Similarly, the γδ T cell activation, as judged by the fraction of cells expressing IFNγ (FIG. 6B) and CD107a (FIG. 6C), was higher with plate-bound heterodimeric protein compared to the activation in soluble format.

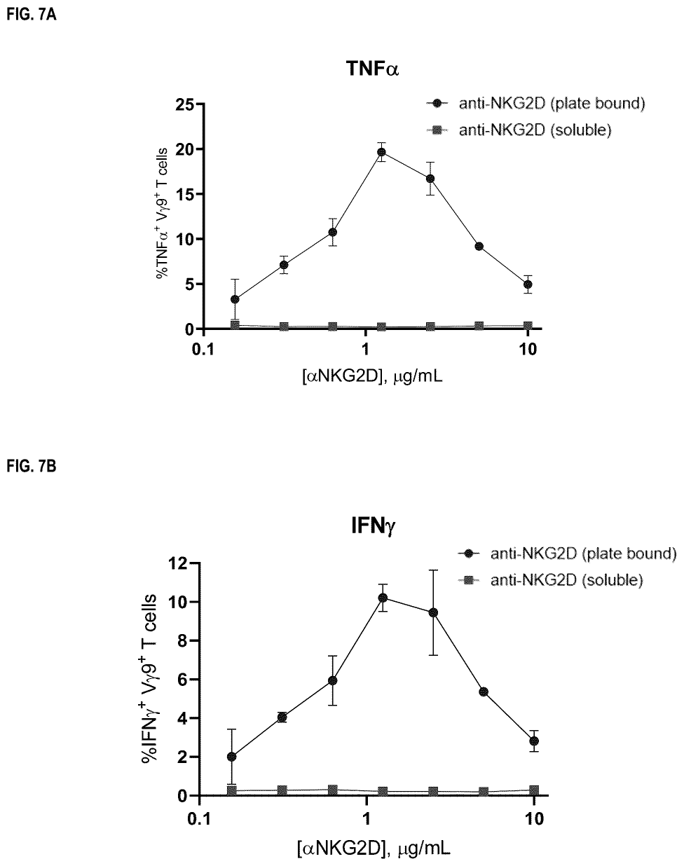

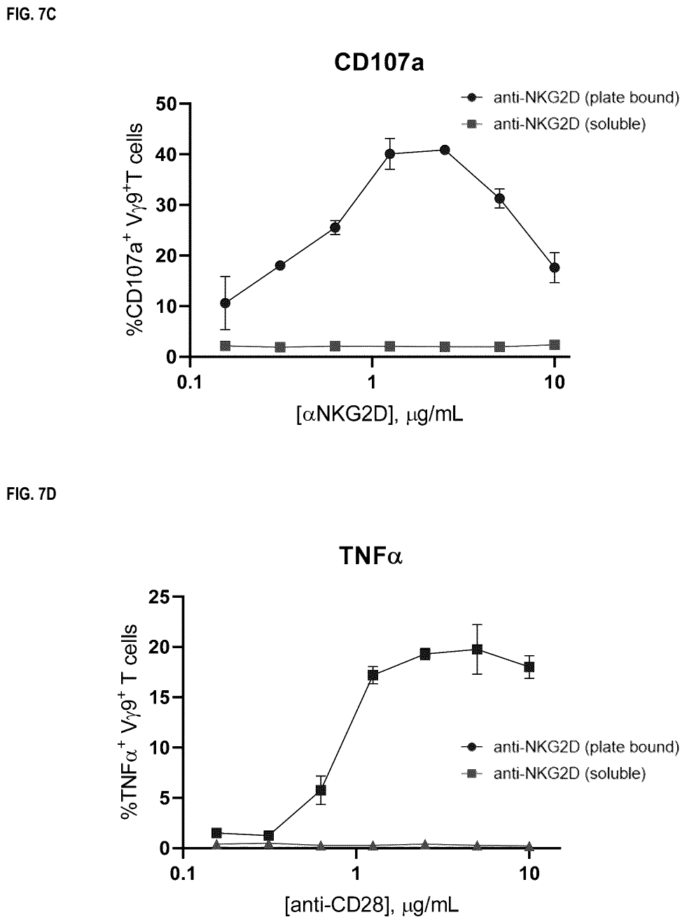

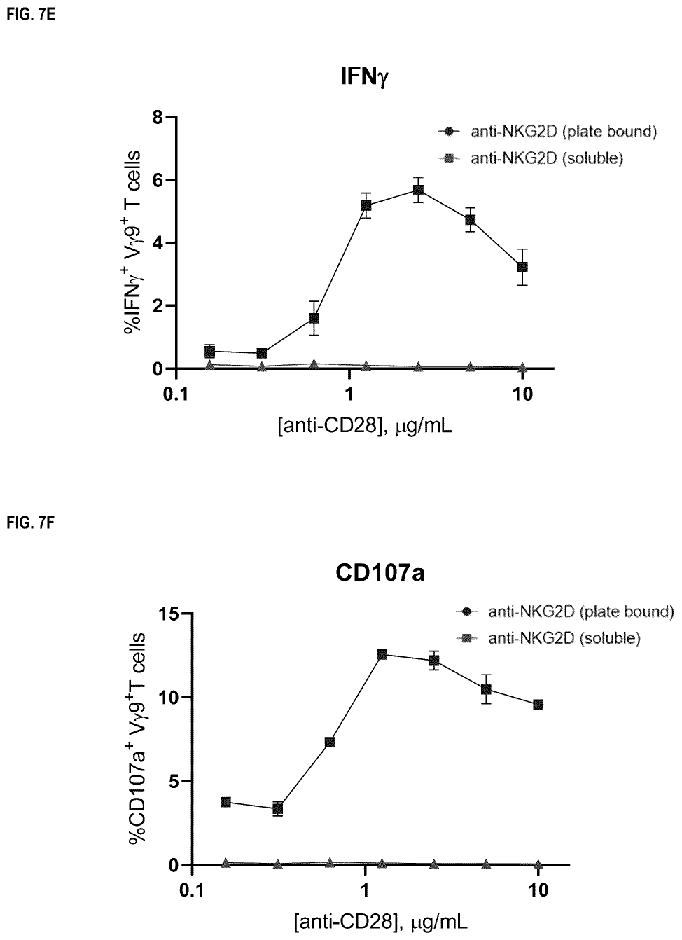

To explore this further, γδ T cells were stimulated with the BTN2A1/3A1-Fc-CD19scFv heterodimeric protein (10 μg/mL) with various concentrations of plate-bound or soluble anti-NKG2D antibody. The % γδ T cell expressing IFNγ, TNFα, and CD107a was assessed by flow cytometry after 4 hours of stimulation. As shown in FIG. 7A, the γδ T cell activation induced by the BTN2A1/3A1-Fc-CD19scFv heterodimeric protein in combination with the anti-NKG2D antibody, as judged by the fraction of cells expressing TNFα, observed in a plate-bound format but remained at a background level in soluble format. Similarly, the γδ T cell activation induced by the BTN2A1/3A1-Fc-CD19scFv heterodimeric protein in combination with the anti-NKG2D antibody, as judged by the fraction of cells expressing IFNγ (FIG. 7B) and CD107a (FIG. 7C), observed in a plate-bound format but remained at a background level in soluble format, was higher with plate-bound heterodimeric protein compared to the activation in soluble format.

In a similar experiment, γδ T cells were stimulated with the BTN2A1/3A1-Fc-CD19scFv heterodimeric protein (10 μg/mL) with various concentrations of plate-bound or soluble anti-CD28 antibody. The % γδ T cell expressing IFNγ, TNFα, and CD107a was assessed by flow cytometry after 4 hours of stimulation. As shown in FIG. 7D, the γδ T cell activation induced by the BTN2A1/3A1-Fc-CD19scFv heterodimeric protein in combination with the anti-CD28 antibody, as judged by the fraction of cells expressing TNFα, observed in a plate-bound format but remained at a background level in soluble format. Similarly, the γδ T cell activation induced by the BTN2A1/3A1-Fc-CD19scFv heterodimeric protein in combination with the anti-CD28 antibody, as judged by the fraction of cells expressing IFNγ (FIG. 7E) and CD107a (FIG. 7F), observed in a plate-bound format but remained at a background level in soluble format, was higher with plate-bound heterodimeric protein compared to the activation in soluble format.

These results demonstrate that γδ T cell activation was significantly higher with plate-bound heterodimeric protein and plate-bound anti-NKG2D. Without wishing to be bound by theory, these data may suggest that crosslinking of the BTN2A1/3A1 receptors and costimulatory receptors may be involved in enhancement in the activity.

### Example 4: Titration of BTN2A1/3A1-Fc-CD19scFv Heterodimeric Protein and the NKG2D Ligands (MICA and MICB) for the Activation of γδ T Cells

Experiments were carried out to compare the ability of MICA, MICB, or a mixture of MICA and MICB, and the anti-NKG2D antibody to costimulate γδ T cells in the presence of the BTN2A1/3A1-Fc-CD19scFv heterodimeric protein.

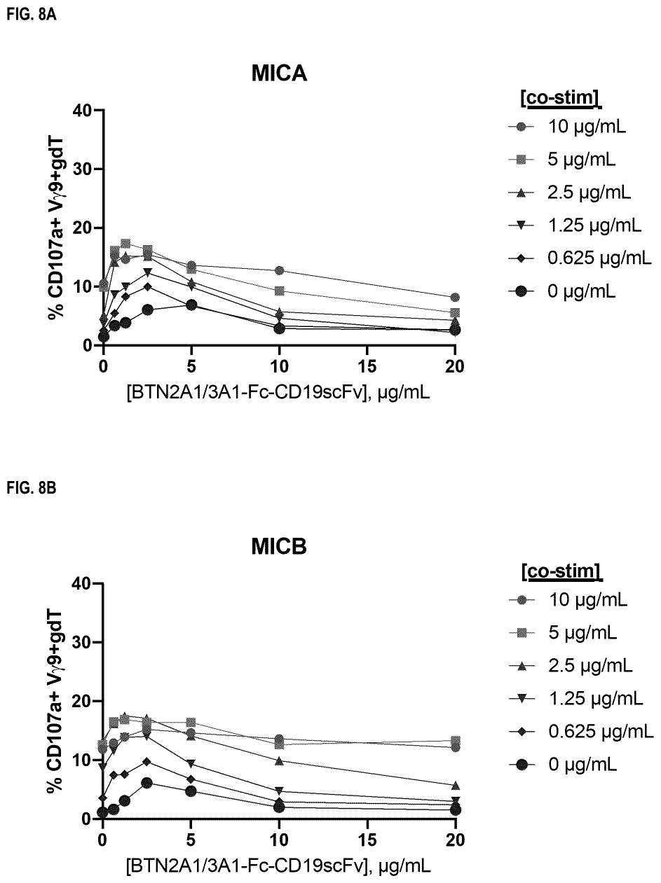

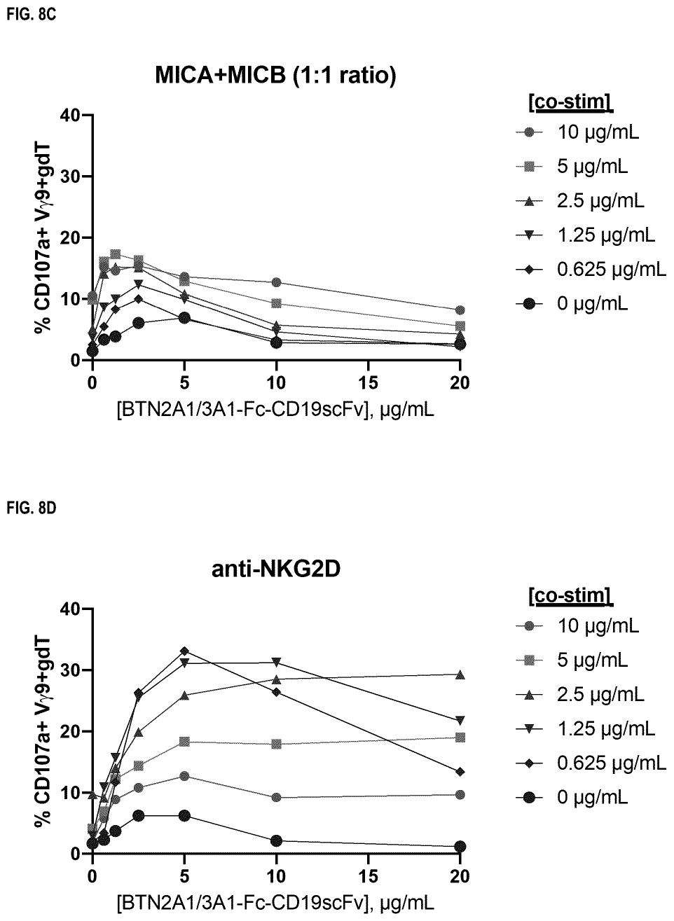

Briefly, plates were coated with increasing amounts of the BTN2A1/3A1-Fc-CD19scFv heterodimeric protein (2-fold serial dilution starting at 20 μg/mL) in combination with increasing amounts of MICA-Fc fusion protein (2-fold serial dilution starting at 10 μg/mL). 1×10⁵ human γδ T cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with an anti-CD107a antibody, the degranulation marker of the activated γδT cells, and analyzed by flow cytometry. The frequency of Vγ9+ T cells expressing CD107a was plotted as a function of the amount of plate bound MICA-Fc or MICB-Fc. As shown in FIG. 8A, MICA-Fc fusion protein showed a dose-dependent co-stimulation of γδ T cells in the presence of the BTN2A1/3A1-Fc-CD19scFv heterodimeric protein. Interestingly, high concentrations of MICA alone caused the activation of γδ T cells (FIG. 8A). Further, as shown in FIG. 8A, activation of about −18% γδ T cells was observed upon co-stimulation with the MICA-Fc fusion protein.

To study the effect of MICB, plates were coated with increasing amounts of the BTN2A1/3A1-Fc-CD19scFv heterodimeric protein (2-fold serial dilution starting at 20 μg/mL) in combination with increasing amounts of MICB-Fc fusion protein (2-fold serial dilution starting at 10 μg/mL). 1×10⁵ human γδ T cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with an anti-CD107a antibody, the degranulation marker of the activated γδ T cells, and analyzed by flow cytometry. The frequency of Vγ9+ T cells expressing CD107a was plotted as a function of the amount of plate bound MICB-Fc or MICB-Fc. As shown in FIG. 8B, the MICB-Fc fusion protein showed a dose-dependent co-stimulation of γδ T cells in the presence of the BTN2A1/3A1-Fc-CD19scFv heterodimeric protein. Interestingly, high concentrations of MICB alone caused the activation of γδ T cells (FIG. 8B). Further, as shown in FIG. 8B, activation of about −18% γδ T cells was observed upon co-stimulation with the MICB-Fc fusion protein.

To study the effect of a 1:1 mixture of MICA and MICB, plates were coated with the increasing amounts of the BTN2A1/3A1-Fc-CD19scFv heterodimeric protein (2-fold serial dilution starting at 20 μg/mL) in combination with increasing amounts of the 1:1 mixture of MICA and MICB-Fc fusion protein (2-fold serial dilution starting at 10 μg/mL). 1×10⁵ human γδ T cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with an anti-CD107a antibody, the degranulation marker of the activated γδ T cells, and analyzed by flow cytometry. The frequency of Vγ9+ T cells expressing CD107a was plotted as a function of the amount of plate bound the 1:1 mixture of MICA and MICB-Fc or the 1:1 mixture of MICA and MICB-Fc. As shown in FIG. 8C, the 1:1 mixture of MICA and MICB-Fc fusion protein showed a dose-dependent co-stimulation of γδ T cells in the presence of the BTN2A1/3A1-Fc-CD19scFv heterodimeric protein. Interestingly, high concentrations of the 1:1 mixture of MICA and MICB alone caused the activation of γδT cells (FIG. 8C). Further, as shown in FIG. 8C, activation of about −18% γδ T cells was observed upon co-stimulation with the 1:1 mixture of MICA and MICB-Fc fusion protein.

To study the effect of the anti-NKG2D antibody, plates were coated with the increasing amounts of the BTN2A1/3A1-Fc-CD19scFv heterodimeric protein (2-fold serial dilution starting at 20 μg/mL) in combination with increasing amounts of the anti-NKG2D antibody-Fc fusion protein (2-fold serial dilution starting at 10 μg/mL). 1×10⁵ human γδ T cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with an anti-CD107a antibody, the degranulation marker of the activated γδ T cells, and analyzed by flow cytometry. The frequency of Vγ9+ T cells expressing CD107a was plotted as a function of the amount of plate bound the anti-NKG2D antibody-Fc or the anti-NKG2D antibody-Fc. As shown in FIG. 8D, the anti-NKG2D antibody-Fc fusion protein showed a dose-dependent co-stimulation of γδ T cells in the presence of the BTN2A1/3A1-Fc-CD19scFv heterodimeric protein. Interestingly, unlike MICA and MICB, high concentrations of the anti-NKG2D antibody alone did not cause significant activation of γδ T cells (FIG. 8D). Further, as shown in FIG. 8D, activation of about −30% γδ T cells was observed upon co-stimulation with the anti-NKG2D antibody-Fc fusion protein.

These results demonstrate that the co-stimulation delivered by anti-NKG2D resulted in degranulation of more γδ T cells (˜30%) compared to by MICA, MICB, or their combination (˜18%). Nonetheless, these results demonstrate that the combination of the BTN2A1/3A1-Fc-CD19scFv heterodimeric protein and a co-stimulation causes enhanced activation of γδ T cells.

### Example 5: Extracellular Domains (ECDs) of Both BTN2A1 and BTN3A1 in Combination with the Anti-NKG2D or Anti-CD28 Antibodies Cause Greater Activation of γδ T Cells Compared to the ECD BTN2A1 Alone or ECD of BTN3A1 Alone

The extracellular domains (ECDs) BTN2A1 alone and BTN3A1 alone (in the presence of costimulation) were 25 next compared with the combination of ECDs of BTN2A1 and BTN3A1 (in the presence of costimulation), or the BTN2A1/3A1-Fc-CD19scFv heterodimeric protein. Briefly, plates were coated with (1) BTN2A1-Fc, or BTN3A1-Fc, BTN2A1-Fc+BTN3A1-Fc (1:1), or the BTN2A1/3A1-Fc-CD19scFv heterodimeric protein in combination with (2) 1 μg/mL anti-NKG2D antibody or 2.5 μg/mL anti-CD28 antibody. 1×10⁵ human γδT cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with an anti-CD107a antibody, the degranulation marker of the activated γδ T cells, and analyzed by flow cytometry. The frequency of Vγ9+ T cells expressing cytotoxic cytokines TNFα, IFNγ or the degranulation marker CD107a was determined by flow cytometry.

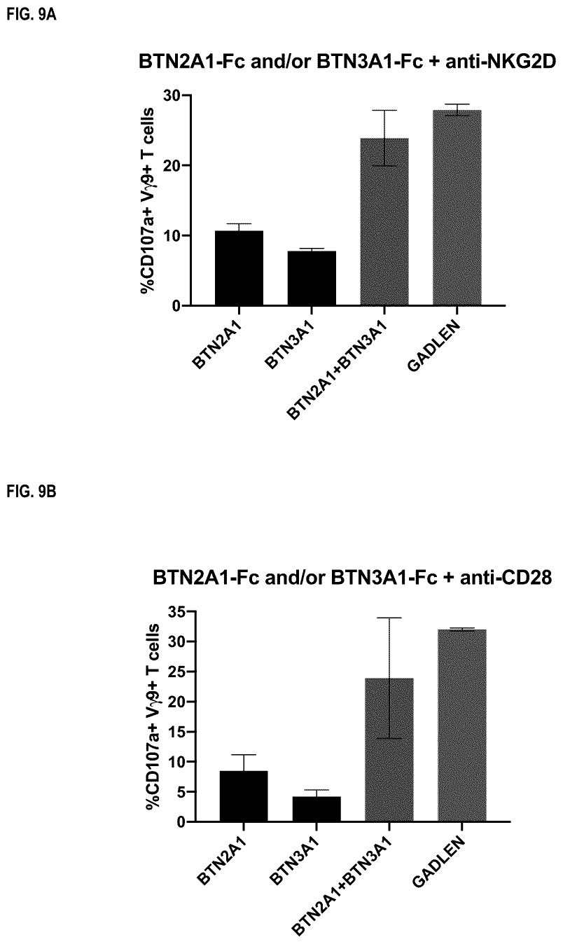

The frequency of Vγ9+ T cells expressing CD107a in cells co-stimulated with anti-NKG2D antibody was plotted. As shown in FIG. 9A, the plate-bound combination of either BTN2A1-Fc and the anti-CD28 antibody or BTN3A1-Fc and the anti-NKG2D antibody produced lower levels of level of Vγ9+ T cells expressing CD107a. However, the plate-bound combination of BTN2A1-Fc, BTN3A1-Fc, or the BTN2A1/3A1-Fc-CD19scFv heterodimeric protein and the anti-NKG2D antibody resulted in an increased fraction of Vγ9+ T cells expressing CD107a.

The frequency of Vγ9+ T cells expressing CD107a in cells co-stimulated with anti-CD28 antibody was plotted. As shown in FIG. 9B, the plate-bound combination of either BTN2A1-Fc and the anti-CD28 antibody or BTN3A1-Fc and the anti-CD28 antibody produced only background level of Vγ9+ T cells expressing CD107a. However, the plate-bound combination of BTN2A1-Fc, BTN3A1-Fc, or the BTN2A1/3A1-Fc-CD19scFv heterodimeric protein and the anti-CD28 antibody resulted in an increased fraction of Vγ9+ T cells expressing CD107a.

These results demonstrate that the activation of γδ T cells by the BTN2A1/3A1-Fc-CD19scFv heterodimeric protein occurs through the engagement of the BTN2A1/3A1 heterodimer with γδ T cells.

### Example 6: Activation of γδ T Cells by the Variable Domains of the BTN2A1 and BTN3A1 in Combination with the Anti-NKG2D Antibody

The activation of γδ T cells by the variable domains of BTN2A1 and BTN3A1 was next studied. Towards that, the BTN2A1V/3A1V-G1-CD19scFv and BTN2A1V/3A1V-G4-CD19scFv homodimeric proteins were constructed. Both included the variable domains of human BTN2A1 and BTN3A1 fused to a CD19scFv via a hinge-CH2-CH3 Fc domain from IgG1 (denoted as G1; SEQ ID NOs: 58, 61, 64, 67, 70, and 73) or IgG4 (denoted as G4; SEQ ID NOs: 59, 60, 62, 63, 65, 66, 68, 69, 71, 72 and 74). The BTN2A1V/3A1V-G1-CD19scFv and BTN2A1V/3A1V-G4-CD19scFv homodimeric proteins were produced via a transient co-transfection in Expi293 cells.

Plates were coated with increasing amounts of the BTN2A1V/3A1V-G1-CD19scFv or BTN2A1V/3A1V-G4-CD19scFv homodimeric protein alone or with 1 μg/mL anti-NKG2D antibody. A combination of the BTN2A1/3A1-Fc-CD19scFv heterodimeric protein and the anti-NKG2D antibody were used as a positive control. 1×10⁵ human γδ T cells were added to the plates for stimulation by the plate-bound agents and incubated in in 10% FBS+100 U/mL recombinant human IL-2 (rhlL-2) for 4 hours at 37° C. in the presence of inhibitors of protein transport to the Golgi complex. After 4 hours, γδ T cells were harvested and stained with anti-TNFα, anti-IFNγ or anti-CD107a, the degranulation marker of the activated γδ T cells, and analyzed by flow cytometry. The frequency of Vγ9+ T cells expressing cytotoxic cytokines TNFα, IFNγ or the degranulation marker CD107a was determined by flow cytometry.

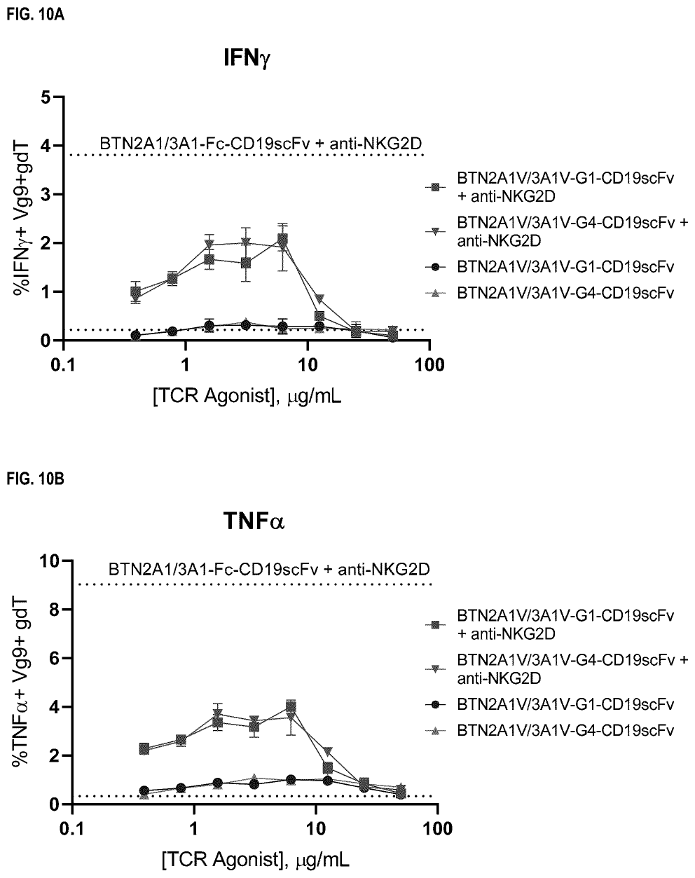

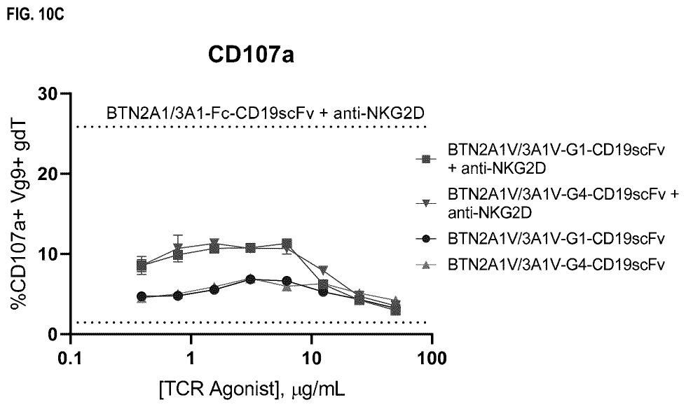

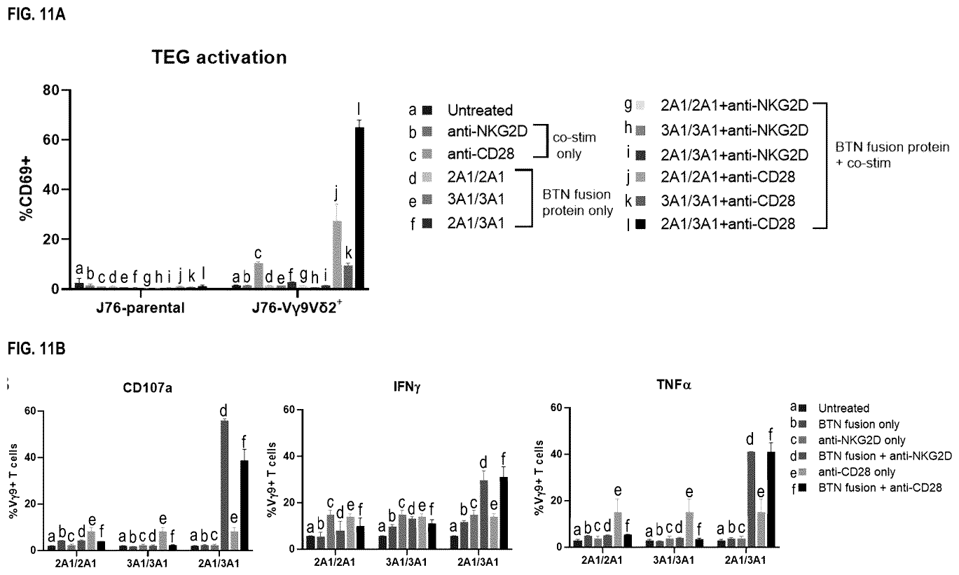

The frequency of Vγ9+ T cells expressing IFNγ as determined by flow cytometry was plotted as a function of the amount of plate bound the BTN2A1V/3A1V-G1-CD19scFv or BTN2A1V/3A1V-G4-CD19scFv homodimeric proteins. As shown in FIG. 10A, the BTN2A1V/3A1V-G1-CD19scFv or BTN2A1V/3A1V-G4-CD19scFv homodimeric proteins alone induced IFNγ in few cells, if any. In comparison, the combination of the BTN2A1V/3A1V-G1-CD19scFv or BTN2A1V/3A1V-G4-CD19scFv homodimeric proteins with the anti-NKG2D antibody produced in increased expression of IFNγ by the Vγ9+ T cells, albeit to a lower degree compared to combination of the BTN2A1/3A1-Fc-CD19scFv heterodimeric protein and the anti-NKG2D antibody (FIG. 10A). The frequency of Vγ9+ T cells expressing TNFα as determined by flow cytometry was plotted as a function of the amount of plate bound the BTN2A1V/3A1V-G1-CD19scFv or BTN2A1V/3A1V-G4-CD19scFv homodimeric proteins. As shown in FIG. 10B, the BTN2A1V/3A1V-G1-CD19scFv or BTN2A1V/3A1V-G4-CD19scFv homodimeric proteins alone induced TNFα in few cells, if any. In comparison, the combination of the BTN2A1V/3A1V-G1-CD19scFv or BTN2A1V/3A1V-G4-CD19scFv homodimeric 20 proteins with the anti-NKG2D antibody produced in increased expression of TNFα by the Vγ9+ T cells, albeit to a lower degree compared to combination of the BTN2A1/3A1-Fc-CD19scFv heterodimeric protein and the anti-NKG2D antibody (FIG. 10B). The frequency of Vγ9+ T cells expressing CD107a as determined by flow cytometry was plotted as a function of the amount of plate bound the BTN2A1V/3A1V-G1-CD19scFv or BTN2A1V/3A1V-G4-CD19scFv homodimeric proteins. As shown in FIG. 10C, the BTN2A1V/3A1V-G1-CD19scFv or BTN2A1V/3A1V-G4-CD19scFv homodimeric proteins alone induced CD107a in few cells, if any. In comparison, the combination of the BTN2A1V/3A1V-G1-CD19scFv or BTN2A1V/3A1V-G4-CD19scFv homodimeric proteins with the anti-NKG2D antibody produced in increased expression of CD107a by the Vγ9+ T cells, albeit to a lower degree compared to combination of the BTN2A1/3A1-Fc-CD19scFv heterodimeric protein and the anti-NKG2D antibody (FIG. 10C).

These results demonstrate that the co-stimulation of γδ T cells by the BTN2A1V/3A1V-G1-CD19scFv or BTN2A1V/3A1V-G4-CD19scFv homodimeric proteins, which contain the variable domains of the BTN2A1 and BTN3A1 proteins, causes the greater γδ T cell activation compared to the amount of activation without costimulation. Although the BTN2A1V/3A1V-G1-CD19scFv or BTN2A1V/3A1V-G4-CD19scFv homodimeric proteins were less functional than the BTN2A1/3A1-Fc-CD19scFv heterodimeric protein (which contain the full extra cellular domains (ECDs) in a heterodimer format) in this format of assay, these results demonstrate that the variable domains of BTN2A1 and BTN3A1 may be sufficient both for co-stimulation of the activation of γδ T cells.

### Example 7: Materials and Methods Cell Lines

Daudi and Raji cell lines were obtained from ATCC and cultured in RPMI-1640 Medium (Gibco) supplemented with 10% (v/v) fetal bovine serum (FBS) and 1×Antibiotic-Antimycotic (Anti/Anti, Gibco). K562 (ATCC) and Jurkat76 (Dr. Heemskerk, Leiden University Medical Center) were maintained in Iscove's Modified Dulbecco's Medium (IMDM, Gibco) with 10% (v/v) fetal bovine serum (FBS) and 1× Antibiotic-Antimycotic (Anti/Anti, Gibco). Heemskerk et al., Redirection of antileukemic reactivity of peripheral T lymphocytes using gene transfer of minor histocompatibility antigen HA-2-specific T-cell receptor complexes expressing a conserved alpha joining region. Blood 102: 3530-3540 (2003).

Jurkat76-Vγ9Vδ2 was generated by transducing Jurkat76 cells with pLenti-EF1a-IRES-GFP lentiviral construct containing full length TCR Vγ9 and TCR Vδ2 flanked by sequence encoding the P2A self-cleaving peptide. The sequence of TCRVγ9 and Vδ2 was derived from γ9δ2 T cell clone A3 as previously published. Vyborova et al., γ9δ2 T cell diversity and the receptor interface with tumor cells. Journal of Clinical Investigation 130: 4637-4651 (2020). Construct generation and lentivirus packaging was performed at ThermoFisher. Single-cell cloning was performed by limiting dilution. Functional GFP+ clones were characterized by surface expression of TCRVγ9, TCRVδ2, and CD3, and the ability of TCR complex to be activated by anti-CD3 or anti-TCRγδ as indicated by CD69 upregulation.

K562-CD19 was generated by transducing K562 cells with lentivirus particles containing full length human CD19 molecule (CD19, NM_001178098.2) and IRES-mediated puromycin resistance gene under the same CMV promoter (purchased from Creative Biogene). Transduced K562-CD19 cells were selected and maintained in IMDM supplemented with 10% FBS and 2 mg/mL puromycin (Sigma).

### In Vitro Expansion of Vγ9V62+-T Cell

Donor-derived Vγ9Vδ2+ T cells were expanded and cultured as previously described. Nussbaumer et al., Essential Requirements of Zoledronate-Induced Cytokine and γδT Cell Proliferative Responses. The Journal of Immunology 191: 1346-1355 (2013). Briefly, total γδ-T cells were isolated from PBMCs using the EasySep Human Gamma/Delta T Cell Isolation Kit (StemCell Technologies). Purified γδ-T cells were cultured in IMDM medium (Gibco) supplemented with 10% pooled human AB serum (Innovative Research), and in the presence of 100 U/mL rhlL-2 (Peprotech), 10 mM zoledronate (Sigma), 10 mM GGPP (Cayman Chemical), and 200 ng/mL rhlL-18 (BioLegend) for seven days to selectively expand Vγ9Vδ2+-T cell population. Expanded cells were cultured for an additional seven days in the presence of 50 U/mL rhlL-2 (R&D Systems), 400 nM zoledronate (Sigma), and 200 ng/mL rhlL-18 (BioLegend) prior to cryopreservation or used for downstream cell-binding, plate-bound stimulation, or tumor co-culture assays.

### Construct Generation and Protein Production

The sequences for BTN2A1-Fc-CD19scFv and BTN3A1-Fc-CD19scFv were codon-optimized and directionally cloned into mammalian expression vectors. Vectors were then transiently co-transfected into CHOS cells or stably transfected into CHO cells, and the resulting heterodimeric fusion protein was purified using affinity chromatography. BTN2A1-Fc and BTN3A1-Fc were either produced in a similar manner or purchased from R&D Systems.

### Western Blot

Human BTN2A1/3A1-Fc-CD19scFv proteins were deglycosylated (Protein Deglycosylation Mix II (NEB) as per manufacturer's instructions. Deglycosylated proteins were subsequently treated with or without reducing agent β-mercaptoethanol prior to SDS-PAGE. The BTN2A1 and BTN3A1 portions were simultaneously probed by rabbit anti-BTN2A1 (ThermoFisher) and sheep anti-BTN3A1 (R&D Systems) on the blot for one hour at room temperature, followed by secondary antibodies goat anti-rabbit Starbright Blue 520 at 1:2500 dilution (BioRad) and donkey anti-goat IRDye 800 CW (Licor) for one hour at room temperature. Image analysis was performed using the ChemiDoc MP Imaging System (BioRad).

### Detection of Heterodimeric BTN2A1/3A1-Fc-CD19scFv by Immunoassay

MSD Multi-Array plates (MesoScale Discovery [MSD]) were pre-coated overnight with 1 μg/mL of Sheep anti human BTN3A1/3A2 (R&D System) at 2-8° C. The plates were blocked with Diluent 100 (MSD) for at least one hour at room temperature on a shaker. After washing, samples were added at a starting concentration of 150 μg/ml, serially diluted 2-fold and loaded in duplicate wells to generate 8 test concentrations. Bound BTN2A1/3A1-Fc-CD19scFv was detected with 1 mg/ml of rabbit anti human BTN2A1 (Invitrogen), followed by goat anti rabbit sulfo tag (MSD). MSD Gold Reading buffer (MSD) was added prior to detection on the MESO QuickPlex SQ 120 MM instrument, (MSD, Model 1300). The resulting relative light units (RLU) were exported and analyzed using GraphPad Prism.

### Cell-Binding Assays

105 Vγ9Vδ2+ T cells or CD19+ lymphoma cells were incubated with an Fc receptor blocking reagent (BioLegend), followed by incubation with various concentrations of BTN2A1/3A1-Fc-CD19scFv for 1 hour in serum-free media at 4° C. After incubation, cells were washed with FACS buffer (1× DPBS containing 1% bovine serum albumin, 0.02% sodium azide and 2 mM EDTA), followed by incubation with and APC conjugated anti-human Fc antibody (Jackson ImmunoResearch) at a 1:100 dilution in FACS buffer for 30 minutes at 4° C. Cells were washed and resuspended in FACS buffer for analysis by flow cytometry. APC+ cells was analyzed using FlowJo to determine the proportion of cells bound to BTN2A1/3A1-Fc-CD19scFv. EC50 was determined using built-in non-linear regression analysis in GraphPad Prism.

### In Vitro Vγ9V62+ T Cell Activation, Degranulation and Cytokine Detection

Recombinant BTNs or TCR agonist(s) were diluted in DPBS (Gibco) at the indicated concentrations and incubated overnight at 4° C. in high-binding 96-well plates (Corning). Wells were washed twice with DPBS, and 0.5 to 1×10⁵ Vγ9Vδ2+-T cells in RPMI+10% FBS, supplemented with 100 U/mL hIL-2. BD FastImmune CD107a APC, GolgiStop, and GolgiPlug reagents (BD Biosciences) were added to cell culture according to the manufacturer's protocols. Cells were incubated at 37° C. for 4 hours and stained for cell surface and intracellular markers for analysis by flow cytometry. The proportion of CD107a+, IFNγ+, and TNFα+ cells was calculated as a fraction of the CD3+Vγ9+ population. Stimulation of Jurkat76-Vγ9Vδ2 cells with plate-bound BTN fusion proteins or TCR agonist(s) was similarly performed. After 24 hours of culture, cells were analyzed by flow cytometry. Cell activation status was determined by CD69 expression.

### Cell Proliferation Analysis of V62+ and Vδ1+ T Cells

Total γδ-T cells were isolated from PBMCs as described above, and labeled with CellTrace Violet (CTV, ThermoFisher) following manufacturer's protocol. 105 labeled γδ-T cells in AIM V serum-free media (ThermoFisher) supplemented with 100 U/mL hIL-2 was added to each well of 96-well plates coated with BTN fusion protein/TCR agonist(s). After 4 days of culture, cells were harvested for evaluation of CTV intensity in CD3+Vδ2+ and CD3+V51+ T cell populations by flow cytometry.

### Vγ9V62+-T and Tumor Cell Co-Culture

For tumor cell killing analysis, 10⁵ target cells (Daudi, Raji, K562, and K562-CD19) were first pre-bound with BTN2A1/3A1-Fc-CD19scFv or anti-BTN3A1/CD277 (clone 20.1, ThermoFisher) at the indicated concentrations for 30 minutes at 4° C. with shaking at 600 rpm. Vγ9Vδ2+ T cells were added at 1:1 ratio to target cells and cultured for 4 hours at 37° C. The proportion of apoptotic tumor cells was analyzed by flow cytometry as percent cells positive for Apoptracker Green (BioLegend) within the CD3− and CD20+ (Daudi and Raji only) population. Supernatant from the same culture was collected for TNF-α and IFNγ cytokine analysis using the human U-PLEX T-Cell Combo immunoassay (MSD). For analysis of granzyme B activity, the GranToxiLux assay kit (Oncolmmunin) was used, following manufacturer's protocol. Briefly, target cells were first labeled with a cell-permeable dye (TFL4) and fluorogenic substrate for granzyme B (GS). After washing, target cells were subsequently bound with BTN2A1/3A1-Fc-CD19scFv or anti-BTN3A1/CD277 and co-cultured with Vγ9Vδ2-T cells at 1:1 ratio as described above for one hour. The proportion of cells with granzyme B activity was determined as % GranToxiLux+ cells within the TLF4+ cell population.

### Flow Cytometry

For cell surface staining, cells were incubated with an Fc receptor blocking reagent (BioLegend) when appropriate and were stained with fluorescent antibodies for 30 minutes on ice in the dark. Cells were washed and resuspended in FACS buffer for analysis on a BD LSRII Fortessa. For intracellular staining of cytokines, cells were incubated with eFluor506 Fixable Viability (ThermoFisher) in DPBS at room temperature for 30 minutes following cell surface staining. Cells were washed, then fixed with 2% paraformaldehyde in DPBS for 10 minutes at room temperature. Following fixation, cells were permeabilized with 1×BD FACS Permeabilization (BD Biosciences) Solution 2 for 10 minutes, washed and stained with intracellular antibodies in FACS buffer for 45 minutes at room temperature. Cells were washed twice and resuspended in FACS buffer for analysis on BD LSRFortessa. Antibodies used in this study are listed in Table below:

| Assay | Reagent | Clone | Vendor |
|---|---|---|---|
| Costimulator | PE-anti-NKG2D | 1D11 | BioLegend |
| y | BV421-anti-DNAM-1 | 11A8 | BioLegend |
| receptor/ligand | APC-anti-NKp30 | P30-15 | BioLegend |
| expression | PE-Cy7-anti-NKp44 | P44.8 | BioLegend |
| | APC-anti-CD28 | CD28.2 | BioLegend |
| | APC-anti-CD27 | M-T271 | BioLegend |
| | BV421-anti-OX40 | ACT35 | BioLegend |
| | BV421-anti-4-1BB | 4B4-1 | BioLegend |
| | APC-anti-CD80 | 2D10 | BioLegend |
| | APC-anti-CD86 | BU63 | BioLegend |
| | APC-anti-MICA/B | 6D4 | BioLegend |
| | APC-anti-ULBP1 | FAB1380A | R&D Systems |
| | APC-anti-ULBP2/5/6 | FAB1298A | R&D Systems |
| Tumor-Free | Ultra-LEAF Purified anti-CD3 | OKT3 | BioLegend |
| GDT | Ultra-LEAF Purified anti-TCRγδ | B1 | BioLegend |
| activation | Purified anti-NKG2D | 149810 | R&D Systems |
| | Ultra-LEAF Purified anti-CD28 | CD28.2 | BioLegend |
| | BD FastImmune CD107a APC | H4A3 | BD Biosciences |
| | FITC-anti-IFNγ | 4S.B3 | BioLegend |
| | PerCP-Cy5.5-anti-TNFα | MAb11 | BioLegend |
| | Pacific Blue-anti-CD3ε | UCHT1 | BioLegend |
| | AF700-anti-Vγ9 | B3 | BioLegend |
| | eBioscience Fixable Viability Dye | N/A | ThermoFisher |
| | eFluor506 | | |
| PBMC/TEG | AF700-anti-CD69 | FN50 | BioLegend |
| activation | FITC-anti-Vδ1 | TS8.2 | ThermoFisher |
| | APC-anti-Vδ2 | B3 | BioLegend |
| | PE-anti-Vγ9 | B3 | BioLegend |
| | BV785-anti-CD3ε | UCHT1 | BioLegend |
| GDT and | Apoptracker Green | Apo-15 | BioLegend |
| Tumor co- | | peptide | |
| culture | APC-anti-human CD20 | 2H7 | BioLegend |
| | Purified anti-BTN3A1/CD277 | eBioBT3.1 | ThermoFisher |
| | | (20.1) | |

Data analysis was performed using FlowJo v10.8.0. Graphing and statistical analysis were performed using GraphPad Prism.

### Example 8: Vγ9V62+ T Cell Activation by Recombinant Butyrophilins (BTNs) in Combination with a Co-Stimulatory Signal Via NK Receptors or T-Cell Costimulatory Receptor

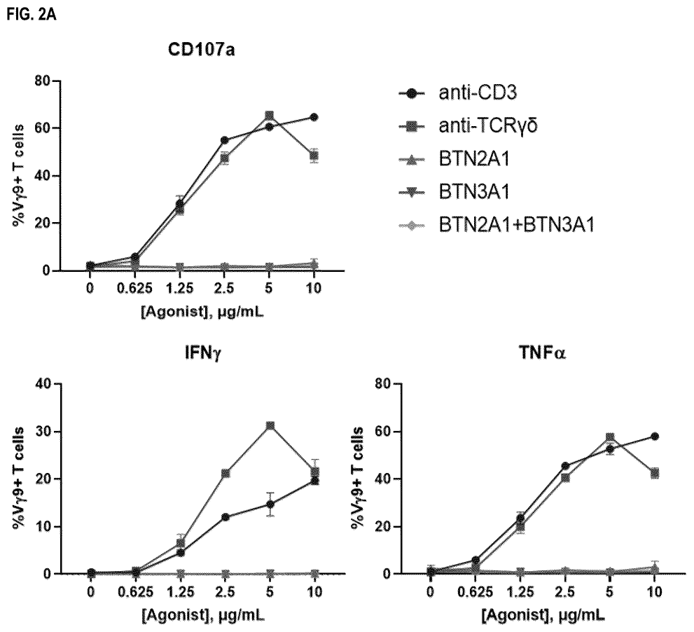

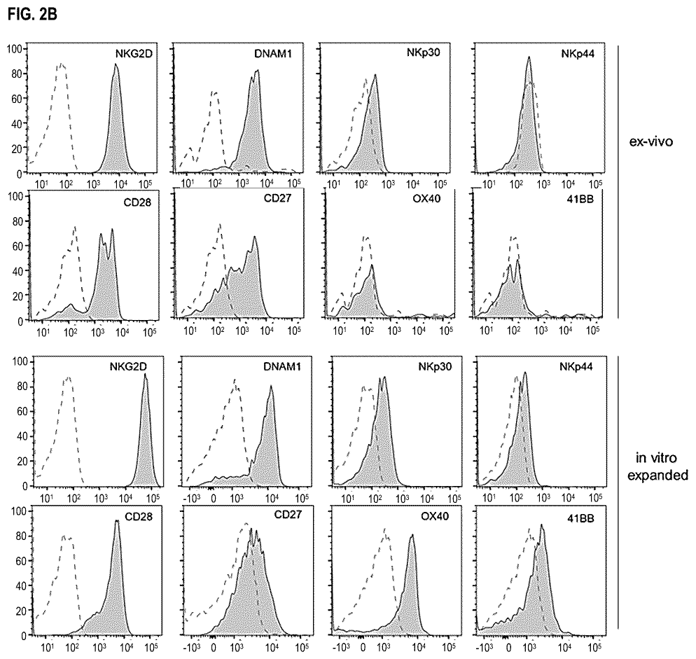

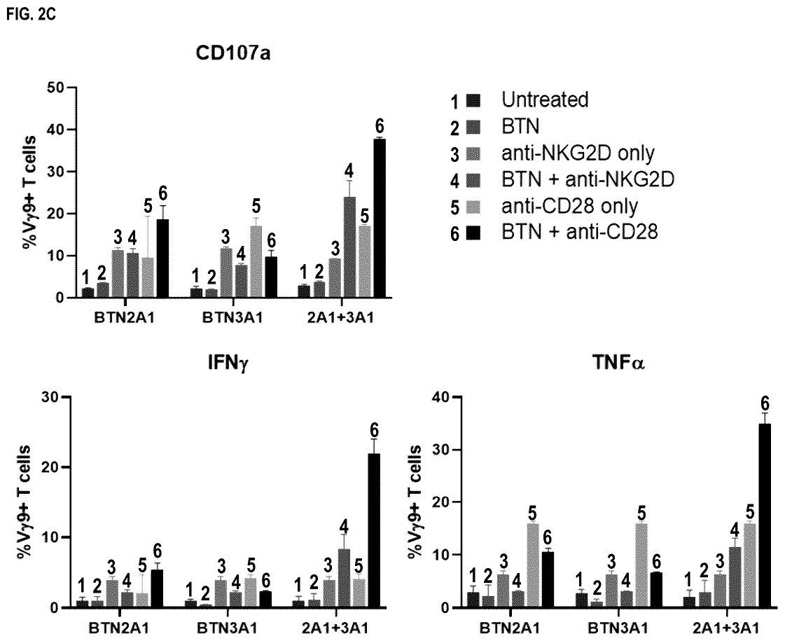

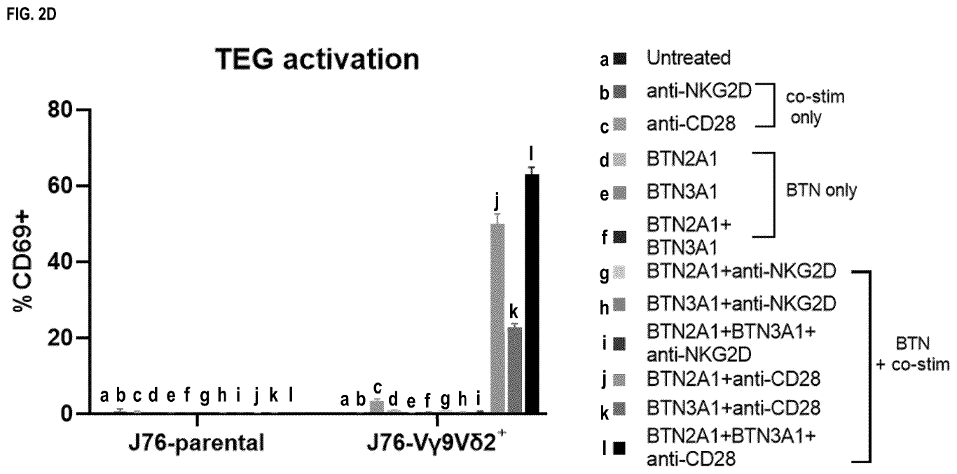

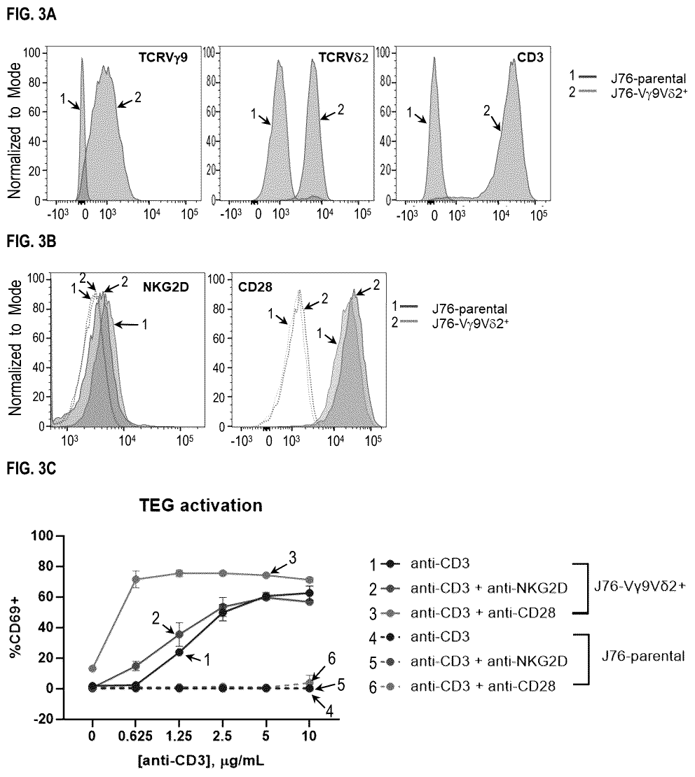

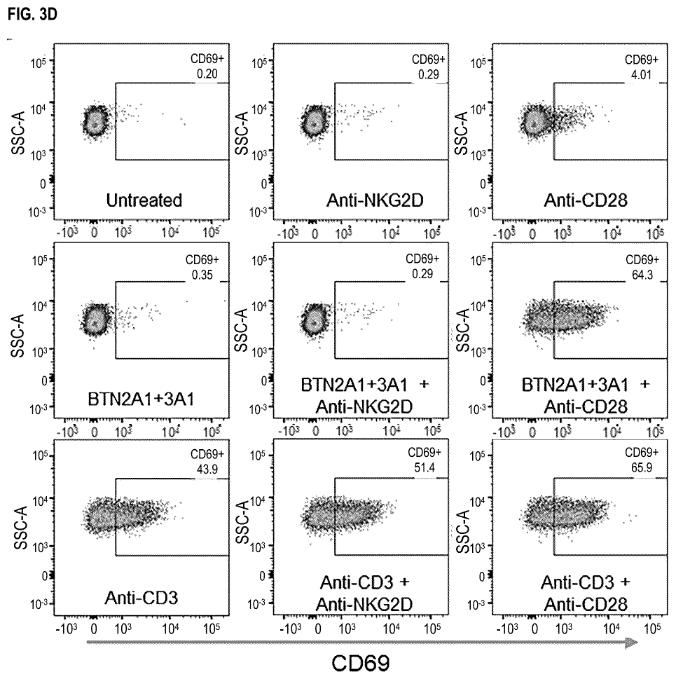

The ability of recombinant BTNs to promote degranulation and cytokine production in Vγ9V62+ T cells was first assessed using homodimeric BTN2A1-Fc and/or BTN3A1-Fc chimera proteins. While both anti-CD3 and anti-pan-TCRγδ potently activated Vγ9V62+ T cells in vitro, neither BTN2A1, BTN3A1, or BTN2A1+BTN3A1 combination (1:1 ratio) led to degranulation or production of cytokines (IFNγ and TNFα) in Vγ9V62+ T cells (FIG. 2A). Without wishing to be bound by theory, the lack of Vγ9V62+ T cell activation by BTN2A1 and BTN3A1 suggested, inter alia, that either recombinant BTNs were not in an “active” conformation to engage with T-cell receptor (TCR), or that a second co-stimulatory signal was needed to induce Vγ9V62+ T cell activation. Expression of known NK receptors (NKRs) and T-cell costimulatory receptors were analyzed on both ex vivo and in vitro expanded Vγ9Vδ2+ T cells to identify possible co-stimulatory receptors that are constitutively expressed. As shown in FIG. 2B, both sources of Vγ9Vδ2+ T cells expressed high levels of NKG2D and DNAM-1 but did not express significant amounts of natural cytotoxicity receptors NKp30 or NKp44. Vγ9Vδ2+ T cells also constitutively expressed T-cell costimulatory receptors CD28 and CD27, but only upregulated OX40 and 4-1BB upon in vitro expansion via pAg stimulation (FIG. 2B). To test the ability of NKR or T-cell costimulatory receptors to provide “signal 2,” agonistic anti-NKG2D and anti-CD28 were tested in combination with recombinant BTNs to activate Vγ9Vδ2+ T cells. BTN2A1+BTN3A1, but not either BTN alone, increased degranulation and cytokine production when combined with either anti-NKG2D or anti-CD28 (FIG. 2C). To further demonstrate that recombinant BTNs activated Vγ9Vδ2+ T cells via TCR activation, a T-cell line expressing γδ TCR (TEG) was generated by introducing Vγ9 and V62 TCR chains in Jurkat (J)76 cells that lack endogenous TCR expression. While the parental J76 did not express any components of the TCR complex, J76-Vγ9Vδ2+ expressed TCRVγ9, TCRVδ2, and CD3 on the cell surface (FIG. 3A). Consistent with primary Vγ9Vδ2+ T cell activation, BTN2A1+BTN3A1 activated J76-Vγ9Vδ2+TEG in the presence of anti-CD28, as indicated by CD69 upregulation (FIG. 2D). Since NKG2D is not expressed on J76-Vγ9Vδ2+(FIG. 3B), BTNs+anti-NKG2D did not lead to TEG activation. As shown in FIG. 3C, the T-cell receptor complex on J76-Vγ9Vδ2+ was functional. When parental J76 or J76-J76-Vγ9Vδ2+ were stimulated with various concentration of plate-bound anti-CD3 alone or in combination with 1 μg/mL anti-NKG2D or 2.5 μg/mL anti-CD28 for 24 hours, TEG activation, as assessed by CD69 expression, was observed anti-CD3 alone or in combination with 1 μg/mL anti-NKG2D or 2.5 μg/mL anti-CD28. FIG. 3D shows the representative FACS plots of CD69 expression of TEG stimulated with 5 μg/mL anti-CD3 alone or in combination with 1 μg/mL anti-NKG2D or 2.5 μg/mL anti-CD28 for 24 hours. While BTN2A1+anti-CD28 also upregulated CD69 in Vγ9Vδ2+TEG, cytokine production was only observed when Vγ9Vδ2+ T cells were stimulated by both BTN2A1 and BTN3A1 (FIG. 2C). These results confirm, inter alia, the involvement of both BTN2A1 and BTN3A1 in TCR-dependent activation of Vγ9Vδ2+ T cells, and demonstrate the requirement of a “signal 2” for BTN-mediated activation of Vγ9Vδ2+ T cells.

### Example 9: Heterodimeric BTN2A1/BTN3A1 Mimicking “Active” BTNs Also Requires the Presence of Co-Stimulatory Signal to Activate Vγ9V62+ T Cells

Without wishing to be bound by theory, it is believed that phosphoantigen sensing by the B30.2 cytoplasmic domain of BTN3A1 results in conformational changes in the extracellular domain, leading to association with BTN2A1 to activate Vγ9Vδ2+ T cells. A bispecific γδ T-cell engager, which contained heterodimeric BTN2A1 and BTN3A1 extracellular domains (ECD) fused via inert Fc linkers to scFv domains specific for CD19 (FIG. 1A), was generated to test its ability to modulate Vγ9Vδ2+ T cells and promote killing of CD19 expressing tumor cells. The presence of BTN2A1 and BTN3A1 ECDs on the two polypeptide chains on the BTN2A1/3A1-Fc-CD19scFv molecule was confirmed by Western blot under non-reduced, reduced, and deglycosylated conditions using specific antibodies (FIG. 1B and FIG. 1C). The formation of a BTN2A1/BTN3A1 heterodimer was confirmed by a dual-binding immunoassay using capture and detection antibodies that bind to the individual BTN domains (FIG. 1D). Only the BTN2A1/3A1-Fc-CD19scFv construct containing CD19scFv, but not an unrelated scFv, bound to a CD19+ lymphoma cell line (FIG. 1E). Furthermore, only the BTN2A1/3A1-Fc-CD19scFv construct (but not a control construct containing a BTN3A1 homodimer) bound to Vγ9Vδ2+ T cells (FIG. 1F) but not Vδ1+(predominately Vγ9−) or CD8+ T cells in PBMCs (FIG. 1H). BTN2A1/3A1-Fc-CD19scFv binding to Vγ9Vδ2+ T cells was partially inhibited by the presence of anti-pan-TCRγδ and completely blocked by anti-Vγ9 (FIG. 1G), confirming the specificity of heterodimeric BTN2A1 and BTN3A1 to the Vγ9 subunit of the TCR complex.

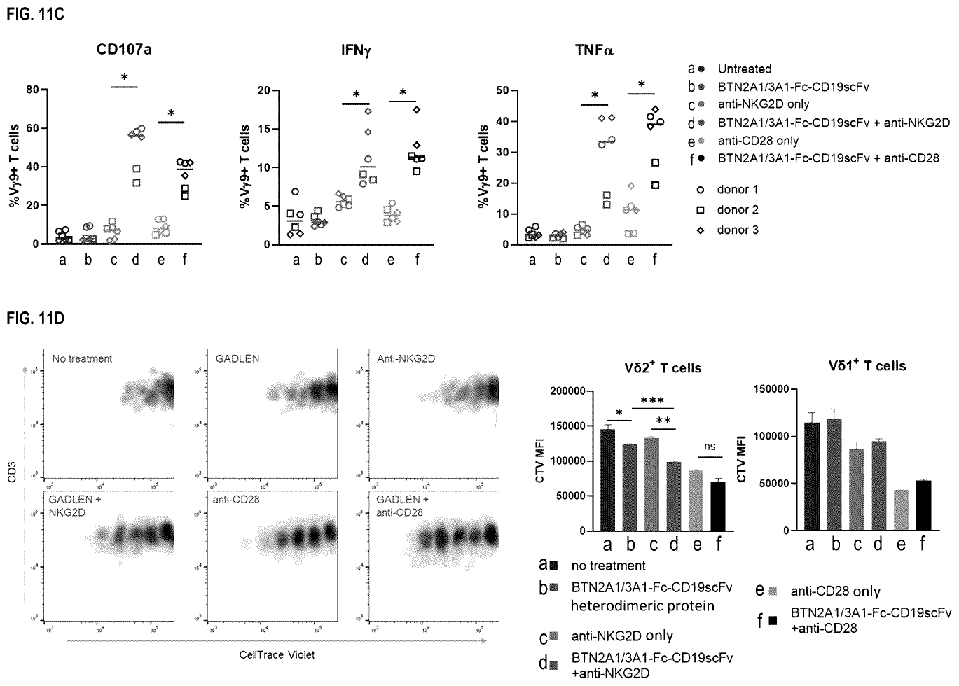

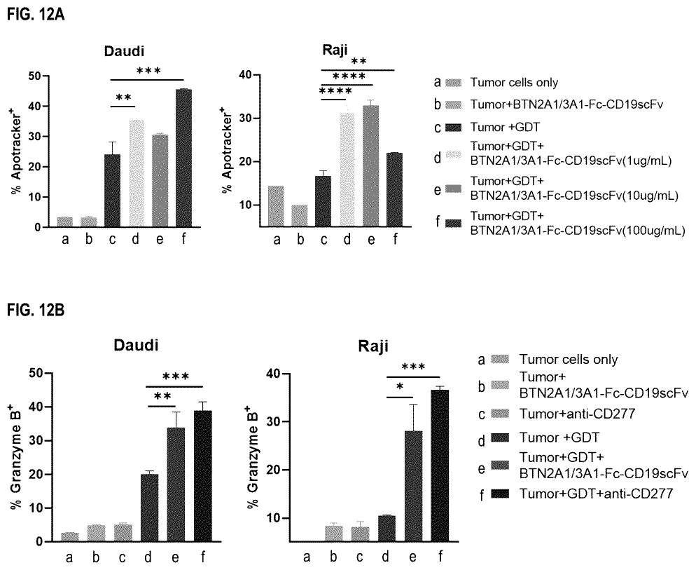

Consistent with cell-binding specificity, stimulation of J76-Vγ9Vδ2+TEG but not parental J76 with BTN2A1/3A1-Fc-CD19scFv led to robust upregulation of CD69, but only in the presence of CD28 co-stimulation (FIG. 11A). BTN fusion protein containing BTN2A1 dimers in combination with anti-CD28 also partially upregulated CD69, suggesting, inter alia, that heterodimeric BTN2A1 and BTN3A1 may improve the affinity of BTN2A1 interaction with Vγ9Vδ2 TCR to deliver a stronger signal for downstream cytokine production (FIG. 11A). Alternatively, without wishing to be bound by theory, BTN3A1 may interact with other cell-surface protein(s) to enhance Vγ9Vδ2+ T cell activation. Similarly, heterodimeric BTN2A1 and BTN3A1, but not BTN2A1 or BTN3A1 dimers, induced Vγ9Vδ2+ T cell degranulation and cytokine production, but again only in the presence of NKG2D or CD28 co-stimulation across multiple donor-derived Vγ9Vδ2+ T cells (FIG. 11B and FIG. 11C). Furthermore, while anti-CD28 stimulation alone led to proliferation in both peripheral Vδ1+ and Vδ2+ T cells, BTN2A1/3A1-Fc-CD19scFv in combination with anti-NKG2D preferentially enhanced proliferation of Vδ2+ T cells but not Vδ1+ T cell (FIG. 11D). Taken together, these results suggest, inter alia, that engagement of “active” heterodimeric BTN2A1 and BTN3A1 with concomitant co-stimulation via NKR or T-cell co-stimulatory receptor is needed to fully activate the cytotoxic properties of Vγ9Vδ2+ T cells.

### Example 10: Addition of BTN2A1/3A1-Fc-CD19scFv Enhanced Vγ9V62+ T Cell Cytotoxicity Against B-Cell Lymphoma

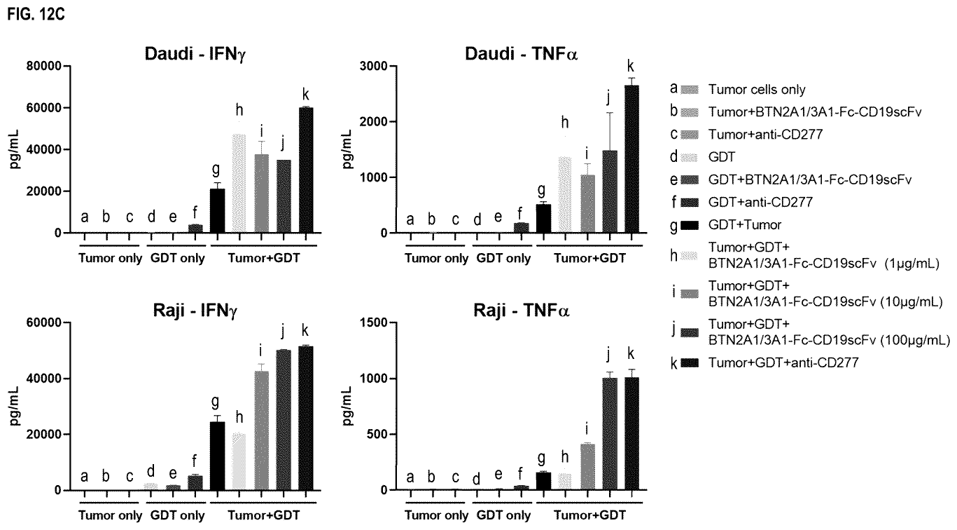

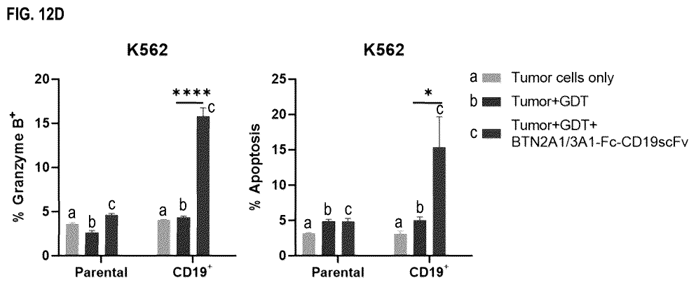

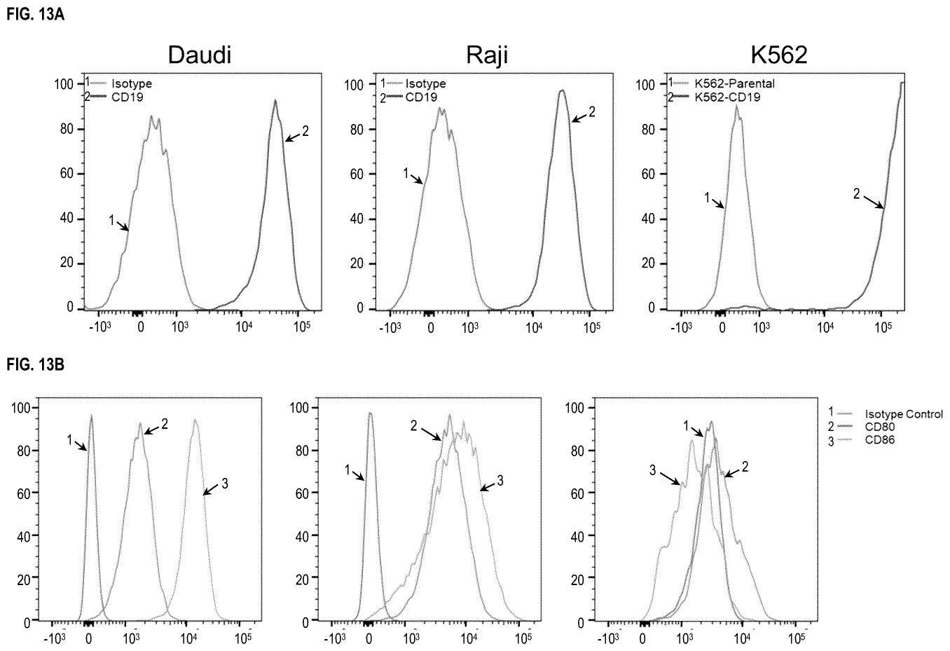

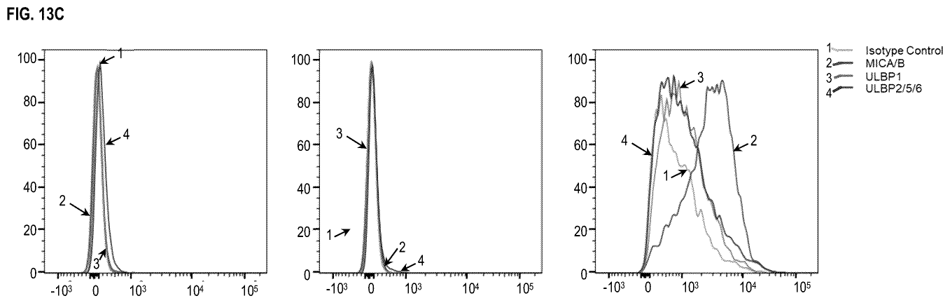

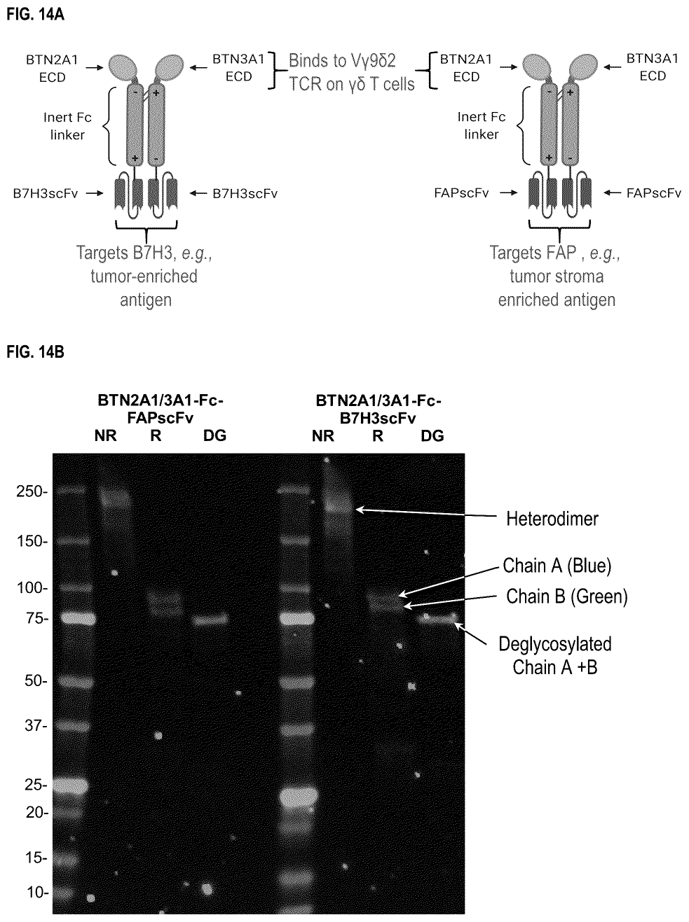

To evaluate the ability of BTN2A1/3A1-Fc-CD19scFv to enhance Vγ9Vδ2+ T cell-mediated killing of tumor cells, CD19+ Daudi and Raji cells (FIG. 13A) were cultured with Vγ9Vδ2+ T cells in the presence of BTN2A1/3A1-Fc-CD19scFv. Addition of BTN2A1/3A1-Fc-CD19scFv to the co-culture resulted in an increased proportion of apoptotic tumor cells, as evidenced by detection of translocated phosphatidylserine residues on the cell surface (FIG. 12A). Similar levels of tumor killing was induced by 1 to 100 μg/mL (6.7 to 670 nM) of BTN2A1/3A1-Fc-CD19scFv, suggesting a concentration at or below EC50 for tumor cell binding by BTN2A1/3A1-Fc-CD19scFv (FIG. 1E) can efficiently induce cytotoxicity in Vγ9Vδ2+ T cells. BTN2A1/3A1-Fc-CD19scFv-mediated Vγ9Vδ2+ T cell cytotoxicity was additionally investigated using a cell-based fluorogenic cytotoxicity assay designed to measure granzyme B activity in live target cells following the successful transfer of granzyme B by cytotoxic lymphocytes. In agreement with tumor cell apoptosis, the proportion of tumor cells exhibiting granzyme B activity (FIG. 12B) as well as secreted levels of cytokines such as IFNγ and TNFα (FIG. 12C) significantly increased when BTN2A1/3A1-Fc-CD19scFv was added to Vγ9Vδ2+T and Daudi or Raji co-culture, but not to tumor cells alone, confirming that the mechanism of action for BTN2A1/3A1-Fc-CD19scFv is through enhancement of Vγ9Vδ2+ T cell cytotoxicity. The frequency of granzyme B+ tumor cells was comparable when BTN2A1/3A1-Fc-CD19scFv or a saturating dose of agonistic anti-CD277/BTN3A1 (20.1) was added to the Vγ9Vδ2+ T cells and tumor co-culture. This further demonstrates that recombinant heterodimeric BTN2A1/3A1 can provide an activating signal to Vγ9Vδ2 TCR in a similar fashion as endogenous BTN2A1 in combination with an active conformation of BTN3A1 induced by the 20.1 antibody. Phenotypic analysis of CD19+ lymphoma cell lines indicated the presence of CD28 ligands CD80 and CD86 (FIG. 13B), suggesting the co-stimulatory signal needed for BTN2A1/3A1-Fc-CD19scFv activity was provided by tumor cells. To assess the ability of BTN2A1/3A1-Fc-CD19scFv to enhance Vγ9Vδ2+ T cell-mediated killing of tumor cells expressing NKG2D ligands, we generated a CD19 expressing K562 cell line with endogenous MICA/B on cell surface (FIG. 13C). While the addition of BTN2A1/3A1-Fc-CD19scFv to Vγ9Vδ2+ T cell+parental K562 co-culture did not enhance tumor cell killing, BTN2A1/3A1-Fc-CD19scFv significantly enhanced Vγ9Vδ2+ T cell-mediated cytotoxicity of CD19-expressing K562 cells (FIG. 12D). These results highlight the ability of BTN2A1/3A1-Fc-CD19scFv in promoting targeted killing of tumor cells expressing the select tumor antigen as well as ligands for CD28 and/or NKG2D to deliver signal 2 to activate Vγ9Vδ2+ T cells.

Collectively, results from this study have demonstrated, inter alia, the feasibility of using recombinant heterodimeric BTN2A1 and BTN3A1 in a bispecific engager format to enhance anti-tumor activity of Vγ9Vδ2+ T cells. While in tumor-free culture system we demonstrated the need for the presence of a co-stimulatory signal to activate Vγ9Vδ2+ T cells by butyrophilins, BTN2A1/3A1-Fc-CD19scFv as a single-agent was sufficient to promote tumor killing, indicating the delivery of co-stimulatory signal(s) by ligands natively expressed by tumor cells. NKG2D and CD28 were identified as two co-stimulatory receptors for Vγ9Vδ2+ T cells and confirmed expression of their corresponding ligands on tumor cells. Phenotypic analysis suggests additional co-stimulatory receptors are present on Vγ9Vδ2+ T cells including DNAM-1 and CD27 (FIG. 2B), which likely contribute to Vγ9Vδ2+ T cell activation. Likewise, multiple stress-induced molecules or ligands for NKRs are likely expressed on infected or transformed cells to promote BTN-mediated killing by Vγ9Vδ2+ T cells. Comprehensive investigation into additional co-stimulatory/co-inhibitory receptors on Vγ9Vδ2+ T cells and regulation of cell-surface expression of the corresponding ligands in different cancer cell types will be invaluable to further our understanding of Vγ9Vδ2+ T cell biology. Additionally, these biological insights will also facilitate translation of effective γδ T cell-targeted therapies and ultimately selecting cancer patients most likely to respond to this emerging class of therapy.

While antibodies against the γδTCR components (i.e., CD3, TCRγ5, and TCR Vδ2) can induce robust Vγ9Vδ2+ T cell activation without the need for signal 2 (FIG. 3C and FIG. 3D), the weaker TCR signal delivered by its natural ligand in the format of BTN heterodimers has the potential to provide a more targeted approach to exert cytotoxicity in cancer cells, at the same time preventing systemic activation of γδ T cells in the clinical setting. Preliminary data comparing anti-tumor activity of Vγ9Vδ2+ T cells in the presence of different agonists suggests comparable levels of IFNγ and TNFα produced by engagers containing heterodimeric BTN2A1/3A1 or anti-Vδ2. However, stimulation by BTNs resulted in a lower production of immunosuppressive cytokine IL-17A by Vγ9Vδ2+ T cells (data not shown).

### Example 11: Vγ9V62+ T Cell Activation by Recombinant Butyrophilins (BTNs) in Combination with a Co-Stimulatory Signal Via NK Receptors or T-Cell Costimulatory Receptor

The ability of recombinant BTNs to promote degranulation and cytokine production in Vγ9Vδ2+ T cells was first assessed using homodimeric BTN2A1-Fc and/or BTN3A1-Fc chimera proteins. While both anti-CD3 and anti-pan-TCRγ5 potently activated Vγ9Vδ2+ T cells in vitro, neither BTN2A1, BTN3A1, or BTN2A1+BTN3A1 combination (1:1 ratio) led to degranulation or production of cytokines (IFNγ and TNFα) in Vγ9Vδ2+ T cells (FIG. 2A). The lack of Vγ9Vδ2+ T cell activation by BTN2A1 and BTN3A1 suggested that either recombinant BTNs were not in an “active” conformation to engage with T-cell receptor (TCR), or that a second co-stimulatory signal was needed to induce Vγ9Vδ2+ T cell activation. Expression of known NKRs and T-cell costimulatory receptors were analyzed on both ex vivo and in vitro expanded Vγ9Vδ2+ T cells to identify possible co-stimulatory receptors that are constitutively expressed. In line with previous reports on NKR expression on γδ T cells, both sources of Vγ9Vδ2+ T cells expressed high levels of NKG2D and DNAM-1 but did not express significant amounts of natural cytotoxicity receptors NKp30 or NKp44 (FIG. 2B) (36). Vγ9Vδ2+ T cells also constitutively expressed T-cell costimulatory receptors CD28 and CD27, but only upregulated OX40 and 4-1BB upon in vitro expansion via pAg stimulation (FIG. 2B). To test the ability of NKR or T-cell costimulatory receptors to provide “signal 2”, agonistic anti-NKG2D and anti-CD28 were tested in combination with recombinant BTNs to activate Vγ9Vδ2+ T cells. BTN2A1+BTN3A1, but not either BTN alone, increased degranulation and cytokine production when combined with either anti-NKG2D or anti-CD28 (FIG. 2C). To further demonstrate that recombinant BTNs activated Vγ9Vδ2+ T cells via TCR activation, we generated a T-cell line expressing γδ TCR (TEG) by introducing Vγ9 and V62 TCR chains in Jurkat (J)76 cells that lack endogenous TCR expression (33, 34). While the parental J76 did not express any components of the TCR complex, J76-Vγ9Vδ2+ expressed TCRVγ9, TCRVδ2, and CD3 on the cell surface (FIG. 13A). Consistent with primary Vγ9Vδ2+ T cell activation, BTN2A1+BTN3A1 activated J76-Vγ9Vδ2+TEG in the presence of anti-CD28, as indicated by CD69 upregulation (FIG. 2D). Since NKG2D is not expressed on J76-Vγ9Vδ2+(FIG. 3B), BTNs+anti-NKG2D did not lead to TEG activation. While BTN2A1+anti-CD28 also upregulated CD69 in Vγ9Vδ2+TEG, cytokine production was only observed when Vγ9Vδ2+ T cells were stimulated by both BTN2A1 and BTN3A1 (FIG. 2C). These results confirm the involvement of both BTN2A1 and BTN3A1 in TCR-dependent activation of Vγ9Vδ2+ T cells, and demonstrate the requirement of a “signal 2” for BTN-mediated activation of Vγ9Vδ2+ T cells.

### Example 12: Construction and Characterization of BTN2A1/3A1-Fc-B7H3scFv Heterodimeric Protein

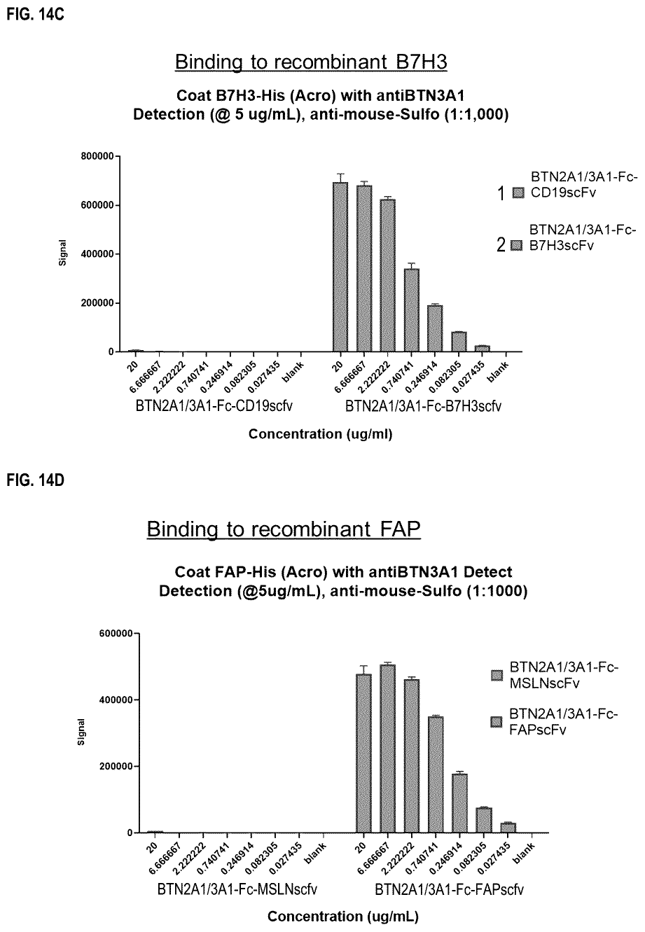

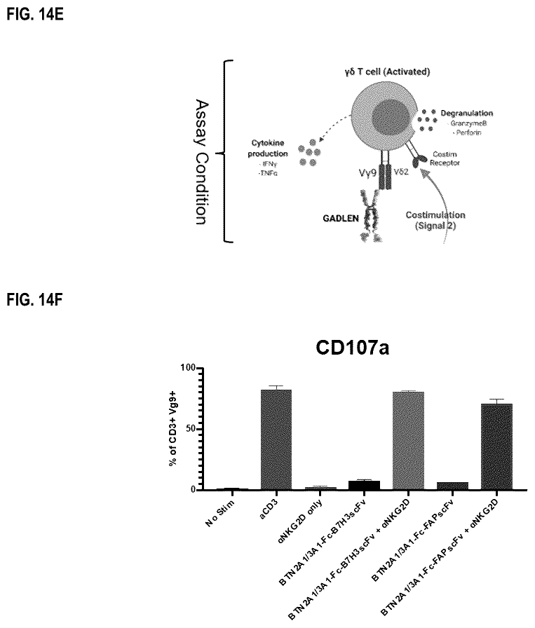

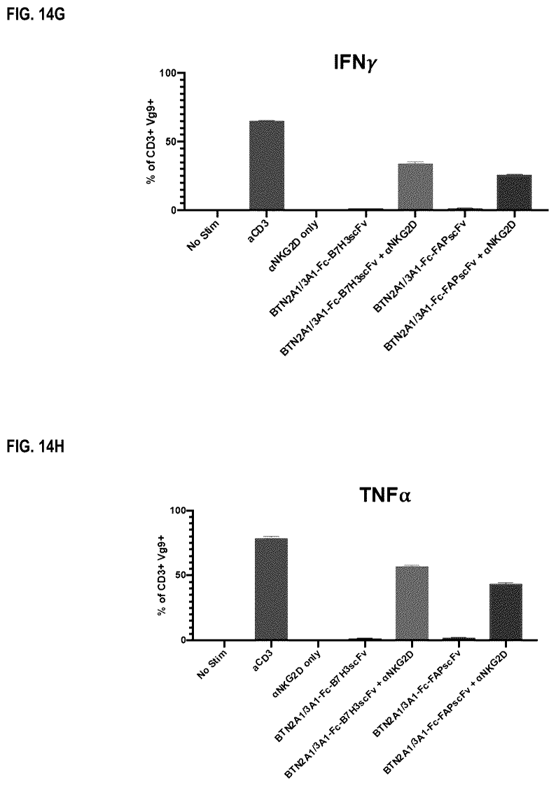

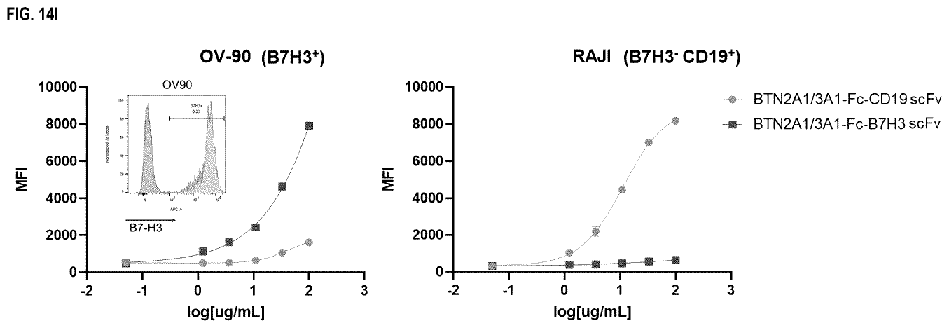

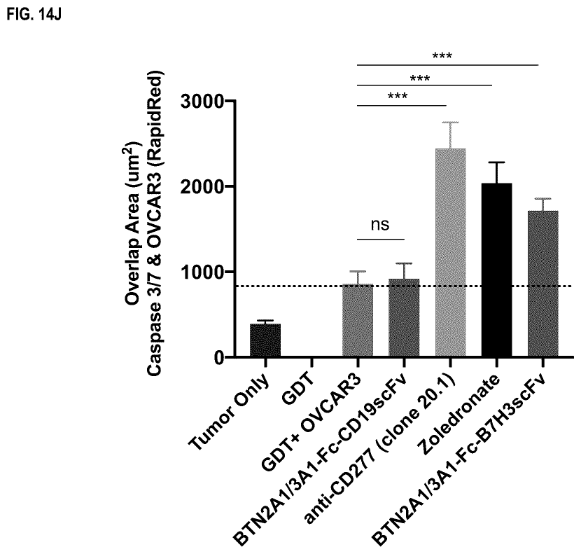

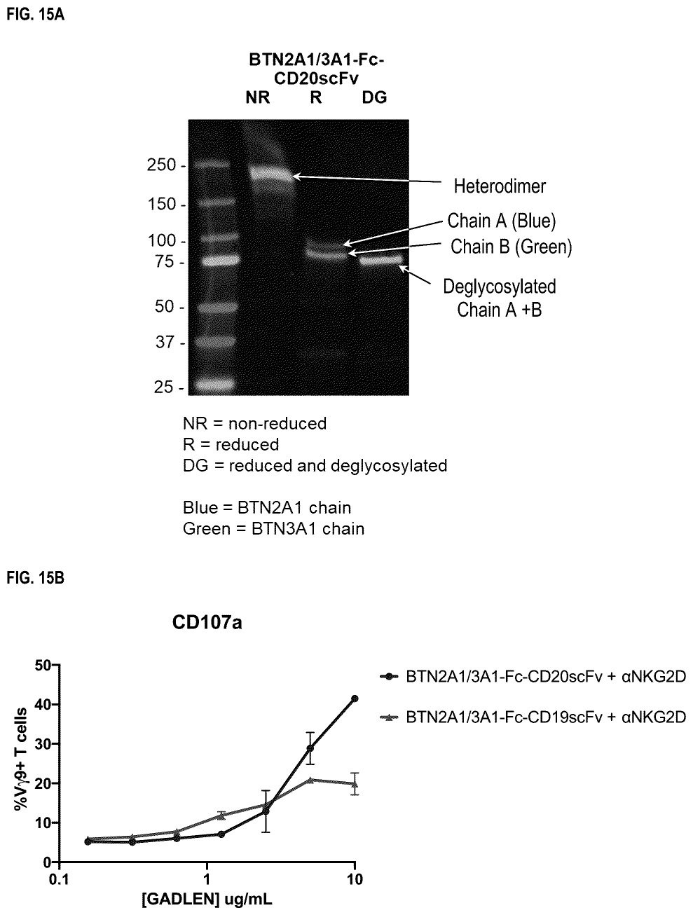

The “BTN2A1/3A1-Fc-B7H3scFv” construct included an alpha chain comprising an extracellular domain (ECD) of human BTN2A1 fused to a B7H3scFv via a hinge-CH2-CH3 Fc domain, and a beta chain comprising an extracellular domain (ECD) of human BTN3A1 fused to a B7H3scFv via a hinge-CH2-CH3 Fc domain. See, FIG. 14A (left panel). Constructs encoding BTN2A1-Fc-B7H3scFv protein (alpha chain) and BTN3A1-Fc-B7H3scFv protein (beta chain) were generated. This GAmma DELta T cell ENgager construct also is referred to herein as the BTN2A1/3A1-Fc-B7H3scFv heterodimeric protein.

The BTN2A1/3A1-Fc-B7H3scFv heterodimer protein that was produced via a transient co-transfection in ExpiCHO cells of two plasmids encoding 1) the BTN2A1-alpha-B7H3scFv protein and 2) the BTN3A1-beta-B7H3scFv protein. The alpha and beta constructs encoded a BTN2A1-Fc-B7H3scFv (‘alpha’ chain) and a BTN3A1-Fc-B7H3scFv (‘beta’ chain). The alpha and beta chains contained charged polarized linker domains which facilitated heterodimerization of the desired the BTN2A1/3A1-Fc-B7H3scFv heterodimeric protein. The charged polarized domains (indicated in FIG. 14A by “+” or “−” on the two chains of the heterodimer), which favors formation of heterodimer because of charge-charge interation, and disfavors the formation of a homodimer. The cell culture supernatant from the transient transfection was harvested 6 days following transfection and purified over an affinity chromatography resin and elution under low pH conditions.

Purity of the protein was assessed using non-reducing sodium dodecyl sulfate polyacrylamide gel electrophoresis (SDS-PAGE). The purified protein was also analyzed by western blots using non-reducing, reducing, and reducing and deglycosylating conditions, following detection with an anti-human BTN2A1 antibody, or an anti-human BTN3A1 antibody. BTN2A1-alpha and BTN3A1-beta chains were detected in the protein prep using specific antibodies conjugated to IR dyes: goat anti Rabbit Starbright Blue (520 CW) and Donkey anti Goat/Sheep Green (800 CW). Non-reduced BTN2A1/3A1-Fc-B7H3scFv heterodimeric protein ran as a single band (See lanes “NR” in FIG. 14B, right panel) indicative of covalent complex formation between the BTN2A1-alpha-B7H3scFv and BTN3A1-beta-B7H3scFv chains. As shown in FIG. 14B (right panel), the blots revealed two bands with the protein prepared under reducing but non-deglycosylated condition (See lane “R” in FIG. 14B, right panel). Interestingly, protein prepared under both reduced and deglycosylated (lane “DG”) conditions resulted in a single band, which could be detected with any of the anti-human BTN2A1, anti-human BTN3A1, or anti-mouse Fc antibodies (See lane “DG” in FIG. 14B, right panel).

To assess contemporaneous binding of the BTN2A1/3A1-Fc-B7H3scFv heterodimeric protein to B7H3 protein, a Meso Scale Discovery (MSD) platform-based assay was used. Briefly, recombinant B7H3-His protein was coated on a plate. Increasing amounts of the BTN2A1/3A1-Fc-B7H3scFv or BTN2A1/3A1-Fc-CD19scFv heterodimeric proteins were added to the plate for capture by the plate-bound recombinant B7H3-His protein. An anti-BTN3A1 antibody was added to the plate for capture by the BTN2A1/3A1-Fc-B7H3scFv or BTN2A1/3A1-Fc-CD19scFv heterodimeric proteins captured by the plate-bound recombinant B7H3 protein. Binding was detected using SULFO-TAG conjugated anti-mouse antibody. Thus, signal could be detected only when the heterodimeric protein contemporaneously binds to both recombinant B7H3-His protein and the anti-BTN3A1 antibody. As shown in FIG. 14C the BTN2A1/3A1-Fc-B7H3scFv/B7H3scFv heterodimeric protein produced a dose-dependent and saturable signal. In comparison, the BTN2A1/3A1-Fc-CD19 scFv heterodimeric protein showed only background signal.

These results demonstrate, inter alia, that the B7H3scFv of the BTN2A1/3A1-Fc-B7H3scFv heterodimeric protein disclosed herein specifically binds to B7H3 protein.

### Example 13: Construction and Characterization of BTN2A1/3A1-Fc-FAPscFv Heterodimeric Protein

The “BTN2A1/3A1-Fc-FAPscFv” construct included an alpha chain comprising an extracellular domain (ECD) of human BTN2A1 fused to a FAPscFv via a hinge-CH2-CH3 Fc domain, and a beta chain comprising an extracellular domain (ECD) of human BTN3A1 fused to a FAPscFv via a hinge-CH2-CH3 Fc domain. See, FIG. 14A (right panel). Constructs encoding BTN2A1-Fc-FAPscFv protein (alpha chain) and BTN3A1-Fc-FAPscFv protein (beta chain) were generated. This GAmma DELta T cell ENgager construct also is referred to herein as the BTN2A1/3A1-Fc-FAPscFv heterodimeric protein.

The BTN2A1/3A1-Fc-FAPscFv heterodimer protein that was produced via a transient co-transfection in ExpiCHO cells of two plasmids encoding 1) the BTN2A1-alpha-FAPscFv protein and 2) the BTN3A1-beta-FAPscFv protein. The alpha and beta constructs encoded a BTN2A1-Fc-FAPscFv (‘alpha’ chain) and a BTN3A1-Fc-FAPscFv (‘beta’ chain). The alpha and beta chains contained charged polarized linker domains which facilitated heterodimerization of the desired the BTN2A1/3A1-Fc-FAPscFv heterodimeric protein. The charged polarized domains (indicated in FIG. 14A by “+” or “−” on the two chains of the heterodimer), which favors formation of heterodimer because of charge-charge interation, and disfavors the formation of a homodimer. The cell culture supernatant from the transient transfection was harvested 6 days following transfection and purified over an affinity chromatography resin and elution under low pH conditions.

Purity of the protein was assessed using non-reducing sodium dodecyl sulfate polyacrylamide gel electrophoresis (SDS-PAGE). The purified protein was also analyzed by western blots using non-reducing, reducing, and reducing and deglycosylating conditions, following detection with an anti-human BTN2A1 antibody, or an anti-human BTN3A1 antibody. BTN2A1-alpha and BTN3A1-beta chains were detected in the protein prep using specific antibodies conjugated to IR dyes: goat anti Rabbit Starbright Blue (520 CW) and Donkey anti Goat/Sheep Green (800 CW). Non-reduced BTN2A1/3A1-Fc-FAPscFv heterodimeric protein ran as a single band (See lanes “NR” in FIG. 14B, left panel) indicative of covalent complex formation between the BTN2A1-alpha-FAPscFv and BTN3A1-beta-FAPscFv chains. As shown in FIG. 14B (left panel), the blots revealed two bands with the protein prepared under reducing but non-deglycosylated condition (See lane “R” in FIG. 14B, left panel). Interestingly, protein prepared under both reduced and deglycosylated (lane “DG”) conditions resulted in a single band, which could be detected with any of the anti-human BTN2A1, anti-human BTN3A1, or anti-mouse Fc antibodies (See lane “DG” in FIG. 14B, left panel).

To assess contemporaneous binding of the BTN2A1/3A1-Fc-FAPscFv heterodimeric protein to FAP protein, a Meso Scale Discovery (MSD) platform-based assay was used. Briefly, recombinant FAP-His protein was coated on a plate. Increasing amounts of the BTN2A1/3A1-Fc-FAPscFv or BTN2A1/3A1-Fc-CD19scFv heterodimeric proteins were added to the plate for capture by the plate-bound recombinant FAP-His protein.

An anti-BTN3A1 antibody was added to the plate for capture by the BTN2A1/3A1-Fc-FAPscFv or BTN2A1/3A1-Fc-CD19scFv heterodimeric proteins captured by the plate-bound recombinant FAP protein.

Binding was detected using SULFO-TAG conjugated anti-mouse antibody. Thus, signal could be detected only when the BTN2A1/3A1-Fc-FAPscFv heterodimeric protein contemporaneously binds to both recombinant FAP-His protein and the anti-BTN3A1 antibody. As shown in FIG. 14D the BTN2A1/3A1-Fc-FAPscFv/FAPscFv heterodimeric protein produced a dose-dependent and saturable signal. In comparison, the BTN2A1/3A1-Fc-CD19 scFv heterodimeric protein showed only background signal.

These results demonstrate, inter alia, that the FAPscFv of the BTN2A1/3A1-Fc-FAPscFv heterodimeric protein disclosed herein specifically binds to FAP protein.

### Example 14: Vγ9V62+ T Cell Activation by BTN2A1/3A1-Fc-B7H3scFv and BTN2A1/3A1-Fc-FAPscFv Heterodimeric Proteins in Combination with a Co-Stimulatory Signal Via NK Receptors or T-Cell Costimulatory Receptor

The ability of BTN2A1/3A1-Fc-B7H3scFv and BTN2A1/3A1-Fc-FAPscFv heterodimeric proteins to promote degranulation and cytokine production in Vγ9Vδ2+ T cells was assessed using the assay discussed in Example 7 and illustrated in FIG. 14E. Briefly, purified BTN2A1/3A1-Fc-B7H3scFv and BTN2A1/3A1-Fc-FAPscFv heterodimeric proteins, an anti-NKG2D antibody or an anti-CD3 antibody were diluted in DPBS (Gibco) and incubated overnight at 4° C. in high-binding 96-well plates (Corning). Wells were washed twice with DPBS, and 0.5 to 1×10⁵ Vγ9Vδ2+-T cells in RPMI+10% FBS, supplemented with 100 U/mL hIL-2. BD FastImmune CD107a APC, GolgiStop, and GolgiPlug reagents (BD Biosciences) were added to cell culture according to the manufacturer's protocols. Cells were incubated at 37° C. for 4 hours and stained for cell surface and intracellular markers for analysis by flow cytometry. The proportion of CD107a+, IFNγ+, and TNFα+ cells was calculated as a fraction of the CD3+Vγ9+ population. As shown in FIG. 14F, BTN2A1/3A1-Fc-B7H3scFv heterodimeric protein alone, BTN2A1/3A1-Fc-FAPscFv heterodimeric protein alone or anti-NKG2D antibody alone induced only a background level of CD107a. On the other hand, the percentage of CD107a-expressing Vγ9V62+-T cells when induced by the combinations of BTN2A1/3A1-Fc-B7H3scFv heterodimeric protein and the anti-NKG2D antibody, and BTN2A1/3A1-Fc-FAPscFv heterodimeric protein and the anti-NKG2D antibody were similar to those in the Vγ9V62+-T cells treated with the anti-CD3 antibody (FIG. 14F).

As shown in FIG. 14G, BTN2A1/3A1-Fc-B7H3scFv heterodimeric protein alone, BTN2A1/3A1-Fc-FAPscFv heterodimeric protein alone or anti-NKG2D antibody alone induced only a background level of IFNγ. On the other hand, the percentage of IFNγ-expressing Vγ9V62+-T cells increased when induced by the combinations of BTN2A1/3A1-Fc-B7H3scFv heterodimeric protein and the anti-NKG2D antibody, and BTN2A1/3A1-Fc-FAPscFv heterodimeric protein and the anti-NKG2D antibody, similar to those in the Vγ9V62+-T cells treated with the anti-CD3 antibody alone (FIG. 14G).

As shown in FIG. 14H, BTN2A1/3A1-Fc-B7H3scFv heterodimeric protein alone, BTN2A1/3A1-Fc-FAPscFv heterodimeric protein alone or anti-NKG2D antibody alone induced only a background level of TNFα. On the other hand, the percentage of TNFα-expressing Vγ9V62+-T cells increased when induced by the combinations of BTN2A1/3A1-Fc-B7H3scFv heterodimeric protein and the anti-NKG2D antibody, and BTN2A1/3A1-Fc-FAPscFv heterodimeric protein and the anti-NKG2D antibody, similar to those in the Vγ9V62+-T cells treated with the anti-CD3 antibody alone (FIG. 14H).

These results confirm, inter alia, the involvement of both BTN2A1/3A1-Fc-B7H3scFv heterodimeric protein alone, BTN2A1/3A1-Fc-FAPscFv heterodimeric protein are able to induce activation of Vγ9V62+ T cells.

### Example 15: Binding of the BTN2A1/3A1-Fc-B7H3scFv Heterodimeric Protein to B7H3-Expressing Cells

To access the binding specificity of the BTN2A1/3A1-Fc-B7H3scFv heterodimeric protein, OV-90 cells, which are known to express B7H3 (See FIG. 14I, left panel, inset), and RAJI cells, which are known not to express B7H3 were used. The BTN2A1/3A1-Fc-CD19scFv heterodimeric protein was used as a negative control for binding to OV-90 cells. Since RAJI cells express CD19, the BTN2A1/3A1-Fc-CD19scFv heterodimeric protein served as a positive control for binding to RAJI cells. Increasing amounts of the BTN2A1/3A1-Fc-B7H3scFv or BTN2A1/3A1-Fc-CD19scFv heterodimeric proteins were incubated with either OV-90 or RAJI cells for 1 hr at 4° C. Binding was detected using an AF647 conjugated anti-Fc reagent with flow cytometry. As shown in FIG. 14I (left panel), the BTN2A1/3A1-Fc-B7H3scFv heterodimeric protein showed a dose-dependent and saturable binding to OV-90 cells. On the other hand, the BTN2A1/3A1-Fc-CD19scFv heterodimeric protein showed a binding above background to OV-90 cells (FIG. 14I (left panel)). As shown in FIG. 14I (right panel), the BTN2A1/3A1-Fc-CD19scFv heterodimeric protein showed a dose-dependent and saturable binding to RAJI cells. On the other hand, the BTN2A1/3A1-Fc-B7H3scFv heterodimeric protein showed a binding above background to OV-90 cells (FIG. 14I (right panel)).

These results indicate, inter alia, that the BTN2A1/3A1-Fc-B7H3scFv construct specifically bound to B7H3+ cells.

### Example 16: Tumor Cell Killing by the BTN2A1/3A1-Fc-B7H3scFv Heterodimeric Protein

B7H3+ OVCAR3 cells were stained with a cell permeable, Cytolight Rapid Red dye and co-cultured with in vitro expanded Vγ9V62+-T cells together with the BTN2A1/3A1-Fc-B7H3scFv heterodimeric protein. The BTN2A1/3A1-Fc-CD19scFv heterodimeric protein served as a negative control since it lacked the B7H3 targeting domain. An anti-CD277 antibody and Zoledronate were used as positive controls. The Caspace 3/7 Green reagent was added to the co-culture to detect apoptosis within the tumor cells that were labeled in red. Live cell imaging was performed using the IncuCyte S3 instrument and the overlap an Caspace 3/7 Green signal and the Red tumor cell signal was quantified at the 6 hour time point in the assay. As shown in FIG. 14J, tumor cells mixed with Vγ9V62+-T cells alone showed low levels of apoptosis. The tumor cells mixed with Vγ9V62+-T cells and BTN2A1/3A1-Fc-CD19scFv heterodimeric protein showed apoptosis at levels that are similar to that in tumor cells mixed with Vγ9V62+-T cells alone. On the other hand, the tumor cells mixed with Vγ9V62+-T cells and BTN2A1/3A1-Fc-B7H3scFv heterodimeric protein showed significantly increased level of apoptosis (p<0.001) compared to that in tumor cells mixed with Vγ9V62+-T cells alone (FIG. 14J). As expected, the anti-CD277 antibody and Zoledronate induced apoptosis high levels of apoptosis. As expected, the tumor alone or T cells alone showed background levels of apoptosis (FIG. 14J).

These results indicate, interalia, that the BTN2A1/3A1-Fc-B7H3scFv construct specifically induces the killing of B7H3+ cells mediated by Vγ9V62+-T cells.

### Example 17: Construction and Characterization of BTN2A1/3A1-Fc-CD20scFv Heterodimeric Protein

The “BTN2A1/3A1-Fc-CD20scFv” construct included an alpha chain comprising an extracellular domain (ECD) of human BTN2A1 fused to a CD20scFv via a hinge-CH2-CH3 Fc domain, and a beta chain comprising an extracellular domain (ECD) of human BTN3A1 fused to a CD20scFv via a hinge-CH2-CH3 Fc domain. Constructs encoding BTN2A1-Fc-CD20scFv protein (alpha chain) and BTN3A1-Fc-CD20scFv protein (beta chain) were generated. This GAmma DELta T cell ENgager construct also is referred to herein as the BTN2A1/3A1-Fc-CD20scFv heterodimeric protein.

The BTN2A1/3A1-Fc-CD20scFv heterodimer protein that was produced via a transient co-transfection in ExpiCHO cells of two plasmids encoding 1) the BTN2A1-alpha-CD20scFv protein and 2) the BTN3A1-beta-CD20scFv protein. The alpha and beta constructs encoded a BTN2A1-Fc-CD20scFv (‘alpha’ chain) and a BTN3A1-Fc-CD20scFv (‘beta’ chain). The alpha and beta chains contained charged polarized linker domains which facilitated heterodimerization of the desired the BTN2A1/3A1-Fc-CD20scFv heterodimeric protein. The charged polarized domains, which favors formation of heterodimer because of charge-charge interation, and disfavors the formation of a homodimer. The cell culture supernatant from the transient transfection was harvested 6 days following transfection and purified over an affinity chromatography resin and elution under low pH conditions.

Purity of the protein was assessed using non-reducing sodium dodecyl sulfate polyacrylamide gel electrophoresis (SDS-PAGE). The purified protein was also analyzed by western blots using non-reducing, reducing, and reducing and deglycosylating conditions, following detection with an anti-human BTN2A1 antibody, or an anti-human BTN3A1 antibody. BTN2A1-alpha and BTN3A1-beta chains were detected in the protein prep using specific antibodies conjugated to IR dyes: goat anti Rabbit Starbright Blue (520 CW) and Donkey anti Goat/Sheep Green (800 CW). Non-reduced BTN2A1/3A1-Fc-CD20scFv heterodimeric protein ran as a single band (See lanes “NR” in FIG. 15A) indicative of covalent complex formation between the BTN2A1-alpha-CD20scFv and BTN3A1-beta-CD20scFv chains. As shown in FIG. 15A, the blots revealed two bands with the protein prepared under reducing but non-deglycosylated condition (See lane “R” in FIG. 15A). Interestingly, protein prepared under both reduced and deglycosylated (lane “DG”) conditions resulted 20 in a single band, which could be detected with any of the anti-human BTN2A1, anti-human BTN3A1, or anti-mouse Fc antibodies (See lane “DG” in FIG. 15A).

The ability of BTN2A1/3A1-Fc-CD20scFv heterodimeric protein to promote degranulation and cytokine production in Vγ9Vδ2+ T cells was assessed using the assay discussed in Example 7. Briefly, purified BTN2A1/3A1-Fc-CD20scFv heterodimeric protein and BTN2A1/3A1-Fc-CD19scFv heterodimeric protein were diluted in DPBS (Gibco) in high-binding 96-well plates (Corning) and a fixed amount of an anti-NKG2D antibody was added to each well. BTN2A1/3A1-Fc-CD19scFv heterodimeric protein was used as a positive control. The plates were incubated overnight at 4° C. Wells were washed twice with DPBS, and 0.5 to 1×10⁵ Vγ9Vδ2+-T cells in RPMI+10% FBS, supplemented with 100 U/mL hIL-2. BD FastImmune CD107a APC, GolgiStop, and GolgiPlug reagents (BD Biosciences) were added to cell culture according to the manufacturer's protocols. Cells were incubated at 37° C. for 4 hours and stained for cell surface and intracellular markers for analysis by flow cytometry. The proportion of CD107a was calculated as a fraction of the CD3+Vγ9+ population. As shown in FIG. 15B, CD107a-expressing Vγ9V62+-T cells increased in a dose-dependent manner when induced by the combinations of BTN2A1/3A1-Fc-CD20scFv heterodimeric protein and the anti-NKG2D antibody or BTN2A1/3A1-Fc-19scFv heterodimeric protein and the anti-NKG2D antibody (FIG. 15B).

These results demonstrate, inter alia, that the 3 BTN2A1/3A1-Fc-CD20scFv heterodimeric protein disclosed herein can activate and degranulate Vγ9V62+ T cells γδ T cells in the presence of CD28 or NKG2D costimulation.

### Example 18: Construction and Characterization of BTN2A1/3A1-Fc-CD33scFv Heterodimeric Protein

The “BTN2A1/3A1-Fc-CD33scFv” construct included an alpha chain comprising an extracellular domain (ECD) of human BTN2A1 fused to a CD33scFv via a hinge-CH2-CH3 Fc domain, and a beta chain comprising an extracellular domain (ECD) of human BTN3A1 fused to a CD33scFv via a hinge-CH2-CH3 Fc domain. Constructs encoding BTN2A1-Fc-CD33scFv protein (alpha chain) and BTN3A1-Fc-CD33scFv protein (beta chain) were generated. This GAmma DELta T cell ENgager construct also is referred to herein as the BTN2A1/3A1-Fc-CD33scFv heterodimeric protein.

The BTN2A1/3A1-Fc-CD33scFv heterodimer protein that was produced via a transient co-transfection in ExpiCHO cells of two plasmids encoding 1) the BTN2A1-alpha-CD33scFv protein and 2) the BTN3A1-beta-CD33scFv protein. The alpha and beta constructs encoded a BTN2A1-Fc-CD33scFv (‘alpha’ chain) and a BTN3A1-Fc-CD33scFv (‘beta’ chain). The alpha and beta chains contained charged polarized linker domains which facilitated heterodimerization of the desired the BTN2A1/3A1-Fc-CD33scFv heterodimeric protein. The charged polarized domains, which favors formation of heterodimer because of charge-charge interation, and disfavors the formation of a homodimer. The cell culture supernatant from the transient transfection was harvested 6 days following transfection and purified over an affinity chromatography resin and elution under low pH conditions.

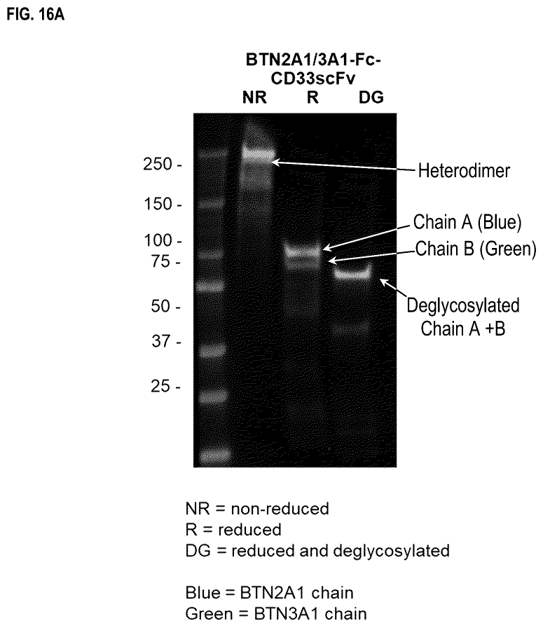

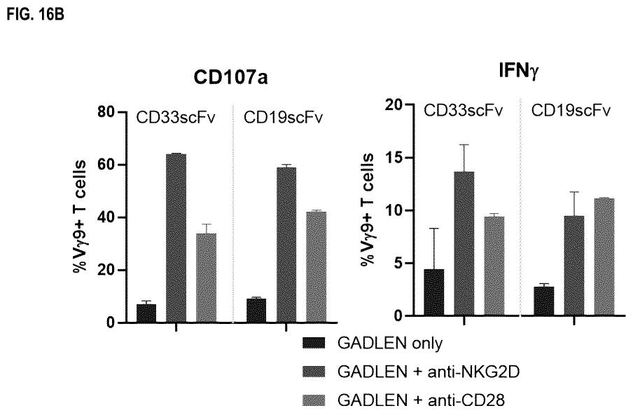

Purity of the protein was assessed using non-reducing sodium dodecyl sulfate polyacrylamide gel electrophoresis (SDS-PAGE). The purified protein was also analyzed by western blots using non-reducing, reducing, and reducing and deglycosylating conditions, following detection with an anti-human BTN2A1 antibody, or an anti-human BTN3A1 antibody. BTN2A1-alpha and BTN3A1-beta chains were detected in the protein prep using specific antibodies conjugated to IR dyes: goat anti Rabbit Starbright Blue (520 CW) and Donkey anti Goat/Sheep Green (800 CW). Non-reduced BTN2A1/3A1-Fc-CD33scFv heterodimeric protein ran as a single band (See lanes “NR” in FIG. 16A) indicative of covalent complex formation between the BTN2A1-alpha-CD33scFv and BTN3A1-beta-CD33scFv chains. As shown in FIG. 15A, the blots revealed two bands with the protein prepared under reducing but non-deglycosylated condition (See lane “R” in FIG. 15A). Interestingly, protein prepared under both reduced and deglycosylated (lane “DG”) conditions resulted in a single band, which could be detected with any of the anti-human BTN2A1, anti-human BTN3A1, or anti-mouse Fc antibodies (See lane “DG” in FIG. 16A).

The ability of BTN2A1/3A1-Fc-CD33scFv heterodimeric protein to promote degranulation and cytokine production in Vγ9Vδ2+ T cells was assessed using the assay discussed in Example 7. Briefly, purified BTN2A1/3A1-Fc-CD33scFv heterodimeric protein and BTN2A1/3A1-Fc-CD19scFv heterodimeric protein were added in high-binding 96-well plates (Corning) and an anti-NKG2D antibody was added to half wells containing BTN2A1/3A1-Fc-CD33scFv heterodimeric protein and all wells containing BTN2A1/3A1-Fc-CD19scFv heterodimeric protein. BTN2A1/3A1-Fc-CD19scFv heterodimeric protein was used as a positive control. The plates were incubated overnight at 4° C. Wells were washed twice with DPBS, and 0.5 to 1×10⁵ Vγ9Vδ2+-T cells in RPMI+10% FBS, supplemented with 100 U/mL hIL-2. BD FastImmune CD107a APC, GolgiStop, and GolgiPlug reagents (BD Biosciences) were added to cell culture according to the manufacturer's protocols. Cells were incubated at 37° C. for 4 hours and stained for cell surface and intracellular markers for analysis by flow cytometry. The proportion of CD107a was calculated as a fraction of the CD3+Vγ9+ population. As shown in FIG. 16B (left panel), CD107a-expressing Vγ9Vδ2+-T cells increased in when induced by the combinations of BTN2A1/3A1-Fc-CD33scFv heterodimeric protein and the anti-NKG2D antibody or BTN2A1/3A1-Fc-19scFv heterodimeric protein and the anti-NKG2D antibody. Vγ9Vδ2+-T cells induced by the combinations of BTN2A1/3A1-Fc-CD33scFv heterodimeric protein alone expressed only a background levels of CD107a (FIG. 16B (left panel)). As shown in FIG. 16B (right panel), IFNγ-expressing Vγ9Vδ2+-T cells increased in when induced by the combinations of BTN2A1/3A1-Fc-CD33scFv heterodimeric protein and the anti-NKG2D antibody or BTN2A1/3A1-Fc-19scFv heterodimeric protein and the anti-NKG2D antibody. Vγ9Vδ2+-T cells induced by the combinations of BTN2A1/3A1-Fc-CD33scFv heterodimeric protein alone expressed only a background levels of IFNγ (FIG. 16B (right panel)) These results demonstrate, inter alia, that the BTN2A1/3A1-Fc-CD20scFv heterodimeric protein disclosed herein can activate and degranulate Vγ9Vδ2+ T cells γδ T cells in the presence of CD28 or NKG2D costimulation.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present disclosure is not entitled to antedate such publication by virtue of prior disclosure.

As used herein, all headings are simply for organization and are not intended to limit the disclosure in any manner. The content of any individual section may be equally applicable to all sections.

### EQUIVALENTS

While the disclosure has been disclosed in connection with specific embodiments thereof, it will be understood that it is capable of further modifications and this application is intended to cover any variations, uses, or adaptations of the disclosure following, in general, the principles of the disclosure and including such departures from the present disclosure as come within known or customary practice within the art to which the disclosure pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

What is claimed is:

1. A method for treating a cancer in a subject in need thereof comprising:
(i) administering to the subject a first pharmaceutical composition comprising a heterodimeric protein comprising an alpha chain and a beta chain,
wherein the alpha chain comprises:
(a) a first domain comprising a BTN2A1 protein, or a fragment thereof;
(b) a second domain comprising a targeting domain; and
(c) a linker that adjoins the first and second domains;
and
wherein the beta chain comprises:
(a) a first domain comprising a BTN3A1 protein, or a fragment thereof;
(b) a second domain comprising a targeting domain; and
(c) a linker that adjoins the first and second domains
and
(ii) administering to the subject a second pharmaceutical composition that costimulates γδ T cells.

2. The method of claim 1, wherein the first pharmaceutical composition and the second pharmaceutical composition are administered simultaneously or contemporaneously.

3. The method of claim 1, wherein the first pharmaceutical composition is administered after the second pharmaceutical composition is administered.

4. The method of claim 1, wherein the first pharmaceutical composition is administered before the second pharmaceutical composition is administered.

5. The method of any one of claims 1 to 4, wherein the dose of the first pharmaceutical composition is less than the dose of the first pharmaceutical composition administered to a subject who has not undergone or is not undergoing treatment with the second pharmaceutical composition.

6. The method of any one of claims 1 to 5, wherein the dose of the second pharmaceutical composition administered is less than the dose of the second pharmaceutical composition administered to a subject who has not undergone or is not undergoing treatment with the first pharmaceutical composition.

7. The method of any one of claims 1 to 6, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

8. The method of any one of claims 1 to 7, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

9. A method for treating a cancer in a subject in need thereof comprising:
administering to the subject a first pharmaceutical composition comprising a heterodimeric protein comprising an alpha chain and a beta chain,
wherein the alpha chain comprises:
(a) a first domain comprising a BTN2A1 protein, or a fragment thereof;
(b) a second domain comprising a targeting domain; and
(c) a linker that adjoins the first and second domains;
and
wherein the beta chain comprises:
(a) a first domain comprising a BTN3A1 protein, or a fragment thereof;
(b) a second domain comprising a targeting domain; and
(c) a linker that adjoins the first and second domains;
wherein the subject has undergone or is undergoing treatment with a second pharmaceutical composition comprising a costimulatory molecule.

10. The method of claim 9, wherein the first pharmaceutical composition and the second pharmaceutical composition are administered simultaneously or contemporaneously.

11. The method of claim 9, wherein the first pharmaceutical composition is administered after the second pharmaceutical composition is administered.

12. The method of claim 9, wherein the first pharmaceutical composition is administered before the second pharmaceutical composition is administered.

13. The method of any one of claims 9 to 12, wherein the dose of the first pharmaceutical composition is less than the dose of the first pharmaceutical composition administered to a subject who has not undergone or is not undergoing treatment with the second pharmaceutical composition.

14. The method of any one of claims 9 to 13, wherein the dose of the second pharmaceutical composition administered is less than the dose of the second pharmaceutical composition administered to a subject who has not undergone or is not undergoing treatment with the first pharmaceutical composition.

15. The method of any one of claims 9 to 14, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

16. The method of any one of claims 9 to 15, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

17. A method for treating a cancer in a subject in need thereof comprising:
administering to the subject a second pharmaceutical composition that costimulates γδ T cells,
wherein the subject has undergone or is undergoing treatment with a first pharmaceutical composition comprising a heterodimeric protein comprising an alpha chain and a beta chain,
wherein the alpha chain comprises:
(a) a first domain comprising a BTN2A1 protein, or a fragment thereof;
(b) a second domain comprising a targeting domain; and
(c) a linker that adjoins the first and second domains;
and
wherein the beta chain comprises:
(a) a first domain comprising a BTN3A1 protein, or a fragment thereof;
(b) a second domain comprising a targeting domain; and
(c) a linker that adjoins the first and second domains.

18. The method of claim 17, wherein the first pharmaceutical composition and the second pharmaceutical composition are administered simultaneously or contemporaneously.

19. The method of claim 17, wherein the first pharmaceutical composition is administered after the second pharmaceutical composition is administered.

20. The method of claim 17, wherein the first pharmaceutical composition is administered before the second pharmaceutical composition is administered.

21. The method of any one of claims 17 to 20, wherein the dose of the first pharmaceutical composition is less than the dose of the first pharmaceutical composition administered to a subject who has not undergone or is not undergoing treatment with the second pharmaceutical composition.

22. The method of any one of claims 17 to 21, wherein the dose of the second pharmaceutical composition administered is less than the dose of the second pharmaceutical composition administered to a subject who has not undergone or is not undergoing treatment with the first pharmaceutical composition.

23. The method of any one of claims 17 to 22, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

24. The method of any one of claims 17 to 23, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

25. The method of any one of claims 1 to 24, wherein the second pharmaceutical composition costimulates a receptor selected from CD28, NKG2D, CD27, CD30, 4-1BB (CD137), IL-2R, IL-15R, IL-7R, IL-21R, NKp30, NKp44, DNAM-1 (CD226), IL-2R, IL-7R, IL-15R, dectins, NLRs, killer Ig-like receptors (e.g., KIR2D, KIR3D), C-type lectins (CD94/NKG2A-C, NKG2D), LFA1, CD2, CD46, Junctional Adhesion Molecule-Like (JAML).

26. The method of claim 25, wherein the second pharmaceutical composition comprises a ligand of the receptor, or a receptor-binding portion thereof.

27. The method of claim 26, wherein the second pharmaceutical composition comprises a fusion protein (e.g., an Fc fusion protein or an albumin fusion protein) comprising a co-stimulatory molecule, or a binding portion thereof, or the ligand of the receptor, or receptor-binding portion thereof.

28. The method of claim 25, wherein the second pharmaceutical composition comprises an antibody, antibody-like molecule or a receptor-binding portion thereof.

29. The method of claim 28, wherein the second pharmaceutical composition comprises an agonistic antibody.

30. The method of any one of claims 25 to 29, wherein the second pharmaceutical composition costimulates CD28 and/or NKG2D.

31. The method of claim 30, wherein the second pharmaceutical composition comprises a CD28 ligand, a CD28-binding portion thereof, an NKG2D ligand, or an NKG2D-binding portion thereof.

32. The method of claim 31, wherein the NKG2D ligand is selected from MICA, MICB, ULBP1, ULBP2, ULBP3, ULBP4, ULBP5, ULBP6, H60, MULT1, and RAE1.

33. The method of claim 31, wherein the NKG2D ligand is an antibody, an antibody-like molecule, or a binding fragment thereof.

34. The method of claim 33, wherein the binding fragment is selected from Fab fragment, heavy variable chain, and single chain variable fragments (scFV).

35. The method of claim 33, wherein the antibody is an agonistic antibody.

36. The method of claim 33 or claim 35, wherein the antibody is a monoclonal antibody.

37. The method of claim 36, wherein the antibody is an anti-NKG2D monoclonal antibody selected from tesnatilimab, 149810, 1D11 and 5C6.

38. The method of claim 31, wherein the CD28 ligand is selected from CD80 and CD86.

39. The method of claim 31, wherein the CD28 ligand of is an antibody, an antibody-like molecule, or a binding fragment thereof.

40. The method of claim 39, wherein the binding fragment is selected from Fab fragment, heavy variable chain, and single chain variable fragments (scFV).

41. The method of claim 39, wherein the antibody is an agonistic antibody.

42. The method of claim 39 or claim 41, wherein the antibody is a monoclonal antibody.

43. The method of claim 42, wherein the antibody is an anti-CD28 monoclonal antibody selected from JJ316, D665, 5.11A1, TGN1412, 37.51, E18, and PV-1.

44. The method of any one of claims 1 to 24, wherein the second pharmaceutical composition inhibits a receptor selected from a receptor selected from PD-1, PD-L1 and BTLA.

45. The method of claim 44, wherein the second pharmaceutical composition comprises a soluble receptor.

46. The method of claim 45, wherein the second pharmaceutical composition comprises an extracellular domain of PD-1, an extracellular domain of BTLA, or a receptor binding domain thereof.

47. The method of claim 44, wherein the second pharmaceutical composition comprises an antibody, an antibody-like molecule, or a binding fragment thereof.

48. The method of claim 47, wherein the binding fragment is selected from Fab fragment, heavy variable chain, and single chain variable fragments (scFV).

49. The method of claim 47, wherein the antibody is an antagonistic antibody.

50. The method of claim 47 or claim 49, wherein the antibody is a monoclonal antibody.

51. The method of claim 50, wherein the monoclonal antibody is an anti-PD-1 antibody selected from pembrolizumab, nivolumab, and cemiplimab.

52. The method of any one of claims 1 to 51, wherein the BTN2A1 protein, or a fragment thereof comprises a variable Ig-like V-type domain.

53. The method of claim 52, wherein the variable Ig-like V-type domain of the BTN2A1 protein comprises an amino acid sequence that is at least 90%, or 93%, or 95%, or 97%, or 98%, or 99% identical to the amino acid sequence of SEQ ID NO: 28.

54. The method of any one of claims 1 to 51, wherein the BTN2A1 protein, or a fragment thereof comprise a extracellular domain (ECD).

55. The method of claim 54, wherein the variable ECD of the BTN2A1 protein comprises an amino acid sequence that is at least 90%, or 93%, or 95%, or 97%, or 98%, or 99% identical to the amino acid sequence of SEQ ID NO: 27.

56. The method of any one of claims 1 to 55, wherein the BTN3A1 protein, or a fragment thereof comprise a variable Ig-like V-type domain.

57. The method of claim 56, wherein the variable Ig-like V-type domain of the BTN3A1 protein comprises an amino acid sequence that is at least 90%, or 93%, or 95%, or 97%, or 98%, or 99% identical to the amino acid sequence of SEQ ID NO: 30.

58. The method of any one of claims 1 to 55, wherein the BTN3A1 protein, or a fragment thereof comprise a extracellular domain (ECD).

59. The method of claim 58, wherein the variable ECD of the BTN3A1 protein comprises an amino acid sequence that is at least 90%, or 93%, or 95%, or 97%, or 98%, or 99% identical to the amino acid sequence of SEQ ID NO: 29.

60. The method of any one of claims 1 to 59, wherein the first domain comprises an amino acid sequence having an amino acid sequence of selected from one or more of SEQ ID NOs: 27-30.

61. The method of any one of claims 1 to 60, wherein the targeting domain is an antibody, or antigen binding fragment thereof.

62. The method of any one of claims 1 to 60, wherein the targeting domain is an antibody-like molecule, or antigen binding fragment thereof.

63. The method of claim 62, wherein the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)2.

64. The method of claim 63, wherein the antibody-like molecule is an scFv.

65. The method of any one of claims 1 to 60, wherein the targeting domain is an extracellular domain.

66. The method of claim 65, wherein the targeting domain is capable of binding an antigen on the surface of a cancer cell.

67. The method of claim 65 or claim 66, wherein the targeting domain specifically binds one or more of CLEC12A, CD307, gpA33, mesothelin, CDH17, CDH3/P-cadherin, CEACAM5/CEA, EPHA2, NY-eso-1, GP100, MAGE-A1, MAGE-A4, MSLN, CLDN18.2, Trop-2, ROR1, CD123, CD33, CD20, GPRC5D, GD2, CD276/B7-H3, DLL3, PSMA, CD19, cMet, HER2, A33, TAG72, 5T4, CA9, CD70, MUC1, NKG2D, CD133, EpCam, MUC17, EGFRvIII, IL13R, CPC3, GPC3, FAP, BCMA, CD171, SSTR2, FOLR1, MUC16, CD274/PDL1, CD44, KDR/VEGFR2, PDCD1/PD1, TEM1/CD248, LeY, CD133, CELEC12A/CLL1, FLT3, IL1RAP, CD22, CD23, CD30/TNFRSF8, FCRH5, SLAMF7/CS1, CD38, CD4, PRAME, EGFR, PSCA, STEAP1, CD174/FUT3/LeY, L1CAM/CD171, CD22, CD5, LGR5, LGR5, and GD3.

68. The method of any one of claim 67, wherein the targeting domain comprises a portion of the extracellular domain of LAG-3, PD-1, TIGIT, CD19, and PCMA.

69. The method of any one of claims 1 to 68, wherein the targeting domain specifically binds of an antigen selected from CD19, PSMA, B7H3, FAP, CD20 and CD33.

70. The method of any one of claims 1 to 68, wherein the targeting domain specifically binds of CD19.

71. The method of any one of claims 1 to 68, wherein the targeting domain specifically binds PSMA.

72. The method of any one of claims 1 to 68, wherein the targeting domain specifically binds B7H3.

73. The method of any one of claims 1 to 68, wherein the targeting domain specifically binds FAP.

74. The method of any one of claims 1 to 68, wherein the targeting domain specifically binds CD20.

75. The method of any one of claims 1 to 68, wherein the targeting domain specifically binds CD33.

76. The method of claim 69 or claim 70, wherein the targeting domain is a polypeptide having an amino acid sequence with at least 90%, or 95%, or 97%, or 98%, or 99% identity with a polypeptide selected from SEQ ID NOs: 31-35, 41, 48, 111 and 112.

77. The method of any one of claim 69 or 70, wherein the targeting domain is a polypeptide having an amino acid sequence of selected from one or more of SEQ ID NOs: 31-35, 41, 48, 111 and 112.

78. The method of any one of claims 1 to 77, wherein the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus.

79. The method of claim 78, wherein the linker forms a heterodimer through electrostatic interactions between positively charged amino acid residues and negatively charged amino acid residues on the first and second charge polarized core domains.

80. The method of claim 79, wherein the first and/or second charge polarized core domain comprises a polypeptide linker, optionally selected from a flexible amino acid sequence, IgG hinge region, or antibody sequence.

81. The method of any one of claims 1 to 80, wherein the linker is a synthetic linker, optionally PEG.

82. The method of any one of claims 78 to 81, wherein the linker comprises the hinge-CH2-CH3 Fc domain derived from IgG1, optionally from human IgG1.

83. The method of any one of claims 78 to 82, wherein the linker comprises the hinge-CH2-CH3 Fc domain derived from IgG4, optionally from human IgG4.

84. The method of any one of claims 78 to 83, wherein the first and/or second charge polarized core domain further comprise peptides having positively and/or negatively charged amino acid residues at the amino and/or carboxy terminus of the charge polarized core domain.

85. The method of claim 84, wherein the positively charged amino acid residues include one or more of amino acids selected from His, Lys, and Arg.

86. The method of claim 84 or claim 85, wherein the positively charged amino acid residues are present in a peptide comprising positively charged amino acid residues in the first and/or the second charge polarized core domains.

87. The method of claim 86, wherein the peptide comprising positively charged amino acid residues comprises the sequence RKGGKR (SEQ ID NO: 11) or GSGSRKGGKRGS (SEQ ID NO: 12).

88. The method of any one of claims 84 to 87, wherein the negatively charged amino acid residues may include one or more amino acids selected from Asp and Glu.

89. The method of any one of claims 84 to 88, wherein the negatively charged amino acid residues are present in a peptide comprising negatively charged amino acid residues in the first and/or the second charge polarized core domains.

90. The method of claim 88 or claim 89, wherein the peptide comprising negatively charged amino acid residues comprises the sequence DEGGED (SEQ ID NO: 13) or GSGSDEGGEDGS (SEQ ID NO: 14).

91. The method of any one of claims 1 to 90, wherein the first domain and/or the heterodimeric protein modulates or is capable of modulating a γδ (gamma delta) T cell, optionally wherein the gamma delta T cell is selected from a cell expressing Vγ4, Vγ9δ2, or Vγ7δ4.

92. The method of any one of claims 1 to 91, wherein the first domain modulates a Vγ9δ2-expressing T cell.

93. A method for treating a cancer in a subject in need thereof comprising:
(i) administering to the subject a first pharmaceutical composition comprising a heterodimeric protein comprising an alpha chain and a beta chain,
wherein the alpha chain comprises:
(a) a first domain comprising a BTN2A1 protein, or a fragment thereof;
(b) a second domain comprising a targeting domain, wherein the targeting domain specifically binds CD19, PSMA, B7H3, FAP, CD20 or CD33; and
(c) a linker that adjoins the first and second domains;
and
wherein the beta chain comprises:
(a) a first domain comprising a BTN3A1 protein, or a fragment thereof;
(b) a second domain comprising a targeting domain, wherein the targeting domain specifically binds CD19, PSMA, B7H3, FAP, CD20 or CD33; and
(c) a linker that adjoins the first and second domains
and
(ii) administering to the subject a second pharmaceutical composition comprising an anti-CD28 antibody and/or and anti-NKG2D antibody.

94. A method for treating a cancer in a subject in need thereof comprising:
administering to the subject a first pharmaceutical composition comprising a heterodimeric protein comprising an alpha chain and a beta chain,
wherein the alpha chain comprises:
(a) a first domain comprising a BTN2A1 protein, or a fragment thereof;
(b) a second domain comprising a targeting domain, wherein the targeting domain specifically binds CD19, PSMA, B7H3, FAP, CD20 or CD33; and
(c) a linker that adjoins the first and second domains;
and
wherein the beta chain comprises:
(a) a first domain comprising a BTN3A1 protein, or a fragment thereof;
(b) a second domain comprising a targeting domain, wherein the targeting domain specifically binds CD19, PSMA, B7H3, FAP, CD20 or CD33; and
(c) a linker that adjoins the first and second domains;
wherein the subject has undergone or is undergoing treatment with a second pharmaceutical composition comprising an anti-CD28 antibody and/or and anti-NKG2D antibody.

95. A method for treating a cancer in a subject in need thereof comprising:
administering to the subject a second pharmaceutical composition comprising an anti-CD28 antibody and/or and anti-NKG2D antibody,
wherein the subject has undergone or is undergoing treatment with a first pharmaceutical composition comprising a heterodimeric protein comprising an alpha chain and a beta chain,
wherein the alpha chain comprises:
(a) a first domain comprising a BTN2A1 protein, or a fragment thereof;
(b) a second domain comprising a targeting domain, wherein targeting domain specifically binds CD19, PSMA, B7H3, FAP, CD20 or CD33; and
(c) a linker that adjoins the first and second domains;
and
wherein the beta chain comprises:
(a) a first domain comprising a BTN3A1 protein, or a fragment thereof;
(b) a second domain comprising a targeting domain, wherein the targeting domain specifically binds CD19, PSMA, B7H3, FAP, CD20 or CD33; and
(c) a linker that adjoins the first and second domains.

96. The method of claim 93, wherein the first pharmaceutical composition and the second pharmaceutical composition are administered simultaneously or contemporaneously.

97. The method of claim 93, wherein the first pharmaceutical composition is administered after the second pharmaceutical composition is administered.

98. The method of claim 93, wherein the first pharmaceutical composition is administered before the second pharmaceutical composition is administered.

99. The method of any one of claims 93 to 98, wherein the dose of the first pharmaceutical composition is less than the dose of the first pharmaceutical composition administered to a subject who has not undergone or is not undergoing treatment with the second pharmaceutical composition.

100. The method of any one of claims 93 to 99, wherein the dose of the second pharmaceutical composition administered is less than the dose of the second pharmaceutical composition administered to a subject who has not undergone or is not undergoing treatment with the first pharmaceutical composition.

101. The method of any one of claims 93 to 100, wherein the subject has an increased chance of survival, without gastrointestinal inflammation and weight loss, and/or a reduction in tumor size or cancer prevalence when compared to a subject who has only undergone or is only undergoing treatment with the first pharmaceutical composition.

102. The method of any one of claims 93 to 101, wherein the subject has an increased chance of survival, without gastrointestinal inflammation and weight loss, and/or a reduction in tumor size or cancer prevalence when compared to a subject who has only undergone or is only undergoing treatment with the second pharmaceutical composition.

103. The method of any one of claims 93 to 102, wherein the antibody is an agonistic antibody.

104. The method of claim 103, wherein the antibody is a monoclonal antibody.

105. The method of claim 104, wherein the antibody is an anti-NKG2D monoclonal antibody selected from tesnatilimab, 149810, 1D11 and 5C6.

106. The method of claim 104, wherein the antibody is an anti-CD28 monoclonal antibody selected from JJ316, D665, 5.11A1, TGN1412, 37.51, E18, and PV-1.

107. The method of any one of claims 93 to 106, wherein the BTN2A1 protein, or a fragment thereof comprises a variable Ig-like V-type domain.

108. The method of claim 107, wherein the variable Ig-like V-type domain of the BTN2A1 protein comprises an amino acid sequence that is at least 90%, or 93%, or 95%, or 97%, or 98%, or 99% identical to the amino acid sequence of SEQ ID NO: 28.

109. The method of any one of claims 93 to 108, wherein the BTN2A1 protein, or a fragment thereof comprise a extracellular domain (ECD).

110. The method of claim 109, wherein the variable ECD of the BTN2A1 protein comprises an amino acid sequence that is at least 90%, or 93%, or 95%, or 97%, or 98%, or 99% identical to the amino acid sequence of SEQ ID NO: 27.

111. The method of any one of claims 93 to 110, wherein the BTN3A1 protein, or a fragment thereof comprise a variable Ig-like V-type domain.

112. The method of claim 111, wherein the variable Ig-like V-type domain of the BTN3A1 protein comprises an amino acid sequence that is at least 90%, or 93%, or 95%, or 97%, or 98%, or 99% identical to the amino acid sequence of SEQ ID NO: 30.

113. The method of any one of claims 93 to 112, wherein the BTN3A1 protein, or a fragment thereof comprise a extracellular domain (ECD).

114. The method of claim 113, wherein the variable ECD of the BTN3A1 protein comprises an amino acid sequence that is at least 90%, or 93%, or 95%, or 97%, or 98%, or 99% identical to the amino acid sequence of SEQ ID NO: 29.

115. The method of any one of claims 93 to 114, wherein the first domain comprises an amino acid sequence having an amino acid sequence of selected from one or more of SEQ ID NOs: 27-30.

116. The method of any one of claims 93 to 115, wherein the targeting domain is an antibody, an antibody-like molecule, or antigen binding fragment thereof.

117. The method of claim 116, wherein the antibody-like molecule is selected from a single-domain antibody, a recombinant heavy-chain-only antibody (VHH), a single-chain antibody (scFv), a shark heavy-chain-only antibody (VNAR), a microprotein (cysteine knot protein, knottin), a DARPin; a Tetranectin; an Affibody; a Transbody; an Anticalin; an AdNectin; an Affilin; an Affimer, a Microbody; an aptamer; an alterase; a plastic antibody; a phylomer; a stradobody; a maxibody; an evibody; a fynomer, an armadillo repeat protein, a Kunitz domain, an avimer, an atrimer, a probody, an immunobody, a triomab, a troybody; a pepbody; a vaccibody, a UniBody; a DuoBody, a Fv, a Fab, a Fab′, and a F(ab′)2.

118. The method of claim 117, wherein the antibody-like molecule is an scFv.

119. The method of claim 117 or claim 118, wherein the targeting domain is a polypeptide having an amino acid sequence with at least 90%, or 95%, or 97%, or 98%, or 99% identity with a polypeptide selected from SEQ ID NOs: 31-35, 41, 48, 111 and 112.

120. The method of any one of claims 117 to 119, wherein the targeting domain is a polypeptide having an amino acid sequence of selected from one or more of SEQ ID NOs: 31-35, 41, 48, 111 and 112.

121. The method of any one of claims 93 to 120, wherein the linker comprises (a) a first charge polarized core domain adjoined to a butyrophilin family protein, optionally at the carboxy terminus, and (b) a second charge polarized core domain adjoined to a butyrophilin family protein, optionally at the carboxy terminus.

122. The method of claim 121, wherein the linker forms a heterodimer through electrostatic interactions between positively charged amino acid residues and negatively charged amino acid residues on the first and second charge polarized core domains.

123. The method of claim 121, wherein the first and/or second charge polarized core domain comprises a polypeptide linker, optionally selected from a flexible amino acid sequence, IgG hinge region, or antibody sequence.

124. The method of any one of claims 93 to 121, wherein the linker is a synthetic linker, optionally PEG.

125. The method of any one of claims 121 to 123, wherein the linker comprises the hinge-CH2-CH3 Fc domain derived from IgG1, optionally from human IgG1.

126. The method of any one of claims 121 to 123, wherein the linker comprises the hinge-CH2-CH3 Fc domain derived from IgG4, optionally from human IgG4.

127. The method of any one of claims 121 to 126, wherein the first and/or second charge polarized core domain further comprise peptides having positively and/or negatively charged amino acid residues at the amino and/or carboxy terminus of the charge polarized core domain.

128. The method of claim 122, wherein the positively charged amino acid residues include one or more of amino acids selected from His, Lys, and Arg.

129. The method of claim 127 or claim 128, wherein the positively charged amino acid residues are present in a peptide comprising positively charged amino acid residues in the first and/or the second charge polarized core domains.

130. The method of claim 129, wherein the peptide comprising positively charged amino acid residues comprises a sequence selected from YₙXₙYₙXₙYₙ (where X is a positively charged amino acid such as arginine, histidine or lysine and Y is a spacer amino acid such as serine or glycine, and where each n is independently an integer 0 to 4) (SEQ ID NO: 1), YYₙXXₙYYₙXXₙYYₙ (where X is a positively charged amino acid such as arginine, histidine or lysine and Y is a spacer amino acid such as serine or glycine, and where each n is independently an integer 0 to 4) (SEQ ID NO: 3), and YₙXₙCYₙXₙYₙ (where X is a positively charged amino acid such as arginine, histidine or lysine and Y is a spacer amino acid such as serine or glycine, and where each n is independently an integer 0 to 4) (SEQ ID NO: 5).

131. The method of claim 130, wherein the peptide comprising positively charged amino acid residues comprises the sequence RKGGKR (SEQ ID NO: 11) or GSGSRKGGKRGS (SEQ ID NO: 12).

132. The method of any one of claims 127 to 131, wherein the negatively charged amino acid residues may include one or more amino acids selected from Asp and Glu.

133. The method of any one of claims 127 to 132, wherein the negatively charged amino acid residues are present in a peptide comprising negatively charged amino acid residues in the first and/or the second charge polarized core domains.

134. The method of claim 133, wherein the peptide comprising negatively charged amino acid residues comprises a sequence selected from YₙZₙYₙZₙYₙ (where Z is a negatively charged amino acid such as aspartic acid or glutamic acid and Y is a spacer amino acid such as serine or glycine, and where each n is independently an integer 0 to 4) (SEQ ID NO: 2), YYₙZZₙYYₙZZₙYYₙ (where Z is a negatively charged amino acid such as aspartic acid or glutamic acid and Y is a spacer amino acid such as serine or glycine, and where each n is independently an integer 0 to 4) (SEQ ID NO: 4), and YₙZₙCYₙZₙYₙ (where Z is a negatively charged amino acid such as aspartic acid or glutamic acid and Y is a spacer amino acid such as serine or glycine, and where each n is independently an integer 0 to 4) (SEQ ID NO: 6).

135. The method of claim 134, wherein the peptide comprising negatively charged amino acid residues comprises the sequence DEGGED (SEQ ID NO: 13) or GSGSDEGGEDGS (SEQ ID NO: 14).

136. The method of any one of claims 93 to 135, wherein the heterodimeric protein comprises amino acid sequences that is at least 90%, or 93%, or 95%, or 97%, or 98%, or 99% identical to the amino acid sequences of SEQ ID NO: 75, and SEQ ID NO: 81.

137. The method of claim 136, wherein the heterodimeric protein comprises amino acid sequences of SEQ ID NO: 75, and SEQ ID NO: 81.

138. A method for treating a cancer in a subject in need thereof comprising:
(i) administering to the subject a first pharmaceutical composition comprising a heterodimeric protein comprising an alpha chain and a beta chain,
wherein the alpha chain comprises:
(a) a first domain comprising an amino acid sequence that is 90%, or 93%, or 95%, or 97%, or 98%, or 99% identical to the amino acid sequence of SEQ ID NO: 27 or 28;
(b) a second domain comprising an amino acid sequence that is 90%, or 93%, or 95%, or 97%, or 98%, or 99% identical to an amino acid sequence selected from SEQ ID NOs: 35, 41, 48,111 and 112; and
(c) a linker comprising an amino acid sequence that is 90%, or 93%, or 95%, or 97%, or 98%, or 99% identical to the amino acid sequence of SEQ ID NO: 16;
and
wherein the beta chain comprises:
(a) a first domain comprising an amino acid sequence that is 90%, or 93%, or 95%, or 97%, or 98%, or 99% identical to the amino acid sequence of SEQ ID NO: 29 or 30;
(b) a second domain comprising an amino acid sequence that is 90%, or 93%, or 95%, or 97%, or 98%, or 99% identical to an amino acid sequence selected from SEQ ID NOs: 35, 41, 48, 111 and 112; and
(c) a linker comprising an amino acid sequence that is 90%, or 93%, or 95%, or 97%, or 98%, or 99% identical to the amino acid sequence of SEQ ID NO: 17;
and
(ii) administering to the subject a second pharmaceutical composition comprising an anti-CD28 antibody and/or and anti-NKG2D antibody.

139. A method for treating a cancer in a subject in need thereof comprising:
administering to the subject a first pharmaceutical composition comprising a heterodimeric protein comprising an alpha chain and a beta chain,
wherein the alpha chain comprises:
(a) a first domain comprising an amino acid sequence that is 90%, or 93%, or 95%, or 97%, or 98%, or 99% identical to the amino acid sequence of SEQ ID NO: 27 or 28;
(b) a second domain comprising an amino acid sequence that is 90%, or 93%, or 95%, or 97%, or 98%, or 99% identical to an amino acid sequence selected from SEQ ID NOs: 35, 41, 48, 111 and 112; and
(c) a linker comprising an amino acid sequence that is 90%, or 93%, or 95%, or 97%, or 98%, or 99% identical to the amino acid sequence of SEQ ID NO: 16;
and
wherein the beta chain comprises:
(a) a first domain comprising an amino acid sequence that is 90%, or 93%, or 95%, or 97%, or 98%, or 99% identical to the amino acid sequence of SEQ ID NO: 29 or 30;
(b) a second domain comprising an amino acid sequence that is 90%, or 93%, or 95%, or 97%, or 98%, or 99% identical to an amino acid sequence selected from SEQ ID NOs: 35, 41, 48, 111 and 112; and
(c) a linker comprising an amino acid sequence that is 90%, or 93%, or 95%, or 97%, or 98%, or 99% identical to the amino acid sequence of SEQ ID NO: 17;
wherein the subject has undergone or is undergoing treatment with a second pharmaceutical composition comprising an anti-CD28 antibody and/or and anti-NKG2D antibody.

140. A method for treating a cancer in a subject in need thereof comprising:
administering to the subject a second pharmaceutical composition comprising an anti-CD28 antibody and/or and anti-NKG2D antibody,
wherein the subject has undergone or is undergoing treatment with a first pharmaceutical composition comprising a heterodimeric protein comprising an alpha chain and a beta chain,
wherein the alpha chain comprises:
(a) a first domain comprising an amino acid sequence that is 90%, or 93%, or 95%, or 97%, or 98%, or 99% identical to the amino acid sequence of SEQ ID NO: 27 or 28;
(b) a second domain comprising an amino acid sequence that is 90%, or 93%, or 95%, or 97%, or 98%, or 99% identical to an amino acid sequence selected from SEQ ID NOs: 35, 41, 48, 111 and 112; and
(c) a linker comprising an amino acid sequence that is 90%, or 93%, or 95%, or 97%, or 98%, or 99% identical to the amino acid sequence of SEQ ID NO: 16;
and
wherein the beta chain comprises:
(a) a first domain comprising an amino acid sequence that is 90%, or 93%, or 95%, or 97%, or 98%, or 99% identical to the amino acid sequence of SEQ ID NO: 29 or 30;
(b) a second domain comprising an amino acid sequence that is 90%, or 93%, or 95%, or 97%, or 98%, or 99% identical to an amino acid sequence selected from SEQ ID NOs: 35, 41, 48, 111 and 112; and
(c) a linker comprising an amino acid sequence that is 90%, or 93%, or 95%, or 97%, or 98%, or 99% identical to the amino acid sequence of SEQ ID NO: 17.

141. The method of any one of claims 138 to 140, wherein the heterodimeric protein comprises an alpha chain and a beta chain, wherein, the alpha chain comprises an amino acid sequence that is at least 90%, or 93%, or 95%, or 97%, or 98%, or 99% identical to an amino acid sequence selected from SEQ ID NOs: 75, 113, 115, 117 and 119; and the beta chain comprises an amino acid sequence that is at least 90%, or 93%, or 95%, or 97%, or 98%, or 99% identical to an amino acid sequence selected from SEQ ID NOs: 81, 114, 116, 118, and 120.

142. The method of claim 141, wherein the alpha chain comprises an amino acid sequence that is at least 90%, or 93%, or 95%, or 97%, or 98%, or 99% identical to the amino acid sequence of SEQ ID NO: 75, and the beta chain comprises an amino acid sequence that is at least 90%, or 93%, or 95%, or 97%, or 98%, or 99% identical to the amino acid sequence of SEQ ID NO: 81.

143. The method of claim 141, wherein the alpha chain comprises an amino acid sequence that is at least 90%, or 93%, or 95%, or 97%, or 98%, or 99% identical to the amino acid sequence of SEQ ID NO: 113, and the beta chain comprises an amino acid sequence that is at least 90%, or 93%, or 95%, or 97%, or 98%, or 99% identical to the amino acid sequence of SEQ ID NO: 114.

144. The method of claim 141, wherein the alpha chain comprises an amino acid sequence that is at least 90%, or 93%, or 95%, or 97%, or 98%, or 99% identical to the amino acid sequence of SEQ ID NO: 115, and the beta chain comprises an amino acid sequence that is at least 90%, or 93%, or 95%, or 97%, or 98%, or 99% identical to the amino acid sequence of SEQ ID NO: 115.

145. The method of claim 141, wherein the alpha chain comprises an amino acid sequence that is at least 90%, or 93%, or 95%, or 97%, or 98%, or 99% identical to the amino acid sequence of SEQ ID NO: 117, and the beta chain comprises an amino acid sequence that is at least 90%, or 93%, or 95%, or 97%, or 98%, or 99% identical to the amino acid sequence of SEQ ID NO: 118.

146. The method of claim 141, wherein the alpha chain comprises an amino acid sequence that is at least 90%, or 93%, or 95%, or 97%, or 98%, or 99% identical to the amino acid sequence of SEQ ID NO: 119, and the beta chain comprises an amino acid sequence that is at least 90%, or 93%, or 95%, or 97%, or 98%, or 99% identical to the amino acid sequence of SEQ ID NO: 120.


SEQUENCE LISTING (120 sequences)

SEQ ID NO: 1 - protein, 20 aa, Artificial Sequence
  Synthetic Sequence
  MISC_FEATURE (1)..(4): Xaa is a spacer amino acid such as serine or glycine, and any of them can be absent.
  MISC_FEATURE (5)..(8): Xaa is a positively charged amino acid such as arginine, histidine or lysine, and any of them can be absent.
  MISC_FEATURE (9)..(12): Xaa is a spacer amino acid such as serine or glycine, and any of them can be absent.
  MISC_FEATURE (13)..(16): Xaa is a  positively charged amino acid such as arginine, histidine or lysine, and any of them can be absent.
  MISC_FEATURE (17)..(20): Xaa is a spacer amino acid such as serine or glycine, and any of them can be absent.
XXXXXXXXXXXXXXXXXXXX

SEQ ID NO: 2 - protein, 18 aa, Artificial Sequence
  Synthetic Sequence
  MISC_FEATURE (1)..(4): Xaa is a spacer amino acid such as serine or glycine, and any of them can be absent.
  MISC_FEATURE (5)..(8): Xaa is a negatively charged amino acid such as aspartic acid or glutamic acid, and any of them can be absent.
  MISC_FEATURE (9)..(12): Xaa is a spacer amino acid such as serine or glycine, and any of them can be absent.
  MISC_FEATURE (13)..(16): Xaa is a negatively charged amino acid such as aspartic acid or glutamic acid, and any of them can be absent.
  MISC_FEATURE (17)..(20): Xaa is a spacer amino acid such as serine or glycine, and any of them can be absent.
XXXXXXXXXXXXXXXXXX

SEQ ID NO: 3 - protein, 25 aa, Artificial Sequence
  Synthetic Sequence
  MISC_FEATURE (1)..(1): Xaa a spacer amino acid such as serine or glycine
  MISC_FEATURE (2)..(5): Xaa is a spacer amino acid such as serine or glycine, and any of them can be absent.
  MISC_FEATURE (6)..(6): Xaa is a positively charged amino acid such as arginine, histidine or lysine.
  MISC_FEATURE (7)..(10): Xaa is a positively charged amino acid such as arginine, histidine or lysine, and any of them can be absent.
  MISC_FEATURE (11)..(11): Xaa is a spacer amino acid such as serine or glycine.
  MISC_FEATURE (12)..(15): Xaa is a spacer amino acid such as serine or glycine, and any of them can be absent.
  MISC_FEATURE (16)..(16): Xaa is a positively charged amino acid such as arginine, histidine or lysine.
  MISC_FEATURE (17)..(20): Xaa is a positively charged amino acid such as arginine, histidine or lysine , and any of them can be absent.
  MISC_FEATURE (21)..(21): Xaa is a spacer amino acid such as serine or glycine.
  MISC_FEATURE (22)..(25): Xaa is a spacer amino acid such as serine or glycine, and any of them can be absent.
XXXXXXXXXXXXXXXXXXXXXXXXX

SEQ ID NO: 4 - protein, 25 aa, Artificial Sequence
  Synthetic Sequence
  MISC_FEATURE (1)..(1): Xaa is a spacer amino acid such as serine or glycine.
  MISC_FEATURE (2)..(5): Xaa is a spacer amino acid such as serine or glycine, and any of them can be absent.
  MISC_FEATURE (6)..(6): Xaa is a negatively charged amino acid such as aspartic acid or glutamic acid.
  MISC_FEATURE (7)..(10): Xaa is a negatively charged amino acid such as aspartic acid or glutamic acid, and any of them can be absent.
  MISC_FEATURE (11)..(11): Xaa is a spacer amino acid such as serine or glycine.
  MISC_FEATURE (12)..(15): Xaa is a spacer amino acid such as serine or glycine, and any of them can be absent.
  MISC_FEATURE (16)..(16): Xaa is a negatively charged amino acid such as aspartic acid or glutamic acid.
  MISC_FEATURE (17)..(20): Xaa is a negatively charged amino acid such as aspartic acid or glutamic acid, and any of them can be absent.
  MISC_FEATURE (21)..(21): Xaa is a spacer amino acid such as serine or glycine.
  MISC_FEATURE (22)..(25): Xaa is a spacer amino acid such as serine or glycine, and any of them can be absent.
XXXXXXXXXXXXXXXXXXXXXXXXX

SEQ ID NO: 5 - protein, 21 aa, Artificial Sequence
  Synthetic Sequence
  MISC_FEATURE (1)..(4): Xaa is a spacer amino acid such as serine or glycine, and any of them can be absent.
  MISC_FEATURE (5)..(8): Xaa is a positively charged amino acid such as arginine, histidine or lysine, and any of them can be absent.
  MISC_FEATURE (10)..(13): Xaa is a spacer amino acid such as serine or glycine, and any of them can be absent.
  MISC_FEATURE (14)..(17): Xaa is a positively charged amino acid such as arginine, histidine or lysine, and any of them can be absent.
  MISC_FEATURE (18)..(21): Xaa is a spacer amino acid such as serine or glycine, and any of them can be absent.
XXXXXXXXCXXXXXXXXXXXX

SEQ ID NO: 6 - protein, 21 aa, Artificial Sequence
  Synthetic Sequence
  MISC_FEATURE (1)..(4): Xaa is a spacer amino acid such as serine or glycine, and any of them can be absent.
  MISC_FEATURE (5)..(8): Xaa is a negatively charged amino acid such as aspartic acid or glutamic acid, and any of them can be absent.
  MISC_FEATURE (10)..(13): Xaa is a spacer amino acid such as serine or glycine, and any of them can be absent.
  MISC_FEATURE (14)..(17): Xaa is a negatively charged amino acid such as aspartic acid or glutamic acid, and any of them can be absent.
  MISC_FEATURE (18)..(21): Xaa is a spacer amino acid such as serine or glycine, and any of them can be absent.
XXXXXXXXCXXXXXXXXXXXX

SEQ ID NO: 7 - protein, 12 aa, Artificial Sequence
  Synthetic Sequence
GSGSRKGGKRGS

SEQ ID NO: 8 - protein, 12 aa, Artificial Sequence
  Synthetic Sequence
GSGSRKCGKRGS

SEQ ID NO: 9 - protein, 12 aa, Artificial Sequence
  Synthetic Sequence
GSGSDEGGEDGS

SEQ ID NO: 10 - protein, 12 aa, Artificial Sequence
  Synthetic Sequence
GSGSDECGEDGS

SEQ ID NO: 11 - protein, 6 aa, Artificial Sequence
  Synthetic Sequence
RKGGKR

SEQ ID NO: 12 - protein, 12 aa, Artificial Sequence
  Synthetic Sequence
GSGSRKGGKRGS

SEQ ID NO: 13 - protein, 6 aa, Artificial Sequence
  Synthetic Sequence
DEGGED

SEQ ID NO: 14 - protein, 12 aa, Artificial Sequence
  Synthetic Sequence
GSGSDEGGEDGS

SEQ ID NO: 15 - protein, 234 aa, Artificial Sequence
  Synthetic Sequence_core domain
SKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMD

SEQ ID NO: 16 - protein, 249 aa, Artificial Sequence
  Synthetic Sequence_charge polarized core domain
GSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSGS

SEQ ID NO: 17 - protein, 249 aa, Artificial Sequence
  Synthetic Sequence_charge polarized core domain
GSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGS

SEQ ID NO: 18 - protein, 222 aa, Artificial Sequence
  Synthetic Sequence_core domain
CPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 19 - protein, 232 aa, Artificial Sequence
  Synthetic Sequence_core domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 232 aa, Artificial Sequence
  Synthetic Sequence_core domain
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 21 - protein, 227 aa, Artificial Sequence
  Synthetic Sequence_core domain
VPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGI

SEQ ID NO: 22 - protein, 222 aa, Artificial Sequence; identical to SEQ ID NO: 18
  Synthetic Sequence_core domain

SEQ ID NO: 23 - protein, 238 aa, Artificial Sequence
  Synthetic Sequence_Fc domain
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKIEGRMD

SEQ ID NO: 24 - protein, 238 aa, Artificial Sequence
  Synthetic Sequence_Fc domain
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKIEGRMD

SEQ ID NO: 25 - protein, 238 aa, Artificial Sequence
  Synthetic Sequence_Fc domain
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGKIEGRMD

SEQ ID NO: 26 - protein, 238 aa, Artificial Sequence
  Synthetic Sequence_Fc domain
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGKIEGRMD

SEQ ID NO: 27 - protein, 220 aa, Homo sapiens
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCA

SEQ ID NO: 28 - protein, 113 aa, Homo sapiens
QFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLV

SEQ ID NO: 29 - protein, 225 aa, Homo sapiens
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAG

SEQ ID NO: 30 - protein, 116 aa, Homo sapiens
AQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVA

SEQ ID NO: 31 - protein, 111 aa, Artificial Sequence
  Synthetic Sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 32 - protein, 109 aa, Artificial Sequence
  Synthetic Sequence
EVQLVESGGGLVQPGGSLTLSCAASRFMISEYHMHWVRQAPGKGLEWVSTINPAGTTDYAESVKGRFTISRDNAKNTLYLQMNSLKPEDTAVYYCDSYGYRGQGTQVTV

SEQ ID NO: 33 - protein, 250 aa, Artificial Sequence
  Synthetic Sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 34 - protein, 238 aa, Artificial Sequence
  Synthetic Sequence
EVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSSGGGGSGGGGSGGGGSDIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELK

SEQ ID NO: 35 - protein, 239 aa, Artificial Sequence
  Synthetic Sequence
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 36 - protein, 238 aa, Artificial Sequence
  Synthetic Sequence
DIQMTQSPASLSTSLGETVTIQCQASEDIYSGLAWYQQKPGKSPQLLIYGASDLQDGVPSRFSGSGSGTQYSLKITSMQTEDEGVYFCQQGLTYPRTFGGGTKLELKGGGGSGGGGSGGGGSEVQLQQSGAELVRPGTSVKLSCKVSGDTITFYYMHFVKQRPGQGLEWIGRIDPEDESTKYSEKFKNKATLTADTSSNTAYLKLSSLTSEDTATYFCIYGGYYFDYWGQGVMVTVSS

SEQ ID NO: 37 - protein, 120 aa, Artificial Sequence
  Synthetic Sequence
RKGGKRGSGSGQTVVTQEPSLTVSPGGTVTLTCASSTGAVTSGNYPNWVQQKPGQAPRGLIGGTKFLVPGTPARFSGSLLGGKAALTLSGVQPEDEAEYYCTLWYSNRWVFGGGTKLTVL

SEQ ID NO: 38 - protein, 166 aa, Artificial Sequence
  Synthetic Sequence
GTDFTLKISRVEAEDLGVYFCSQSTHVPPLTFGAGTKLELKGGGSGGGSGGGSEVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMKYWGQGTSVTVSS

SEQ ID NO: 39 - protein, 249 aa, Artificial Sequence
  Synthetic Sequence
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKEDTIRGPNYYYYGMDVWGQGTTVTVSSASGGGGSGGGGSGGGGSETTLTQSPSSVSASVGDRVSITCRASQDIDTWLAWYQLKPGKAPKLLMYAASNLQGGVPSRFSGSGSGTDFILTISSLQPEDFATYYCQQASIFPPTFGGGTKVDIK

SEQ ID NO: 40 - protein, 250 aa, Artificial Sequence
  Synthetic Sequence
QVQLVQSGAEVKKPGESLKISCKGSGYSFTSYWIGWVRQMPGKGLEWMGIIYPGDSDTRYSPSFQGQVTISADKSITTAYLQWSSLRASDSAMYYCARGGYSDYDYYFDFWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGGGTKVEIK

SEQ ID NO: 41 - protein, 240 aa, Artificial Sequence
  Synthetic Sequence
QVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 42 - protein, 248 aa, Artificial Sequence
  Synthetic Sequence
QVQLVQSGGGVVQPGKSLRLSCAASGFTFSIFAMHWVRQAPGKGLEWVATISYDGSNAFYADSVEGRFTISRDNSKDSLYLQMDSLRPEDTAVYYCVKAGDGGYDVFDSWGQGTLVTVSSASGGGGSGGGGSGGGGSEIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPTFGPGTKVDIK

SEQ ID NO: 43 - protein, 245 aa, Artificial Sequence
  Synthetic Sequence
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKETDYYGSGTFDYWGQGTLVTVSSASGGGGSGGGGSGGGGSDIQMTQSPSSLSASVGDRVTISCRASQGIGIYLAWYQQRSGKPPQLLIHGASTLQSGVPSRFSGSGSGTDFTLTISSLQPEDFASYWCQQSNNFPPTFGQGTKVEIK

SEQ ID NO: 44 - protein, 244 aa, Artificial Sequence
  Synthetic Sequence
QVQLVQSGAEVKKPGESLKISCKGSGYSFTNYWIGWVRQMPGKGLEWMGIIYPGDSDTRYSPSFQGQVTISADKSISTAYLQWSSLKASDTAMYYCARHGPSSWGEFDYWGQGTLVTVSSASGGGGSGGGGSGGGGSDIRLTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVDIK

SEQ ID NO: 45 - protein, 244 aa, Artificial Sequence
  Synthetic Sequence
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSASGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVK

SEQ ID NO: 46 - protein, 255 aa, Artificial Sequence
  Synthetic Sequence
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSGGGGSGGGGSGGGGSGGGGSAEIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIK

SEQ ID NO: 47 - protein, 247 aa, Artificial Sequence
  Synthetic Sequence
EVQLVQSGAEVKKPGESLKISCKGSGRTFTSYNMHWVRQMPGKGLEWMGAIYPLTGDTSYNQKSKLQVTISADKSISTAYLQWSSLKASDTAMYYCARSTYVGGDWQFDVWGKGTTVTVSSGGGGSGGGGSGGGGSGGGGSEIVLTQSPGTLSLSPGERATLSCRASSSVPYIHWYQQKPGQAPRLLIYATSALASGIPDRFSGSGSGTDFTLTISRLEPEDFAVYYCQQWLSHPPTFGQGTKLEIK

SEQ ID NO: 48 - protein, 247 aa, Artificial Sequence
  Synthetic Sequence
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSGGGGSGGGGSGGGGSGGGGSQIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 49 - protein, 249 aa, Artificial Sequence
  Synthetic Sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSGGGGSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 50 - protein, 250 aa, Artificial Sequence
  Synthetic Sequence
SSELTQDPAVSVALGQTVRITCQGDSLRSYYASWYQQKPGQAPVLVIYGKNNRPSGIPDRFSGSSSGNTASLTITGAQAEDEADYYCNSRDSSGNPPVVFGGGTKLTVLGSRGGGGSGGGGSGGGGSLEMAQVQLVESGGGLVHPGGSLRLSCAASGFTFRSHSMNWVRQAPGKGLEWVSSISSDSTYTYYADSVKGRFTISRDNAKNSLYLQMNSLRAEDTAVYYCARSGGQWKYYDYWGQGTLVTVSS

SEQ ID NO: 51 - protein, 248 aa, Artificial Sequence
  Synthetic Sequence
QSVVTQPPSMSAAPGQQVTISCSGGNSNIERNYVSWYLQLPGTAPKLVIFDNDRRPSGIPDRFSGSKSGTSATLGITGLQTGDEADYYCGTWDSSLRGWVFGGGTKLTVLGSRGGGGSGGGGSGGGGSLEMAEVQLVESGGGLIQPGGSLRLSCAASGFTFSNYAMNWVRQAPGKGLEWVSTINGRGSSTIYADSVKGRFTISRDNSKNTLYLQMNSLRAEDTATYYCARYISRGLGDSWGQGTLVTV

SEQ ID NO: 52 - protein, 244 aa, Artificial Sequence
  Synthetic Sequence
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSGGGGSGGGGSGGGGSDIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 53 - protein, 244 aa, Artificial Sequence
  Synthetic Sequence
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRGGGGSGGGGSGGGGSQVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 54 - protein, 243 aa, Artificial Sequence
  Synthetic Sequence
QIQLVQSGPELKKPGETVKISCKASGYTFTNYGMNWVKQAPGKGLKWMGWINTKTGEPTYAEEFKGRFAFSLETSASTAYLQINNLKKEDTATYFCGRGGYGSSYWYFDVWGAGTTVTVSSGGGGSGGGGSGGGGSDIVMTQSHKFMSTSVGDRVSITCKASQDVSIAVAWYQQKPGQSPKVLIYSASYRYTGVPDRFTGSGSGTDFTFTISRVQAEDLAVYYCQQHYITPLTFGAGTKLELK

SEQ ID NO: 55 - protein, 243 aa, Artificial Sequence
  Synthetic Sequence
DIVMTQSHKFMSTSVGDRVSITCKASQDVSIAVAWYQQKPGQSPKVLIYSASYRYTGVPDRFTGSGSGTDFTFTISRVQAEDLAVYYCQQHYITPLTFGAGTKLELKGGGGSGGGGSGGGGSQIQLVQSGPELKKPGETVKISCKASGYTFTNYGMNWVKQAPGKGLKWMGWINTKTGEPTYAEEFKGRFAFSLETSASTAYLQINNLKKEDTATYFCGRGGYGSSYWYFDVWGAGTTVTVSS

SEQ ID NO: 56 - protein, 241 aa, Artificial Sequence
  Synthetic Sequence
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSGGGGSGGGGSGGGGSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKR

SEQ ID NO: 57 - protein, 241 aa, Artificial Sequence
  Synthetic Sequence
DIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGGGSGGGGSGGGGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSS

SEQ ID NO: 58 - protein, 242 aa, Artificial Sequence
  Synthetic Sequence
EVQLQESGPGLVKPGGSLSLSCAASGFVFSSYDMSWVRQTPERRLEWVAYISSGGGITYFPSTVKGRFTVSRDNAKNTLYLQMNSLTSEDTAIYYCAAHYFGSSGPFAYWGQGTLVTVSAGGGGSGGGGSGGGGSDIQMTQSPASLSASVGDTVTITCRASENIFSYLAWYQQKPGKSPKLLVYNTKTLAEGVPSRFSGSGSGTQFSLTISSLQPEDFGSYYCQHHYGTPFTFGSGTKLEIK

SEQ ID NO: 59 - protein, 241 aa, Artificial Sequence
  Synthetic Sequence
DIQMTQSPASLSASVGDTVTITCRASENIFSYLAWYQQKPGKSPKLLVYNTKTLAEGVPSRFSGSGSGTQFSLTISSLQPEDFGSYYCQHHYGTPFTFGSGTKLEIKGGGGSGGGGSGGGGSVQLQESGPGLVKPGGSLSLSCAASGFVFSSYDMSWVRQTPERRLEWVAYISSGGGITYFPSTVKGRFTVSRDNAKNTLYLQMNSLTSEDTAIYYCAAHYFGSSGPFAYWGQGTLVTVSA

SEQ ID NO: 60 - protein, 252 aa, Artificial Sequence
  Synthetic Sequence
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 61 - protein, 252 aa, Artificial Sequence
  Synthetic Sequence
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 62 - protein, 240 aa, Artificial Sequence
  Synthetic Sequence
QVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 63 - protein, 240 aa, Artificial Sequence
  Synthetic Sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIKGGGGSGGGGSGGGGSQVQLVQSGGGVVQPGRSLRLSCVASGFTFSSYGMHWVRQAPGKGLEWVAAIWYNGRKQDYADSVKGRFTISRDNSKNTLYLQMNSLRAEDTAVYYCTRGTGYNWFDPWGQGTLVTVSS

SEQ ID NO: 64 - protein, 245 aa, Artificial Sequence
  Synthetic Sequence
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKR

SEQ ID NO: 65 - protein, 245 aa, Artificial Sequence
  Synthetic Sequence
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKRGGGGSGGGGSGGGGSQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 66 - protein, 240 aa, Artificial Sequence
  Synthetic Sequence
QVQLVQSGAEVKKPGASVKVSCKASGYTFTSYYMHWVRQAPGQGLEWMGIINPNGGSTSYAQKFQGRVTMTRDTSTSTVYMELSSLRSEDTAVYYCARDSSYDAFDIWGQGTMVTVSSGGGGSGGGGSGGGGSAIQLTQSPSTLSASVGDRVTITCQASQDISNYLNWYQQKPGKAPKLLINDASYLETGVPSRFSGSGSGTDFTLTISSLQPEDIATYYCQQYESLPYTFGQGTKLEIK

SEQ ID NO: 67 - protein, 240 aa, Artificial Sequence
  Synthetic Sequence
AIQLTQSPSTLSASVGDRVTITCQASQDISNYLNWYQQKPGKAPKLLINDASYLETGVPSRFSGSGSGTDFTLTISSLQPEDIATYYCQQYESLPYTFGQGTKLEIKGGGGSGGGGSGGGGSQVQLVQSGAEVKKPGASVKVSCKASGYTFTSYYMHWVRQAPGQGLEWMGIINPNGGSTSYAQKFQGRVTMTRDTSTSTVYMELSSLRSEDTAVYYCARDSSYDAFDIWGQGTMVTVSS

SEQ ID NO: 68 - protein, 245 aa, Artificial Sequence
  Synthetic Sequence
QVTLKESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARDFGRWSYYFDYWSQGTLVTVSSGGGGSGGGGSGGGGSQSVLTQPSSVSGTPGQRVTISCSGSSSNIGSNYVYWYQQLPGTAPKWYRNNQRPSGVPDRFSGSKSGTSASLAISGLRSEDEADYYCAAWDDSLSGVVFGGGTKLTVL

SEQ ID NO: 69 - protein, 245 aa, Artificial Sequence
  Synthetic Sequence
QSVLTQPSSVSGTPGQRVTISCSGSSSNIGSNYVYWYQQLPGTAPKWYRNNQRPSGVPDRFSGSKSGTSASLAISGLRSEDEADYYCAAWDDSLSGVVFGGGTKLTVLGGGGSGGGGSGGGGSQVTLKESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCARDFGRWSYYFDYWSQGTLVTVSS

SEQ ID NO: 70 - protein, 241 aa, Artificial Sequence
  Synthetic Sequence
EVQLVESGGGLVQPGRSLRLSCTASGFTFGDYAMSWVRQAPGKGLEWVSSISGSGRSTDHADYVKGRFTISRDNSKNTVYLQMNRLRAEDTAVYYCAKVSNYEYYFDYWAQGTLTVSSGGGGSGGGGSGGGGSEIVLTQSPSVSVAPGQTARITCGGSNIGSESVNWYQWKSGQVPVLVVSDTTDPRSGIPGRFTGTRSGTTATLTISGVEAGDEADYHCQVWDDTGDHPVFGGGTKLTVL

SEQ ID NO: 71 - protein, 241 aa, Artificial Sequence
  Synthetic Sequence
EIVLTQSPSVSVAPGQTARITCGGSNIGSESVNWYQWKSGQVPVLVVSDTTDPRSGIPGRFTGTRSGTTATLTISGVEAGDEADYHCQVWDDTGDHPVFGGGTKLTVLGGGGSGGGGSGGGGSEVQLVESGGGLVQPGRSLRLSCTASGFTFGDYAMSWVRQAPGKGLEWVSSISGSGRSTDHADYVKGRFTISRDNSKNTVYLQMNRLRAEDTAVYYCAKVSNYEYYFDYWAQGTLTVSS

SEQ ID NO: 72 - protein, 19 aa, Artificial Sequence
  Synthetic Sequence
MEFGLSWVFLVAIIKGVQC

SEQ ID NO: 73 - protein, 741 aa, Homo sapiens
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSGGGGSGGGGSGGGGSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 74 - protein, 730 aa, Homo sapiens
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 75 - protein, 725 aa, Homo sapiens
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 76 - protein, 2178 aa, Homo sapiens
ATGGAGTTCGGCCTGAGCTGGGTGTTTCTGGTGGCCATCATCAAGGGCGTGCAGTGCCAGTTCATCGTGGTGGGCCCTACCGACCCAATCCTGGCCACAGTGGGCGAGAACACCACACTGAGGTGTCACCTGTCCCCAGAGAAGAATGCCGAGGATATGGAGGTGCGGTGGTTCAGATCTCAGTTTAGCCCCGCCGTGTTCGTGTATAAGGGCGGCCGGGAGAGAACCGAGGAGCAGATGGAGGAGTACAGGGGCCGCACCACATTTGTGAGCAAGGACATCTCCCGCGGCTCTGTGGCCCTGGTCATCCACAACATCACCGCCCAGGAGAATGGCACATATCGGTGCTACTTTCAGGAGGGCAGATCCTACGATGAGGCCATCCTGCACCTGGTGGTGGCAGGCCTGGGATCTAAGCCCCTGATCAGCATGAGGGGACACGAGGACGGAGGAATCAGGCTGGAGTGTATCAGCAGAGGCTGGTATCCCAAGCCTCTGACCGTGTGGAGAGATCCCTACGGAGGAGTGGCACCTGCCCTGAAGGAGGTGTCCATGCCAGACGCCGATGGCCTGTTCATGGTGACCACAGCCGTGATCATCCGGGACAAGTCTGTGAGAAATATGTCTTGCAGCATCAACAATACACTGCTGGGCCAGAAGAAGGAGAGCGTGATCTTCATCCCCGAGTCCTTTATGCCATCCGTGTCTCCATGTGCAGGAAGCGGCTCCAGGAAGGGAGGCAAGAGGGGAAGCAAGTATGGACCACCTTGCCCACCATGTCCAGCACCAGAGTTTCTGGGAGGACCATCCGTGTTCCTGTTTCCTCCAAAGCCCAAGGACCAGCTGATGATCTCCAGGACCCCAGAGGTGACATGCGTGGTGGTGGACGTGTCTCAGGAGGATCCTGAGGTGCAGTTCAACTGGTACGTGGACGGCGTGGAGGTGCACAATGCCAAGACCAAGCCCAGGGAGGAGCAGTTTAACTCCACCTATCGCGTGGTGTCTGTGCTGACAGTGCTGCACCAGGACTGGCTGAGCGGCAAGGAGTACAAGTGCAAGGTGAGCTCCAAGGGCCTGCCTTCTAGCATCGAGAAGACCATCTCCAACGCCACAGGCCAGCCCAGAGAGCCTCAGGTGTATACCCTGCCCCCTAGCCAGGAGGAGATGACCAAGAATCAGGTGTCCCTGACATGTCTGGTGAAGGGCTTCTACCCTTCCGATATCGCCGTGGAGTGGGAGTCTAACGGCCAGCCAGAGAACAATTATAAGACCACACCACCCGTGCTGGACAGCGATGGCTCCTTCTTTCTGTACTCTAGGCTGACCGTGGACAAGAGCCGCTGGCAGGAGGGCAACGTGTTTTCTTGCAGCGTGCTGCACGAGGCCCTGCACAATCACTACACACAGAAGTCCCTGTCTCTGAGCCTGGGCAAGGATGAGGGAGGAGAGGACGGAAGCGATATCCAGATGACCCAGACCACATCCTCTCTGTCCGCCTCTCTGGGCGACAGGGTGACAATCTCCTGTCGCGCCTCTCAGGATATCAGCAAGTATCTGAATTGGTATCAGCAGAAGCCTGACGGCACCGTGAAGCTGCTGATCTATCACACATCCCGGCTGCACTCTGGCGTGCCAAGCAGATTCAGCGGATCCGGATCTGGCACCGACTACTCCCTGACAATCTCTAACCTGGAGCAGGAGGATATCGCCACCTATTTCTGCCAGCAGGGCAATACCCTGCCTTACACATTTGGCGGCGGCACCAAGCTGGAGATCACAGGCGGAGGAAGCGGAGGAGGATCCGGAGGAGGATCTGAGGTGAAGCTGCAGGAGAGCGGACCTGGCCTGGTGGCACCAAGCCAGTCCCTGTCTGTGACCTGTACAGTGTCTGGCGTGAGCCTGCCCGATTACGGCGTGTCTTGGATCAGGCAGCCTCCAAGGAAGGGCCTGGAGTGGCTGGGCGTGATCTGGGGCAGCGAGACAACATACTATAACAGCGCCCTGAAGTCCCGGCTGACCATCATCAAGGACAACAGCAAGTCCCAGGTGTTCCTGAAGATGAATTCCCTGCAGACCGACGATACAGCCATCTACTATTGTGCCAAGCACTACTATTACGGCGGCTCTTATGCCATGGATTACTGGGGCCAGGGCACCAGCGTGACAGTGAGCTCCTGA

SEQ ID NO: 77 - protein, 716 aa, Homo sapiens
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 78 - protein, 2151 aa, Homo sapiens
ATGGAGTTTGGCCTGTCTTGGGTGTTTCTGGTGGCTATCATCAAGGGAGTGCAGTGTCAGTTCATCGTTGTGGGACCTACCGATCCTATCTTGGCTACAGTGGGCGAGAATACAACCCTGAGATGTCACTTGTCTCCTGAGAAGAACGCCGAGGATATGGAGGTTAGGTGGTTCAGATCCCAGTTCTCTCCTGCCGTGTTTGTGTATAAGGGAGGCAGAGAGAGAACAGAAGAGCAGATGGAGGAGTACAGAGGAAGAACCACCTTCGTGTCTAAGGACATCAGCAGAGGCTCTGTGGCTCTGGTGATCCACAATATCACAGCTCAGGAGAATGGCACCTACAGATGCTACTTTCAGGAGGGCAGGTCCTACGATGAGGCTATTTTGCATCTGGTGGTTGCTGGACTGGGATCTAAACCTCTGATCAGCATGAGGGGACACGAGGATGGAGGAATTAGACTGGAGTGCATCTCTAGAGGCTGGTATCCTAAACCACTGACAGTGTGGAGAGACCCTTATGGAGGAGTTGCTCCTGCTCTGAAAGAGGTGTCTATGCCTGATGCTGATGGCCTGTTTATGGTGACAACAGCCGTGATCATCCGGGACAAATCCGTGAGGAACATGTCTTGCTCCATCAACAACACACTGTTGGGACAGAAGAAGGAGAGCGTGATCTTCATCCCCGAGAGCTTCATGCCTAGCGTTTCTCCTTGTGCTGAACCTAAGTCTTGCGACAAGACCCATACATGCCCTCCTTGTCCTGCTCCTGAAGCTGCTGGAGGACCTTCTGTGTTTTTGTTTCCTCCTAAGCCTAAGGATACCCTGATGATCTCCAGAACCCCCGAGGTGACCTGTGTGGTGGTTGATGTTTCTCATGAGGATCCTGAAGTGAAGTTCAACTGGTACGTGGACGGCGTGGAGGTGCACAACGCTAAGACAAAACCTAGAGAAGAGCAGTACAACTCTACCTACAGAGTGGTGTCCGTGCTGACCGTGCTGCACCAAGATTGGTTGAATGGAAAGGAGTACAAGTGCAAGGTGTCCAACAAGGCTCTGCCTGCCCCTATCGAGAAGACCATCTCTAAGGCTAAAGGACAACCTAGAGAACCTCAGGTGTATACCCTGCCTCCCTGCAGAGATGAGCTGACCAAGAATCAGGTTTCTCTGTGGTGTCTGGTGAAGGGCTTTTACCCTAGCGACATCGCTGTGGAGTGGGAGTCTAATGGACAACCTGAGAACAACTACAAGACCACACCTCCTGTGCTGGACTCTGATGGCTCCTTCTTCCTGTACTCTAAGCTGACCGTGGATAAGTCTAGATGGCAACAGGGCAACGTGTTCTCCTGCTCCGTGATGCATGAAGCTCTGCACAACCACTATACACAGAAGTCTCTGAGCCTGTCTCCTGGCAAGATCGAGGGCAGAATGGACGATATCCAGATGACACAGACAACCTCTTCTCTGTCTGCTTCTCTGGGCGATAGAGTGACCATCAGCTGCAGAGCTTCTCAGGACATCAGCAAGTATCTGAACTGGTATCAGCAGAAGCCTGATGGCACCGTGAAGCTGCTGATCTACCACACCTCCAGATTGCATTCTGGAGTTCCTTCCAGATTTTCTGGCTCTGGCTCTGGCACCGACTATTCTCTGACCATCAGCAATCTGGAACAGGAGGACATCGCTACCTACTTTTGCCAGCAGGGCAACACACTGCCTTACACATTTGGAGGAGGAACAAAGCTGGAGATCACAGGAGGAGGATCTGGAGGAGGATCTGGAGGAGGATCTGAAGTTAAACTGCAGGAATCTGGACCAGGATTAGTGGCCCCATCTCAGTCTCTGTCTGTGACCTGTACCGTTTCTGGAGTTTCTTTGCCTGATTACGGAGTGTCCTGGATCAGACAGCCCCCTAGAAAGGGACTGGAATGGTTAGGAGTGATTTGGGGATCTGAGACCACCTACTACAACTCTGCCCTGAAGAGCAGACTGACCATCATCAAGGACAACAGCAAGTCTCAGGTGTTCCTGAAGATGAACTCCCTGCAGACCGACGATACCGCCATCTACTACTGTGCTAAGCACTACTACTATGGCGGCTCTTATGCCATGGACTATTGGGGACAGGGCACCTCTGTGACAGTGTCTTCTTAA

SEQ ID NO: 79 - protein, 716 aa, Homo sapiens
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 80 - protein, 2151 aa, Homo sapiens
ATGGAGTTTGGCCTGTCTTGGGTGTTTCTGGTGGCTATCATCAAGGGAGTGCAGTGTCAGTTCATCGTTGTGGGACCTACCGATCCTATCTTGGCTACAGTGGGCGAGAATACAACCCTGAGATGTCACTTGTCTCCTGAGAAGAACGCCGAGGATATGGAGGTTAGGTGGTTCAGATCCCAGTTCTCTCCTGCCGTGTTTGTGTATAAGGGAGGCAGAGAGAGAACAGAAGAGCAGATGGAGGAGTACAGAGGAAGAACCACCTTCGTGTCTAAGGACATCAGCAGAGGCTCTGTGGCTCTGGTGATCCACAATATCACAGCTCAGGAGAATGGCACCTACAGATGCTACTTTCAGGAGGGCAGGTCCTACGATGAGGCTATTTTGCATCTGGTGGTTGCTGGACTGGGATCTAAACCTCTGATCAGCATGAGGGGACACGAGGATGGAGGAATTAGACTGGAGTGCATCTCTAGAGGCTGGTATCCTAAACCACTGACAGTGTGGAGAGACCCTTATGGAGGAGTTGCTCCTGCTCTGAAAGAGGTGTCTATGCCTGATGCTGATGGCCTGTTTATGGTGACAACAGCCGTGATCATCCGGGACAAATCCGTGAGGAACATGTCTTGCTCCATCAACAACACACTGTTGGGACAGAAGAAGGAGAGCGTGATCTTCATCCCCGAGAGCTTCATGCCTAGCGTTTCTCCTTGTGCTGAACCTAAGTCTTGCGACAAGACCCATACATGCCCTCCTTGTCCTGCTCCTGAAGCTGCTGGAGGACCTTCTGTGTTTTTGTTTCCTCCTAAGCCTAAGGATACCCTGATGATCTCCAGAACCCCCGAGGTGACCTGTGTGGTGGTTGATGTTTCTCATGAGGATCCTGAAGTGAAGTTCAACTGGTACGTGGACGGCGTGGAGGTGCACAACGCTAAGACAAAACCTAGAGAAGAGCAGTACAACTCTACCTACAGAGTGGTGTCCGTGCTGACCGTGCTGCACCAAGATTGGTTGAATGGAAAGGAGTACAAGTGCAAGGTGTCCAACAAGGCTCTGCCTGCCCCTATCGAGAAGACCATCTCTAAGGCTAAAGGACAACCTAGAGAACCTCAGGTGTATACCCTGCCTCCCTGCAGAGATGAGCTGACCAAGAATCAGGTTTCTCTGTGGTGTCTGGTGAAGGGCTTTTACCCTAGCGACATCGCTGTGGAGTGGGAGTCTAATGGACAACCTGAGAACAACTACAAGACCACACCTCCTGTGCTGGACTCTGATGGCTCCTTCTTCCTGTACTCTAAGCTGACCGTGGATAAGTCTAGATGGCAACAGGGCAACGTGTTCTCCTGCTCTGTGCTGCATGAAGCTCTGCACTCTCACTATACACAGAAGTCTCTGTCCCTGTCTCCTGGCAAGATCGAGGGCAGAATGGACGATATCCAGATGACACAGACAACCTCTTCTCTGTCTGCTTCTCTGGGCGATAGAGTGACCATCAGCTGCAGAGCTTCTCAGGACATCAGCAAGTATCTGAACTGGTATCAGCAGAAGCCTGATGGCACCGTGAAGCTGCTGATCTACCACACCTCCAGATTGCATTCTGGAGTTCCTTCCAGATTTTCTGGCTCTGGCTCTGGCACCGACTATTCTCTGACCATCAGCAATCTGGAACAGGAGGACATCGCTACCTACTTTTGCCAGCAGGGCAACACACTGCCTTACACATTTGGAGGAGGAACAAAGCTGGAGATCACAGGAGGAGGATCTGGAGGAGGATCTGGAGGAGGATCTGAAGTTAAACTGCAGGAATCTGGACCAGGATTAGTGGCCCCATCTCAGTCTCTGTCTGTGACCTGTACCGTTTCTGGAGTTTCTTTGCCTGATTACGGAGTGTCCTGGATCAGACAGCCCCCTAGAAAGGGACTGGAATGGTTAGGAGTGATTTGGGGATCTGAGACCACCTACTACAACTCTGCCCTGAAGAGCAGACTGACCATCATCAAGGACAACAGCAAGTCTCAGGTGTTCCTGAAGATGAACTCCCTGCAGACCGACGATACCGCCATCTACTACTGTGCTAAGCACTACTACTATGGCGGCTCTTATGCCATGGACTATTGGGGACAGGGCACCTCTGTGACAGTGTCTTCTTAA

SEQ ID NO: 81 - protein, 732 aa, Homo sapiens
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 82 - protein, 2199 aa, Homo sapiens
ATGGAGTTCGGCCTGTCCTGGGTGTTTCTGGTGGCCATCATCAAGGGCGTGCAGTGCCAGTTCTCCGTGCTGGGCCCTTCTGGCCCAATCCTGGCAATGGTGGGAGAGGACGCAGATCTGCCATGTCACCTGTTTCCCACCATGAGCGCCGAGACAATGGAGCTGAAGTGGGTGAGCTCCTCTCTGCGGCAGGTGGTGAACGTGTACGCCGACGGCAAGGAGGTGGAGGATAGACAGTCCGCCCCCTACCGGGGCAGAACCTCTATCCTGAGGGACGGAATCACAGCAGGCAAGGCCGCCCTGAGAATCCACAACGTGACCGCCAGCGATTCCGGCAAGTATCTGTGCTACTTCCAGGACGGCGACTTCTACGAGAAGGCCCTGGTGGAGCTGAAGGTGGCCGCCCTGGGAAGCGACCTGCATGTGGATGTGAAGGGCTATAAGGACGGCGGCATCCACCTGGAGTGTCGGAGCACCGGCTGGTATCCCCAGCCTCAGATCCAGTGGTCCAACAATAAGGGCGAGAATATCCCTACAGTGGAGGCCCCAGTGGTGGCAGATGGAGTGGGCCTGTACGCAGTGGCCGCCTCTGTGATCATGAGGGGAAGCTCCGGAGAGGGCGTGAGCTGCACCATCCGCTCTAGCCTGCTGGGCCTGGAGAAGACAGCCTCTATCAGCATCGCCGACCCCTTCTTTAGGAGCGCCCAGCGGTGGATCGCCGCCCTGGCAGGCGGCTCCGGCTCTGACGAGGGCGGCGAGGATGGCTCCAAGTATGGACCACCTTGCCCACCATGTCCAGCACCAGAGTTCCTGGGAGGACCAAGCGTGTTCCTGTTTCCTCCAAAGCCCAAGGACCAGCTGATGATCTCCAGGACCCCAGAGGTGACCTGCGTGGTGGTGGACGTGTCTCAGGAGGATCCTGAGGTGCAGTTCAACTGGTACGTGGATGGCGTGGAGGTGCACAATGCCAAGACCAAGCCTAGGGAGGAGCAGTTTAACAGCACCTATCGCGTGGTGTCCGTGCTGACAGTGCTGCACCAGGATTGGCTGTCCGGCAAGGAGTACAAGTGCAAGGTGTCCTCTAAGGGCCTGCCAAGCTCCATCGAGAAGACCATCAGCAACGCAACAGGCCAGCCCCGCGAGCCTCAGGTGTATACCCTGCCCCCTTCTCAGGAGGAGATGACCAAGAATCAGGTGAGCCTGACATGTCTGGTGAAGGGCTTCTACCCTAGCGACATCGCAGTGGAGTGGGAGTCCAACGGACAGCCAGAGAACAATTATAAGACCACACCACCCGTGCTGGACTCTGATGGCAGCTTCTTTCTGTACTCTAGGCTGACCGTGGATAAGAGCCGCTGGCAGGAGGGCAACGTGTTTAGCTGCTCCGTGCTGCACGAGGCCCTGCACAATCACTACACACAGAAGTCTCTGAGCCTGTCCCTGGGCAAGAGGAAGGGAGGCAAGAGGGGATCTGGAAGCGACATCCAGATGACCCAGACCACATCTAGCCTGTCCGCCTCTCTGGGCGACCGGGTGACAATCAGCTGTAGAGCCTCCCAGGATATCTCTAAGTATCTGAATTGGTATCAGCAGAAGCCAGATGGCACCGTGAAGCTGCTGATCTATCACACAAGCAGGCTGCACTCCGGCGTGCCCTCTAGATTCAGCGGATCCGGATCTGGCACCGACTACAGCCTGACAATCTCCAACCTGGAGCAGGAGGATATCGCCACCTATTTCTGCCAGCAGGGCAATACCCTGCCCTACACATTTGGCGGCGGCACCAAGCTGGAGATCACAGGCGGAGGATCTGGAGGAGGAAGCGGAGGAGGCTCCGAGGTGAAGCTGCAGGAGTCCGGACCAGGCCTGGTGGCACCTAGCCAGTCCCTGTCTGTGACCTGTACAGTGTCCGGCGTGTCTCTGCCTGACTACGGCGTGTCCTGGATCCGGCAGCCTCCAAGAAAGGGCCTGGAGTGGCTGGGCGTGATCTGGGGCAGCGAGACAACATACTATAACTCTGCCCTGAAGAGCAGACTGACCATCATCAAGGACAACAGCAAGTCCCAGGTGTTTCTGAAGATGAATAGCCTGCAGACCGACGATACAGCCATCTACTATTGCGCCAAGCACTACTATTACGGCGGCTCCTATGCCATGGATTACTGGGGCCAGGGCACCTCTGTGACAGTGTCCTCTTGA

SEQ ID NO: 83 - protein, 720 aa, Homo sapiens
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 84 - protein, 2163 aa, Homo sapiens
ATGGAGTTTGGCCTGTCTTGGGTGTTTCTGGTGGCTATCATCAAGGGAGTGCAGTGTCAGTTCTCTGTTCTGGGACCTTCTGGACCTATCTTGGCTATGGTGGGAGAAGATGCTGATCTGCCTTGTCACCTGTTTCCTACCATGTCTGCTGAGACCATGGAGCTGAAATGGGTGTCCTCTTCTCTGAGACAGGTGGTGAATGTGTACGCTGATGGAAAGGAGGTGGAGGACAGACAATCTGCCCCTTATAGAGGAAGAACCAGCATCCTGAGAGATGGCATCACAGCTGGAAAGGCTGCTCTGAGAATCCACAATGTGACCGCTTCTGATTCTGGCAAGTACCTGTGCTACTTCCAGGATGGCGACTTCTACGAGAAGGCTCTGGTTGAGCTGAAAGTTGCTGCTTTGGGCTCTGATCTGCATGTTGACGTGAAGGGCTACAAGGATGGAGGCATCCATCTGGAATGTAGATCTACCGGCTGGTATCCTCAACCTCAGATTCAGTGGAGCAACAACAAGGGCGAGAACATCCCTACAGTTGAAGCCCCTGTTGTGGCTGATGGAGTTGGACTGTATGCTGTTGCTGCTAGCGTGATCATGAGAGGATCTTCTGGAGAAGGCGTGTCTTGCACCATCAGATCTTCTCTGTTGGGACTGGAGAAGACCGCTAGCATCTCTATCGCTGACCCCTTCTTCAGATCTGCTCAAAGATGGATTGCTGCTCTGGCTGAGCCTAAGTCTTGCGATAAGACCCACACCTGTCCTCCTTGTCCTGCTCCTGAAGCTGCTGGAGGACCTTCTGTGTTTTTGTTTCCTCCTAAGCCTAAGGATACCCTGATGATCTCCAGAACCCCCGAGGTGACCTGTGTGGTGGTTGATGTTTCTCATGAGGATCCTGAAGTGAAGTTCAACTGGTACGTGGACGGCGTGGAGGTGCACAACGCTAAGACAAAACCTAGAGAAGAGCAGTACAACTCTACCTACAGAGTGGTGTCCGTGCTGACCGTGCTGCACCAAGATTGGTTGAATGGAAAGGAGTACAAGTGCAAGGTGTCCAACAAGGCTCTGCCTGCCCCTATCGAGAAGACCATCTCTAAGGCTAAAGGACAACCTAGAGAACCTCAGGTGTGTACACTGCCCCCCTCTAGAGATGAGCTGACCAAGAATCAGGTTTCTCTGTCTTGTGCTGTGAAGGGCTTTTACCCCTCCGACATCGCTGTGGAATGGGAGTCTAATGGACAACCTGAGAACAACTACAAGACCACACCTCCTGTGCTGGACTCTGACGGCTCCTTCTTTCTGGTGTCTAAGCTGACAGTGGATAAGTCTAGATGGCAACAGGGCAACGTGTTCAGCTGCTCCGTGATGCATGAAGCTCTGCACAACCACTATACACAGAAGTCTCTGAGCCTGTCTCCTGGCAAGATCGAGGGCAGAATGGACGATATCCAGATGACACAGACAACCTCTTCTCTGTCTGCTTCTCTGGGCGATAGAGTGACCATCAGCTGCAGAGCTTCTCAGGACATCAGCAAGTATCTGAACTGGTATCAGCAGAAGCCTGATGGCACCGTGAAGCTGCTGATCTACCACACCTCCAGATTGCATTCTGGAGTTCCTTCCAGATTTTCTGGCTCTGGCTCTGGCACCGACTATTCTCTGACCATCAGCAATCTGGAACAGGAGGACATCGCTACCTACTTTTGCCAGCAGGGCAACACACTGCCTTACACATTTGGAGGAGGAACAAAGCTGGAGATCACAGGAGGAGGATCTGGAGGAGGATCTGGAGGAGGATCTGAAGTTAAACTGCAGGAATCTGGACCAGGATTAGTGGCCCCATCTCAGTCTCTGTCTGTGACCTGTACCGTTTCTGGAGTTTCTTTGCCTGATTACGGAGTGTCCTGGATCAGACAGCCCCCTAGAAAGGGACTGGAATGGTTAGGAGTGATTTGGGGATCTGAGACCACCTACTACAACTCTGCCCTGAAGAGCAGACTGACCATCATCAAGGACAACAGCAAGTCTCAGGTGTTCCTGAAGATGAACTCCCTGCAGACCGACGATACCGCCATCTACTACTGTGCTAAGCACTACTACTATGGCGGCTCTTATGCCATGGACTATTGGGGACAGGGCACCTCTGTGACAGTGTCTTCTTAA

SEQ ID NO: 85 - protein, 720 aa, Homo sapiens
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 86 - protein, 2163 aa, Homo sapiens
ATGGAGTTTGGCCTGTCTTGGGTGTTTCTGGTGGCTATCATCAAGGGAGTGCAGTGTCAGTTCTCTGTTCTGGGACCTTCTGGACCTATCTTGGCTATGGTGGGAGAAGATGCTGATCTGCCTTGTCACCTGTTTCCTACCATGTCTGCTGAGACCATGGAGCTGAAATGGGTGTCCTCTTCTCTGAGACAGGTGGTGAATGTGTACGCTGATGGAAAGGAGGTGGAGGACAGACAATCTGCCCCTTATAGAGGAAGAACCAGCATCCTGAGAGATGGCATCACAGCTGGAAAGGCTGCTCTGAGAATCCACAATGTGACCGCTTCTGATTCTGGCAAGTACCTGTGCTACTTCCAGGATGGCGACTTCTACGAGAAGGCTCTGGTTGAGCTGAAAGTTGCTGCTTTGGGCTCTGATCTGCATGTTGACGTGAAGGGCTACAAGGATGGAGGCATCCATCTGGAATGTAGATCTACCGGCTGGTATCCTCAACCTCAGATTCAGTGGAGCAACAACAAGGGCGAGAACATCCCTACAGTTGAAGCCCCTGTTGTGGCTGATGGAGTTGGACTGTATGCTGTTGCTGCTAGCGTGATCATGAGAGGATCTTCTGGAGAAGGCGTGTCTTGCACCATCAGATCTTCTCTGTTGGGACTGGAGAAGACCGCTAGCATCTCTATCGCTGACCCCTTCTTCAGATCTGCTCAAAGATGGATTGCTGCTCTGGCTGAGCCTAAGTCTTGCGATAAGACCCACACCTGTCCTCCTTGTCCTGCTCCTGAAGCTGCTGGAGGACCTTCTGTGTTTTTGTTTCCTCCTAAGCCTAAGGATACCCTGATGATCTCCAGAACCCCCGAGGTGACCTGTGTGGTGGTTGATGTTTCTCATGAGGATCCTGAAGTGAAGTTCAACTGGTACGTGGACGGCGTGGAGGTGCACAACGCTAAGACAAAACCTAGAGAAGAGCAGTACAACTCTACCTACAGAGTGGTGTCCGTGCTGACCGTGCTGCACCAAGATTGGTTGAATGGAAAGGAGTACAAGTGCAAGGTGTCCAACAAGGCTCTGCCTGCCCCTATCGAGAAGACCATCTCTAAGGCTAAAGGACAACCTAGAGAACCTCAGGTGTGTACACTGCCCCCCTCTAGAGATGAGCTGACCAAGAATCAGGTTTCTCTGTCTTGTGCTGTGAAGGGCTTTTACCCCTCCGACATCGCTGTGGAATGGGAGTCTAATGGACAACCTGAGAACAACTACAAGACCACACCTCCTGTGCTGGACTCTGACGGCTCCTTCTTTCTGGTGTCTAAGCTGACAGTGGATAAGTCTAGATGGCAACAGGGCAACGTGTTCAGCTGCAGCGTTCTGCATGAAGCTCTGCATTCCCACTATACACAGAAGTCTCTGTCCCTGTCTCCTGGCAAGATCGAGGGCAGAATGGATGACATCCAGATGACACAGACAACCTCTTCTCTGTCTGCTTCTCTGGGCGATAGAGTGACCATCAGCTGCAGAGCTTCTCAGGACATCAGCAAGTATCTGAACTGGTATCAGCAGAAGCCTGATGGCACCGTGAAGCTGCTGATCTACCACACCTCCAGATTGCATTCTGGAGTTCCTTCCAGATTTTCTGGCTCTGGCTCTGGCACCGACTATTCTCTGACCATCAGCAATCTGGAACAGGAGGACATCGCTACCTACTTTTGCCAGCAGGGCAACACACTGCCTTACACATTTGGAGGAGGAACAAAGCTGGAGATCACAGGAGGAGGATCTGGAGGAGGATCTGGAGGAGGATCTGAAGTTAAACTGCAGGAATCTGGACCAGGATTAGTGGCCCCATCTCAGTCTCTGTCTGTGACCTGTACCGTTTCTGGAGTTTCTTTGCCTGATTACGGAGTGTCCTGGATCAGACAGCCCCCTAGAAAGGGACTGGAATGGTTAGGAGTGATTTGGGGATCTGAGACCACCTACTACAACTCTGCCCTGAAGAGCAGACTGACCATCATCAAGGACAACAGCAAGTCTCAGGTGTTCCTGAAGATGAACTCCCTGCAGACCGACGATACCGCCATCTACTACTGTGCTAAGCACTACTACTATGGCGGCTCTTATGCCATGGACTATTGGGGACAGGGCACCTCTGTGACAGTGTCTTCTTAA

SEQ ID NO: 87 - protein, 747 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGSGGSGSGGSGGSGAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 88 - protein, 752 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGSGGSGSGGSGGSGAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 89 - protein, 738 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 90 - protein, 742 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 91 - protein, 747 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 92 - protein, 742 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 93 - protein, 746 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 94 - protein, 751 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 95 - protein, 746 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 96 - protein, 750 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 97 - protein, 755 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGSGGGSGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 98 - protein, 739 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 99 - protein, 739 aa, Homo sapiens; identical to SEQ ID NO: 98

SEQ ID NO: 100 - protein, 748 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 101 - protein, 749 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVASKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 102 - protein, 753 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 103 - protein, 758 aa, Homo sapiens
CCGCCACCMEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVGGGGSGGGGSGGGGSGGGGSAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGGSCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGSGGGSGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 104 - protein, 9 aa, Artificial Sequence
  Synthetic Sequence
GGGGSGGGS

SEQ ID NO: 105 - protein, 15 aa, Artificial Sequence
  Synthetic Sequence
GGGGSGGGGSGGGGS

SEQ ID NO: 106 - protein, 13 aa, Artificial Sequence
  Synthetic Sequence
GGGGSGGGSGGGS

SEQ ID NO: 107 - protein, 16 aa, Artificial Sequence
  Synthetic Sequence
GGGSGGGSGGGSGGGS

SEQ ID NO: 108 - protein, 17 aa, Artificial Sequence
  Synthetic Sequence
GGGGSGGGSGGGSGGGS

SEQ ID NO: 109 - protein, 10 aa, Artificial Sequence
  Synthetic Sequence
GGGGSGGGGS

SEQ ID NO: 110 - protein, 20 aa, Artificial Sequence
  Synthetic Sequence
GGGGSGGGGSGGGGSGGGGS

SEQ ID NO: 111 - protein, 243 aa, Artificial Sequence
  Synthetic Sequence
DIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIKSSGGGGSGGGGSGGGGSEVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 112 - protein, 239 aa, Artificial Sequence
  Synthetic Sequence
EVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 113 - protein, 729 aa, Artificial Sequence
  Synthetic Sequence
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSDIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIKSSGGGGSGGGGSGGGGSEVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 114 - protein, 736 aa, Artificial Sequence
  Synthetic Sequence
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSDIQMTQSPSFLSASVGDRVTITCKASQNVDTNVAWYQQKPGKAPKALIYSASYRYSGVPSRFSGSGSGTDFTLTISSLQPEDFAEYFCQQYNNYPFTFGQGTKLEIKSSGGGGSGGGGSGGGGSEVQLVESGGGLVQPGGSLRLSCAASGFTFSSFGMHWVRQAPGKGLEWVAYISSGSGTIYYADTVKGRFTISRDNAKNSLYLQMNSLRAEDTAVYYCARHGYRYEGFDYWGQGTTVTVSS

SEQ ID NO: 115 - protein, 725 aa, Artificial Sequence
  Synthetic Sequence
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 116 - protein, 732 aa, Artificial Sequence
  Synthetic Sequence
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSEVQLLESGGGLVQPGGSLRLSCAASGFTFSSHAMSWVRQAPGKGLEWVSAIWASGEQYYADSVKGRFTISRDNSKNTLYLQMNSLRAEDTAVYYCAKGWLGNFDYWGQGTLVTVSSGGGGSGGGGSGGGGSEIVLTQSPGTLSLSPGERATLSCRASQSVSRSYLAWYQQKPGQAPRLLIIGASTRATGIPDRFSGSGSGTDFTLTISRLEPEDFAVYYCQQGQVIPPTFGQGTKVEIK

SEQ ID NO: 117 - protein, 733 aa, Artificial Sequence
  Synthetic Sequence
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSQVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSGGGGSGGGGSGGGGSGGGGSQIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 118 - protein, 740 aa, Artificial Sequence
  Synthetic Sequence
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSQVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSSGGGGSGGGGSGGGGSGGGGSQIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 119 - protein, 726 aa, Artificial Sequence
  Synthetic Sequence
MEFGLSWVFLVAIIKGVQCQFIVVGPTDPILATVGENTTLRCHLSPEKNAEDMEVRWFRSQFSPAVFVYKGGRERTEEQMEEYRGRTTFVSKDISRGSVALVIHNITAQENGTYRCYFQEGRSYDEAILHLVVAGLGSKPLISMRGHEDGGIRLECISRGWYPKPLTVWRDPYGGVAPALKEVSMPDADGLFMVTTAVIIRDKSVRNMSCSINNTLLGQKKESVIFIPESFMPSVSPCAGSGSRKGGKRGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKDEGGEDGSQVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK

SEQ ID NO: 120 - protein, 733 aa, Artificial Sequence
  Synthetic Sequence
MEFGLSWVFLVAIIKGVQCQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGGSGSDEGGEDGSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKRKGGKRGSGSQVQLVQSGGDLAQPGRSLRLSCAASGFTFDDYAMHWVRQAPGKGLEWVAVIWPDGGQKYYGDSVKGRFTVSRDNPKNTLYLQMNSLRAEDTAIYYCVRHFNAWDYWGQGTLVTVSSASGGGGSGGGGSGGGGSDIQLTQSPSSLSAYVGGRVTITCQASQGISQFLNWFQQKPGKAPKLLISDASNLEPGVPSRFSGSGSGTDFTFTITNLQPEDIATYYCQQYDDLPLTFGGGTKVEIK